(19) United States
(12) Patent Application Publication
(10) Pub. No.: US 2026/0242881 A1
(43) Pub. Date: Aug. 20, 2026

(54) METHYLATION SIGNATURES OF PATHOGEN AND CHEMICAL EXPOSURES IN HUMAN CELLS

(71) Applicant: SALK INSTITUTE FOR BIOLOGICAL STUDIES, La Jolla, CA (US)

(72) Inventors: Wenliang Wang, La Jolla, CA (US); Manoj Hariharan, La Jolla, CA (US); Joseph R. Ecker, La Jolla, CA (US)

(73) Assignee: SALK INSTITUTE FOR BIOLOGICAL STUDIES, (US)

(21) Appl. No.: 19/164,167

(22) PCT Filed: Mar. 11, 2024
(86) PCT No.: PCT/US2024/019451

§ 371 Date: Sep. 10, 2025

Related U.S. Application Data
(60) Provisional application No. 63/489,546, filed on Mar. 10, 2023.

(51) Int. Cl. C12Q 1/70; A61K 31/403; A61K 31/43; A61K 31/47; A61K 31/65; A61K 31/7068; A61K 45/06; C12Q 1/48; C12Q 1/6806; C12Q 1/689

(52) U.S. Cl. CPC C12Q 1/703; A61K 31/403; A61K 31/43; A61K 31/47; A61K 31/65; A61K 31/7068; A61K 45/06; C12Q 1/48; C12Q 1/6806; C12Q 1/689; C12Y 207/07; C12Q 2600/154

(57) ABSTRACT

Methods are provided for determining whether a subject has been exposed to or infected with Bacillus anthracis, SARS-CoV-2, influenza A, human immunodeficiency virus type 1 (HIV-1), methicillin-susceptible Staphylococcus aureus (MSSA), or methicillin-resistant Staphylococcus aureus (MRSA), or has been exposed to an organophosphate (OP). Such methods include determining the measuring methylation status of numerous differentially methylated regions (DMRs) in the genomic DNA of particular PBMCs, such as those provided in Table 1, and in some examples also determining an amount of accessible chromatin in the isolated immune cells. Also provided are nucleic acid probes, arrays, solid supports (such a chip or nanosphere) and kits that can be used with such methods.

### CROSS-REFERENCE TO RELATED APPLICATIONS

This application claims priority to U.S. Provisional Application No. 63/489,546 filed Mar. 10, 2023, herein incorporated by reference in its entirety.

### ACKNOWLEDGMENT OF GOVERNMENT SUPPORT

This invention was made with government support under W911NF-19-2-0185 awarded by Defense Advanced Research Projects Agency. The government has certain rights in the invention.

### FIELD

Methods are provided for determining if a cell has been exposed to a pathogen (e.g., bacteria, virus) or a chemical agent, for example by determining the methylation status of multiple methylation markers in genomic DNA. Also provided are arrays, solid supports (such as a chip or bead, such as a nanosphere) and kits that can be used for such methods.

### BACKGROUND

Infectious diseases are among the greatest threats to human and animal health worldwide and are among the top ten causes of death (Bloom and Cadarette 2019). The severe acute respiratory syndrome coronavirus 2 (SARS-CoV-2) is still a major public health issue and has caused millions of deaths, dramatically impacting worldwide economies (Sachs et al., 2022). Human Immunodeficiency Virus type 1 (HIV-1) is a lentivirus that causes acquired immunodeficiency syndrome (AIDS), a condition in which the immune system fails to defend the host, increasing susceptibility to opportunistic infectious diseases and malignancies (Powell et al., 2016; McLaren and Fellay, 2021). Since the first report in 1981, HIV has been a major public health issue for over four decades and has claimed more than 40 million lives, according to the Joint United Nations Programme on HIV/AIDS (UNAIDS) (Unaids and UNAIDS 2022). Influenza is an acute respiratory infection caused by the influenza viruses, which circulate in all parts of the world and infects 5%-15% of the population in the United States each year (Tokars, Olsen, and Reed 2018). Besides viral infections, bacterial infections are also a significant threat to public health. Methicillin-resistant Staphylococcus aureus (MRSA) is a common bacteria resistant to multiple drugs and antibiotics. It is reported by the United States Centers for Disease Control and Prevention (CDC) that approximately 5% of patients in U.S. hospitals develop nosocomial MRSA infection (Nandhini et al. 2022). In contrast, methicillin-susceptible Staphylococcus aureus (MSSA), is sensitive to methicillin and other antibiotics. Both MRSA and MSSA can cause life-threatening infections. Anthrax infection has been known in cattle and mankind for several centuries, caused mainly by the Bacillus anthracis bacteria which harbors both pXO1 and pXO2 virulence plasmids. While naturally occurring human infections are less common compared to outbreaks in animals, this pathogen is notorious for its potential as a bioweapon (Swartz 2001). The toxin can inhibit the immune responses and lead to severe respiratory illness, pneumonia, and even death (Bower et al. 2022). Chemical exposures also pose ongoing threats to human health, such as organophosphate pesticides, which are associated with neurodevelopmental and congenital disabilities (Hertz-Picciotto et al. 2018). Furthermore, exposure to organophosphate esters can reduce the immune response to childhood vaccines in children (Hammel et al. 2022).

Cytosine DNA methylation plays a crucial role in regulating immune cell lineage and the host's response to pathogens (Calle-Fabregat, Morante-Palacios, and Ballestar 2020; Roy et al. 2021). During the development of immune cells from hematopoietic stem cells (HSCs), both the global methylation level and lineage-specific methylation undergo changes that are influenced by lineage-specific transcription factors (Ji et al. 2010; Calle-Fabregat, Morante-Palacios, and Ballestar 2020; Roy et al. 2021). Although DNA methylation was previously considered relatively stable compared to other epigenetic modifications, recent studies have shown that it can be altered immediately after pathogen infection or exposure to environmental factors (Pacis et al., n.d.; Chang et al. 2021; Martin and Fry 2018).

### SUMMARY

To investigate the DNA methylation alterations during the immune response to pathogens and toxic chemicals in major innate and adaptive immune cell types, the inventors isolated seven immune cell types (B cells, monocytes, NK cells, CD8 memory T cells, CD8 naïve T cells, CD4 memory T cells, and CD4 naïve T cells) from PBMCs of patients and healthy controls, and performed single-nucleus methylation sequencing (snmC-seq2) (Luo et al. 2017, 2018). The cell types were characterized based on fluorescence-activated cell sorting (FACS) and genome-wide methylation profiles in the CG context of each cell. This analysis revealed two sub-clusters within B cells and NK cells. Differentially methylated regions (DMRs) within each of these nine cell types were identified. For the cohorts exposed to viruses, bacteria, and chemicals, snmC-seq2 was conducted on the seven cell types using 173 PBMC samples collected from 112 donors, resulting in 111,180 PBMC methylation profiles. The impact of each exposure on the methylome of the immune cell types was examined and a model using these DMRs was trained to predict different types of exposures based on the methylation status of several thousand loci. Additionally, single-nucleus ATAC-seq data was generated from patients with the same exposures and a high correlation between these two modalities as observed. Minimal overlap between the exposure-associated changes in methylation and chromatin accessibility was observed, therefor epigenomic remodeling associated with exposures was analyzed using both techniques.

The data herein provides informative resources and new knowledge on the impact of different exposures on DNA methylation in immune cells. This is the first large-scale single-cell DNA methylation data on major immune cells, which can be used to generate portable field-deployable devices to detect and contain infections at source and take appropriate action based on the temporal information. The gene regulatory effects of differential methylation at intergenic regions also provide new insights into both human immune cell development and pathogenesis studies. Furthermore, the unique DNA methylation signatures aid in understanding the epigenetic regulation of immune processes and provide a new approach for disease diagnosis of exposures to known and unknown pathogens.

Based on these observations, provided are methods for determining whether a subject (such as a human) has been exposed to or infected with Bacillus anthracis, SARS-CoV-2, influenza A, human immunodeficiency virus type 1 (HIV-1), methicillin-susceptible S. aureus (MSSA), or methicillin-resistant Staphylococcus aureus (MRSA), as well as methods for determining whether a subject has been exposed to an organophosphate (OP). Such methods can include determining methylation status of at least 20, at least 25, at least 30, at least 40, at least 50, at least 100, at least 500, at least 1000, at least 5000, or at least 5400 differentially methylated regions (DMRs) in genomic DNA of isolated immune cells obtained from the subject, wherein the immune cells and DMRs are provided in Table 1. For example, the method can include treating isolated immune cells (or their isolated genomic DNA) with bisulfite to convert unmethylated cytosines of CpG dinucleotides in the genomic DNA to uracil, amplifying the treated genomic DNA (which will convert any uracil nucleotides present to thymine), and comparing the amplified genomic DNA to a control reference sequence(s), such a sequence for the immune cell expected when no relevant exposure or infection has occurred. Non-limiting methods of determining the methylation level of the DMRs include sequencing, hybridizing polynucleotide probes complementary to DMRs (such as using probes attached to a solid support or bead arrays having probes attached to the beads), and qPCR. Nucleic acid probes and primers can be designed based on the DMRs provided in in Table 1. Thus, whether particular DMR is or is not methylated is determined.

In some aspects, the method further includes determining an amount of accessible chromatin in the isolated immune cells, for example using single-nucleus ATAC-sequencing. In some examples, genomic DNA from isolated immune cells is treated with transpose to generate fragments of the genomic DNA, and the fragments sequenced. The sequence of the fragments can be compared to a control, such as sequence fragments from a reference sequence(s), such a sequence for the immune cell expected when no relevant exposure or infection has occurred. Thus, the relative or absolute amount of accessible chromatin for the test immune cell is determined.

Based on the detected methylation status and in some aspects also the amount of accessible chromatin in the isolated immune cells, it is determined whether the subject has been exposed to or infected with Bacillus anthracis, SARS-CoV-2, HIV-1, influenza A, MSSA, or MRSA, or has been exposed to an OP. In some examples, the methods including treating the subject, by administering a therapeutically effective amount of an agent, wherein the agent used is based on whether the subject has been exposed to or infected with Bacillus anthracis, SARS-CoV-2, HIV-1, influenza A, MSSA, or MRSA, or has been exposed to an OP. For example, if the method indicates that the subject has been exposed to or is infected with MRSA, the subject can be administered a therapeutically effective amount of one or more of vancomycin, ceftaroline, teicoplanin, clindamycin, and linezolid.

In some aspects, the isolated immune cells to be tested are obtained from a blood sample, such as PBMCs. In some aspects, immune cells are isolated by sorting based on cell surface markers, for example by flow cytometry, into particular cell types, such as naive helper T cells (CD3+, CD4+, CCR7+, CD45RA+), memory helper T cells (CD3+, CD4+, CD45RA−), naive cytotoxic T cells (CD3+, CD8+, CCR7+, CD45RA+), memory cytotoxic T cells (CD3+, CD8+, CD45RA−), B cells (CD3−, CD19+), and monocytes (CD3−, CD19−, CD14+), NK cells (CD3−, CD19−, CD14−, CD16+, CD56+). In one example, the isolated immune cells analyzed include or are enriched for (i.e., is a purified cell population) one or more of the following immune cell types: memory B cells, naive B cells, monocytes, active natural killer cells, naive natural killer cells, memory cytotoxic T cells, naive cytotoxic T cells, memory T cells, and naïve T cells.

Also provided are kits that include nucleic acid probes and/or primers for detecting at least 20, at least 25, at least 30, at least 40, at least 50, at least 100, at least 500, at least 1000, at least 5000, or at least 5400, of the DMRs in Table 1. Based on the chromosomal locations provided in Table 1, probes can be designed (for example taking into consideration the GC composition, length, etc.). Such kits can include additional elements, such as bisulfite, transposase, one or more antibodies to sort immune cells, or combinations thereof.

Also provided are arrays or solid supports (such a chip or nanosphere) containing nucleic acid probes and/or primers for detecting least 20, at least 25, at least 30, at least 40, at least 50, at least 100, at least 500, at least 1000, at least 5000, or at least 5400 DMRs, wherein the nucleic acid probes are specific for least 20, at least 25, at least 30, at least 40, at least 50, at least 100, at least 500, at least 1000, at least 5000, or at least 5400 of the DMRs in Table 1.

The foregoing and other objects and features of the disclosure will become more apparent from the following detailed description, which proceeds with reference to the accompanying figures.

### BRIEF DESCRIPTION OF THE DRAWINGS

FIGS. 1A-1C. Single-nucleus methylation sequencing of PBMCs from individuals that have different exposures. A. Samples from cohorts with viral, bacterial, or chemical exposures. B. Experimental and analysis pipelines. PBMCs were collected from individuals with different exposures, sorted into seven immune cell types on 384-well plates, and subjected to single-nucleus methylation sequencing. Data analysis was performed using our pipeline. C. Single nucleus clustering of cells from all exposures, colored by cell surface markers (i), global mCG levels (ii), and different exposures (iii).

FIGS. 2A-2G. Quality control of single-cell analysis. A. Distribution of genome coverage with reads from each cell. The X-axis shows the coverage of the genome, and the Y-axis shows the number of cells with that coverage. B. Distribution of the mapping rate of reads from each cell. The X-axis shows the mapping rate of the reads, and the Y-axis shows the number of cells with that mapping rate. C. Distribution of the final mC reads from each cell. The X-axis shows the number of final mC reads, and the Y-axis shows the number of cells with that number of mC reads. D. Distribution of global mCG levels in each cell. The X-axis shows the global mCG level, and the Y-axis shows the number of cells with that mCG level. E. t-SNE embeddings of single-nucleus methylomes from control samples, including ‘HIV_pre,’ ‘Flu_pre,’ and ‘Ctrl’ groups. Colors indicate donors. F. t-SNE embeddings of single-nucleus methylomes from control samples, including ‘HIV_pre,’ ‘Flu_pre,’ and ‘Ctrl’ groups. Colors indicate the Leiden clusters generated based on the global mCG levels within each cell. G. Total number of DMRs/DMSs for each cell type (horizontal bars) as well as the number of DMRs/DMSs shared across the cell types (vertical bars). The vertical lines show the connection between the cell types that share the DMRs/DMSs

FIGS. 3A-3G. Cell type-specific methylation signatures of immune cells from healthy controls. A. t-distributed stochastic neighbor embedding (t-SNE) embeddings of single-nucleus methylomes from healthy controls, colored by cell surface markers. B. Same embedding of cells colored by global mCG levels. C. Global mCG level of different cell types defined by cell surface markers and single-nucleus methylome. D. Heatmap showing the mCG methylation level at cell type-specific DMRs. E. Bar plot showing the count of DMRs located in intergenic, intragenic, and promoter regions for each cell type. F. Scatter plot showing the enrichment of motifs at DMRs for each cell type. The size of the dot indicates the enrichment p-value, while the colors represent the cell types. G. Genome browser view of DNA methylation levels at T cell marker genes CD3D, CD3E, and CD3G, which are hypomethylated in the T cell lineage and hypermethylated in other cell types.

FIGS. 4A-4J. Quality control and methylation changes after HIV-1 infection. A. t-SNE embeddings of single-nucleus methylomes from the HIV-1 cohort, including ‘pre,’ ‘acute,’ and ‘chronic’ stages. Colors indicate the Leiden clusters generated based on the global mCG levels within each cell. B. t-SNE embeddings of single-nucleus methylomes from the HIV-1 cohort, including ‘pre,’ ‘acute,’ and ‘chronic’ stages. Color indicates the HIV-1 infection stages (‘pre,’ ‘acute,’ and ‘chronic’). C. t-SNE embeddings of single-nucleus methylomes from the HIV-1 cohort, including ‘pre,’ ‘acute,’ and ‘chronic’ stages. Color indicates the global mCG level of each cell. D. Proportions of naive B cells and memory B cells, as well as naive NK cells and active NK cells among the cells from the three stages (‘pre,’ ‘acute,’ and ‘chronic’). The enrichment of cells in the clusters was performed using the Chi-square test. E. Global mCG levels of cells in the nine cell types and three stages. F. Boxplot shows the CG densities at the exposure and cell type DMRs, at both the multi-CpG and single-CpG DMRs. Statistical test was done with Mann-Whitney-Wilcoxon test. G. Bar plots showing the count of hypo-DMRs and hyper-DMRs in each stage. H. Scatter plots showing the differentially methylated genes (DMGs) between each pair of stages, with the y-axis representing the p-value and the x-axis representing the difference in methylation level. I. Functional enrichment of the DMGs. J. Heatmap showing the normalized methylation level at the DMGs.

FIGS. 5A-5H. Impact of HIV-1 and influenza infection on methylomes of immune cells. A. A. t-SNE embeddings of single-nucleus methylomes from the HIV-1 cohort, representing ‘pre,’ ‘acute,’ and ‘chronic’ stages. Color indicates the cell surface markers of these cells. B. Heatmap showing the mCG level at DMRs between ‘pre,’ ‘acute,’ and ‘chronic’ samples. The color scale represents methylation levels. DMRs are grouped by hypomethylation and further grouped by cell types. C. Scatter plot showing enriched motifs of DMRs between the three conditions (‘pre,’ ‘acute,’ and ‘chronic’) in all cell types. The color represents cell types defined by cell surface markers, and the size of the dots indicates the enrichment p-value. D. Heatmap showing genome-wide correlations between pseudo-bulk signals from each cell type in single-nucleus methylation and matched single-nucleus ATAC-seq. Red indicates stronger correlation, while blue indicates weaker correlation. E. Genome browser view of pseudo-bulk single-nucleus ATAC and methylation signals from memory CD8 T cells at the intron of gene DGKH, showing signals from three stages of HIV-1 infection, with colors indicating different stages. F. t-SNE embeddings of single-nucleus methylomes from the influenza cohort, representing ‘pre’ and ‘post’ stages. Color indicates the cell surface markers of these cells. G. Heatmap showing the mCG level at DMRs between ‘pre’ and ‘post’ samples. The color scale represents methylation levels. DMRs are grouped by hypomethylation and further grouped by cell types. H. Scatter plot showing enriched motifs of DMRs between the two conditions (‘pre’ and ‘post’) in all cell types. The color represents cell types defined by cell surface markers, and the size of the dots indicates the enrichment p-value.

FIGS. 6A-6G. Single-cell analysis quality control and other methylation changes after influenza infection. t-SNE embeddings of single-nucleus methylomes from the influenza cohort, including ‘pre’ and ‘post’ stages. Colors indicate the Leiden clusters generated based on the global mCG levels within each cell. B. t-SNE embeddings of single-nucleus methylomes from the influenza cohort, including ‘pre’ and ‘post’ stages. Colors indicate the influenza infection stages (‘pre’ and ‘post’). C. t-SNE embeddings of single-nucleus methylomes from the influenza cohort, including ‘pre’ and ‘post’ stages. Colors indicate the global mCG level of each cell. D. Proportions of naive B cells and memory B cells, as well as naive NK cells and active NK cells among the cells from the two stages (‘pre’ and ‘post’). The enrichment of cells in the clusters was performed using the Chi-square test. E. Global mCG levels of cells in the nine cell types and two different stages. F. Bar plots showing the count of DMRs in each stage, either ‘hypo’ or ‘hyper’. G. Heatmap showing the genome-wide correlations between pseudo-bulk signals from each cell type in single-nucleus methylation and matched single-nucleus ATAC-seq. Red indicates a higher correlation, while blue indicates a lower correlation. 1, 2, 3 and 4 in the labels indicate different stages of influenza infection.

FIGS. 7A-7K. Single-cell analysis quality control and other methylation changes after SARS-Cov2 infection. A. A. t-SNE embeddings of single-nucleus methylomes from the COVID-19 cohort, together with the healthy controls from this study. Colors indicate the Leiden clusters generated based on the global mCG levels within each cell. B. t-SNE embeddings of single-nucleus methylomes from the COVID-19 cohort, together with the healthy controls from this study. Colors indicate the groups of the samples (severe, moderate, and control). C. t-SNE embeddings of single-nucleus methylomes from the COVID-19 cohort, together with the healthy controls from this study. Colors indicate the global mCG level of each cell. D. Proportions of naive NK cells and active NK cells among the NK cells from the three groups (moderate, severe, and controls). The enrichment of cells in the clusters was performed using the Chi-square test. E. Proportions of naive B cells and memory B cells among the B cells from the three groups (moderate, severe, and controls). The enrichment of cells in the clusters was performed using the Chi-square test. F. Heatmap showing the column-normalized (Z-score) methylation level at the differentially methylated genes (DMGs) between two clusters of monocytes. G. Heatmap showing the methylation level of all samples at the differentially methylated regions (DMRs) between two clusters of monocytes. H. Great analysis results on the DMRs between the two monocytes; the upper panel shows the results of DMRs hypomethylated in ‘Monocytel’, and the lower panel shows the results of DMRs hypomethylated in ‘Monocyte2’. I. Global mCG levels of cells in the nine cell types and three groups (moderate, severe, and controls). J. Bar plots showing the count of DMRs in each stage, either ‘hypo’ or ‘hyper’. K. Heatmap showing the DMRs identified between randomly grouped samples, which are less distinctive between groups compared to COVID-19-associated DMRs.

FIGS. 8A-8G. Impact of SARS-Cov2 infection on the methylomes of immune cells. A. t-SNE embeddings of single-nucleus methylomes from the COVID-19 cohort, together with the healthy controls from this study. Colors indicate cell surface markers. B. Bar plot showing the proportion of cells from two clusters of monocytes. A chi-square test was used to determine the enrichment of moderate or severe COVID-19 samples in these two clusters of monocytes. C. Functional enrichment of differentially methylated genes between the two clusters of monocytes. D. Heatmap showing the methylation level at DMRs between moderate, severe, and control samples. The color scale represents methylation levels. DMRs are grouped by hypomethylation and further grouped by cell types. E. Heatmap showing the correlation of samples from moderate, severe, and control groups at DMRs between the three groups. F. Scatter plot showing enriched motifs of DMRs between moderate, severe COVID-19 samples and controls in all cell types. The color represents cell types defined by cell surface markers, while the size of the dots represents the enrichment p-value. G. Heatmap showing genome-wide correlations between pseudo-bulk signals from each cell type in single-nucleus methylation and matched single-nucleus ATAC-seq. Red indicates stronger correlation, while blue indicates weaker correlation.

FIGS. 9A-9H. Impact of MRSA/MSSA and BA on the methylome of immune cells. A. t-SNE embeddings of single-nucleus methylomes from the MRSA/MSSA cohort, together with the healthy controls from this study. Color indicates the cell surface markers. B. Heatmap showing the mCG level at DMRs between MRSA, MSSA, and control samples. The color scale represents methylation levels. DMRs are grouped by hypomethylation and further grouped by cell types. C. Heatmap showing the correlation of samples from MRSA, MSSA, and control groups at DMRs between the three groups. D. Scatter plot showing enriched motifs of DMRs between MRSA, MSSA, and control samples in all cell types. The color represents cell types defined by cell surface markers, while the size of the dots represents the enrichment p-value. E. t-SNE embeddings of single-nucleus methylomes from the BA cohort, together with the healthy controls from this study. Color indicates the cell surface markers of these cells. F. Heatmap showing the mCG level at DMRs between frequent, infrequent, and control samples. The color scale represents methylation levels. G. Scatter plot showing enriched motifs of DMRs between frequent, infrequent, and control samples in all cell types. The color represents cell types defined by cell surface markers, while the size of the dots represents the enrichment p-value. H. Genome browser view of pseudo-bulk single-nucleus ATAC and methylation signals from T cells at gene MTCO1P12. One highlighted region shows an associated gain of accessibility and loss of DNA methylation, while the other region shows a gain of methylation and loss of accessibility.

FIGS. 10A-10G. Single-cell analysis quality control and other methylation changes after MRSA/MSSA infection. A. t-SNE embeddings of single-nucleus methylomes from the MRSA/MSSA cohort, together with the healthy controls from this study. The color shows the Leiden clusters. B. t-SNE embeddings of single-nucleus methylomes from the MRSA/MSSA cohort, together with the healthy controls from this study. The color shows the groups of the samples (MRSA, MSSA, and controls). C. t-SNE embeddings of single-nucleus methylomes from the MRSA/MSSA cohort, together with the healthy controls from this study. The color shows the global mCG level of each cell. D. Proportions of naive B cells and memory B cells, as well as naive NK cells and active NK cells among the cells from the three groups (MRSA, MSSA, and control). The enrichment of cells in the clusters was performed using the Chi-square test. E. Global mCG levels of cells in the nine cell types and three different groups (MRSA, MSSA, and control). F. Bar plots showing the count of DMRs in each stage, either ‘hypo’ or ‘hyper’. G. Heatmap showing the DMRs identified between randomly grouped samples, which are less distinctive between groups compared to MRSA or MSSA-associated DMRs.

FIGS. 11A-11I. Single-cell analysis quality control and other methylation changes work frequently or infrequently in high-risk facilities. A. t-SNE embeddings of single-nucleus methylomes from the BA cohort and the healthy controls from this study. The color shows the Leiden clusters. B. t-SNE embeddings of single-nucleus methylomes from the BA cohort, together with the healthy controls from this study. The color shows the groups of the samples (frequent, infrequent, and controls). C. t-SNE embeddings of single-nucleus methylomes from the BA cohort, together with the healthy controls from this study. The color shows the global mCG level of each cell. D. Proportions of naive B cells and memory B cells, as well as naive NK cells and active NK cells among the cells from the three groups (frequent, infrequent, and control). The enrichment of cells in the clusters was performed using the Chi-square test. E. Global mCG levels of cells in the nine cell types and three groups (frequent, infrequent, and control). F. Bar plots showing the count of DMRs in each stage, either ‘hypo’ or ‘hyper’. G. Heatmap showing the DMRs identified between randomly grouped samples, which are less distinctive between groups compared to the frequency of working in high-risk facilities associated DMRs. H. Heatmap showing the correlation of methylation levels of samples from different groups at the DMRs. I. Heatmap showing the genome-wide correlations between pseudo-bulk signals from each cell type in single-nucleus methylation and matched single-nucleus ATAC-seq. Red indicates a higher correlation, while blue indicates a lower correlation.

FIGS. 12A-12D. Impact of OP infection on the methylome of immune cells. A. t-SNE embeddings of single-nucleus methylomes from the OP cohort, together with the healthy controls from this study. Color indicates the cell surface markers of these cells. B. Heatmap showing the mCG level at DMRs between low, median, high OP exposures, and control samples. The color scale represents methylation levels. DMRs are grouped by hypomethylation and further grouped by cell types. C. Heatmap showing the correlation of samples from low, median, high OP exposures, and control groups at DMRs between the four groups. D. Scatter plot showing enriched motifs of DMRs between low, median, high OP exposure, and control samples in all cell types. The color represents cell types defined by cell surface markers, while the size of the dots represents the enrichment p-value.

FIGS. 13A-13H. Single-cell analysis quality control and methylation changes after OP infection. A. t-SNE embeddings of single-nucleus methylomes from the OP cohort, together with the healthy controls from this study. The color shows the Leiden clusters. B. t-SNE embeddings of single-nucleus methylomes from the OP cohort, together with the healthy controls from this study. The color shows the groups of the samples (Low, Medium, High, and controls). C. t-SNE embeddings of single-nucleus methylomes from the OP cohort, together with the healthy controls from this study. The color shows the global mCG level of each cell. D. Proportions of naive B cells and memory B cells, as well as naive NK cells and active NK cells among the cells from the four groups (Low, Medium, High, and controls). The enrichment of cells in the clusters was performed using the Chi-square test. E. Global mCG levels of cells in the nine cell types and four groups (Low, Medium, High, and controls). F. Bar plots showing the count of DMRs in each stage, either ‘hypo’ or ‘hyper’. G. Heatmap showing the DMRs identified between randomly grouped samples, which are less distinctive between groups compared to OP-associated DMRs. H. Heatmap showing the genome-wide correlations between pseudo-bulk signals from each cell type in single-nucleus methylation and matched single-nucleus ATAC-seq. Red indicates a higher correlation, while blue indicates a lower correlation.

FIGS. 14A-14B. Integrative analysis of DMRs from all exposures. A. Upset plot showing the hypo-DMRs shared between different exposures. B. Upset plot showing the shared hyper-DMRs between different exposures.

FIGS. 15A-15H. Hypo-methylated DMRs across exposures. A-H: Each plot shows the total number of hypomethylated DMRs for the respective cell type (horizontal bars), as well as the number of DMRs that are shared across the cell types (vertical bars). The vertical lines show the connection between the cell types that share the DMRs.

FIGS. 16A-16H. Hyper-methylated DMRs across exposures. A-H: Each plot shows the total number of hypermethylated DMRs for the respective cell type (horizontal bars), as well as the number of DMRs that are shared across the cell types (vertical bars). The vertical lines show the connection between the cell types that share the DMRs.

FIGS. 17A-17D. Prediction different exposures based on methylation signatures. A. ROC curve of HIV-1 stages prediction using 714 DMRs (10%) in Monocytes. B. ROC curve of HIV-1 stages prediction using 1428 DMRs (20%) in Monocytes. C. ROC curve of HIV-1 stages prediction using 2856 DMRs (40%) in Monocytes. D. The minimum number of DMRs required in each cell type to discriminate different exposures with AUC >0.95.

FIGS. 18A-18E. Comparison of CG context at the single-CpG and multi-CpG DMRs. A. Boxplot shows the CG densities at the Flu associated and cell type DMRs, at both the multi-CpG and single-CpG DMRs. Statistical test was done with Mann-Whitney-Wilcoxon test. B. Boxplot shows the CG densities at the COVID-19 associated and cell type DMRs, at both the multi-CpG and single-CpG DMRs. Statistical test was done with Mann-Whitney-Wilcoxon test. C. Boxplot shows the CG densities at the MRSA/MSSA associated and cell type DMRs, at both the multi-CpG and single-CpG DMRs. Statistical test was done with Mann-Whitney-Wilcoxon test. D. Boxplot shows the CG densities at the high-risk facility working frequency associated and cell type DMRs, at both the multi-CpG and single-CpG DMRs. Statistical test was done with Mann-Whitney-Wilcoxon test. E. Boxplot shows the CG densities at the OP associated and cell type DMRs, at both the multi-CpG and single-CpG DMRs. Statistical test was done with Mann-Whitney-Wilcoxon test.

FIG. 19. Motif enrichment results in all cell types and exposures. The x-axis shows the hypo-DMRs from different groups and cell types in all the exposures, and the y-axis shows the enriched motifs. The color indicates the cell types defined by cell surface markers, and the size of the dots demonstrates the enrichment p-value.

### DETAILED DESCRIPTION

### I. Terms

Unless otherwise noted, technical terms are used according to conventional usage. Definitions of many common terms in molecular biology may be found in Krebs et at. (eds.), Lewin's genes XII, published by Jones & Bartlett Learning, 2017. As used herein, the singular forms “a,” “an,” and “the,” refer to both the singular as well as plural, unless the context indicates otherwise. For example, the term “a cell” includes single or plural cells and can be considered equivalent to the phrase “at least one cell.” As used herein, the term “comprises” means “includes.” Unless otherwise indicated “about” indicates within five percent. It is further to be understood that any and all base sizes or amino acid sizes, and all molecular weight or molecular mass values, given for nucleic acids or polypeptides are approximate, and are provided for descriptive purposes, unless otherwise indicated. Although many methods and materials similar or equivalent to those described herein can be used, particular suitable methods and materials are described below. In case of conflict, the present specification, including explanations of terms, will control. In addition, the materials, methods, and examples are illustrative only and not intended to be limiting. To facilitate review of the various embodiments, the following explanations of terms are provided:

Administration: The introduction of a composition (such as one containing an agent that treats an infection or disorder) into a subject by a chosen route. Administration can be local or systemic. For example, if the route is intravenous, the composition is administered by introducing the composition into a vein of the subject. Similarly, if the route is intramuscular, the composition is administered by introducing the composition into a muscle of the subject. If the chosen route is oral, the composition is administered by ingesting the composition. Exemplary routes of administration of use in the methods disclosed herein include, but are not limited to, oral, injection (such as subcutaneous, intramuscular, intradermal, intraperitoneal, intraosseous, and intravenous), sublingual, rectal, transdermal (for example, topical), intranasal, vaginal, and inhalation routes. In one example, treatment is local, such as topical.

As used herein, the term “co-administer” (or “co-administration”) refers to administration of two or more agents within about 2 hours of each other, for example, as part of a clinical treatment regimen. In other embodiments, “co-administer” refers to administration of two or more agents within 1 hour of each other. In other embodiments, “co-administer” refers to administration of two or more agents within 30 minutes of each other. In other embodiments, “co-administer” refers to administration of two or more agents within 15 minutes of each other. In other embodiments, “co-administer” refers to administration of two or more agents at the same time, either as part of a single formulation or as multiple formulations that are administered by the same or different routes. A single “dose” refers to co-administration of agents at the same time.

Array/microarray: An intentionally created collection of molecules (such as peptides or nucleic acid molecules) in addressable locations on or in a substrate (such as plastic or glass). The molecules in the array can be identical or different from each other. A “microarray” is an array that is miniaturized so as to require or be aided by microscopic examination for evaluation or analysis.

The array of molecules (“features”) makes it possible to carry out a very large number of analyses on a sample at one time. In certain example arrays, one or more molecules (such as nucleic acid molecules) will occur on the array a plurality of times (such as twice), for instance to provide internal controls. The number of addressable locations on the array can vary, for example from at least one, to at least 2, to at least 3, at least 4, at least 5, at least 6, at least 10, at least 20, at least 30, at least 50, at least 75, at least 100, at least 150, at least 200, at least 300, at least 500, least 550, at least 600, at least 800, at least 1000, at least 10,000, or more. In some examples, arrays include positive and/or negative controls. In particular examples, an array includes nucleic acid molecules, such as oligonucleotide sequences that are at least 15 nucleotides (nt) in length, at least 20 nt, at least 30 nt, or at least 40 nt, at least 50 nt, such as about 50 nucleotides in length. In particular examples, an array includes probes or primers to detect methylation of genomic DNA at particular locations in the genome, such as at least 20, at least 25, at least 30, at least 40, at least 50, at least 100, at least 500, at least 1000, at least 5000, or at least 5400 of the locations listed in Table 1.

Bacillus anthracis: A gram-positive and rod-shaped bacterium that causes anthrax, a deadly disease to livestock and, occasionally, to humans. B. anthracis has a single circular chromosome and two circular, extrachromosomal, double-stranded DNA plasmids, pXO1 and pXO2. Untreated B. anthracis infection is usually deadly. Infection is indicated by inflammatory, black, necrotic lesions (eschars). The sores usually appear on the face, neck, arms, or hands. Fatal symptoms include a flu-like fever, chest discomfort, diaphoresis, and body aches. The infection can be treated with antibiotics such as penicillins, quinolones, and tetracyclines.

Complementarity: The ability of a nucleic acid to form hydrogen bond(s) with another nucleic acid sequence by either traditional Watson-Crick base pairing or other non-traditional types. Complementary nucleotides are, generally, A and T (or A and U), or C and G. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence. “Substantially complementary” as used herein refers to a degree of complementarity that is at least 60%, 65%, 70%, 75%, 80%, 85%, 90%, 95%, 97%, 98%, 99%, or 100% over a region of 8, 9, 10, 11, 12, 13, 14, 15, 16, 17, 18, 19, 20, 21, 22, 23, 24, 25, 30, 35, 40, 45, 50, or more nucleotides, or refers to two nucleic acids that hybridize under stringent conditions.

Coronavirus (CoV): A large family of positive-sense, single-stranded RNA viruses that can infect humans and non-human animals. Coronaviruses have been organized into four groups: alphacoronaviruses (α-CoVs), betacoronaviruses (β-CoVs), gammacoronaviruses (γ-CoVs), and deltacoronaviruses (Δ-CoVs). Non-limiting examples of betacoronaviruses include SARS-CoV-2, Middle East respiratory syndrome coronavirus (MERS-CoV), Severe Acute Respiratory Syndrome coronavirus (SARS-CoV), Human coronavirus HKU1 (HKU1-CoV), Human coronavirus OC43 (OC43-CoV), Murine Hepatitis Virus (MHV-CoV), Bat SARS-like coronavirus WIV1 (WIV1-CoV), and Human coronavirus HKU9 (HKU9-CoV). Non-limiting examples of alphacoronaviruses include human coronavirus 229E (229E-CoV), human coronavirus NL63 (NL63-CoV), porcine epidemic diarrhea virus (PEDV), and Transmissible gastroenteritis coronavirus (TGEV). A non-limiting example of a deltacoronavirus is the Swine Delta Coronavirus (SDCV).

Coronaviruses get their name from the crown-like spikes on their surface. The viral envelope is comprised of a lipid bilayer containing the viral membrane (M), envelope (E) and spike (S) proteins. Most coronaviruses cause mild to moderate upper respiratory tract illness, such as the common cold. However, three coronaviruses have emerged that can cause more serious illness and death: severe acute respiratory syndrome coronavirus (SARS-CoV), SARS-CoV-2, and Middle East respiratory syndrome coronavirus (MERS-CoV). Other coronaviruses that infect humans include human coronavirus HKU1 (HKU1-CoV), human coronavirus OC43 (OC43-CoV), human coronavirus 229E (229E-CoV), and human coronavirus NL63 (NL63-CoV).

In some examples, a SARS-CoV-2 genome is a variant of SARS-CoV-2, (such as: alpha (B.1.1.7 and Q lineages); beta (B.1.351 and descendent lineages); delta (B.1.617.2 and AY lineages); gamma (P.1 and descendent lineages); epsilon (B.1.427 and B.1.429); eta (B.1.525); iota (B.1.526); kappa (B.1.617.1); 1.617.3; mu (B.1.621, B.1.621.1); zeta (P.2); and omicron (such as original lineage: B.1.1.529 and lineages: BA.2, BA.4, BA.5, BQ.1, BQ.1.1, BA.4.6, and BF.7)).

COVID-19: A contagious disease caused by severe acute respiratory syndrome coronavirus 2 (SARS-CoV-2). Symptoms of COVID-19 are variable, but often include fever, cough, fatigue, breathing difficulties, and loss of smell and taste. Symptoms can begin one to fourteen days after exposure to the virus. Around one in five infected individuals do not develop any symptoms. While most people have mild symptoms, some people develop acute respiratory distress syndrome (ARDS). ARDS can be precipitated by cytokine storms, multi-organ failure, septic shock, and blood clots. Longer-term damage to organs (in particular, the lungs and heart) has been observed. A significant number of patients recover from the acute phase of the disease but continue to experience a range of effects—known as long COVID—for months afterwards. These effects include severe fatigue, memory loss and other cognitive issues, low-grade fever, muscle weakness, and breathlessness. In some examples, COVID-19 is treated with an anti-viral medication, such as paxlovid, molnupiravir, and/or remdesivir. Such antivirals can be administered orally or iv. In some examples, COVID-19 treatments include administration of one or more immune modulators, such as Olumiant (baricitinib) and Actemra (tocilizumab).

Epigenetic: Relating to, being, or involving a modification in gene expression that is independent of DNA sequence. Epigenetic factors include modifications in gene expression that are controlled by changes in DNA methylation and chromatin structure. For example, methylation patterns may correlate with gene expression.

Gene: The basic physical and functional unit of heredity, which is part of the genomic DNA. Includes particular gene sequences (such as protein coding sequence exons, intervening introns and associated expression control sequences) and its flanking sequence. Methylation in a particular region is generally indicative of the methylation status at proximal genomic sites. Accordingly, determining a methylation status of a gene region (such as those groups/categories provided in Table 1) can include determining a methylation status of a methylation marker shown therein, such as within or flanking about 10 bp to 50 bp, about 50 to 100 bp, about 100 bp to 200 bp, about 200 bp to 300 bp, about 300 to 400 bp, about 400 bp to 500 bp, about 500 bp to 600 bp, about 600 to 700 bp, about 700 bp to 800 bp, about 800 to 900 bp, 900 bp to 1 kb, about 1 kb to 2 kb, about 2 kb to 5 kb, or more of a named gene, or CpG position.

Genome/genomic: All of the genetic material in the chromosomes of an organism. DNA derived from the genetic material in the chromosomes of a particular organism is genomic DNA.

Human Immunodeficiency Virus Type 1 (HIV-1): A retrovirus that causes immunosuppression in humans (HIV-1 disease) and leads to a disease complex known as the acquired immunodeficiency syndrome (AIDS). “HIV-1 disease” refers to a well-recognized constellation of signs and symptoms (including the development of opportunistic infections) in persons who are infected by an HIV-1 virus, as determined by antibody or western blot studies. Laboratory findings associated with this disease include a progressive decline in T cells. Related viruses that are used as animal models include simian immunodeficiency virus (SIV), and feline immunodeficiency virus (FIV). Treatment of HIV-1 with HAART (highly active anti-retroviral drugs) has been effective in reducing the viral burden and ameliorating the effects of HIV-1 infection in infected individuals. Antiretroviral (ARV) drugs are broadly classified by the phase of the retrovirus life-cycle that the drug inhibits. Typical combinations include two nucleoside reverse-transcriptase inhibitors (NRTI) as a “backbone” along with one non-nucleoside reverse-transcriptase inhibitor (NNRTI), protease inhibitor (PI) or integrase inhibitors (also known as integrase nuclear strand transfer inhibitors or INSTIs) as a “base” (e.g., raltegravir, elvitegravir, dolutegravir, bictegravir, and cabotegravir).

Hybridization: To form non-covalent base pairs between complementary regions of two strands of DNA, RNA, or between DNA and RNA, thereby forming a duplex molecule. Triple-stranded hybridization is also possible. Hybridization conditions resulting in particular degrees of stringency will vary depending upon the nature of the hybridization method and the composition and length of the hybridizing nucleic acid molecules. Generally, the temperature of hybridization and the ionic strength (such as the Na⁺ concentration) of the hybridization buffer determine the stringency of hybridization. Calculations regarding hybridization conditions for attaining particular degrees of stringency are discussed in Sambrook et al., (1989) Molecular Cloning, second edition, Cold Spring Harbor Laboratory, Plainview, NY (chapters 9 and 11). The following is an exemplary set of hybridization conditions and is not limiting:

Very High Stringency (Allows Hybridization Between Sequences that Share at Least 90% Identity)
Hybridization: 5×SSC at 65° C. for 16 hours
Wash twice: 2×SSC at room temperature (RT) for 15 minutes each
Wash twice: 0.5×SSC at 65° C. for 20 minutes each
   High Stringency (Allows Hybridization Between Sequences that Share at Least 80% Identity)
Hybridization: 5×-6×SSC at 65° C.-70° C. for 16-20 hours
Wash twice: 2×SSC at RT for 5-20 minutes each
Wash twice: 1×SSC at 55° C.-70° C. for 30 minutes each
   Low Stringency (Allows Hybridization Between Sequences that Share at Least 50% Identity)
Hybridization: 6×SSC at RT to 55° C. for 16-20 hours
Wash at least twice: 2×-3×SSC at RT to 55° C. for 20-30 minutes each.

Influenza virus: A segmented negative-strand RNA virus that belongs to the Orthomyxoviridae family. There are three types of Influenza viruses, A, B and C. Influenza A viruses infect a wide variety of birds and mammals, including humans, horses, marine mammals, pigs, ferrets, and chickens. In some examples, influenza infection is treated with oseltamivir (Tamiflu®), zanamivir (Relenza®), or peramivir (Rapivab®).

Influenza A viruses are categorized into subtypes based on the type of two proteins, hemagglutinin (HA) and neuraminidase (NA) that are on the surface of the viral envelope. Different influenza viruses encode for different HA and NA proteins. There are 18 different HA subtypes and 11 different NA subtypes, H1 through H18 and N1 through N11 respectively.

In animals, most influenza A viruses cause mild localized infections of the respiratory and intestinal tract. However, highly pathogenic influenza A strains, such as H5N1, cause systemic infections in poultry in which mortality may reach 100%. Influenza A viruses that have caused disease in humans include the following subtypes: H1N1, H2N2, and H3N2. Other subtypes of viruses that have infected humans but are not transmitted from person to person are: H5N1 (bird flu), H6N1, H7N7, H1N2, H9N2, H7N2, H7N3, H10N7 and H10N8. There are different lineages of HA and NA within each subtype that are distinguished by amino acid sequence. For example, H1N1 viruses that circulated before 2009 and swine flu H1N1 viruses that have circulated amongst humans since 2009, represent different HA and NA lineages. Within each lineage, virus variants of different “clades” circulate in the population each winter.

Inhibiting or treating a disease: Inhibiting a disease, such as, but not limited to, an infection, refers to inhibiting the full development of a disease. In several examples, inhibiting a disease refers to lessening symptoms of the particular disease. “Treatment” refers to a therapeutic intervention that ameliorates a sign or symptom of a disease or pathological condition related to the disease. Treatment can be measured using success or indicia of success in the attenuation or amelioration of an injury, pathology or condition, including any objective or subjective parameter such as abatement, remission, diminishing of symptoms or making the condition more tolerable to the patient, slowing in the rate of degeneration or decline, making the final point of degeneration less debilitating, improving a subject's physical state. The treatment may be assessed by objective or subjective parameters; including the results of a physical examination or tests.

Isolated: An “isolated” biological component (such as a nucleic acid molecule or protein or organelle) has been substantially separated or purified away from other biological components in the cell of the organism in which the component naturally occurs, i.e., other chromosomal and extra-chromosomal DNA and RNA, proteins and organelles. Nucleic acids and proteins that have been “isolated” include nucleic acids and proteins purified by standard purification methods. The term also embraces nucleic acids and proteins prepared by recombinant expression in a host cell as well as chemically synthesized nucleic acids and proteins.

Label: A detectable compound or composition that is conjugated directly or indirectly to another molecule, such as a nucleic acid molecule, to facilitate detection of that molecule. Specific, non-limiting examples of labels include fluorophores, luminescent molecules, enzymatic linkages, and radioactive isotopes. In one example, a “labeled antibody” refers to incorporation of another molecule in the antibody. Various methods of labeling nucleic acid molecules are known and may be used. Exemplary labels that can be used, include, but are not limited to, the following: radioisotopes or radionucleotides (such as ³⁵S, ¹¹C, ¹³N, ¹⁵O, ¹⁸F, ¹⁹F, ^{99m}Tc, ¹³¹I, ³H, ¹⁴C, ¹⁵N, ⁹⁰Y ⁹⁹Tc, ¹¹¹In and ¹²⁵I), fluorescent labels (such as fluorescein isothiocyanate (FITC), rhodamine, lanthanide phosphors), enzymatic labels (such as horseradish peroxidase, beta-galactosidase, luciferase, alkaline phosphatase), chemiluminescent markers, biotinyl groups, or magnetic agents, such as gadolinium chelates.

Mammal: This term includes both human and non-human mammals. Similarly, the term “subject” includes both human and veterinary subjects, such as non-human primates, rats, mice, dogs, cats, horses, cows and pigs. In an example, a subject is a human. In an additional example, a subject is selected that has or is suspected of having been exposed to or infected with Bacillus anthracis, SARS-CoV-2, influenza A, human immunodeficiency virus type 1 (HIV-1), or methicillin-resistant Staphylococcus aureus (MRSA), or exposed to an organophosphate (OP).

Nucleic acid molecule: Any polymer or oligomer composed of nucleotide units (ribonucleotides, deoxyribonucleotides, peptide nucleic acids, related naturally occurring structural variants, and synthetic non-naturally occurring analogs thereof) linked via phosphodiester bonds, related naturally occurring structural variants, and synthetic non-naturally occurring analogs thereof. Includes chemical variants thereof, such as methylated, hydroxymethylated or glucosylated forms of these bases, and the like. The polymers or oligomers may be heterogeneous or homogeneous in composition. Exemplary nucleic acids include DNA and RNA, and mixtures thereof, and may exist permanently or transitionally in single-stranded or double-stranded form, including homoduplex, heteroduplex, and hybrid states.

Includes nucleotide polymers in which the nucleotides and the linkages between them include non-naturally occurring synthetic analogs, such as, for example and without limitation, phosphorothioates, phosphoramidates, methyl phosphonates, chiral-methyl phosphonates, 2-O-methyl ribonucleotides, peptide-nucleic acids (PNAs), and the like. It will be understood that when a nucleotide sequence is represented by a DNA sequence (i.e., A, T, G, C), this also includes an RNA sequence (i.e., A, U, G, C) in which “U” replaces “T.”

Conventional notation is used herein to describe nucleotide sequences: the left-hand end of a single-stranded nucleotide sequence is the 5′-end; the left-hand direction of a double-stranded nucleotide sequence is referred to as the 5-direction. The direction of 5′ to 3′ addition of nucleotides to nascent RNA transcripts is referred to as the transcription direction. The DNA strand having the same sequence as an mRNA is referred to as the “coding strand;” sequences on the DNA strand having the same sequence as an mRNA transcribed from that DNA and which are located 5′ to the 5′-end of the RNA transcript are referred to as “upstream sequences;” sequences on the DNA strand having the same sequence as the RNA and which are 3′ to the 3′ end of the coding RNA transcript are referred to as “downstream sequences.” Methylation marker: A CpG position, for example in a genome, that is potentially methylated.

Methylation typically occurs in a CpG containing nucleic acid. The CpG containing nucleic acid may be present in, e.g., in a CpG island, a CpG doublet, a promoter, an intron, or an exon of gene. In one example, in the genetic regions provided herein the potential methylation sites encompass the promoter/enhancer regions of the indicated genes. Thus, the regions can begin upstream of a gene promoter and extend downstream into the transcribed region. Exemplary regions of the human genome where methylation markers for determining whether a subject has been exposed to or infected with Bacillus anthracis, SARS-CoV-2, human immunodeficiency virus type 1 (HIV-1), influenza A, methicillin-resistant Staphylococcus aureus (MSSA), or methicillin-resistant Staphylococcus aureus (MRSA), or has been exposed to an organophosphate (OP) are provided in Table 1.

Oligonucleotide/polynucleotide: A plurality of joined nucleotides joined by native phosphodiester bonds, at least 6 nucleotides in length, such as at least 8, or at least 20 nucleotides in length. Include sequences of deoxyribonucleic acid (DNA) or ribonucleic acid (RNA) which may be isolated from natural sources, recombinantly produced or artificially synthesized and mimetics thereof. An oligonucleotide analog refers to moieties that function similarly to oligonucleotides but have non-naturally occurring portions. For example, oligonucleotide analogs can contain non-naturally occurring portions, such as altered sugar moieties or inter-sugar linkages, such as a phosphorothioate oligodeoxynucleotide.

Particular oligonucleotides and oligonucleotide analogs can include linear sequences up to about 200 nucleotides in length, for example a sequence (such as DNA or RNA) that is at least 6 nucleotides, for example at least 8, at least 10, at least 15, at least 20, at least 21, at least 25, at least 30, at least 35, at least 40, at least 45, at least 50, at least 100 or even at least 200 nucleotides long, or from about 6 to about 50 nucleotides, for example about 10-25 nucleotides, such as 12, 15 or 20 nucleotides.

Organophosphate (OP): A class of organophosphorus compounds with the general structure O=P(OR)₃, a central phosphate molecule with alkyl or aromatic substituents. They can be considered as esters of phosphoric acid. Examples include pesticides (e.g., parathion, malathion, methyl parathion, chlorpyrifos, diazinon, dichlorvos, phosmet, fenitrothion, tetrachlorvinphos, azamethiphos, azinphos-methyl, terbufos, ethion, trichlorfon), herbicides (e.g., tribufos [DEF], merphos), nerve agents (e.g., soman, sarin, tabun, VX), flame retardants, plasticizers, and performance additives to engine oil. Organophosphate poisoning is poisoning due to organophosphates. Symptoms include increased saliva and tear production, diarrhea, vomiting, small pupils, sweating, muscle tremors, and confusion. While onset of symptoms is often within minutes to hours, some symptoms can take weeks to appear. Symptoms can last for days to weeks. Treatments can include pretreatment with carbamates to protect AChE from inhibition by OP compounds and post-exposure treatments with anti-cholinergic drugs. In some examples atropine and/or oximes such as pralidoxime, and diazepam, are used. General measures such as oxygen and intravenous fluids can also be provided.

Primer: A single-stranded oligonucleotide capable of acting as a point of initiation for template-directed DNA synthesis under suitable conditions for example, buffer and temperature, in the presence of four different nucleoside triphosphates and an agent for polymerization, such as, for example, DNA or RNA polymerase or reverse transcriptase. The length of the primer can depend on, for example, the intended use of the primer, and generally ranges from 15 to 30 nucleotides. A primer need not reflect the exact sequence of the template but must be sufficiently complementary to hybridize with such template. The primer site is the area of the template to which a primer hybridizes. The primer pair is a set of primers including a 5′ upstream primer that hybridizes with the 5′ end of the sequence to be amplified and a 3′ downstream primer that hybridizes with the complement of the 3′ end of the sequence to be amplified.

Methods for preparing and using nucleic acid primers are described, for example, in Sambrook et al. (In Molecular Cloning: A Laboratory Manual, CSHL, New York, 1989), Ausubel et al. (ed.) (In Current Protocols in Molecular Biology, John Wiley & Sons, New York, 1998), and Innis et al. (PCR Protocols. A Guide to Methods and Applications, Academic Press, Inc., San Diego, CA, 1990). PCR primer pairs can be derived from a known sequence, for example, by using computer programs intended for that purpose.

Probe: Oligonucleotide capable of binding in a base-specific manner to a complementary strand of nucleic acid, and can include a detectable label. In some examples, a probe is a surface-immobilized molecule that can be recognized by a particular target.

SARS-CoV-2: Also known as 2019-nCoV or 2019 novel coronavirus, SARS-CoV-2 is a positive-sense, single stranded RNA virus of the genus betacoronavirus that has emerged as a highly fatal cause of severe acute respiratory infection, such as COVID-19. The viral genome is capped, polyadenylated, and covered with nucleocapsid proteins. The SARS-CoV-2 virion includes a viral envelope with large spike glycoproteins. The SARS-CoV-2 genome, like most coronaviruses, has a common genome organization with the replicase gene included in the 5′-two thirds of the genome, and structural genes included in the 3′-third of the genome. The SARS-CoV-2 genome encodes the canonical set of structural protein genes in the order 5′-spike (S)-envelope (E)-membrane (M) and nucleocapsid (NP)-3′. An exemplary native SARS-CoV-2 genome is provided in GenBank Accession No. MN985325.1. Symptoms of SARS-CoV-2 infection include fever and respiratory illness, such as dry cough and shortness of breath. Cases of severe infection can progress to severe pneumonia, multi-organ failure, and death. The time from exposure to onset of symptoms is approximately 2 to 14 days.

In one example, a SARS-CoV-2 is a naturally occurring variant thereof, such as alpha (B.1.1.7 and Q lineages); beta (B.1.351 and descendent lineages); delta (B.1.617.2 and AY lineages); gamma (P.1 and descendent lineages); epsilon (B.1.427 and B.1.429); eta (B.1.525); iota (B.1.526); kappa (B.1.617.1); 1.617.3; mu (B.1.621, B.1.621.1), zeta (P.2), and omicron (such as original lineage: B.1.1.529 and lineages: BA2, BA.4, BA.5, BQ.1, BQ.1.1, BA.4.6, BF.7, XBB, and XBB.1.5).

In some examples SARS-CoV-2 is treated with an anti-viral, such as Paxlovid.

Staphylococcus aureus: A Gram-positive spherically shaped bacterium, frequently found in the upper respiratory tract and on the skin. Although S. aureus usually acts as a commensal of the human microbiota, it can become an opportunistic pathogen, being a common cause of skin infections including abscesses, respiratory infections such as sinusitis, and food poisoning. Strains include those that are resistant to antimicrobials (methicillin-resistant Staphylococcus aureus, MRSA), particularly a multiple drug resistance to beta-lactam antibiotics. Beta-lactam (β-lactam) antibiotics are a broad-spectrum group that include some penams (penicillin derivatives such as methicillin and oxacillin) and cephems such as the cephalosporins. Strains unable to resist these antibiotics are classified as methicillin-susceptible/sensitive S. aureus, or MSSA.

For susceptible strains, S. aureus infection can be treated with penicillin. In some examples, an S. aureus infection is treated with a penicillinase-resistant β-lactam antibiotic (for example, oxacillin or flucloxacillin) or vancomycin. In one example, a skin infection is treated topically with a triple antibiotic ointment (Neomycin/polymyxin B/bacitracin). Treatment of MRSA infection is urgent and delays can be fatal. The location and history related to the infection can determine the treatment. The route of administration of an antibiotic varies. Antibiotics effective against MRSA can be given by IV, oral, or a combination of both, and depend on the specific circumstances and patient characteristics. The use of concurrent treatment with vancomycin or other beta-lactam agents may have a synergistic effect.

Sample (or biological sample): A biological specimen containing genomic DNA, RNA (e.g., mRNA), protein, or combinations thereof, which can be obtained from a subject, such as a human. Examples include, but are not limited to, sputum, saliva, mucus, nasal wash, peripheral blood, tissue (such as brain tissue), cells, urine, tissue biopsy, fine needle aspirate, surgical specimen, feces, cerebral spinal fluid (CSF), bronchoalveolar lavage (BAL) fluid, nasopharyngeal samples, oropharyngeal samples, and autopsy material. In one example the sample is a peripheral blood sample, such as one including PBMCs. In one example, a sample is a purified population of cells, such as a population at least 80%, at least 90%, at least 95%, at least 99%, or at least 99.9% pure, such as purified/isolated population of memory B cells, naive B cells, monocytes, active natural killer cells, naive natural killer cells, memory cytotoxic T cells, naive cytotoxic T cells, memory T cells, or naïve T cells.

Selectively measuring: Methods wherein only a finite number of methylation markers or genes (comprising methylation markers) are measured rather than assaying essentially all potential methylation marker (or genes) in a genome. In some examples, “selectively measuring” methylation markers or genes comprising such markers can refer to measuring no more than 400,000, 300,000, 275,000, 253,000, 252,512, 200,000, 100,000, 50,000, 10,000, 5000, 2500, 1000, 500, 100, 50, 25, or 10 different methylation markers or genes comprising methylation markers (such as those listed in Table 1).

Solid support/support/substrate: A material or group of materials having a rigid or semi-rigid surface or surfaces. In one example, at least one surface of the solid support is substantially flat, although in some embodiments it may include physically separate synthesis regions for different compounds with, for example, wells, raised regions, pins, etched trenches, or the like. In some examples, the solid support is a bead, resin, gel, microsphere, plate or other geometric configurations. In some examples a solid support is composed of glass or plastic.

Therapeutically effective amount: A quantity of a composition or a cell to achieve a desired effect in a subject being treated. For instance, this can be the amount necessary to treat Bacillus anthracis or anthrax exposure or infection, SARS-CoV-2 exposure or infection, influenza A exposure or infection, HIV-1 exposure or infection, MRSA exposure or infection, MSSA exposure or infection, or organophosphate exposure. When administered to a subject, a dosage will generally be used that will achieve target tissue concentrations that has been shown to achieve an in vitro effect.

A therapeutically effective amount may vary depending upon one or more of: the subject and disease condition being treated, the weight and age of the subject, the severity of the brain disease, the manner of administration and the like, which can readily be determined. The beneficial therapeutic effect can include enablement of diagnostic determinations; amelioration of the symptoms associated with infection or exposure. In one embodiment, an “effective amount” is an amount sufficient to reduce symptoms associated with infection or exposure (e.g., to Bacillus anthracis or anthrax, SARS-CoV-2, HIV-1, influenza A, MSSA, MRSA, or organophosphate, for example by at least 10%, at least 20%, at least 50%, at least 70%, at least 90%, at least 95%, or even 100% (as compared to no administration of the therapeutic agent), or that delays onset or progression.

### II. Overview

Provided herein is an analysis of single-cell methylomes in major immune cell types from PBMCs obtained from both healthy donors and patients exposed to bacteria, viruses, and chemicals. The methylomes of 104,876 high-quality cells representing nine immune cell types were analyzed. The analysis identified 790,662 differentially methylated regions (DMRs) associated with these exposures, which are mostly individual CpG sites. Additionally, methylation and ATAC-seq data was integrated from same samples and strong correlations between the two modalities were observed. The epigenomic remodeling in these two modalities are complementary. A set of DMRs with the highest level of prediction is provided in Table 1.

These results provide the first comprehensive dataset of single immune cell methylation profiles, along with unique methylation biomarkers for various biological and chemical exposures. This dataset was used to examine the epigenomes and epigenetic features of immune cells in response to various exposures. This study represents the first human immune cell methylome atlas at single-cell resolution. Using this comprehensive resource, DMRs associated with each exposure were identified and unsupervised models developed to predict exposure based on these DMRs. These findings increase the understanding of the pathogenesis of various exposures and establish DMRs as signatures of different exposures, which can aid in diagnosis and treatments using methylation markers.

The diagnosis of different exposures based on selected CpG sites in immune cells has broad applications in public health and can be accomplished using the prediction model provided herein. In addition to providing biological insights into different exposures, the DMRs that exhibit high predictive weights for different exposures can be used as biomarkers for infectious disease diagnosis, offering a valuable clinical resource. Other investigations explored host DNA methylation in virus and bacterial infections, revealing associations between methylation markers and various infections (Qin, Scicluna, and van der Poll, 2021). For instance, a study discovered methylation signatures associated with persistent MRSA (Chang et al., 2021), while another study demonstrated that a bacterial infection can remodel the methylation profile of dendritic cells (Pacis et al., n.d.). The data herein provide signatures at single-cell resolution in a cell type-specific manner, extending the diagnostic capability to multiple exposures, including viruses, bacteria, and chemicals.

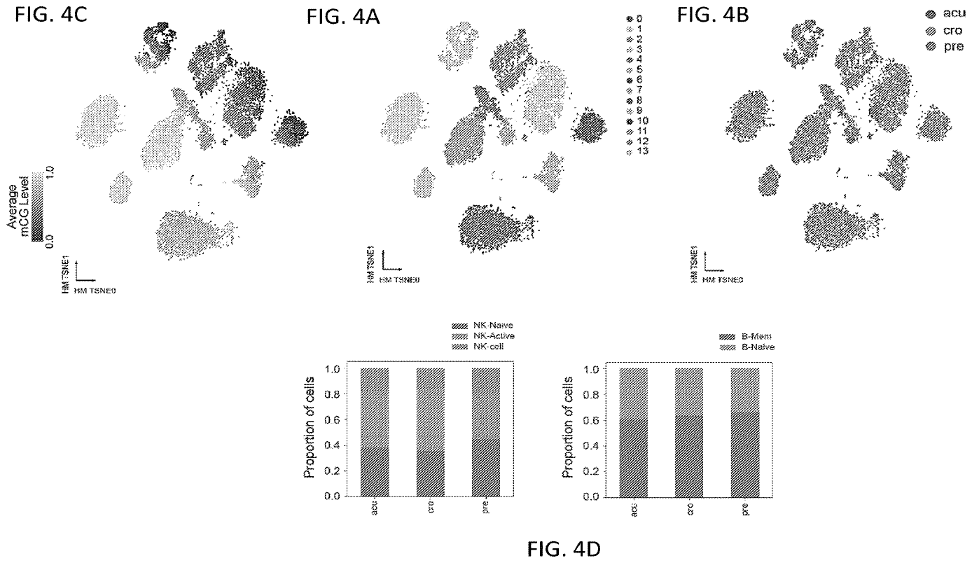

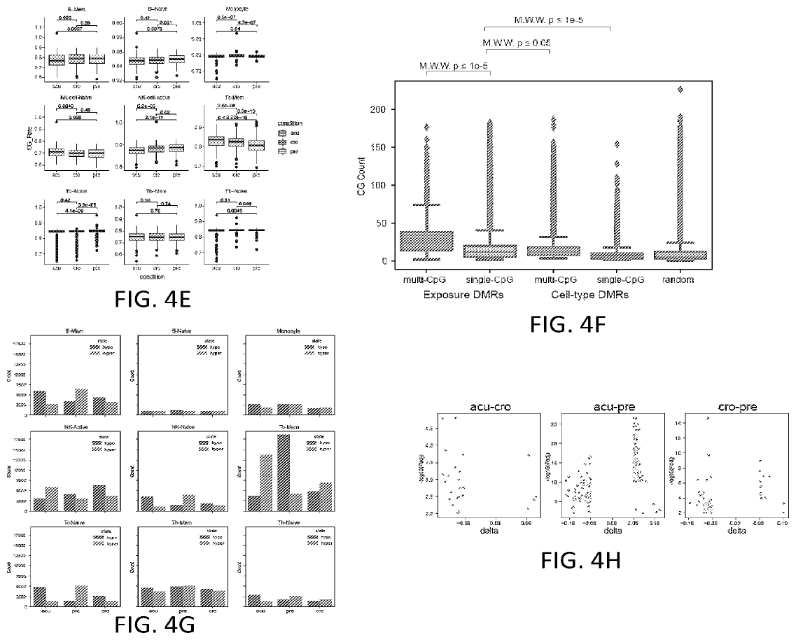

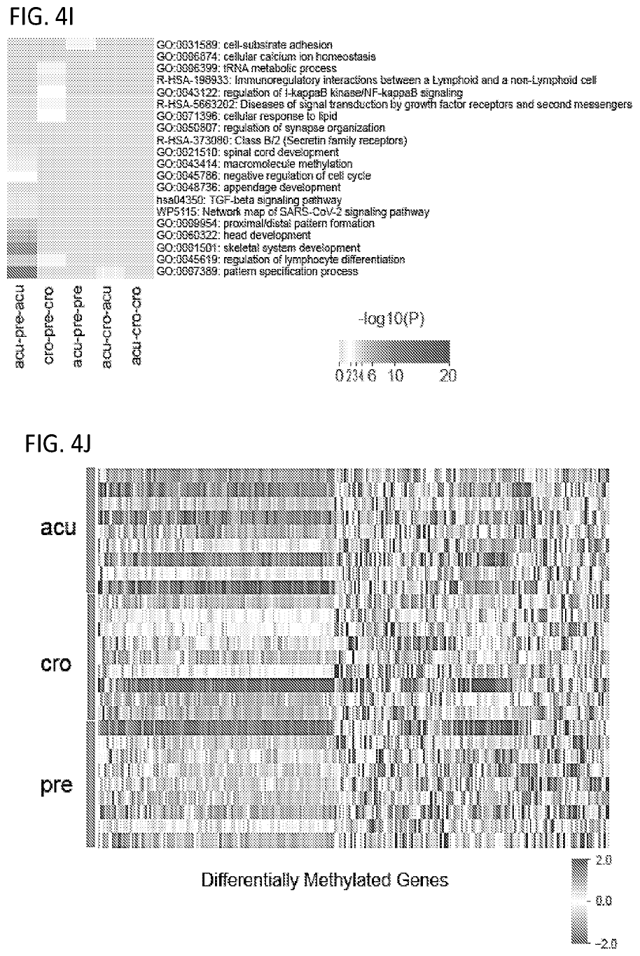

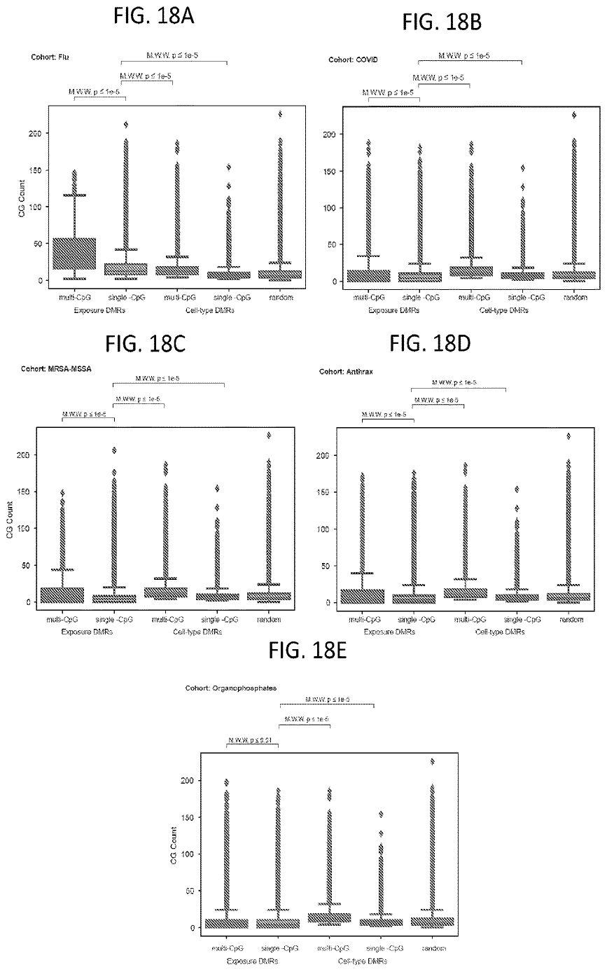

The majority of DMRs associated with exposures (~99% for most exposures) comprise single CpG sites primarily located in intergenic and inaccessible regions. This is in contrast to the 36% of single CpG DMRs observed between different cell types in control cohorts. Genomic analysis revealed that exposure-associated single CpG DMRs generally have fewer CGs compared to multi-CpG exposure DMRs. However, in some exposures, single CpG DMRs even exhibit a higher number of CGs than multi-CpG cell type DMRs (FIGS. 4F, 18). This indicates that the density of CGs alone cannot explain the substantial proportion of single CpG DMRs observed in each exposure. Consequently, it indicates the involvement of novel methylation or demethylation mechanisms in the infection-induced changes in single CpG DNA methylation. Single CpG methylation or demethylation occurs and has regulatory functions. Previous studies demonstrated that methylation of single CpG sites can regulate gene expression (Zhang et al., 2010; Wang et al., 2011), while demethylation of single CpG sites can trigger gene expression and regulate splicing (Ceccarelli et al., 2011; Venza et al., 2012; Sobiak and Lesniak, 2019).

One of the major signatures of the immune cells in these exposures is that the cell proportions of the two B cell and NK cell clusters vary in different exposures. These B cells and NK cells are sorted from PBMCs, and there can be some bias in the sorting process. However, the proportions of the two subtypes in B cells and NK cells identified with single-cell methylation profiles can reflect their relative abundance in PBMCs. The relative abundance of naive and memory B cells varies at different ages (Morbach et al. 2010), suggesting that the different age compositions of the donors from different exposures might contribute to the variabilities of B cell subtypes. However, there are significant changes in B cell subtypes during infections like SARS-CoV-2 (Sosa-Hernindez et al. 2020). Alterations of NK cell subtype frequencies were also reported in HIV and HCV infections (Meier et al. 2005), indicating that the relative abundance of the B cell and NK cell subtypes might be associated with different exposures.

A unique cluster of monocytes specific to COVID-19 samples confirms the association of these monocytes with COVID-19. Single-cell RNA-seq studies on COVID-19 samples have also found that classical monocytes are the main source of major COVID-19 mediating cytokines (Vanderbeke et al., 2021; N. Liu et al., 2021). The results identified the cytokines mediated by monocytes based on single-cell epigenomes. The epigenetic features associated with this unique cluster of monocytes provides insights into the mechanisms of the cytokine storm related to monocytes. Considerable changes in methylation were also observed in the common monocyte cluster in COVID-19 patients, indicating that the two clusters of monocytes are unique in SARS-CoV-2 infection.

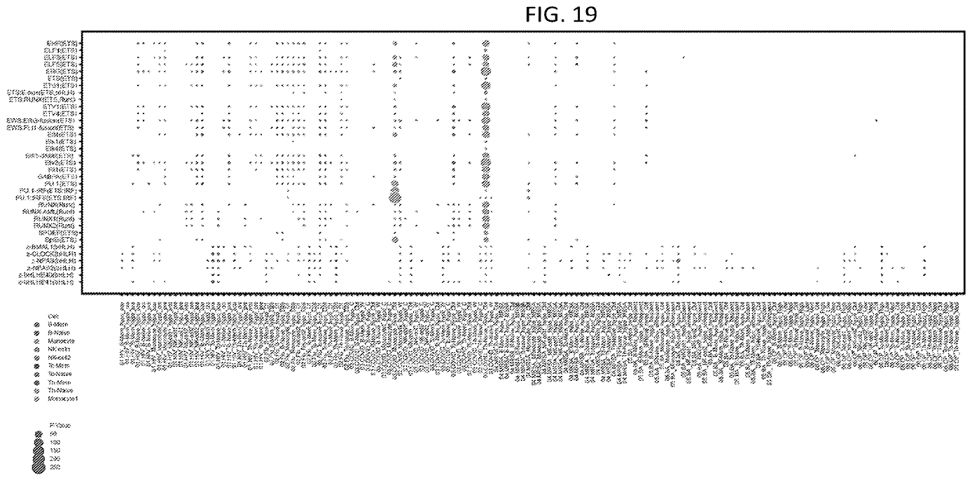

Specific TF binding motifs were enriched in the DMRs associated with each exposure, indicating exposure-specific changes in transcription factor activities across distinct cell types. TFs known to regulate cell differentiation and function, such as RUNX, ETS family transcription factors, and PU.1, were also enriched at certain DMRs. These TFs play key roles in the function and differentiation of immune cells (Sharrocks 2001; Korinfskaya et al. 2021; Hollenhorst, McIntosh, and Graves 2011; Hosokawa and Rothenberg 2020). ETS and RUNX motifs were generally enriched across most cell types in the virus exposures, but only enriched in monocytes and active NK cells in the MRSA/MSSA cohort (FIG. 19). Circadian transcription factor motifs were enriched in all cohorts, with higher enrichment in T cells for HIV-1, influenza, and OP cohorts, and in NK cells and monocytes for other exposures (FIG. 19). This indicates that different exposures may alter the activities of circadian transcription factors, which in turn can impact the functions of immune cells (Hergenhan et al. 2020; Wang et al. 2022).

Based on these observations, provided herein are methods of determining whether a subject has been exposed to or infected with Bacillus anthracis, SARS-CoV-2, influenza A, human immunodeficiency virus type 1 (HIV-1), methicillin-susceptible Staphylococcus aureus (MSSA), or methicillin-resistant Staphylococcus aureus (MRSA), or has been exposed to an organophosphate (OP). Such methods can include determining the methylation status of at least 20, at least 25, at least 30, at least 40, at least 50, at least 100, at least 500, at least 1000, at least 5000, or at least 5400 differentially methylated regions (DMRs) in genomic DNA of isolated immune cells obtained from the subject, wherein the immune cells and DMRs are provided in Table 1. Thus, the method can include determining for one or more particular immune cell types (such as 1, 2, 3, 4, 5, 6, 7, 8, or 9 of memory B cells, naive B cells, active natural killer cells, naive natural killer cells, memory cytotoxic T cells, naïve cytotoxic T cells, memory T cells, naïve T cell, and monocytes), whether at least 20, at least 25, at least 30, at least 40, at least 50, or all 100 DMRs listed in Table 1 for a particular immune cell and infection/exposure, are or are not methylated. Using Table 1, the DMRs selected are based on the infection/exposure of interest (Bacillus anthracis, SARS-CoV-2, influenza A, HIV-1, MSSA, MRSA, or OP) and the immune cell(s) to be analyzed. For example, to determine whether a subject has been infected with HIV-1, and memory B cells are to be analyzed, those DMRs listed for “HIV Memory B Cells” can be analyzed to determine if the DMR at the chromosomal position provided is or is not methylated. Any method can be used to determine methylation status at the DMRs provided. Non-limiting methods of determining the methylation level of the DMRs include sequencing, hybridizing polynucleotide probes complementary to DMRs (such as using probes attached to a solid support or bead arrays having probes attached to the beads), and qPCR. Nucleic acid probes and primers can be designed based on the DMRs provided in in Table 1. In one aspect, the method can include treating the isolated immune cells (or their isolated genomic DNA) with bisulfite to convert unmethylated cytosines in the genomic DNA to uracil, amplifying the treated genomic DNA (which will convert any uracil nucleotides present to thymine), and comparing the amplified genomic DNA to a control reference sequence(s), such a sequence for the same type of immune cell expected when no relevant exposure or infection has occurred.

In some aspects, the method also includes determining an amount of accessible chromatin in the isolated immune cells, for example using single-nucleus ATAC-sequencing. In some examples, genomic DNA from isolated immune cells is treated with transpose to generate fragments of the genomic DNA, and the fragments sequenced. The sequence of the fragments can be compared to a control, such as fragment sequences from a reference sequence(s), such a sequence for the immune cell expected when no relevant exposure or infection has occurred. Thus, the relative or absolute amount of accessible chromatin for the test immune cell can be determined.

In some aspects, determining methylation status and optionally an amount of accessible chromatin in the isolated immune cells is performed at the single cell level. In some examples, at least 10, at least 25, at least 50, at least 75, at least 100, at least 250, at least 500, at least 900, at least 1000, at least 5000, or at least 10,000 individual immune cells are analyzed per subject. In some aspects, no more than 10,000, no more than 5000, no more than 1000, no more than 500, or no more than 100 individual immune cells are analyzed per subject. In some aspects, 10-100,000, 10-10,000, 10-1000, 10-100, 50-500, 50-200, 50-100, or 100-500 individual immune cells per subject are analyzed.

Based on the measured/detected methylation status of the at least 20, at least 25, at least 30, at least 40, at least 50, at least 100, at least 500, at least 1000, at least 5000, or at least 5400 DMRs, and in some aspects also the amount of accessible chromatin, it is determined whether the subject has been exposed to or infected with Bacillus anthracis, SARS-CoV-2, influenza A, HIV-1, MSSA, or MRSA, or has been exposed to an organophosphate (OP). Such methods can further include a treatment/administration step depending on the determination made.

In some examples, determining whether a subject has been exposed to or infected with Bacillus anthracis, SARS-CoV-2, HIV-1, influenza A, MSSA, or MRSA, or has been exposed to an OP, includes applying a statistical prediction algorithm to the determined methylation status of the at least 20, at least 25, at least 30, at least 40, at least 50, at least 100, at least 500, at least 1000, at least 5000, or at least 5400 DMRs in Table 1. In some examples, applying the statistical prediction algorithm includes (a) obtaining a linear combination of the methylation marker status of the at least 20, at least 25, at least 30, at least 40, at least 50, at least 100, at least 500, at least 1000, at least 5000, or at least 5400 in Table 1, and (b) applying a transformation to the linear combination to determining whether a subject has been exposed to or infected with Bacillus anthracis, SARS-CoV-2, HIV-1, influenza A, MSSA, or MRSA, or has been exposed to an OP.

In some examples, the method determines whether the subject has been exposed to or is infected with Bacillus anthracis. The method can include determining the methylation status of at least 20, at least 25, at least 30, at least 40, at least 50, at least 100, at least 200, at least 300, at least 400, at least 500, at least 600, at least 700, at least 800, or all 900, DMRs listed in Table 1 for Bacillus anthracis. For example, the method can include determining methylation status of at least 20, at least 25, at least 30, at least 40, at least 50, or at least 100 of the DMRs in B memory cells in Table 1 listed as BA B-Mem; determining methylation status of at least 20, at least 25, at least 30, at least 40, at least 50, or at least 100 of the DMRs in naïve B cells in Table 1 listed as BA B-Naive; determining methylation status of at least 20, at least 25, at least 30, at least 40, at least 50, or at least 100 of the DMRs in monocytes in Table 1 listed as BA monocyte; determining methylation status of at least at least 20, at least 25, at least 30, at least 40, at least 50, or at least 100 of the DMRs in active natural killer cells in Tablet listed as BA NK-cell1; determining methylation status of at least at least 20, at least 25, at least 30, at least 40, at least 50, or at least 100 of the DMRs in naive natural killer cells in Table 1 listed as BA NK-cell2; determining methylation status of at least at least 20, at least 25, at least 30, at least 40, at least 50, or at least 100 of the DMRs in memory cytotoxic T cells in Table 1 listed as BA Tc-mem; determining methylation status of at least at least 20, at least 25, at least 30, at least 40, at least 50, or at least 100 of the DMRs in naive cytotoxic T cells in Table 1 listed as BA Tc-naive; determining methylation status of at least at least 20, at least 25, at least 30, at least 40, at least 50, or at least 100 of the DMRs in memory T cells in Table 1 listed as BA Th-mem; and/or determining methylation status of at least at least 20, at least 25, at least 30, at least 40, at least 50, or at least 100 of the DMRs in naïve T cells in Table 1 listed as BA Th-naive, wherein detection of the DMRs in Table 1 (for BA exposure) indicates that the subject has been exposed to or is infected with Bacillus anthracis. The method can further include determining an amount of accessible chromatin in same type of isolated immune cell(s) used to determine DMR methylation status. Thus in some examples, a sample of isolated immune cells is divided wherein one fraction is analyzed for DMR methylation status, and another fraction used to determine an amount of accessible chromatin (e.g., using single nucleus ATAC sequencing). In some aspects, the method can further include administering to the subject a therapeutically effective amount of one or more of ciprofloxacin, doxycycline, quinolone, and penicillin, when it is indicated that the subject has been exposed to or is infected with Bacillus anthracis.

In some examples, the method determines whether the subject has been exposed to or is infected with SARS-CoV-2. The method can include determining the methylation status of at least 20, at least 25, at least 30, at least 40, at least 50, at least 100, at least 200, at least 300, at least 400, at least 500, at least 600, at least 700, at least 800, or all 900, DMRs listed in Table 1 for COVID. For example, the method can include determining methylation status of at least 20, at least 25, at least 30, at least 40, at least 50, or at least 100 of the DMRs in B memory cells in Table 1 listed as COVID B-Mem; determining methylation status of at least 20, at least 25, at least 30, at least 40, at least 50, or at least 100 of the DMRs in naïve B cells in Table 1 listed as COVID B-Naïve; determining methylation status of at least 20, at least 25, at least 30, at least 40, at least 50, or at least 100 of the DMRs in monocytes in Table 1 listed as COVID monocyte; determining methylation status of at least at least 20, at least 25, at least 30, at least 40, at least 50, or at least 100 of the DMRs in active natural killer cells in Table1 listed as COVID NK-cell1; determining methylation status of at least at least 20, at least 25, at least 30, at least 40, at least 50, or at least 100 of the DMRs in naive natural killer cells in Table 1 listed as COVID NK-cell2; determining methylation status of at least at least 20, at least 25, at least 30, at least 40, at least 50, or at least 100 of the DMRs in memory cytotoxic T cells in Table 1 listed as COVID Tc-mem; determining methylation status of at least at least 20, at least 25, at least 30, at least 40, at least 50, or at least 100 of the DMRs in naive cytotoxic T cells in Table 1 listed as COVID Tc-naive; determining methylation status of at least at least 20, at least 25, at least 30, at least 40, at least 50, or at least 100 of the DMRs in memory T cells in Table 1 listed as COVID Th-mem; and/or determining methylation status of at least at least 20, at least 25, at least 30, at least 40, at least 50, or at least 100 of the DMRs in naïve T cells in Table 1 listed as COVID Th-naive, wherein detection of the DMRs in Table 1 (for COVID exposure) indicates that the subject has been exposed to or is infected with SARS-CoV-2. The method can further include determining an amount of accessible chromatin in same type of isolated immune cell(s) used to determine DMR methylation status. Thus in some examples, a sample of isolated immune cells is divided wherein one fraction is analyzed for DMR methylation status, and another fraction used to determine an amount of accessible chromatin (e.g., using single nucleus ATAC sequencing). Such a method can further include administering to the subject a therapeutically effective amount of one or more of paxlovid, molnupiravir, remdesivir, baricitinib and tocilizumab, when it is indicated that the subject has been exposed to or is infected with SARS-CoV-2.

In some examples, the method determines whether the subject has been exposed to or is infected with influenza. The method can include determining the methylation status of at least 20, at least 25, at least 30, at least 40, at least 50, at least 100, at least 200, at least 300, at least 400, at least 500, at least 600, at least 700, at least 800, or all 900, DMRs listed in Table 1 for Flu. For example, the method can include determining methylation status of at least 20, at least 25, at least 30, at least 40, at least 50, or at least 100 of the DMRs in B memory cells in Table I listed as Flu B-Mem; measuring methylation status of at least 20, at least 25, at least 30, at least 40, at least 50, or at least 100 of the DMRs in naïve B cells in Table 1 listed as Flu B-Naive; determining methylation status of at least 20, at least 25, at least 30, at least 40, at least 50, or at least 100 of the DMRs in monocytes in Table 1 listed as Flu monocyte; determining methylation status of at least at least 20, at least 25, at least 30, at least 40, at least 50, or at least 100 of the DMRs in active natural killer cells in Tablet listed as Flu NK-cell1; determining methylation status of at least at least 20, at least 25, at least 30, at least 40, at least 50, or at least 100 of the DMRs in naive natural killer cells in Table 1 listed as Flu NK-cell2; determining methylation status of at least at least 20, at least 25, at least 30, at least 40, at least 50, or at least 100 of the DMRs in memory cytotoxic T cells in Table 1 listed as Flu Tc-mem; determining methylation status of at least at least 20, at least 25, at least 30, at least 40, at least 50, or at least 100 of the DMRs in naive cytotoxic T cells in Table 1 listed as Flu Tc-naive; determining methylation status of at least at least 20, at least 25, at least 30, at least 40, at least 50, or at least 100 of the DMRs in memory T cells in Table 1 listed as Flu Th-mem; and/or determining methylation status of at least at least 20, at least 25, at least 30, at least 40, at least 50, or at least 100 of the DMRs in naïve T cells in Table I listed as Flu Th-naïve, wherein detection of the DMRs in Table 1 (for FLU exposure) indicates that the subject has been exposed to or is infected with influenza. The method can further include determining an amount of accessible chromatin in same type of isolated immune cell(s) used to determine DMR methylation status. Thus in some examples, a sample of isolated immune cells is divided wherein one fraction is analyzed for DMR methylation status, and another fraction used to determine an amount of accessible chromatin (e.g., using single nucleus ATAC sequencing). Such a method can further include administering to the subject a therapeutically effective amount of one or more of oseltamivir, zanamivir, and peramivir, when it is indicated that the subject has been exposed to or is infected with influenza, such as influenza A (such as H3N2).

In some examples, the method determines whether the subject has been exposed to or is infected with HIV-1. The method can include determining the methylation status of at least 20, at least 25, at least 30, at least 40, at least 50, at least 100, at least 200, at least 300, at least 400, at least 500, at least 600, at least 700, at least 800, or all 900, DMRs listed in Table 1 for HIV. For example, the method can include determining methylation status of at least 20, at least 25, at least 30, at least 40, at least 50, or at least 100 of the DMRs in B memory cells in Table 1 listed as HIV B-Mem; measuring methylation status of at least 20, at least 25, at least 30, at least 40, at least 50, or at least 100 of the DMRs in naïve B cells in Table 1 listed as HIV B-Naive; determining methylation status of at least 20, at least 25, at least 30, at least 40, at least 50, or at least 100 of the DMRs in monocytes in Table 1 listed as HIV monocyte; determining methylation status of at least at least 20, at least 25, at least 30, at least 40, at least 50, or at least 100 of the DMRs in active natural killer cells in Table1 listed as HIV NK-cell1; determining methylation status of at least at least 20, at least 25, at least 30, at least 40, at least 50, or at least 100 of the DMRs in naïve natural killer cells in Table 1 listed as HIV NK-cell2; determining methylation status of at least at least 20, at least 25, at least 30, at least 40, at least 50, or at least 100 of the DMRs in memory cytotoxic T cells in Table 1 listed as HIV Tc-mem; determining methylation status of at least at least 20, at least 25, at least 30, at least 40, at least 50, or at least 100 of the DMRs in naive cytotoxic T cells in Table 1 listed as HIV Tc-naive; determining methylation status of at least at least 20, at least 25, at least 30, at least 40, at least 50, or at least 100 of the DMRs in memory T cells in Table 1 listed as HIV Th-mem; and/or determining methylation status of at least at least 20, at least 25, at least 30, at least 40, at least 50, or at least 100 of the DMRs in naïve T cells in Table 1 listed as HIV Th-naive, wherein detection of the DMRs in Table 1 (for exposure HIV) indicates that the subject has been exposed to or is infected with HIV-1. Such a method can further include administering to the subject a therapeutically effective amount of a highly active antiretroviral therapy (HAART), when it is indicated that the subject has been exposed to or is infected with HIV-1.

In some examples, the method determines whether the subject has been exposed to or is infected with MSSA or MRSA. The method can include determining the methylation status of at least 20, at least 25, at least 30, at least 40, at least 50, at least 100, at least 200, at least 300, at least 400, at least 500, at least 600, at least 700, at least 800, or all 900, DMRs listed in Table 1 for MRSA. For example, the method can include determining methylation status of at least 20, at least 25, at least 30, at least 40, at least 50, or at least 100 of the DMRs in B memory cells in Table 1 listed as MRSA B-Mem; determining methylation status of at least 20, at least 25, at least 30, at least 40, at least 50, or at least 100 of the DMRs in naïve B cells in Table 1 listed as MRSA B-Naive; determining methylation status of at least 20, at least 25, at least 30, at least 40, at least 50, or at least 100 of the DMRs in monocytes in Table 1 listed as MRSA monocyte; determining methylation status of at least at least 20, at least 25, at least 30, at least 40, at least 50, or at least 100 of the DMRs in active natural killer cells in Table1 listed as MRSA NK-cell1; determining methylation status of at least at least 20, at least 25, at least 30, at least 40, at least 50, or at least 100 of the DMRs in naïve natural killer cells in Table 1 listed as MRSA NK-cell2; determining methylation status of at least at least 20, at least 25, at least 30, at least 40, at least 50, or at least 100 of the DMRs in memory cytotoxic T cells in Table 1 listed as MRSA Tc-mem; determining methylation status of at least at least 20, at least 25, at least 30, at least 40, at least 50, or at least 100 of the DMRs in naïve cytotoxic T cells in Table 1 listed as MRSA Tc-naive; determining methylation status of at least at least 20, at least 25, at least 30, at least 40, at least 50, or at least 100 of the DMRs in memory T cells in Table 1 listed as MRSA Th-mem; and/or determining methylation status of at least at least 20, at least 25, at least 30, at least 40, at least 50, or at least 100 of the DMRs in naïve T cells in Table 1 listed as MRSA Th-naive, wherein detection of the DMRs in Table 1 (exposure MRSA) indicates that the subject has been exposed to or is infected with MSSA or MRSA. The method can further include determining an amount of accessible chromatin in same type of isolated immune cell(s) used to determine DMR methylation status. Thus in some examples, a sample of isolated immune cells is divided wherein one fraction is analyzed for DMR methylation status, and another fraction used to determine an amount of accessible chromatin (e.g., using single nucleus ATAC sequencing). Such a method can further include administering to the subject a therapeutically effective amount of one or more of vancomycin, ceftaroline, teicoplanin, clindamycin, and linezolid, when it is indicated that the subject has been exposed to or is infected with MRSA. Such a method can further include administering to the subject a therapeutically effective amount of one or more of a Beta-lactam (D-lactam) antibiotic (such as penicillin and its derivatives (such as methicillin and oxacillin)) and cephems such as the cephalosporins; flucloxacillin; and a triple antibiotic ointment, when it is indicated that the subject has been exposed to or is infected with MSSA.

In some examples, the method determines whether the subject has been exposed to one or more organophosphates (OPs). The method can include determining the methylation status of at least 20, at least 25, at least 30, at least 40, at least 50, at least 100, at least 200, at least 300, at least 400, at least 500, at least 600, at least 700, at least 800, or all 900, DMRs listed in Table 1 for MRSA. For example, the method can include determining methylation status of at least 20, at least 25, at least 30, at least 40, at least 50, or at least 100 of the DMRs in B memory cells in Table 1 listed as OP B-Mem; determining methylation status of at least 20, at least 25, at least 30, at least 40, at least 50, or at least 100 of the DMRs in naïve B cells in Table 1 listed as OP B-Naïve; determining methylation status of at least 20, at least 25, at least 30, at least 40, at least 50, or at least 100 of the DMRs in monocytes in Table 1 listed as OP monocyte; determining methylation status of at least at least 20, at least 25, at least 30, at least 40, at least 50, or at least 100 of the DMRs in active natural killer cells in Table1 listed as OP NK-cell1; determining methylation status of at least at least 20, at least 25, at least 30, at least 40, at least 50, or at least 100 of the DMRs in naïve natural killer cells in Table 1 listed as OP NK-cell2; determining methylation status of at least at least 20, at least 25, at least 30, at least 40, at least 50, or at least 100 of the DMRs in memory cytotoxic T cells in Table I listed as OP Tc-mem; determining methylation status of at least at least 20, at least 25, at least 30, at least 40, at least 50, or at least 100 of the DMRs in naive cytotoxic T cells in Table 1 listed as OP Tc-naive; determining methylation status of at least at least 20, at least 25, at least 30, at least 40, at least 50, or at least 100 of the DMRs in memory T cells in Table 1 listed as OP Th-mem; and/or determining methylation status of at least at least 20, at least 25, at least 30, at least 40, at least 50, or at least 100 of the DMRs in naïve T cells in Table 1 listed as OP Th-naive, wherein detection of the DMRs in Table 1 (exposure OP) indicates that the subject has been exposed one or more OPs. The method can further include determining an amount of accessible chromatin in same type of isolated immune cell(s) used to determine DMR methylation status. Thus in some examples, a sample of isolated immune cells is divided wherein one fraction is analyzed for DMR methylation status, and another fraction used to determine an amount of accessible chromatin (e.g., using single nucleus ATAC sequencing). Such a method can further include administering to the subject a therapeutically effective amount of one or more of carbamate, anti-cholinergic drugs (e.g., atropine, oximes (such as pralidoxime, and diazepam)); cholinesterases (ChEs), such as human serum BChE (HuBChE); and class II anti-arrhythimic agents, when it is indicated that the subject has been exposed to one or more OPs.

In some aspects, the isolated immune cells are obtained from a blood sample, such as one including PBMCs. In some aspects, the method further includes obtaining the sample from the subject, such as a blood sample. In some aspects, the method further includes extracting genomic DNA from the sample, for example from the isolated immune cells.

In some aspects, the sample analyzed includes or is enriched for (i.e., is a purified cell population, such as one at least 90%, at least 95, at least 99%, at least 99.9%, or at least 99.99% pure) memory B cells, naive B cells, monocytes, active natural killer cells, naive natural killer cells, memory cytotoxic T cells, naive cytotoxic T cells, memory T cells, or naïve T cells. In some examples, multiple of such cell types are measured in parallel.

In some aspects, the subject is a human.

In some aspects, the method further includes selecting the subject, wherein the subject has been or is suspected of having been exposed to or infected with Bacillus anthracis, SARS-CoV-2, HIV-1, influenza A, MSSA, or MRSA, or exposed to an OP.

In some examples, determining the methylation status of the genomic DNA includes hybridizing the genomic DNA to polynucleotides that can discriminate methylation status at the desired DMRs attached to a solid support. In some examples, the polynucleotides attached to a solid support include at least 20, at least 25, at least 30, at least 40, at least 50, at least 100, at least 200, at least 300, at least 400, at least 500, at least 600, at least 700, at least 800, at least 900, at least 1000, at least 5000, or at least 5400 nucleic acid probes, wherein the nucleic acid probes are specific for at least 20, at least 25, at least 30, at least 40, at least 50, at least 100, at least 200, at least 300, at least 400, at least 500, at least 600, at least 700, at least 800, at least 900, at least 1000, at least 5000, or at least 5400 of the DMRs in Table 1.

In some examples, the method further includes reporting whether the subject has been exposed to or infected with Bacillus anthracis, SARS-CoV-2, influenza A, HIV-1, MSSA, or MRSA, or has been exposed to an OP. In some examples, the reporting includes preparing a written or electronic report.

Also provided are kits that can be used with the disclosed methods. Such kits can include nucleic acid probes and/or primers for detecting at least 20, at least 25, at least 30, at least 40, at least 50, at least 100, at least 200, at least 300, at least 400, at least 500, at least 600, at least 700, at least 800, at least 900, at least 1000, at least 5000, or at least 5400 DMRs provided in Table 1, wherein the nucleic acid probes and/or primers are specific for (e.g., can distinguish between methylation or non-methylation at the DMR listed) for at least 20, at least 25, at least 30, at least 40, at least 50, at least 100, at least 200, at least 300, at least 400, at least 500, at least 600, at least 700, at least 800, at least 900, at least 1000, at least 5000, or at least 5400 of the DMRs in Table 1. Based on the chromosomal positions provided in Table 1, probes can be designed (for example taking into consideration the CC composition, length, etc.). In some examples, such a kit also includes sodium bisulfite, transposase, labeled antibodies for isolating immune cells, or combinations thereof. The reagents of the kit can be in separate containers. In some examples, the kit includes a mixture of primers and/or probes in a single container.

Over 400,000 unique DMRs were identified for the six exposures (B. anthracis, SARS-CoV-2, influenza A, HIV-1, influenza A, MSSA/MRSA, or organophosphate). This list was reduced to identify the DMRs that achieved at least 95% AUROC. Table I shows the top 100 DMRs identified for each exposure condition (B. anthracis=BA, SARS-CoV-2=COVID, influenza A=Flu, HIV-1=HIV, MRSA/MSSA=MRSA, organophosphate=O), and DMRs were identified in particular cell types (Memory B cells (B-Mem), naive B cells (B-Naïve), active natural killer cells (NK cell 1), naive natural killer cells (NK cell 2), memory cytotoxic T cells (Tc-Mem), naive cytotoxic T cells (T-Naïve), memory T cells (Th-Mem), naïve T cell (Th-Naïve), Monocytes). The location of each DMR is provided in Table 1 by its chromosomnal location in the human genome, and a sequence is provided. For each DMR, in tbe respective cell type, the genomic DNA is methylated (positive weight value) or is not methylated (negative weigbt value) when the particular cell is infected/exposed to the particular agent listed.

TABLE 1
List of DMRs

| | | | | |
|---|---|---|---|---|
| Chromosome | Start Position | End position | Cell ID | Weight |

HIV Memory B Cells

| | | | | |
|---|---|---|---|---|
| chr10 | 49259640 | 49259640 | B-Mem_1418 | −0.1099916994518482 |
| chr5 | 136131050 | 136131050 | B-Mem_10605 | −0.0983757660535765 |
| chr4 | 68029283 | 68029283 | B-Mem_9598 | −0.0962808319597364 |
| chr4 | 42240298 | 42240298 | B-Mem_9501 | −0.0902466288051953 |
| chr12 | 106174834 | 106174834 | B-Mem_3027 | −0.0821755074588564 |
| chr1 | 165205512 | 165205512 | B-Mem_762 | −0.0784291630090022 |
| chr5 | 4207485 | 4207485 | B-Mem_10087 | −0.0744704333564821 |
| chrX | 112470613 | 112470613 | B-Mem_14118 | −0.0706319558172924 |
| chr4 | 24369228 | 24369228 | B-Mem_9416 | −0.0693658744595545 |
| chr1 | 73283106 | 73283106 | B-Mem_481 | −0.0669886779808305 |
| chr3 | 73526089 | 73526089 | B-Mem_8757 | −0.0656426456352602 |
| chr20 | 56574908 | 56574908 | B-Mem_7817 | −0.0650369189181459 |
| chr6 | 67238825 | 67238825 | B-Mem_11163 | −0.0641560299572996 |
| chr8 | 55386122 | 55386122 | B-Mem_12693 | −0.063837013810175 |
| chr18 | 2134339 | 2134339 | B-Mem_5607 | −0.0638065018906196 |
| chr2 | 126184614 | 126184614 | B-Mem_6990 | −0.0615473030364852 |
| chr3 | 105993285 | 105993285 | B-Mem_8867 | −0.060131285787008 |
| chr7 | 7805035 | 7805035 | B-Mem_11693 | −0.0598201217909016 |
| chr5 | 121259211 | 121259211 | B-Mem_10536 | −0.0586480553747796 |
| chrX | 28043141 | 28043141 | B-Mem_13865 | −0.0584448972470934 |
| chr4 | 136128648 | 136128648 | B-Mem_9813 | −0.0574882350265458 |
| chr1 | 239753502 | 239753502 | B-Mem_1112 | −0.0570837297990923 |
| chr5 | 2438435 | 2438435 | B-Mem_10065 | −0.0562850276653177 |
| chr11 | 112002588 | 112002588 | B-Mem_2423 | −0.0561310197842982 |
| chr5 | 12795733 | 12795733 | B-Mem_10154 | −0.0559638487868307 |
| chr5 | 167732004 | 167732004 | B-Mem_10764 | 0.05555679848852 |
| chr2 | 160172425 | 160172425 | B-Mem_7116 | −0.0545044143365444 |
| chr1 | 30352596 | 30352596 | B-Mem_263 | −0.0536334715375332 |
| chr10 | 69918289 | 69918289 | B-Mem_1502 | −0.0532581239054869 |
| chr2 | 2833102 | 2833102 | B-Mem_6449 | −0.053076949551412 |
| chrX | 143767320 | 143767320 | B-Mem_14212 | −0.0526240081431423 |
| chr11 | 16357506 | 16357506 | B-Mem_1950 | −0.0519518236313743 |
| chr12 | 29592506 | 29592506 | B-Mem_2714 | −0.0519353644889644 |
| chr1 | 209128557 | 209128557 | B-Mem_951 | −0.0518031939851091 |
| chrX | 52435087 | 52435087 | B-Mem_13940 | −0.0509137972732381 |
| chr13 | 88516584 | 88516584 | B-Mem_3506 | −0.0501803572805506 |
| chrX | 97711085 | 97711085 | B-Mem_14062 | −0.0493078860695874 |
| chr1 | 1284561 | 1284561 | B-Mem_10 | −0.049097519764921 |
| chr3 | 160070792 | 160070792 | B-Mem_9070 | −0.0489961354703347 |
| chr19 | 33725231 | 33725231 | B-Mem_6242 | −0.0489249220183919 |
| chr6 | 23630970 | 23630970 | B-Mem_10994 | −0.0487069003004302 |
| chrX | 104290834 | 104290834 | B-Mem_14087 | −0.0482456740581471 |
| chr1 | 247767472 | 247767472 | B-Mem_1155 | −0.0481116097567108 |
| chr10 | 78729501 | 78729501 | B-Mem_1543 | −0.0477876698927405 |
| chr14 | 38079082 | 38079082 | B-Mem_3727 | −0.046889693592498 |
| chr9 | 26015394 | 26015394 | B-Mem_13232 | −0.0456816327748288 |
| chr3 | 20922927 | 20922927 | B-Mem_8515 | −0.0455112651909185 |
| chr11 | 113050684 | 113050684 | B-Mem_2431 | −0.0453015732400375 |
| chrX | 21560240 | 21560240 | B-Mem_13838 | −0.0447201766507704 |
| chr8 | 16944619 | 16944619 | B-Mem_12493 | −0.0446969447874434 |
| chr16 | 75125028 | 75125028 | B-Mem_4951 | −0.0445440008036302 |
| chr10 | 38836766 | 38836766 | B-Mem_1368 | −0.0440082835953286 |
| chr3 | 134773347 | 134773347 | B-Mem_8995 | −0.0438569331132984 |
| chr19 | 15219247 | 15219247 | B-Mem_6125 | −0.0435108328121681 |
| chr5 | 154395775 | 154395775 | B-Mem_10695 | −0.0432068442211692 |
| chrX | 105345101 | 105345101 | B-Mem_14091 | −0.0427938495850246 |
| chr6 | 1409617 | 1409617 | B-Mem_10882 | −0.0423844243703253 |
| chr6 | 140094176 | 140094176 | B-Mem_11435 | −0.0419536179859056 |
| chr5 | 90048298 | 90048298 | B-Mem_10422 | −0.0417868766039841 |
| chr8 | 57924477 | 57924477 | B-Mem_12707 | 0.0418627865035295 |
| chr17 | 48722170 | 48722170 | B-Mem_5365 | 0.0422139555712601 |
| chrY | 7716021 | 7716021 | B-Mem_14266 | 0.0422231062608574 |
| chr2 | 108452371 | 108452371 | B-Mem_6922 | 0.0437799190669269 |
| chr1 | 108435907 | 108435907 | B-Mem_605 | 0.0438326659124042 |
| chr2 | 230738034 | 230738034 | B-Mem_7396 | 0.0441507579797801 |
| chr18 | 77987846 | 77987846 | B-Mem_5948 | 0.044225079033451 |
| chr2 | 240873806 | 240873806 | B-Mem_7477 | 0.0450179122786119 |
| chr9 | 116453376 | 116453376 | B-Mem_13570 | 0.0459120222691285 |
| chr9 | 11726232 | 11726232 | B-Mem_13158 | 0.0464012505224703 |
| chr8 | 57143280 | 57143280 | B-Mem_12706 | 0.0493109666079726 |
| chr8 | 110394691 | 110394691 | B-Mem_12910 | 0.0493876626478177 |
| chr1 | 8902039 | 8902039 | B-Mem_84 | 0.0494611166954529 |
| chr18 | 71577357 | 71577357 | B-Mem_5912 | 0.0496456130164937 |
| chr17 | 70523671 | 70523671 | B-Mem_5470 | 0.0497299719556413 |
| chr16 | 51355433 | 51355433 | B-Mem_4825 | 0.0508159939870781 |
| chr8 | 75449575 | 75449575 | B-Mem_12774 | 0.0526992190715112 |
| chrX | 121358764 | 121358764 | B-Mem_14145 | 0.0541565763549395 |
| chr1 | 231666338 | 231666338 | B-Mem_1061 | 0.0546600607479062 |
| chr6 | 841288 | 841288 | B-Mem_10873 | 0.0555107782181583 |
| chr8 | 6680069 | 6680069 | B-Mem_12451 | 0.0559031104512594 |
| chrX | 23271331 | 23271331 | B-Mem_13840 | 0.059396694802091 |
| chr4 | 69268679 | 69268679 | B-Mem_9603 | 0.0606694950274817 |
| chr1 | 224983295 | 224983295 | B-Mem_1017 | 0.0611469233251787 |
| chr16 | 74925657 | 74925657 | B-Mem_4950 | 0.0612339390711402 |
| chr1 | 181476943 | 181476943 | B-Mem_821 | 0.0621133712320676 |
| chr17 | 50872764 | 50872764 | B-Mem_5378 | 0.0636462981363888 |
| chr11 | 121487652 | 121487652 | B-Mem_2501 | 0.0648284959875424 |
| chr5 | 144598886 | 144598886 | B-Mem_10657 | 0.0683819817102511 |
| chrX | 51603517 | 51603517 | B-Mem_13938 | 0.0688736332996317 |
| chr9 | 9403270 | 9403270 | B-Mem_13145 | 0.0692606663802278 |
| chr2 | 97056419 | 97056419 | B-Mem_6860 | 0.0698083455677585 |
| chr6 | 169156120 | 169156120 | B-Mem_11584 | 0.0721476095814922 |
| chr19 | 55549982 | 55549982 | B-Mem_6405 | 0.0722550046156664 |
| chr1 | 20691027 | 20691027 | B-Mem_194 | 0.0723209543690516 |
| chr16 | 75260688 | 75260688 | B-Mem_4954 | 0.0845548333712443 |
| chrX | 8737640 | 8737640 | B-Mem_13789 | 0.0902874585557331 |
| chr8 | 90094253 | 90094253 | B-Mem_12827 | 0.0931638597955386 |
| chr2 | 58702928 | 58702928 | B-Mem_6720 | 0.098726014551393 |
| chr15 | 68086800 | 68086800 | B-Mem_4325 | 0.1034357532151878 |
| chr5 | 91567806 | 91567806 | B-Mem_10428 | 0.1220837601026858 |

HIV Naïve B Cells

| | | | | |
|---|---|---|---|---|
| chr8 | 15895742 | 15895742 | B-Naive_2979 | −0.4798446894380508 |
| chr3 | 51243113 | 51243113 | B-Naive_2096 | −0.3603133893710195 |
| chr4 | 15375589 | 15375589 | B-Naive_2248 | −0.3413269899199878 |
| chrX | 11707391 | 11707391 | B-Naive_3276 | −0.3350027763927224 |
| chr2 | 150886970 | 150886970 | B-Naive_1750 | −0.3168409581774747 |
| chr13 | 112107547 | 112107547 | B-Naive_870 | −0.3120098520854547 |
| chr9 | 87527290 | 87527290 | B-Naive_3161 | −0.2549731482151102 |
| chr3 | 96513949 | 96513949 | B-Naive_2134 | −0.2528360723201446 |
| chr8 | 37479942 | 37479942 | B-Naive_3006 | −0.2466881768887577 |
| chr20 | 54671417 | 54671417 | B-Naive_1907 | −0.2372045869878904 |
| chr2 | 240965439 | 240965439 | B-Naive_1831 | −0.2346162995705382 |
| chr4 | 152787506 | 152787506 | B-Naive_2344 | −0.2316451365622941 |
| chr21 | 33308270 | 33308270 | B-Naive_1961 | −0.2053377803290866 |
| chr1 | 235073878 | 235073878 | B-Naive_259 | −0.1863733684319155 |
| chr3 | 2156406 | 2156406 | B-Naive_2058 | −0.1810546784599331 |
| chr9 | 36991374 | 36991374 | B-Naive_3139 | −0.1781206817699536 |
| chr11 | 78778997 | 78778997 | B-Naive_558 | −0.1759242387791995 |
| chr11 | 55981584 | 55981584 | B-Naive_528 | −0.1598485394472766 |
| chr10 | 133263642 | 133263642 | B-Naive_468 | −0.1588457102057332 |
| chr2 | 138772446 | 138772446 | B-Naive_1739 | −0.1461642394437906 |
| chr10 | 19845152 | 19845152 | B-Naive_294 | −0.1441384990862265 |
| chr9 | 134881709 | 134881709 | B-Naive_3226 | −0.1407455015919559 |
| chr20 | 51417515 | 51417515 | B-Naive_1903 | −0.1372992391717403 |
| chr20 | 40217593 | 40217593 | B-Naive_1882 | −0.1323626649458411 |
| chr19 | 4164475 | 4164475 | B-Naive_1463 | −0.1308190943251119 |
| chr17 | 40014092 | 40014092 | B-Naive_1294 | −0.1280341003406137 |
| chr19 | 27274722 | 27274722 | B-Naive_1506 | −0.1269553457792565 |
| chr17 | 40466414 | 40466425 | B-Naive_1295 | −0.1197337302002183 |
| chr8 | 108528828 | 108528828 | B-Naive_3068 | −0.112640392528695 |
| chr4 | 138323093 | 138323093 | B-Naive_2333 | −0.109545513638737 |
| chr20 | 34997469 | 34997469 | B-Naive_1878 | −0.1022681155634576 |
| chr10 | 103661325 | 103661325 | B-Naive_403 | −0.0973639664393413 |
| chr11 | 59753870 | 59753870 | B-Naive_533 | −0.0972824015013893 |
| chr13 | 73793113 | 73793113 | B-Naive_837 | −0.0915678488475537 |
| chr7 | 13522609 | 13522609 | B-Naive_2783 | −0.0833701857961202 |
| chr4 | 25063336 | 25063336 | B-Naive_2256 | −0.0820823141085345 |
| chr7 | 124774137 | 124774137 | B-Naive_2894 | −0.0783394367049365 |
| chr7 | 134943954 | 134943954 | B-Naive_2910 | −0.0782433338626324 |
| chr10 | 96391685 | 96391685 | B-Naive_388 | −0.0765604687116368 |
| chr10 | 62072519 | 62072519 | B-Naive_350 | −0.0762303031210164 |
| chr3 | 186215715 | 186215715 | B-Naive_2206 | −0.0758545201206999 |
| chr4 | 119808063 | 119808063 | B-Naive_2325 | −0.0749747428031347 |
| chr15 | 48031274 | 48031274 | B-Naive_1006 | −0.0731316018454697 |
| chr6 | 160939665 | 160939665 | B-Naive_2732 | −0.0710715066689747 |
| chr6 | 54846380 | 54846380 | B-Naive_2654 | −0.06987110744921 |
| chr15 | 88234726 | 88234726 | B-Naive_1047 | −0.0672083000295326 |
| chr19 | 1107085 | 1107085 | B-Naive_1452 | −0.0649053885797118 |
| chr7 | 36649931 | 36649931 | B-Naive_2806 | −0.0643835293813844 |
| chr5 | 142272768 | 142272768 | B-Naive_2514 | −0.0636557369371208 |
| chr16 | 56494257 | 56494257 | B-Naive_1157 | −0.0630864422197253 |
| chr20 | 47176884 | 47176884 | B-Naive_1893 | −0.0628075054287832 |
| chr8 | 1203346 | 1203346 | B-Naive_2958 | −0.0617954628011151 |
| chr8 | 69587218 | 69587218 | B-Naive_3039 | −0.0580646518928603 |
| chr16 | 73265942 | 73265942 | B-Naive_1178 | −0.0575912068214912 |
| chr15 | 39286883 | 39286883 | B-Naive_1004 | −0.0570616159942833 |
| chr9 | 22786441 | 22786441 | B-Naive_3125 | 0.0559742849100084 |
| chr14 | 90843637 | 90843637 | B-Naive_944 | −0.050777998240935 |
| chr10 | 97836906 | 97836906 | B-Naive_390 | −0.0505478663049537 |
| chr2 | 144452597 | 144452597 | B-Naive_1743 | 0.0523176029214069 |
| chr4 | 46753160 | 46753160 | B-Naive_2274 | 0.0546216718127044 |
| chr7 | 3722625 | 3722625 | B-Naive_2771 | 0.0565237097086148 |
| chr1 | 179536452 | 179536452 | B-Naive_204 | 0.0599406594893531 |
| chrX | 56994888 | 56994888 | B-Naive_3294 | 0.0692427585033971 |
| chr1 | 95711474 | 95711474 | B-Naive_144 | 0.0707483326945296 |
| chr7 | 67871927 | 67871927 | B-Naive_2844 | 0.0731150533689978 |
| chr3 | 178709164 | 178709164 | B-Naive_2202 | 0.0769795056268898 |
| chr7 | 56762468 | 56762468 | B-Naive_2825 | 0.0784779143675655 |
| chr5 | 99664563 | 99664563 | B-Naive_2466 | 0.0839293211417712 |
| chr11 | 128716800 | 128716800 | B-Naive_612 | 0.0840164094431101 |
| chr13 | 73041038 | 73041038 | B-Naive_835 | 0.0919548227774508 |
| chr19 | 33865309 | 33865309 | B-Naive_1520 | 0.0946186319684824 |
| chr10 | 120825112 | 120825112 | B-Naive_427 | 0.0961565630998356 |
| chr2 | 226811136 | 226811136 | B-Naive_1807 | 0.0967425773119986 |
| chr4 | 161847682 | 161847682 | B-Naive_2349 | 0.0989647361394248 |
| chr1 | 84436957 | 84436957 | B-Naive_138 | 0.1000903211124086 |
| chr5 | 73119603 | 73119603 | B-Naive_2445 | 0.1024370667468955 |
| chr19 | 1254123 | 1254123 | B-Naive_1453 | 0.1129401922983041 |
| chr6 | 38510884 | 38510884 | B-Naive_2636 | 0.1138913705034232 |
| chr19 | 43686509 | 43686509 | B-Naive_1531 | 0.1167546989558111 |
| chr8 | 126236432 | 126236432 | B-Naive_3083 | 0.1274992869657887 |
| chr14 | 76780415 | 76780415 | B-Naive_926 | 0.129259785377856 |
| chr1 | 28181484 | 28181484 | B-Naive_76 | 0.1292598008392033 |
| chr6 | 47873254 | 47873254 | B-Naive_2646 | 0.1382013915929075 |
| chr13 | 111583194 | 111583194 | B-Naive_869 | 0.1438621089141829 |
| chr7 | 109237189 | 109237189 | B-Naive_2877 | 0.1492905966659372 |
| chr10 | 7800595 | 7800595 | B-Naive_282 | 0.1535868044424965 |
| chr11 | 135076423 | 135076423 | B-Naive_624 | 0.1633234738074486 |
| chr14 | 25300716 | 25300716 | B-Naive_888 | 0.1649100472851574 |
| chr3 | 6771418 | 6771418 | B-Naive_2065 | 0.1708047128571622 |
| chr12 | 102419312 | 102419312 | B-Naive_727 | 0.1838873131889968 |
| chr2 | 208359741 | 208359741 | B-Naive_1784 | 0.1858739695075335 |
| chr11 | 111409578 | 111409578 | B-Naive_587 | 0.1888511752049065 |
| chrX | 47916556 | 47916556 | B-Naive_3293 | 0.191584688482566 |
| chr1 | 105218394 | 105218394 | B-Naive_150 | 0.2166863852332018 |
| chr1 | 201260222 | 201260222 | B-Naive_219 | 0.2286157281054771 |
| chr15 | 68558733 | 68558733 | B-Naive_1024 | 0.2580040046317804 |
| chr20 | 61773166 | 61773166 | B-Naive_1924 | 0.2938915235638216 |
| chr3 | 129953683 | 129953683 | B-Naive_2163 | 0.3351471831351672 |
| chr16 | 35691539 | 35691539 | B-Naive_1137 | 0.3608175520663908 |
| chr16 | 9008502 | 9008502 | B-Naive_1094 | 0.3834179275855069 |

HIV Monocyte

| | | | | |
|---|---|---|---|---|
| chr13 | 82332807 | 82332807 | Monocyte_1733 | −0.2930191043721925 |
| chr14 | 33289753 | 33289753 | Monocyte_1858 | −0.2719724799032443 |
| chr6 | 101463605 | 101463605 | Monocyte_5750 | −0.178587918196469 |
| chrX | 25239737 | 25239737 | Monocyte_6959 | −0.1580415235961627 |
| chr6 | 75083014 | 75083014 | Monocyte_5693 | −0.1450162575732124 |
| chrX | 71915405 | 71915405 | Monocyte_7011 | −0.1428848886616682 |
| chr3 | 150493321 | 150493321 | Monocyte_4635 | −0.1417911765749341 |
| chr8 | 11258474 | 11258474 | Monocyte_6288 | −0.1289066727548886 |
| chr10 | 10205628 | 10205628 | Monocyte_541 | −0.1214500548904539 |
| chr4 | 33000188 | 33000188 | Monocyte_4842 | −0.1185152048465919 |
| chr17 | 49580352 | 49580352 | Monocyte_2695 | −0.1182258320338273 |
| chr20 | 44281484 | 44281484 | Monocyte_3962 | −0.1117680232287829 |
| chr15 | 82043879 | 82043879 | Monocyte_2183 | −0.1103628773194567 |
| chr1 | 207888161 | 207888161 | Monocyte_418 | −0.1095366937757299 |
| chr4 | 175553410 | 175553410 | Monocyte_5066 | −0.1085860093260827 |
| chr15 | 45631943 | 45631943 | Monocyte_2110 | −0.1071139426985909 |
| chr1 | 4407748 | 4407748 | Monocyte_18 | −0.1039745399597522 |
| chrX | 30598287 | 30598287 | Monocyte_6967 | −0.1034104680390005 |
| chr16 | 24851876 | 24851876 | Monocyte_2350 | −0.0963065552722283 |
| chr3 | 13749211 | 13749211 | Monocyte_4347 | −0.0958041486357558 |
| chr1 | 78619477 | 78619477 | Monocyte_208 | −0.0923519127194986 |
| chr6 | 153350023 | 153350023 | Monocyte_5831 | −0.0922812220555399 |
| chr3 | 124719753 | 124719753 | Monocyte_4585 | −0.0913854889935742 |
| chr10 | 20877688 | 20877688 | Monocyte_569 | −0.0911066580074569 |
| chr2 | 117706082 | 117706082 | Monocyte_3536 | −0.091073864579034 |
| chr6 | 103908352 | 103908352 | Monocyte_5756 | −0.0894466960437925 |
| chr21 | 18748631 | 18748631 | Monocyte_4103 | −0.0889842097997314 |
| chr4 | 69380122 | 69380122 | Monocyte_4898 | −0.0887177923987681 |
| chr18 | 34620304 | 34620304 | Monocyte_2883 | −0.0876143332558527 |
| chr20 | 62824584 | 62824584 | Monocyte_4055 | −0.0860909816275778 |
| chr20 | 36305459 | 36305459 | Monocyte_3928 | −0.0853208621610271 |
| chr6 | 103368889 | 103368889 | Monocyte_5755 | −0.0849778307178369 |
| chr6 | 161513947 | 161513947 | Monocyte_5844 | −0.0838847730818411 |
| chr7 | 19803058 | 19803058 | Monocyte_5934 | −0.0838276732337847 |
| chr13 | 18289290 | 18289290 | Monocyte_1594 | −0.0837160038317105 |
| chr14 | 56665803 | 56665803 | Monocyte_1906 | −0.0835286870155519 |
| chr1 | 81710688 | 81710688 | Monocyte_216 | −0.0826334700093074 |
| chr18 | 40637233 | 40637233 | Monocyte_2901 | −0.0825743683388144 |
| chr2 | 229479981 | 229479981 | Monocyte_3766 | −0.0797708139213748 |
| chr3 | 38818527 | 38818527 | Monocyte_4392 | −0.0769251804909851 |
| chr9 | 133587289 | 133587289 | Monocyte_6876 | −0.0757451979919122 |
| chr7 | 53556083 | 53556083 | Monocyte_6007 | −0.075056706242874 |
| chr14 | 57060531 | 57060531 | Monocyte_1908 | −0.0747688537415955 |
| chr14 | 64746496 | 64746496 | Monocyte_1925 | −0.0734663572563963 |
| chr19 | 56905096 | 56905096 | Monocyte_3246 | −0.0732742983781762 |
| chr16 | 67171383 | 67171383 | Monocyte_2458 | −0.0718441829628289 |
| chrX | 112186492 | 112186492 | Monocyte_7056 | −0.0682756772122668 |
| chr4 | 121932714 | 121932714 | Monocyte_4980 | −0.0682258366141946 |
| chr6 | 7225261 | 7225261 | Monocyte_5531 | −0.0676550674533138 |
| chr6 | 168109288 | 168109288 | Monocyte_5866 | −0.0671828631267255 |
| chr19 | 9704407 | 9704407 | Monocyte_3054 | −0.0670884306535342 |
| chr11 | 44709948 | 44709948 | Monocyte_970 | −0.0665257604907234 |
| chr15 | 26865756 | 26865756 | Monocyte_2072 | −0.0657362798357814 |
| chr10 | 79671902 | 79671902 | Monocyte_711 | 0.066118607163657 |
| chr8 | 71632890 | 71632890 | Monocyte_6447 | 0.0664856895953162 |
| chr9 | 133667140 | 133667140 | Monocyte_6877 | 0.0665071902432857 |
| chr19 | 3577258 | 3577258 | Monocyte_3029 | 0.0668565819763344 |
| chr5 | 167570535 | 167570535 | Monocyte_5456 | 0.0677798158999225 |
| chr16 | 84876025 | 84876025 | Monocyte_2502 | 0.0682461696535705 |
| chr2 | 12560823 | 12560823 | Monocyte_3314 | 0.0706546211916355 |
| chr10 | 22334834 | 22334834 | Monocyte_574 | 0.0712205786691391 |
| chr5 | 130734220 | 130734220 | Monocyte_5368 | 0.0733898492848839 |
| chr4 | 28986849 | 28986849 | Monocyte_4837 | 0.0738593092024926 |
| chr19 | 28485533 | 28485533 | Monocyte_3108 | 0.0744525019653099 |
| chr19 | 10131770 | 10131770 | Monocyte_3056 | 0.0762614001152459 |
| chr10 | 394566 | 394566 | Monocyte_511 | 0.0788633008800391 |
| chr20 | 8726099 | 8726099 | Monocyte_3846 | 0.0863756286865914 |
| chr11 | 65774207 | 65774207 | Monocyte_1042 | 0.0884402057209937 |
| chr2 | 128038556 | 128038556 | Monocyte_3578 | 0.0891134170139894 |
| chr17 | 21946780 | 21946780 | Monocyte_2619 | 0.0932710145966758 |
| chr14 | 74989758 | 74989758 | Monocyte_1940 | 0.093686791128061 |
| chr22 | 46556530 | 46556530 | Monocyte_4288 | 0.0938179805153442 |
| chr8 | 119542944 | 119542944 | Monocyte_6526 | 0.0941934142856325 |
| chr9 | 130471953 | 130471953 | Monocyte_6858 | 0.098240778066906 |
| chr2 | 121871959 | 121871959 | Monocyte_3554 | 0.0998180082979276 |
| chr21 | 16583814 | 16583814 | Monocyte_4098 | 0.1006225596932821 |
| chr16 | 88527358 | 88527358 | Monocyte_2531 | 0.1009939088098846 |
| chr11 | 61534457 | 61534457 | Monocyte_1023 | 0.1028605932158334 |
| chr1 | 36627211 | 36627211 | Monocyte_148 | 0.1032496461308105 |
| chr21 | 10742788 | 10742788 | Monocyte_4090 | 0.1045696457981672 |
| chr4 | 3430855 | 3430855 | Monocyte_4749 | 0.1099350439158371 |
| chr18 | 79648421 | 79648421 | Monocyte_2996 | 0.1119798449162411 |
| chrY | 17136237 | 17136237 | Monocyte_7132 | 0.1147837709571315 |
| chr12 | 34231599 | 34231599 | Monocyte_1339 | 0.118885396142177 |
| chrX | 118826280 | 118826280 | Monocyte_7064 | 0.1201954162711475 |
| chr4 | 185199322 | 185199322 | Monocyte_5093 | 0.120310369537455 |
| chr16 | 85919591 | 85919591 | Monocyte_2515 | 0.1249378029433658 |
| chr7 | 155796881 | 155796881 | Monocyte_6239 | 0.1284940911498871 |
| chr3 | 11887892 | 11887892 | Monocyte_4344 | 0.1297223130307888 |
| chr15 | 56555945 | 56555945 | Monocyte_2128 | 0.1351814240045896 |
| chr11 | 26212995 | 26212995 | Monocyte_933 | 0.1409344743101421 |
| chr12 | 49116091 | 49116091 | Monocyte_1368 | 0.1486515226552874 |
| chr7 | 126313145 | 126313145 | Monocyte_6160 | 0.1556662986596574 |
| chr21 | 9580116 | 9580116 | Monocyte_4081 | 0.1619873938361868 |
| chr12 | 73166579 | 73166579 | Monocyte_1419 | 0.169428793101113 |
| chr2 | 98311962 | 98311962 | Monocyte_3501 | 0.1787704497299039 |
| chr8 | 58795751 | 58795751 | Monocyte_6418 | 0.2112442881333038 |
| chr7 | 8267889 | 8267889 | Monocyte_5910 | 0.2138698878714496 |
| chr19 | 23002148 | 23002148 | Monocyte_3101 | 0.2266837938454677 |
| chrX | 33596579 | 33596579 | Monocyte_6973 | 0.2859978870162928 |

HIV active natural killer cells

| | | | | |
|---|---|---|---|---|
| chr11 | 93912102 | 93912102 | NK-cell1_2347 | −0.0833535007624519 |
| chr2 | 132670691 | 132670691 | NK-cell1_6948 | −0.0736279062863821 |
| chr10 | 89165746 | 89165746 | NK-cell1_1549 | −0.0700159474518299 |
| chrX | 123978090 | 123978090 | NK-cell1_14053 | −0.0651127363712532 |
| chr20 | 26199580 | 26199580 | NK-cell1_7560 | −0.0646904132526417 |
| chr10 | 26168408 | 26168408 | NK-cell1_1253 | −0.060416386355002 |
| chr6 | 111621019 | 111621019 | NK-cell1_11273 | −0.0571100029983332 |
| chr5 | 180730683 | 180730683 | NK-cell1_10760 | −0.0528751299279213 |
| chr8 | 52928549 | 52928549 | NK-cell1_12655 | −0.0497696033785995 |
| chr11 | 62164585 | 62164585 | NK-cell1_2175 | −0.0493485984270575 |
| chr16 | 67185184 | 67185184 | NK-cell1_4853 | −0.0445189530134502 |
| chr22 | 38938989 | 38938989 | NK-cell1_8142 | −0.0429942542520031 |
| chr19 | 38185019 | 38185019 | NK-cell1_6191 | −0.0428593851975682 |
| chr3 | 70383839 | 70383839 | NK-cell1_8555 | −0.0421295176150146 |
| chrX | 104932294 | 104932294 | NK-cell1_13996 | −0.0420334466498237 |
| chr14 | 90109600 | 90109600 | NK-cell1_3937 | −0.0416999820415712 |
| chr10 | 18095373 | 18095373 | NK-cell1_1210 | −0.0414510343712402 |
| chr20 | 2212202 | 2212202 | NK-cell1_7437 | −0.0410916771369257 |
| chr14 | 45791719 | 45791719 | NK-cell1_3762 | −0.0410907209145105 |
| chr3 | 60956626 | 60956626 | NK-cell1_8520 | −0.0410027101406161 |
| chr7 | 19817806 | 19817806 | NK-cell1_11664 | −0.0406017261575908 |
| chr16 | 14015489 | 14015489 | NK-cell1_4642 | −0.0397940661567567 |
| chr7 | 138685371 | 138685371 | NK-cell1_12226 | −0.0394943118619099 |
| chr6 | 147747727 | 147747727 | NK-cell1_11403 | −0.0389234352218515 |
| chr17 | 8695822 | 8695822 | NK-cell1_5084 | −0.0386227971004056 |
| chr3 | 110182210 | 110182210 | NK-cell1_8720 | 0.0386005630926333 |
| chr19 | 13785424 | 13785424 | NK-cell1_6040 | 0.0389793671452973 |
| chr10 | 3319048 | 3319048 | NK-cell1_1114 | 0.0392246666005117 |
| chr21 | 27631119 | 27631119 | NK-cell1_7883 | 0.0392335870904466 |
| chr1 | 14346529 | 14346529 | NK-cell1_145 | 0.0394928233982074 |
| chr5 | 13698408 | 13698408 | NK-cell1_10037 | 0.0399297808007406 |
| chr15 | 101328231 | 101328231 | NK-cell1_4512 | 0.0407730185075701 |
| chr17 | 21717213 | 21717213 | NK-cell1_5148 | 0.0409044448900196 |
| chr3 | 154605672 | 154605672 | NK-cell1_8904 | 0.0413263363772497 |
| chr6 | 89123992 | 89123992 | NK-cell1_11192 | 0.041720716763322 |
| chr2 | 195892885 | 195892885 | NK-cell1_7174 | 0.0418738998034541 |
| chr5 | 16098268 | 16098268 | NK-cell1_10058 | 0.0418891390682485 |
| chr19 | 23195468 | 23195468 | NK-cell1_6104 | 0.0423359002189662 |
| chr4 | 161900181 | 161900181 | NK-cell1_9789 | 0.0427023377114216 |
| chr15 | 81618851 | 81618851 | NK-cell1_4383 | 0.0432642422136738 |
| chr14 | 59145814 | 59145814 | NK-cell1_3819 | 0.0433003380903545 |
| chr10 | 48854173 | 48854173 | NK-cell1_1363 | 0.0435834419566063 |
| chr10 | 35469204 | 35469204 | NK-cell1_1299 | 0.0441287553302119 |
| chr5 | 124472851 | 124472851 | NK-cell1_10445 | 0.0441296032920018 |
| chr8 | 72707407 | 72707407 | NK-cell1_12750 | 0.0444186990049776 |
| chr7 | 103431422 | 103431422 | NK-cell1_12069 | 0.0451086734772307 |
| chr21 | 41120986 | 41120986 | NK-cell1_7963 | 0.0452105058840431 |
| chr3 | 57526683 | 57526683 | NK-cell1_8500 | 0.0455710094038956 |
| chrX | 105325985 | 105325985 | NK-cell1_14000 | 0.045781018844211 |
| chr9 | 89070516 | 89070516 | NK-cell1_13404 | 0.0461478109425853 |
| chr12 | 100582411 | 100582411 | NK-cell1_3012 | 0.0462021795099641 |
| chr19 | 3807189 | 3807189 | NK-cell1_5966 | 0.0463133002397214 |
| chr1 | 241211669 | 241211669 | NK-cell1_1040 | 0.0467677617877474 |
| chr11 | 69533734 | 69533734 | NK-cell1_2229 | 0.0469146041731404 |
| chr2 | 138400579 | 138400579 | NK-cell1_6975 | 0.0469655681609991 |
| chr19 | 2556574 | 2556574 | NK-cell1_5949 | 0.04717019558102 |
| chr4 | 106819982 | 106819982 | NK-cell1_9570 | 0.0474612410640251 |
| chr18 | 52743298 | 52743298 | NK-cell1_5750 | 0.0476421307527611 |
| chr21 | 34166903 | 34166903 | NK-cell1_7919 | 0.0476824479783156 |
| chr7 | 49004292 | 49004292 | NK-cell1_11817 | 0.0489337700245107 |
| chrX | 70744870 | 70744870 | NK-cell1_13925 | 0.0493118250846786 |
| chr6 | 849772 | 849772 | NK-cell1_10767 | 0.0494726722774849 |
| chr8 | 49783559 | 49783559 | NK-cell1_12634 | 0.0501450290357823 |
| chr1 | 216183591 | 216183591 | NK-cell1_929 | 0.0501524611268133 |
| chr21 | 40363505 | 40363505 | NK-cell1_7953 | 0.0509337377878225 |
| chr2 | 203660310 | 203660310 | NK-cell1_7194 | 0.0509348686039759 |
| chr11 | 119912478 | 119912478 | NK-cell1_2471 | 0.0512455542976901 |
| chr22 | 40752148 | 40752148 | NK-cell1_8154 | 0.0527399740459766 |
| chr2 | 167120507 | 167120507 | NK-cell1_7064 | 0.0529263122472467 |
| chr2 | 228524464 | 228524464 | NK-cell1_7318 | 0.0532370437092635 |
| chr4 | 66224039 | 66224039 | NK-cell1_9421 | 0.0532633980223332 |
| chr8 | 17487146 | 17487146 | NK-cell1_12451 | 0.053650272044234 |
| chr2 | 225998158 | 225998158 | NK-cell1_7302 | 0.0540926826032707 |
| chr11 | 74314251 | 74314251 | NK-cell1_2264 | 0.0541664276057896 |
| chrX | 130709546 | 130709546 | NK-cell1_14073 | 0.0547729737497545 |
| chr2 | 122067997 | 122067997 | NK-cell1_6887 | 0.0553947482669578 |
| chr1 | 176715294 | 176715294 | NK-cell1_762 | 0.0554611065943018 |
| chr6 | 46679517 | 46679517 | NK-cell1_11031 | 0.0555042182448205 |
| chrX | 77331515 | 77331515 | NK-cell1_13939 | 0.0557706359499872 |
| chr1 | 84858472 | 84858472 | NK-cell1_509 | 0.0559976489773227 |
| chr12 | 129220785 | 129220785 | NK-cell1_3189 | 0.0561140572255539 |
| chr3 | 79257799 | 79257799 | NK-cell1_8598 | 0.0561862563679414 |
| chr8 | 108282353 | 108282353 | NK-cell1_12904 | 0.0563261325466505 |
| chr1 | 5483969 | 5483969 | NK-cell1_70 | 0.0565440578394282 |
| chr5 | 174435067 | 174435067 | NK-cell1_10712 | 0.05803132494753 |
| chr18 | 15868108 | 15868108 | NK-cell1_5589 | 0.0597792242152192 |
| chr18 | 68133902 | 68133902 | NK-cell1_5841 | 0.06319862411976 |
| chr7 | 98515204 | 98515204 | NK-cell1_12041 | 0.0634647449613816 |
| chr14 | 79066070 | 79066070 | NK-cell1_3899 | 0.0675092196304105 |
| chrX | 111397647 | 111397647 | NK-cell1_14018 | 0.0693975221927765 |
| chr5 | 167769577 | 167769577 | NK-cell1_10653 | 0.0696538365222249 |
| chr19 | 54782858 | 54782858 | NK-cell1_6324 | 0.0700660087890344 |
| chrX | 153662173 | 153662173 | NK-cell1_14152 | 0.0711697411522802 |
| chr20 | 52709407 | 52709407 | NK-cell1_7721 | 0.0715356681673534 |
| chr12 | 125264424 | 125264424 | NK-cell1_3158 | 0.0730429341539928 |
| chr10 | 7413149 | 7413149 | NK-cell1_1136 | 0.0816900635310933 |
| chr17 | 45613488 | 45613488 | NK-cell1_5253 | 0.0842096667238704 |
| chr5 | 127864929 | 127864929 | NK-cell1_10464 | 0.0960885768961076 |
| chr18 | 35655010 | 35655010 | NK-cell1_5657 | 0.0979515924720178 |
| chr7 | 123809595 | 123809595 | NK-cell1_12155 | 0.1029456639987578 |

HIV naive natural killer cells

| | | | | |
|---|---|---|---|---|
| chr11 | 129637024 | 129637024 | NK-cell2_1257 | −0.1841090741028504 |
| chr7 | 123957537 | 123957537 | NK-cell2_6075 | −0.1432957903174804 |
| chr7 | 143413147 | 143413147 | NK-cell2_6118 | −0.1425825638863496 |
| chr6 | 34009357 | 34009357 | NK-cell2_5483 | −0.1356163473544303 |
| chr5 | 6735917 | 6735917 | NK-cell2_4995 | −0.133302656787858 |
| chr13 | 102431465 | 102431465 | NK-cell2_1783 | −0.1215002680038103 |
| chr8 | 132363237 | 132363237 | NK-cell2_6449 | −0.1193952204196966 |
| chr10 | 65735985 | 65735985 | NK-cell2_726 | −0.1126325167361914 |
| chr9 | 127591499 | 127591499 | NK-cell2_6718 | −0.1119829524102959 |
| chr18 | 8976431 | 8976431 | NK-cell2_2811 | −0.1112048789547344 |
| chr11 | 116694684 | 116694684 | NK-cell2_1219 | −0.10971478765483 |
| chr4 | 18607827 | 18607827 | NK-cell2_4639 | −0.1079577371616505 |
| chr2 | 120655167 | 120655167 | NK-cell2_3475 | −0.1013469818061313 |
| chr13 | 28920614 | 28920614 | NK-cell2_1623 | −0.1001326941278267 |
| chr2 | 207156970 | 207156970 | NK-cell2_3624 | −0.0998367680149052 |
| chr6 | 169958899 | 169958899 | NK-cell2_5767 | −0.0965299817592714 |
| chr10 | 75372212 | 75372212 | NK-cell2_752 | −0.093256484185785 |
| chr3 | 117453728 | 117453728 | NK-cell2_4384 | −0.0919826389998691 |
| chr3 | 30975367 | 30975367 | NK-cell2_4208 | −0.0909547273680416 |
| chr2 | 140414941 | 140414941 | NK-cell2_3524 | −0.0907277647583073 |
| chr11 | 16471630 | 16471630 | NK-cell2_986 | −0.0855337319042554 |
| chr18 | 74563602 | 74563602 | NK-cell2_2961 | −0.0841857004391194 |
| chr2 | 96287469 | 96287469 | NK-cell2_3413 | −0.0834322339075079 |
| chr1 | 230106123 | 230106123 | NK-cell2_497 | −0.0816100931514141 |
| chr1 | 234996757 | 234996757 | NK-cell2_508 | −0.0813910409348132 |
| chr13 | 61616607 | 61616607 | NK-cell2_1697 | −0.0806656297053448 |
| chr1 | 17556504 | 17556504 | NK-cell2_67 | −0.0805247989790749 |
| chr16 | 84040488 | 84040488 | NK-cell2_2523 | −0.0800799021207344 |
| chr2 | 4787523 | 4787523 | NK-cell2_3223 | −0.0798401261974358 |
| chr20 | 1817939 | 1817939 | NK-cell2_3729 | −0.0797166731927177 |
| chr10 | 119762943 | 119762943 | NK-cell2_873 | −0.0789313080942076 |
| chr1 | 179542315 | 179542315 | NK-cell2_387 | −0.0774050426267861 |
| chr1 | 67764893 | 67764893 | NK-cell2_1113 | −0.0732161494870106 |
| chr7 | 118487210 | 118487210 | NK-cell2_6065 | −0.0720985505535364 |
| chr5 | 62101768 | 62101768 | NK-cell2_5107 | −0.0719544109011443 |
| chr19 | 1552963 | 1552963 | NK-cell2_2985 | −0.0711689197753164 |
| chr2 | 177848424 | 177848424 | NK-cell2_3583 | −0.0711020613729695 |
| chr10 | 62810390 | 62810390 | NK-cell2_719 | −0.0703963630486612 |
| chr12 | 110012905 | 110012905 | NK-cell2_1504 | −0.0699507828698378 |
| chr9 | 9380583 | 9380583 | NK-cell2_6519 | −0.0688368364235593 |
| chr20 | 62392444 | 62392444 | NK-cell2_3901 | −0.0684995900242114 |
| chr13 | 50365831 | 50365867 | NK-cell2_1667 | −0.068432527912935 |
| chr12 | 12006941 | 12006941 | NK-cell2_1326 | −0.0683339713685419 |
| chrX | 105667204 | 105667204 | NK-cell2_6915 | −0.0681625909506061 |
| chr1 | 68086286 | 68086286 | NK-cell2_198 | −0.0681268097726475 |
| chr6 | 16306129 | 16306129 | NK-cell2_5441 | −0.0673649579502059 |
| chr3 | 124827615 | 124827615 | NK-cell2_4404 | −0.0652300888379829 |
| chr2 | 7924562 | 7924562 | NK-cell2_3229 | −0.06404049477932 |
| chr6 | 91965785 | 91965785 | NK-cell2_5594 | −0.0633293861279127 |
| chr15 | 88192906 | 88192906 | NK-cell2_2216 | −0.0627414735246452 |
| chr4 | 26197975 | 26197975 | NK-cell2_4657 | −0.0626136256638679 |
| chr4 | 38310261 | 38310261 | NK-cell2_4685 | −0.0613887304796112 |
| chr19 | 48266899 | 48266899 | NK-cell2_3162 | −0.0605966406550433 |
| chr7 | 25968223 | 25968223 | NK-cell2_5850 | −0.060182316058309 |
| chr11 | 105523989 | 105523989 | NK-cell2_1198 | −0.059946290178219 |
| chr2 | 21289027 | 21289027 | NK-cell2_3265 | −0.0594111928116084 |
| chr15 | 93334049 | 93334049 | NK-cell2_2231 | 0.0585565301651588 |
| chr3 | 22900780 | 22900780 | NK-cell2_4194 | 0.0594230568046866 |
| chr6 | 109453901 | 109453901 | NK-cell2_5630 | 0.0595057088979306 |
| chrX | 6403450 | 6403450 | NK-cell2_6807 | 0.0617431463507768 |
| chr7 | 63114637 | 63114637 | NK-cell2_5945 | 0.0620946891259382 |
| chr5 | 132748191 | 132748191 | NK-cell2_5231 | 0.0641206754733411 |
| chr7 | 2523565 | 2523565 | NK-cell2_5790 | 0.0644722253974956 |
| chr16 | 29155502 | 29155502 | NK-cell2_2357 | 0.0645826046194457 |
| chr2 | 202089788 | 202089788 | NK-cell2_3613 | 0.0647095401754017 |
| chr22 | 26839708 | 26839708 | NK-cell2_4055 | 0.0667537593377814 |
| chr17 | 21936931 | 21936931 | NK-cell2_2617 | 0.0672018459143395 |
| chr9 | 77455514 | 77455514 | NK-cell2_6597 | 0.067399131434621 |
| chr19 | 43826759 | 43826759 | NK-cell2_3148 | 0.0674725419444808 |
| chr11 | 68650344 | 68650344 | NK-cell2_1119 | 0.0690027291757045 |
| chr17 | 5112687 | 5112687 | NK-cell2_2569 | 0.0705411972966305 |
| chrX | 116688040 | 116688040 | NK-cell2_6927 | 0.0705696077176676 |
| chr14 | 55890554 | 55890554 | NK-cell2_1925 | 0.0717848921975581 |
| chr13 | 60668254 | 60668254 | NK-cell2_1694 | 0.0748459713061699 |
| chrX | 43732254 | 43732254 | NK-cell2_6854 | 0.0782315670002413 |
| chr16 | 80159354 | 80159354 | NK-cell2_2507 | 0.0784160360442732 |
| chr2 | 156898831 | 156898831 | NK-cell2_3549 | 0.0792014274080189 |
| chr20 | 30919657 | 30919657 | NK-cell2_3790 | 0.0804271350810052 |
| chr13 | 54211499 | 54211499 | NK-cell2_1678 | 0.0822007850966213 |
| chr3 | 194040637 | 194040637 | NK-cell2_4540 | 0.084792916116673 |
| chr11 | 130859691 | 130859691 | NK-cell2_1259 | 0.0852947778757782 |
| chr16 | 351507 | 351507 | NK-cell2_2263 | 0.0860852685701018 |
| chr8 | 111797349 | 111797349 | NK-cell2_6403 | 0.0894517863618687 |
| chr13 | 89372410 | 89372410 | NK-cell2_1741 | 0.0900630468896267 |
| chr5 | 114561595 | 114561595 | NK-cell2_5193 | 0.0913040476106116 |
| chr6 | 154937844 | 154937844 | NK-cell2_5715 | 0.0922643873472748 |
| chr1 | 46486361 | 46486361 | NK-cell2_158 | 0.0968027334307119 |
| chr8 | 79352140 | 79352140 | NK-cell2_6348 | 0.1010324608186454 |
| chrX | 14291717 | 14291717 | NK-cell2_6811 | 0.1022597514743814 |
| chr17 | 21048549 | 21048549 | NK-cell2_2613 | 0.1112354960259606 |
| chr10 | 103764323 | 103764323 | NK-cell2_830 | 0.1163170854774477 |
| chr3 | 20751297 | 20751297 | NK-cell2_4191 | 0.1172095389057559 |
| chr1 | 36912195 | 36912258 | NK-cell2_137 | 0.1173711562994077 |
| chr5 | 178656797 | 178656797 | NK-cell2_5369 | 0.137468415515986 |
| chr13 | 23757763 | 23757763 | NK-cell2_1612 | 0.1487475670373395 |
| chr13 | 93243564 | 93243564 | NK-cell2_1755 | 0.1490859976978417 |
| chr10 | 130213720 | 130213720 | NK-cell2_906 | 0.1530054254540286 |
| chrX | 134159177 | 134159177 | NK-cell2_6949 | 0.1570117496556686 |
| chr7 | 117468614 | 117468614 | NK-cell2_6062 | 0.1593833053456748 |
| chr22 | 20269506 | 20269506 | NK-cell2_4038 | 0.2186385373326783 |

HIV memory cytotoxic T cells

| | | | | |
|---|---|---|---|---|
| chr11 | 95574264 | 95574264 | Tc-Mem_4825 | −0.0764558115292998 |
| chrX | 71043648 | 71043648 | Tc-Mem_28370 | −0.0742780156473741 |
| chr13 | 85468555 | 85468555 | Tc-Mem_7169 | −0.0736793804654602 |
| chr22 | 18152167 | 18152167 | Tc-Mem_16511 | −0.0725220824518234 |
| chrX | 137898483 | 137898483 | Tc-Mem_28653 | −0.0608583854474552 |
| chr11 | 115626114 | 115626114 | Tc-Mem_5010 | −0.0597826895860528 |
| chr2 | 82008965 | 82008965 | Tc-Mem_13674 | −0.0584765892100027 |
| chr6 | 55746688 | 55746688 | Tc-Mem_22763 | −0.0578133557938523 |
| chr2 | 131831486 | 131831486 | Tc-Mem_14239 | −0.0577862420772524 |
| chr4 | 62348170 | 62348170 | Tc-Mem_19377 | −0.0529605916087438 |
| chr10 | 45712226 | 45712226 | Tc-Mem_2881 | −0.0528483092609747 |
| chr2 | 108693028 | 108693028 | Tc-Mem_13963 | −0.0517682445128645 |
| chr5 | 141836419 | 141836419 | Tc-Mem_21712 | −0.0517423423649661 |
| chrX | 27831004 | 27831004 | Tc-Mem_28167 | −0.0515803149092263 |
| chr22 | 22054185 | 22054185 | Tc-Mem_16558 | −0.0505666786668664 |
| chr16 | 80808732 | 80808732 | Tc-Mem_10029 | −0.0504544019510767 |
| chr22 | 31508678 | 31508678 | Tc-Mem_16686 | −0.0501711126255898 |
| chr5 | 142222866 | 142222866 | Tc-Mem_21721 | 0.0498293053793521 |
| chr2 | 223765052 | 223765052 | Tc-Mem_14958 | −0.0496148166090722 |
| chr3 | 85321242 | 85321242 | Tc-Mem_17828 | −0.0495713408142705 |
| chr6 | 136810457 | 136810457 | Tc-Mem_23369 | −0.0490749611970648 |
| chr11 | 94184824 | 94184824 | Tc-Mem_4811 | −0.0486896270894471 |
| chr21 | 28027240 | 28027240 | Tc-Mem_16227 | −0.0483206557944967 |
| chr6 | 94040820 | 94040820 | Tc-Mem_23046 | −0.0480655263936357 |
| chr17 | 56915468 | 56915468 | Tc-Mem_10890 | −0.0471439811275538 |
| chr17 | 78883952 | 78883952 | Tc-Mem_11166 | −0.0466175336038313 |
| chr13 | 29879409 | 29879409 | Tc-Mem_6736 | −0.0462255506294607 |
| chr5 | 148929810 | 148929810 | Tc-Mem_21774 | −0.0446333540740266 |
| chr5 | 35840513 | 35840513 | Tc-Mem_20898 | −0.0434217644035462 |
| chrX | 129551680 | 129551680 | Tc-Mem_28621 | −0.0430705361486538 |
| chr11 | 46353685 | 46353685 | Tc-Mem_4299 | −0.0428848825363129 |
| chr1 | 198930595 | 198930595 | Tc-Mem_1818 | −0.0424644906339507 |
| chrX | 54877280 | 54877280 | Tc-Mem_28304 | −0.0423968259511401 |
| chr4 | 136639528 | 136639528 | Tc-Mem_19975 | −0.0420415572027396 |
| chr2 | 25323764 | 25323764 | Tc-Mem_13158 | −0.0411581040766391 |
| chr5 | 126848588 | 126848588 | Tc-Mem_21538 | −0.0409692737588198 |
| chrX | 22043906 | 22043906 | Tc-Mem_28143 | −0.0406759165563174 |
| chr3 | 81561160 | 81561160 | Tc-Mem_17800 | −0.0406576648711952 |
| chr11 | 125351332 | 125351332 | Tc-Mem_5158 | −0.0404385899227104 |
| chr19 | 8593343 | 8593343 | Tc-Mem_12215 | −0.0404347032007827 |
| chr3 | 180181986 | 180181986 | Tc-Mem_18565 | −0.0399710665997542 |
| chr19 | 34232432 | 34232432 | Tc-Mem_12522 | −0.0396401202179761 |
| chr2 | 234373128 | 234373128 | Tc-Mem_15059 | −0.0390133706954159 |
| chr1 | 143194375 | 143194375 | Tc-Mem_1340 | −0.0389708090012018 |
| chr9 | 23851297 | 23851297 | Tc-Mem_26967 | −0.0381061462604954 |
| chrX | 121047650 | 121047652 | Tc-Mem_28578 | −0.0381025537174944 |
| chrX | 73447670 | 73447670 | Tc-Mem_28379 | −0.0380228029729432 |
| chr5 | 16998734 | 16998734 | Tc-Mem_20716 | −0.037977886113475 |
| chr2 | 102262475 | 102262475 | Tc-Mem_13868 | −0.0378530481655434 |
| chr10 | 124615830 | 124615830 | Tc-Mem_3651 | −0.0374602404518914 |
| chr4 | 155884145 | 155884145 | Tc-Mem_20135 | −0.0373948290981074 |
| chr8 | 22690504 | 22690504 | Tc-Mem_25598 | −0.0373172190600404 |
| chr10 | 339009 | 339009 | Tc-Mem_2351 | −0.0367906456419145 |
| chr2 | 20412035 | 20412035 | Tc-Mem_13109 | −0.0367820382949087 |
| chr3 | 18369886 | 18369886 | Tc-Mem_17198 | −0.0364737965724392 |
| chr14 | 31899552 | 31899552 | Tc-Mem_7593 | −0.0364687767233344 |
| chr8 | 3816453 | 3816453 | Tc-Mem_25367 | −0.0363789889875036 |
| chr1 | 39589830 | 39589830 | Tc-Mem_652 | −0.0361770985509459 |
| chrX | 43974165 | 43974165 | Tc-Mem_28248 | −0.036097752585033 |
| chr10 | 101770761 | 101770761 | Tc-Mem_3404 | −0.0360670158839654 |
| chr5 | 7677235 | 7677235 | Tc-Mem_20617 | −0.0354491433468396 |
| chr11 | 84692679 | 84692679 | Tc-Mem_4744 | −0.0352571567702344 |
| chr5 | 5891878 | 5891878 | Tc-Mem_20589 | −0.0351925005326587 |
| chr22 | 48299055 | 48299055 | Tc-Mem_16959 | 0.0353372434806673 |
| chr10 | 117105600 | 117105600 | Tc-Mem_3555 | 0.0355175430452599 |
| chr13 | 35711265 | 35711265 | Tc-Mem_6794 | 0.0356654045177907 |
| chr5 | 120834008 | 120834008 | Tc-Mem_21488 | 0.0358524147173022 |
| chr13 | 69522093 | 69522093 | Tc-Mem_7050 | 0.0360163373036797 |
| chrY | 4162699 | 4162699 | Tc-Mem_28738 | 0.036073034397972 |
| chr13 | 29225716 | 29225716 | Tc-Mem_6726 | 0.0368208032770627 |
| chr3 | 109456023 | 109456023 | Tc-Mem_17984 | 0.0376568144845525 |
| chrX | 139570576 | 139570576 | Tc-Mem_28661 | 0.0382468990515992 |
| chr2 | 137486257 | 137486257 | Tc-Mem_14291 | 0.0383443482945466 |
| chr8 | 48443324 | 48443324 | Tc-Mem_25876 | 0.03932839532783 |
| chr22 | 24663862 | 24663862 | Tc-Mem_16590 | 0.040153682515336 |
| chr2 | 147794468 | 147794468 | Tc-Mem_14368 | 0.0404169883164463 |
| chr10 | 65211774 | 65211774 | Tc-Mem_3034 | 0.0414439260213844 |
| chr22 | 23882428 | 23882428 | Tc-Mem_16580 | 0.0415016564495633 |
| chr5 | 17171063 | 17171063 | Tc-Mem_20718 | 0.042862113301991 |
| chr5 | 26993755 | 26993755 | Tc-Mem_20807 | 0.0440877822991477 |
| chrX | 133423338 | 133423338 | Tc-Mem_28639 | 0.044144520767884 |
| chr1 | 191999064 | 191999064 | Tc-Mem_1778 | 0.0444562210953553 |
| chr18 | 27531293 | 27531293 | Tc-Mem_11502 | 0.0452456027242246 |
| chr20 | 12164506 | 12164506 | Tc-Mem_15364 | 0.0459666385273212 |
| chr3 | 51872819 | 51872819 | Tc-Mem_17469 | 0.0488055778853113 |
| chr3 | 79505273 | 79505273 | Tc-Mem_17784 | 0.0488914777972062 |
| chr22 | 49519784 | 49519784 | Tc-Mem_16992 | 0.050293354099474 |
| chr3 | 78469990 | 78469990 | Tc-Mem_17777 | 0.0509483081802789 |
| chr3 | 45629775 | 45629775 | Tc-Mem_17418 | 0.0512065139894767 |
| chr19 | 7258060 | 7258060 | Tc-Mem_12186 | 0.0516470039996925 |
| chr6 | 80971566 | 80971566 | Tc-Mem_22926 | 0.0524811055810681 |
| chr7 | 67760446 | 67760446 | Tc-Mem_24446 | 0.0533342066840213 |
| chr2 | 227093540 | 227093540 | Tc-Mem_14989 | 0.0534464234315375 |
| chr1 | 237215703 | 237215703 | Tc-Mem_2244 | 0.0545209359796692 |
| chr1 | 113636146 | 113636146 | Tc-Mem_1276 | 0.0546200220691103 |
| chr21 | 35257621 | 35257621 | Tc-Mem_16299 | 0.0565655676942736 |
| chr1 | 155274302 | 155274302 | Tc-Mem_1438 | 0.0570638400280232 |
| chr2 | 5899488 | 5899488 | Tc-Mem_12934 | 0.0572269898635671 |
| chr3 | 40401150 | 40401150 | Tc-Mem_17364 | 0.0618725324376812 |
| chr11 | 67984273 | 67984273 | Tc-Mem_4540 | 0.0639712926076213 |

HIV naive cytotoxic T cells

| | | | | |
|---|---|---|---|---|
| chr13 | 36318001 | 36318001 | Tc-Naive_2070 | −0.1656533797924642 |
| chr1 | 219844110 | 219844110 | Tc-Naive_586 | −0.1444780709217854 |
| chr10 | 49134813 | 49134813 | Tc-Naive_831 | −0.1238543767479578 |
| chr1 | 240785916 | 240785917 | Tc-Naive_661 | −0.1137891459160716 |
| chr13 | 23738604 | 23738604 | Tc-Naive_2035 | −0.1104160029780324 |
| chr13 | 19382290 | 19382290 | Tc-Naive_2013 | −0.1096581598532204 |
| chrX | 40104630 | 40104630 | Tc-Naive_8691 | −0.1085946687073998 |
| chr7 | 8522232 | 8522232 | Tc-Naive_7331 | −0.1066582832592752 |
| chr2 | 11602604 | 11602604 | Tc-Naive_4049 | −0.0985635103548915 |
| chr6 | 113887230 | 113887230 | Tc-Naive_7128 | −0.0974749730437222 |
| chr10 | 24408435 | 24408435 | Tc-Naive_760 | −0.0964062404339319 |
| chr7 | 9850653 | 9850653 | Tc-Naive_7335 | −0.0952411820144401 |
| chrX | 122031756 | 122031756 | Tc-Naive_8788 | −0.0922367796191234 |
| chrX | 117619368 | 117619368 | Tc-Naive_8781 | −0.0889429542284146 |
| chr2 | 113540954 | 113540954 | Tc-Naive_4321 | −0.0873547471296005 |
| chr4 | 175741095 | 175741095 | Tc-Naive_6238 | −0.0868829826373229 |
| chr18 | 59551754 | 59551754 | Tc-Naive_3668 | −0.0861641821570922 |
| chr11 | 116704972 | 116704972 | Tc-Naive_1490 | −0.0861316380635264 |
| chr12 | 31571686 | 31571686 | Tc-Naive_1675 | −0.0861011402124342 |
| chr5 | 127913502 | 127913502 | Tc-Naive_6613 | −0.0854650443680662 |
| chr6 | 67886847 | 67886847 | Tc-Naive_7013 | −0.0841406203856455 |
| chr15 | 45952792 | 45952792 | Tc-Naive_2640 | −0.0832466762102671 |
| chr16 | 61207035 | 61207035 | Tc-Naive_3024 | −0.0818756440612394 |
| chrY | 8835250 | 8835250 | Tc-Naive_8842 | −0.081864529737694 |
| chr16 | 83257470 | 83257470 | Tc-Naive_3103 | −0.0818541481198568 |
| chr8 | 22658874 | 22658874 | Tc-Naive_7876 | −0.0794208277087797 |
| chr12 | 110619579 | 110619579 | Tc-Naive_1880 | −0.0790659769861767 |
| chr5 | 5826426 | 5826426 | Tc-Naive_6350 | −0.0790063566580015 |
| chrX | 67556595 | 67556595 | Tc-Naive_8721 | −0.0779236945199299 |
| chr11 | 15480530 | 15480530 | Tc-Naive_1182 | −0.0764461554352465 |
| chrX | 68065678 | 68065678 | Tc-Naive_8723 | −0.0753435699561516 |
| chr9 | 88731996 | 88731996 | Tc-Naive_8415 | −0.0748503368692004 |
| chr5 | 166216389 | 166216389 | Tc-Naive_6733 | −0.0738730122006205 |
| chr2 | 7749237 | 7749237 | Tc-Naive_4036 | −0.0738675297581518 |
| chr11 | 71272627 | 71272627 | Tc-Naive_1369 | −0.0732608185940859 |
| chrX | 10676859 | 10676859 | Tc-Naive_8639 | −0.0730517561748329 |
| chr11 | 31211678 | 31211678 | Tc-Naive_1225 | −0.0692720491618017 |
| chr8 | 36445661 | 36445661 | Tc-Naive_7927 | −0.0688097771472866 |
| chr16 | 69767584 | 69767584 | Tc-Naive_3062 | −0.068373159723364 |
| chr22 | 29307412 | 29307412 | Tc-Naive_5178 | −0.0669116709193988 |
| chr17 | 72575551 | 72575551 | Tc-Naive_3423 | −0.0665548456200421 |
| chr10 | 23158892 | 23158892 | Tc-Naive_754 | −0.0663604320218867 |
| chr16 | 29381891 | 29381891 | Tc-Naive_2928 | −0.0662291826450503 |
| chr9 | 114406092 | 114406092 | Tc-Naive_8493 | −0.0660284730286194 |
| chr1 | 98629694 | 98629694 | Tc-Naive_326 | −0.0658882175260273 |
| chr8 | 131696194 | 131696194 | Tc-Naive_8177 | −0.0657318496266427 |
| chr11 | 134762843 | 134762843 | Tc-Naive_1581 | −0.0648246455027748 |
| chr2 | 106587418 | 106587418 | Tc-Naive_4304 | −0.0643417974120025 |
| chr18 | 30650392 | 30650392 | Tc-Naive_3578 | −0.0638981442244086 |
| chr4 | 184993360 | 184993360 | Tc-Naive_6269 | −0.0629602844386712 |
| chr14 | 105124719 | 105124719 | Tc-Naive_2543 | −0.0620518056966362 |
| chr7 | 132307640 | 132307640 | Tc-Naive_7678 | −0.0618332620870029 |
| chr2 | 210551170 | 210551170 | Tc-Naive_4575 | −0.0617307411630294 |
| chr4 | 40942631 | 40942631 | Tc-Naive_5959 | 0.0628245962957462 |
| chr22 | 49350993 | 49350993 | Tc-Naive_5288 | 0.0633426525735555 |
| chr6 | 123695627 | 123695627 | Tc-Naive_7164 | 0.0653593186986664 |
| chr8 | 63144072 | 63144072 | Tc-Naive_8026 | 0.0665452624033475 |
| chr21 | 26161010 | 26161010 | Tc-Naive_5027 | 0.0693222626834543 |
| chr2 | 21587385 | 21587385 | Tc-Naive_4084 | 0.0701799371918608 |
| chr3 | 135561189 | 135561189 | Tc-Naive_5655 | 0.070265676399005 |
| chr3 | 69199250 | 69199250 | Tc-Naive_5483 | 0.0705256743097652 |
| chr10 | 133277097 | 133277097 | Tc-Naive_1115 | 0.0709062999539395 |
| chrX | 143482134 | 143482134 | Tc-Naive_8818 | 0.072049672184078 |
| chr6 | 152489476 | 152489476 | Tc-Naive_7217 | 0.0732186358562453 |
| chr18 | 28939088 | 28939088 | Tc-Naive_3574 | 0.0734727052635946 |
| chr2 | 116061079 | 116061079 | Tc-Naive_4331 | 0.0739424754118819 |
| chr1 | 207918178 | 207918178 | Tc-Naive_555 | 0.0742679939464863 |
| chr15 | 99496485 | 99496485 | Tc-Naive_2793 | 0.0749215230466994 |
| chr1 | 248944970 | 248944970 | Tc-Naive_686 | 0.0753259119665259 |
| chr12 | 73671663 | 73671663 | Tc-Naive_1791 | 0.0763306774852149 |
| chr8 | 24826063 | 24826063 | Tc-Naive_7887 | 0.0773219775965902 |
| chr3 | 177193804 | 177193804 | Tc-Naive_5747 | 0.0775146349821536 |
| chr15 | 32484704 | 32484704 | Tc-Naive_2605 | 0.0787469125401996 |
| chr3 | 169180060 | 169180060 | Tc-Naive_5723 | 0.0788960131472575 |
| chr7 | 49522436 | 49522436 | Tc-Naive_7450 | 0.0803917488466278 |
| chr1 | 37996290 | 37996300 | Tc-Naive_212 | 0.0807653526679527 |
| chr7 | 118794504 | 118794504 | Tc-Naive_7640 | 0.081107526244297 |
| chr11 | 69539791 | 69539791 | Tc-Naive_1352 | 0.0862579339029351 |
| chr6 | 137107243 | 137107243 | Tc-Naive_7183 | 0.0864074944933533 |
| chr13 | 76440880 | 76440880 | Tc-Naive_2148 | 0.0864963856785863 |
| chrX | 20903181 | 20903181 | Tc-Naive_8658 | 0.0871216985971228 |
| chr2 | 219529458 | 219529458 | Tc-Naive_4600 | 0.0882946428239016 |
| chr6 | 118595463 | 118595463 | Tc-Naive_7147 | 0.0926060370740557 |
| chrX | 25458907 | 25458907 | Tc-Naive_8668 | 0.0936662464158183 |
| chr11 | 26720294 | 26720294 | Tc-Naive_1212 | 0.0944764985449932 |
| chrY | 6001673 | 6001673 | Tc-Naive_8839 | 0.0946992132043629 |
| chr2 | 64661906 | 64661906 | Tc-Naive_4194 | 0.0960951059419406 |
| chr19 | 40190546 | 40190546 | Tc-Naive_3910 | 0.0968281018032633 |
| chr10 | 59389489 | 59389489 | Tc-Naive_864 | 0.097422188633321 |
| chr11 | 303784 | 303784 | Tc-Naive_1119 | 0.0981241058088785 |
| chr22 | 27475500 | 27475500 | Tc-Naive_5174 | 0.1048605896366293 |
| chr3 | 63159107 | 63159107 | Tc-Naive_5462 | 0.1068164114563307 |
| chrY | 20946217 | 20946217 | Tc-Naive_8856 | 0.1084483389916461 |
| chr2 | 3480100 | 3480100 | Tc-Naive_4024 | 0.1110166128257258 |
| chr16 | 57004147 | 57004147 | Tc-Naive_3009 | 0.1199911583327275 |
| chr10 | 34252804 | 34252804 | Tc-Naive_786 | 0.1213824249788863 |
| chr11 | 119978679 | 119978679 | Tc-Naive_1505 | 0.1249381366666472 |
| chr10 | 5042783 | 5042783 | Tc-Naive_711 | 0.1355723771379346 |
| chr1 | 167938086 | 167938086 | Tc-Naive_468 | 0.1365394651371586 |
| chrX | 46125696 | 46125696 | Tc-Naive_8697 | 0.1368276817799909 |

HIV Memory T cells

| | | | | |
|---|---|---|---|---|
| chr2 | 122972080 | 122972080 | Th-Mem_7058 | −0.1135203819273826 |
| chr15 | 81707824 | 81707824 | Th-Mem_4483 | −0.1089512515259799 |
| chr8 | 13692136 | 13692136 | Th-Mem_12721 | −0.1057598785900898 |
| chr1 | 85771178 | 85771178 | Th-Mem_550 | −0.1004295896937793 |
| chr6 | 470611 | 470611 | Th-Mem_11119 | −0.0888190599596938 |
| chr5 | 76456892 | 76456892 | Th-Mem_10615 | −0.0803537100203017 |
| chr9 | 100615637 | 100615637 | Th-Mem_13701 | 0.0801151044413893 |
| chr19 | 34606064 | 34606064 | Th-Mem_6288 | −0.0770966540673786 |
| chr5 | 132000412 | 132000412 | Th-Mem_10829 | −0.0755360315195527 |
| chrX | 132730813 | 132730813 | Th-Mem_14351 | −0.0734882406594434 |
| chr12 | 69179612 | 69179612 | Th-Mem_2965 | −0.073326338817475 |
| chr2 | 224167603 | 224167603 | Th-Mem_7487 | −0.0720025300477695 |
| chr20 | 20372789 | 20372789 | Th-Mem_7754 | −0.0715026284477462 |
| chr7 | 119466910 | 119466910 | Th-Mem_12417 | −0.0705090627203868 |
| chr5 | 108003990 | 108003990 | Th-Mem_10730 | −0.0695448036662488 |
| chr4 | 36679546 | 36679546 | Th-Mem_9687 | −0.068760035942762 |
| chr3 | 166689833 | 166689833 | Th-Mem_9301 | −0.0681664601034425 |
| chrX | 153803824 | 153803824 | Th-Mem_14431 | −0.0676289712505731 |
| chr7 | 156061141 | 156061141 | Th-Mem_12595 | −0.066454856394121 |
| chr6 | 161639336 | 161639336 | Th-Mem_11793 | −0.0659894573944754 |
| chr11 | 4514091 | 4514091 | Th-Mem_1934 | −0.0638953709828754 |
| chr11 | 22121438 | 22121438 | Th-Mem_2032 | −0.063647298679931 |
| chr11 | 77406653 | 77406653 | Th-Mem_2341 | −0.0632072839835413 |
| chr9 | 25713629 | 25713629 | Th-Mem_13467 | −0.0619817896024301 |
| chr1 | 207649510 | 207649510 | Th-Mem_962 | −0.0619788513429199 |
| chr5 | 125150377 | 125150377 | Th-Mem_10795 | −0.0619784966993959 |
| chr11 | 99008834 | 99008834 | Th-Mem_2421 | −0.0607454666595928 |
| chr9 | 69645170 | 69645170 | Th-Mem_13552 | −0.0599122875061804 |
| chr5 | 63219961 | 63219961 | Th-Mem_10576 | −0.059328823359303 |
| chr4 | 124476810 | 124476810 | Th-Mem_10023 | −0.0582945705754486 |
| chr6 | 11038396 | 11038396 | Th-Mem_11192 | −0.0582490136290598 |
| chr7 | 44229675 | 44229675 | Th-Mem_12078 | −0.0581444427655041 |
| chr13 | 110843953 | 110843953 | Th-Mem_3748 | −0.0573966342548246 |
| chr1 | 192901149 | 192901149 | Th-Mem_899 | −0.0572487929011222 |
| chr9 | 63648198 | 63648198 | Th-Mem_13534 | −0.056784269566565 |
| chr3 | 22853605 | 22853605 | Th-Mem_8692 | −0.0567164132768998 |
| chr17 | 75048466 | 75048466 | Th-Mem_5509 | −0.0567143362868365 |
| chr5 | 135058276 | 135058276 | Th-Mem_10855 | −0.0553409808391863 |
| chr16 | 31122738 | 31122738 | Th-Mem_4806 | −0.0548882691971788 |
| chr2 | 50740021 | 50740021 | Th-Mem_6728 | −0.0546184472596123 |
| chr12 | 130084101 | 130084101 | Th-Mem_3298 | −0.0536824618931326 |
| chr16 | 86127370 | 86127370 | Th-Mem_5061 | −0.052931951810954 |
| chr1 | 16044571 | 16044571 | Th-Mem_163 | −0.0527776764176687 |
| chr2 | 183629899 | 183629899 | Th-Mem_7316 | −0.0527048797673321 |
| chr18 | 79597146 | 79597146 | Th-Mem_5992 | −0.0525770698035138 |
| chrX | 10548581 | 10548581 | Th-Mem_13995 | −0.0525120925797412 |
| chr14 | 82624386 | 82624386 | Th-Mem_4049 | −0.0517103736700737 |
| chr2 | 227899747 | 227899747 | Th-Mem_7513 | −0.0511785739205336 |
| chr9 | 94839280 | 94839280 | Th-Mem_13671 | −0.0511170929187988 |
| chr11 | 75542842 | 75542842 | Th-Mem_2324 | −0.0505802859943924 |
| chrX | 91206254 | 91206254 | Th-Mem_14235 | −0.0495168163064135 |
| chrX | 87856466 | 87856466 | Th-Mem_14229 | −0.048547616643683 |
| chr12 | 128462412 | 128462412 | Th-Mem_3285 | −0.0483470214097181 |
| chr3 | 152565393 | 152565393 | Th-Mem_9251 | 0.0481916289380521 |
| chr2 | 112940385 | 112940385 | Th-Mem_6990 | 0.0485478192173123 |
| chrX | 4807308 | 4807308 | Th-Mem_13967 | 0.0486101744884156 |
| chr2 | 46530775 | 46530775 | Th-Mem_6705 | 0.0489812126283965 |
| chr21 | 17049478 | 17049478 | Th-Mem_8101 | 0.0489896382746944 |
| chr2 | 217905801 | 217905801 | Th-Mem_7450 | 0.0490192661970073 |
| chr17 | 34133344 | 34133344 | Th-Mem_5305 | 0.0504811733938547 |
| chrX | 32835608 | 32835608 | Th-Mem_14077 | 0.050531730446077 |
| chr7 | 34147750 | 34147750 | Th-Mem_12032 | 0.0507660209421634 |
| chr9 | 134428567 | 134428567 | Th-Mem_13905 | 0.0538383536511417 |
| chr1 | 64110967 | 64110967 | Th-Mem_466 | 0.0551616871819978 |
| chr10 | 122680985 | 122680985 | Th-Mem_1783 | 0.0561464832807336 |
| chr10 | 45375241 | 45375241 | Th-Mem_1420 | 0.0564226965921168 |
| chr5 | 180135527 | 180135527 | Th-Mem_11107 | 0.0572837634042523 |
| chr15 | 43203850 | 43203850 | Th-Mem_4294 | 0.0578170910989564 |
| chr10 | 118531381 | 118531381 | Th-Mem_1757 | 0.058550699422786 |
| chr10 | 131652655 | 131652655 | Th-Mem_1868 | 0.0586840140774262 |
| chr9 | 137031858 | 137031858 | Th-Mem_13941 | 0.0590562607260486 |
| chr22 | 20435263 | 20435263 | Th-Mem_8331 | 0.0598368560653159 |
| chr16 | 20303692 | 20303692 | Th-Mem_4740 | 0.0613465650965441 |
| chr22 | 48008570 | 48008570 | Th-Mem_8543 | 0.0617160461444254 |
| chr18 | 79589837 | 79589837 | Th-Mem_5991 | 0.0620090252782583 |
| chr4 | 109702149 | 109702149 | Th-Mem_9968 | 0.0622639778601644 |
| chr5 | 107263873 | 107263873 | Th-Mem_10722 | 0.0632740756106355 |
| chr9 | 130444380 | 130444380 | Th-Mem_13863 | 0.0638476543256855 |
| chr12 | 111946608 | 111946608 | Th-Mem_3159 | 0.064515502261377 |
| chr19 | 58557239 | 58557239 | Th-Mem_6474 | 0.0650378963824656 |
| chr19 | 720709 | 720709 | Th-Mem_6005 | 0.0654170168117787 |
| chr6 | 162633540 | 162633540 | Th-Mem_11802 | 0.0656024160534395 |
| chr19 | 50069353 | 50069353 | Th-Mem_6408 | 0.0659421546498653 |
| chrX | 82323666 | 82323666 | Th-Mem_14211 | 0.0666439852890746 |
| chr16 | 2991114 | 2991114 | Th-Mem_4637 | 0.0692704032440831 |
| chrX | 21605751 | 21605751 | Th-Mem_14041 | 0.0722009363789055 |
| chr3 | 15572466 | 15572466 | Th-Mem_8668 | 0.073133672703146 |
| chrX | 124002616 | 124002616 | Th-Mem_14319 | 0.073736349754165 |
| chr18 | 23756414 | 23756414 | Th-Mem_5709 | 0.0764120968712691 |
| chr6 | 94272785 | 94272785 | Th-Mem_11550 | 0.0770801439385013 |
| chr12 | 25943010 | 25943010 | Th-Mem_2770 | 0.0783265155950914 |
| chr6 | 55193482 | 55193482 | Th-Mem_11411 | 0.0783577547449218 |
| chr6 | 76747588 | 76747588 | Th-Mem_11487 | 0.0795976238797622 |
| chrX | 119356964 | 119356964 | Th-Mem_14306 | 0.0829523816590894 |
| chr5 | 155900530 | 155900530 | Th-Mem_10957 | 0.0871818250052899 |
| chrX | 94275909 | 94275909 | Th-Mem_14239 | 0.0919453214787488 |
| chrX | 146280373 | 146280373 | Th-Mem_14402 | 0.0921223968424835 |
| chr1 | 82980304 | 82980304 | Th-Mem_541 | 0.1050758195478173 |
| chr2 | 168264194 | 168264194 | Th-Mem_7269 | 0.1064653846985852 |
| chr12 | 95971738 | 95971738 | Th-Mem_3068 | 0.1475687357087419 |

HIV naïve T cell

| | | | | |
|---|---|---|---|---|
| chr2 | 75785635 | 75785635 | Th-Naive_3091 | −0.3205877658369488 |
| chrX | 77274714 | 77274714 | Th-Naive_6132 | −0.2296597254665233 |
| chrX | 154401369 | 154401369 | Th-Naive_6216 | −0.2101327647442869 |
| chr17 | 12291959 | 12291959 | Th-Naive_2294 | −0.2055496803612559 |
| chr4 | 63966863 | 63966863 | Th-Naive_4269 | −0.18165357891613 |
| chr12 | 7735534 | 7735534 | Th-Naive_1190 | −0.1711463434835189 |
| chr2 | 91571460 | 91571460 | Th-Naive_3119 | −0.1633990440594523 |
| chr17 | 40321131 | 40321131 | Th-Naive_2363 | −0.1582223949876905 |
| chr11 | 55373657 | 55373657 | Th-Naive_938 | −0.1481371520828593 |
| chrX | 16032129 | 16032129 | Th-Naive_6077 | −0.1478481390076353 |
| chr8 | 71701332 | 71701332 | Th-Naive_5647 | −0.1430349921518674 |
| chr14 | 105661448 | 105661448 | Th-Naive_1786 | −0.1347284245438253 |
| chr5 | 79101200 | 79101200 | Th-Naive_4606 | −0.134382378771893 |
| chr10 | 47019918 | 47019918 | Th-Naive_576 | −0.1271107115844683 |
| chr9 | 29057928 | 29057928 | Th-Naive_5844 | −0.1266916390016565 |
| chrX | 4837926 | 4837926 | Th-Naive_6060 | −0.1259164514849247 |
| chr14 | 57067778 | 57067778 | Th-Naive_1666 | −0.1224081583570196 |
| chr2 | 124774056 | 124774056 | Th-Naive_3182 | −0.1208788763210138 |
| chr10 | 84899672 | 84899672 | Th-Naive_652 | −0.1148111531556962 |
| chr13 | 49956704 | 49956704 | Th-Naive_1489 | −0.1137507816495727 |
| chr3 | 14632848 | 14632848 | Th-Naive_3850 | −0.1126775192267808 |
| chr6 | 98481748 | 98481748 | Th-Naive_5024 | −0.1095300781286082 |
| chr16 | 1113746 | 1113746 | Th-Naive_1992 | −0.1091737350411542 |
| chr6 | 11299995 | 11299995 | Th-Naive_4854 | −0.1054076592428996 |
| chr4 | 124435782 | 124435782 | Th-Naive_4346 | −0.1035606837720131 |
| chr5 | 141485979 | 141485979 | Th-Naive_4725 | −0.1031912965693432 |
| chrX | 7648578 | 7648578 | Th-Naive_6067 | −0.1030141576399422 |
| chr10 | 97974404 | 97974404 | Th-Naive_679 | −0.1019389954664597 |
| chr8 | 1288767 | 1288767 | Th-Naive_5490 | −0.1014633854896497 |
| chrX | 70436470 | 70436470 | Th-Naive_6124 | −0.100044273915351 |
| chr2 | 34069600 | 34069600 | Th-Naive_3014 | −0.0995241403891663 |
| chrX | 152429535 | 152429535 | Th-Naive_6210 | −0.0977223604254978 |
| chr5 | 9559109 | 9559109 | Th-Naive_4499 | −0.0971724468472938 |
| chr13 | 110921513 | 110921513 | Th-Naive_1577 | −0.0960538760161607 |
| chr12 | 88744430 | 88744430 | Th-Naive_1318 | −0.0902381997430241 |
| chr2 | 172985555 | 172985555 | Th-Naive_3265 | −0.0887538493881525 |
| chr11 | 116534115 | 116534115 | Th-Naive_1094 | −0.0831285967995522 |
| chr11 | 64385798 | 64385798 | Th-Naive_970 | −0.0817895505858085 |
| chr6 | 80743916 | 80743916 | Th-Naive_5005 | −0.0816449945293841 |
| chr4 | 146607681 | 146607681 | Th-Naive_4373 | −0.0798392216575091 |
| chr7 | 130294582 | 130294582 | Th-Naive_5403 | −0.079416271331911 |
| chr17 | 21562239 | 21562239 | Th-Naive_2318 | −0.0784787857139613 |
| chr12 | 69566751 | 69566751 | Th-Naive_1295 | −0.0753658815357295 |
| chr13 | 26412977 | 26412977 | Th-Naive_1458 | −0.074285653570877 |
| chr3 | 94755454 | 94755454 | Th-Naive_3980 | −0.0735052016177881 |
| chr8 | 32322626 | 32322626 | Th-Naive_5570 | −0.0733572727995228 |
| chr4 | 149837927 | 149837927 | Th-Naive_4378 | −0.0717710137353729 |
| chr6 | 1523955 | 1523955 | Th-Naive_4828 | −0.0715095729262898 |
| chr3 | 110298659 | 110298659 | Th-Naive_3999 | −0.0707765472316295 |
| chr11 | 49863095 | 49863095 | Th-Naive_924 | −0.0704678297168946 |
| chr5 | 146630249 | 146630249 | Th-Naive_4739 | −0.0695094712496997 |
| chr7 | 99579954 | 99579954 | Th-Naive_5347 | −0.0691303139589278 |
| chr10 | 108686054 | 108686054 | Th-Naive_709 | −0.0674111215157743 |
| chr4 | 96757778 | 96757778 | Th-Naive_4316 | −0.0662325048625809 |
| chr2 | 232639134 | 232639134 | Th-Naive_3351 | −0.0646557928599109 |
| chr7 | 56497610 | 56497610 | Th-Naive_5260 | 0.0640601703258501 |
| chr16 | 81739083 | 81739083 | Th-Naive_2189 | 0.0641395091916352 |
| chr16 | 28840056 | 28840056 | Th-Naive_2079 | 0.0642458555725745 |
| chr8 | 31576998 | 31576998 | Th-Naive_5568 | 0.0647185026796874 |
| chr17 | 28433479 | 28433479 | Th-Naive_2333 | 0.0673662818144748 |
| chr2 | 180529144 | 180529144 | Th-Naive_3275 | 0.0674188406417447 |
| chr6 | 135668345 | 135668345 | Th-Naive_5079 | 0.070017355315585 |
| chrX | 21656324 | 21656324 | Th-Naive_6085 | 0.0714361029185668 |
| chr1 | 6589535 | 6589535 | Th-Naive_40 | 0.0717433933439941 |
| chr9 | 73851279 | 73851279 | Th-Naive_5893 | 0.0719870780579516 |
| chr18 | 53107069 | 53107069 | Th-Naive_2607 | 0.0726605339140131 |
| chr10 | 79230561 | 79230561 | Th-Naive_635 | 0.0734981040366461 |
| chr9 | 34475305 | 34475305 | Th-Naive_5855 | 0.0753066034662683 |
| chr2 | 102348282 | 102348282 | Th-Naive_3140 | 0.0832851004771727 |
| chr5 | 80181093 | 80181093 | Th-Naive_4609 | 0.0862851054024502 |
| chr19 | 10754191 | 10754191 | Th-Naive_2732 | 0.086566442882832 |
| chr15 | 88340505 | 88340505 | Th-Naive_1943 | 0.0870448360243432 |
| chr3 | 70323367 | 70323367 | Th-Naive_3951 | 0.0906210120405375 |
| chr6 | 338143 | 338143 | Th-Naive_4820 | 0.0923278490029761 |
| chr17 | 38967735 | 38967735 | Th-Naive_2357 | 0.0962704421318794 |
| chr2 | 127563714 | 127563714 | Th-Naive_3192 | 0.101993039590335 |
| chr22 | 45497415 | 45497415 | Th-Naive_3778 | 0.1080032750501737 |
| chr3 | 50859954 | 50859954 | Th-Naive_3901 | 0.1083399091899789 |
| chr3 | 64774858 | 64774858 | Th-Naive_3940 | 0.114514489859385 |
| chr15 | 52112781 | 52112781 | Th-Naive_1858 | 0.1146861403853496 |
| chrX | 64235432 | 64235432 | Th-Naive_6120 | 0.1202510246729855 |
| chr15 | 18007343 | 18007343 | Th-Naive_1791 | 0.1256931749394541 |
| chr12 | 16407808 | 16407808 | Th-Naive_1205 | 0.126103022046722 |
| chrX | 40148313 | 40148313 | Th-Naive_6100 | 0.1399562558530613 |
| chrX | 48523750 | 48523750 | Th-Naive_6104 | 0.1400400925833337 |
| chr2 | 241479453 | 241479453 | Th-Naive_3393 | 0.1409088538024041 |
| chr5 | 128814166 | 128814166 | Th-Naive_4679 | 0.1489206886071366 |
| chr13 | 55146280 | 55146280 | Th-Naive_1503 | 0.1518035571273739 |
| chr10 | 40098425 | 40098425 | Th-Naive_558 | 0.1537674611122405 |
| chr6 | 124963509 | 124963509 | Th-Naive_5062 | 0.1590768220836705 |
| chr15 | 34496950 | 34496950 | Th-Naive_1833 | 0.1600201410067331 |
| chr18 | 63204719 | 63204719 | Th-Naive_2625 | 0.1725456494779357 |
| chr2 | 39665270 | 39665270 | Th-Naive_3027 | 0.1940101213690122 |
| chrY | 10178966 | 10178966 | Th-Naive_6221 | 0.1954084677544503 |
| chr3 | 7986138 | 7986138 | Th-Naive_3828 | 0.1957304867094556 |
| chr9 | 128082872 | 128082872 | Th-Naive_6004 | 0.2020534407676747 |
| chr17 | 36930515 | 36930515 | Th-Naive_2351 | 0.2042593091230727 |
| chr2 | 192875201 | 192875201 | Th-Naive_3288 | 0.2235089390038277 |
| chr15 | 75102176 | 75102176 | Th-Naive_1911 | 0.22558416005632 |
| chrX | 120575920 | 120575920 | Th-Naive_6178 | 0.2704667386587777 |

MRSA B-Mem

| | | | | |
|---|---|---|---|---|
| chr17 | 15296636 | 15296636 | B-Mem_4071 | −0.2289987295139121 |
| chr1 | 183122828 | 183122828 | B-Mem_679 | −0.2280262668933382 |
| chr2 | 10795735 | 10795735 | B-Mem_5037 | −0.1759242591453332 |
| chr20 | 29233607 | 29233607 | B-Mem_6023 | −0.173059546058048 |
| chr17 | 81227406 | 81227406 | B-Mem_4391 | −0.1602924489778771 |
| chr7 | 2217564 | 2217564 | B-Mem_9223 | −0.1601083672737711 |
| chr8 | 8241054 | 8241054 | B-Mem_9839 | −0.1534764100543203 |
| chr11 | 117968592 | 117968592 | B-Mem_1955 | −0.1504606949693221 |
| chr10 | 19698715 | 19698715 | B-Mem_1041 | −0.1454847909303266 |
| chr2 | 3606700 | 3606700 | B-Mem_5012 | −0.1450468530147636 |
| chr8 | 81259796 | 81259796 | B-Mem_10099 | −0.1402650700899006 |
| chr8 | 7037468 | 7037468 | B-Mem_9838 | −0.1335814276546568 |
| chr2 | 8581385 | 8581385 | B-Mem_5027 | −0.124069351511758 |
| chr1 | 1753033 | 1753033 | B-Mem_8 | −0.1162130422726715 |
| chr16 | 272935 | 272935 | B-Mem_3518 | −0.1116613922473185 |
| chr22 | 20685517 | 20685517 | B-Mem_6409 | −0.1089584501635997 |
| chr14 | 50519412 | 50519412 | B-Mem_2924 | −0.1084844480273459 |
| chr13 | 31061655 | 31061655 | B-Mem_2546 | −0.1067646768203196 |
| chr5 | 120591342 | 120591342 | B-Mem_8345 | −0.1046736659539073 |
| chr1 | 29684388 | 29684388 | B-Mem_183 | −0.1035085720378389 |
| chr10 | 132174724 | 132174724 | B-Mem_1494 | −0.1034941524581127 |
| chr1 | 216829849 | 216829849 | B-Mem_793 | −0.10174469736628 |
| chr4 | 26585695 | 26585695 | B-Mem_7387 | −0.1002245139233094 |
| chr16 | 4759007 | 4759007 | B-Mem_3552 | −0.1001415380482136 |
| chr5 | 143110228 | 143110228 | B-Mem_8432 | −0.0983968599124922 |
| chr8 | 48216281 | 48216281 | B-Mem_10001 | −0.0949081567622666 |
| chr1 | 1792926 | 1792926 | B-Mem_10 | −0.0944134796611691 |
| chr11 | 4592463 | 4592463 | B-Mem_1554 | −0.0943688737236673 |
| chr4 | 54637645 | 54637645 | B-Mem_7471 | −0.0929915015520255 |
| chrX | 63755162 | 63755162 | B-Mem_10994 | −0.0920087002853465 |
| chr6 | 24313994 | 24313994 | B-Mem_8746 | −0.0878061550028404 |
| chr3 | 59484197 | 59484197 | B-Mem_6817 | −0.0854188186053938 |
| chr22 | 41244655 | 41244655 | B-Mem_6512 | −0.0821299521921312 |
| chr10 | 12146807 | 12146807 | B-Mem_1017 | −0.0787990295233153 |
| chr12 | 124366873 | 124366873 | B-Mem_2442 | −0.0771561810835083 |
| chr6 | 44056524 | 44056524 | B-Mem_8852 | −0.0741926660049038 |
| chr3 | 152141153 | 152141153 | B-Mem_7109 | −0.0722444269605401 |
| chr18 | 3385901 | 3385901 | B-Mem_4418 | −0.0684458906154051 |
| chr12 | 86210170 | 86210170 | B-Mem_2307 | −0.0639640444288974 |
| chr2 | 205306982 | 205306982 | B-Mem_5734 | −0.0627217274323578 |
| chr21 | 33331291 | 33331291 | B-Mem_6314 | −0.0591704904870249 |
| chr1 | 17035846 | 17035846 | B-Mem_106 | −0.0591348430026328 |
| chr12 | 3195704 | 3195704 | B-Mem_2056 | −0.0584122013983493 |
| chrX | 101446999 | 101446999 | B-Mem_11085 | 0.0588448638147107 |
| chrY | 8578049 | 8578049 | B-Mem_11273 | 0.0598723901970974 |
| chr20 | 4333188 | 4333188 | B-Mem_5926 | 0.0607177120147246 |
| chr13 | 98988231 | 98988231 | B-Mem_2751 | 0.0616962841251002 |
| chr11 | 5763462 | 5763462 | B-Mem_1562 | 0.0620310876669244 |
| chr14 | 41115429 | 41115429 | B-Mem_2894 | 0.0639179769081858 |
| chr2 | 113779757 | 113779757 | B-Mem_5442 | 0.0642048322402495 |
| chr4 | 22962121 | 22962121 | B-Mem_7373 | 0.0646409154602408 |
| chr13 | 110853676 | 110853676 | B-Mem_2799 | 0.0685120180510659 |
| chr21 | 31857990 | 31857990 | B-Mem_6308 | 0.0689839687491577 |
| chr1 | 4078164 | 4078164 | B-Mem_24 | 0.0694522190451988 |
| chr2 | 141677722 | 141677722 | B-Mem_5554 | 0.070796609099339 |
| chr3 | 90853207 | 90853207 | B-Mem_6936 | 0.0709000394668955 |
| chr2 | 117626962 | 117626962 | B-Mem_5453 | 0.0721247957161355 |
| chr7 | 97923081 | 97923081 | B-Mem_9559 | 0.0724815088362317 |
| chrX | 78159815 | 78159815 | B-Mem_11026 | 0.073420874884027 |
| chr1 | 178570512 | 178570512 | B-Mem_660 | 0.0744443188395293 |
| chr8 | 111617400 | 111617400 | B-Mem_10195 | 0.0747849008684658 |
| chrX | 11701444 | 11701444 | B-Mem_10817 | 0.0775466989170125 |
| chr1 | 7463404 | 7463404 | B-Mem_43 | 0.0854021886221037 |
| chr2 | 46181201 | 46181201 | B-Mem_5199 | 0.0855543343182102 |
| chr2 | 87992814 | 87992814 | B-Mem_5345 | 0.087488798379379 |
| chr21 | 18539032 | 18539032 | B-Mem_6254 | 0.0884350919691784 |
| chr4 | 114863941 | 114863941 | B-Mem_7691 | 0.0884747679656103 |
| chr4 | 72595764 | 72595764 | B-Mem_7536 | 0.089001508445966 |
| chr10 | 52735425 | 52735425 | B-Mem_1171 | 0.089256257081031 |
| chr1 | 214553133 | 214553133 | B-Mem_786 | 0.0950669176978814 |
| chrY | 7501223 | 7501223 | B-Mem_11270 | 0.0960070171392975 |
| chr5 | 2111350 | 2111350 | B-Mem_7976 | 0.0981745048650272 |
| chrX | 88394501 | 88394501 | B-Mem_11049 | 0.1007105478442928 |
| chr4 | 157176032 | 157176032 | B-Mem_7840 | 0.1008093152125645 |
| chr2 | 55427815 | 55427815 | B-Mem_5222 | 0.1031253174932682 |
| chr6 | 160508305 | 160508305 | B-Mem_9148 | 0.1043028525231406 |
| chrY | 20620370 | 20620370 | B-Mem_11285 | 0.1048543598698043 |
| chr8 | 30270458 | 30270458 | B-Mem_9943 | 0.1056673462604601 |
| chr10 | 35909130 | 35909130 | B-Mem_1099 | 0.1094749125049247 |
| chrY | 4295107 | 4295107 | B-Mem_11266 | 0.1166269954451404 |
| chr17 | 71125149 | 71125149 | B-Mem_4326 | 0.1197236222206425 |
| chr2 | 103499624 | 103499624 | B-Mem_5405 | 0.1201879006141767 |
| chr10 | 40846329 | 40846329 | B-Mem_1117 | 0.1222972090833608 |
| chr3 | 90334743 | 90334743 | B-Mem_6930 | 0.126623670229096 |
| chrY | 13679935 | 13679935 | B-Mem_11278 | 0.1283080066525392 |
| chr8 | 19656840 | 19656840 | B-Mem_9895 | 0.1303300493464766 |
| chr9 | 103243792 | 103243792 | B-Mem_10604 | 0.1346131577656344 |
| chrY | 11879993 | 11879993 | B-Mem_11276 | 0.1416487852051351 |
| chr3 | 69363490 | 69363490 | B-Mem_6859 | 0.1465912163849388 |
| chrY | 7837992 | 7837992 | B-Mem_11272 | 0.1471970792989361 |
| chrX | 132277327 | 132277327 | B-Mem_11180 | 0.1517962055471871 |
| chr3 | 109133555 | 109133555 | B-Mem_6981 | 0.1538727694440567 |
| chr9 | 103606576 | 103606576 | B-Mem_10606 | 0.158001940129439 |
| chrY | 20980147 | 20980147 | B-Mem_11286 | 0.2000951368218172 |
| chrY | 5621467 | 5621467 | B-Mem_11268 | 0.2113236390823585 |
| chr9 | 64457498 | 64457498 | B-Mem_10476 | 0.2207370392825119 |
| chrY | 21704538 | 21704538 | B-Mem_11287 | 0.2338356954203429 |
| chrY | 16517439 | 16517439 | B-Mem_11282 | 0.2546023793976226 |
| chrY | 16059463 | 16059463 | B-Mem_11281 | 0.2555863293205433 |
| chrY | 4836316 | 4836316 | B-Mem_11267 | 0.3057910986842665 |

MRSA B-Naïve

| | | | | |
|---|---|---|---|---|
| chr4 | 160480478 | 160480478 | B-Naive_3673 | −0.2958594454384776 |
| chr11 | 105778472 | 105778472 | B-Naive_841 | −0.2946914208087604 |
| chr5 | 20415689 | 20415689 | B-Naive_3771 | −0.2853453200018174 |
| chr19 | 43001557 | 43001557 | B-Naive_2250 | −0.2756192237110305 |
| chr12 | 131509811 | 131509811 | B-Naive_1136 | −0.2238121431328402 |
| chr8 | 17584626 | 17584626 | B-Naive_4686 | −0.2214951696640151 |
| chr3 | 59183360 | 59183360 | B-Naive_3167 | 0.2158061835341498 |
| chr2 | 129699892 | 129699892 | B-Naive_2525 | −0.2044814058269514 |
| chr9 | 100037475 | 100037475 | B-Naive_5037 | −0.1919160302497972 |
| chr12 | 13378351 | 13378351 | B-Naive_952 | −0.1795494301622326 |
| chr7 | 131285012 | 131285012 | B-Naive_4580 | −0.1767470133843733 |
| chr11 | 7810085 | 7810085 | B-Naive_684 | −0.1595663347005383 |
| chr17 | 343396 | 343396 | B-Naive_1796 | −0.1591373811346803 |
| chrX | 142425229 | 142425229 | B-Naive_5323 | −0.1554625356195769 |
| chr2 | 201680507 | 201680507 | B-Naive_2637 | −0.1538405774004541 |
| chr15 | 100459444 | 100459444 | B-Naive_1597 | −0.1497070571715809 |
| chr8 | 21619239 | 21619239 | B-Naive_4704 | −0.1450549257805316 |
| chr16 | 55256721 | 55256721 | B-Naive_1713 | 0.1448296875002291 |
| chr1 | 216829849 | 216829849 | B-Naive_337 | −0.1433584587752986 |
| chr8 | 132400453 | 132400453 | B-Naive_4874 | −0.14251878114521 |
| chr15 | 85173793 | 85173793 | B-Naive_1560 | −0.1412676764736458 |
| chr11 | 36520568 | 36520568 | B-Naive_736 | −0.1409400897568674 |
| chr3 | 83598055 | 83598055 | B-Naive_3205 | −0.137200339368646 |
| chr12 | 31576404 | 31576404 | B-Naive_998 | −0.1353551961958473 |
| chr2 | 150219094 | 150219094 | B-Naive_2556 | −0.126398727146151 |
| chr15 | 78714099 | 78714099 | B-Naive_1553 | −0.1192435902796516 |
| chr13 | 48738938 | 48738938 | B-Naive_1202 | −0.1191371344252559 |
| chr3 | 18889643 | 18889643 | B-Naive_3095 | −0.1160476593164075 |
| chr13 | 49528673 | 49528673 | B-Naive_1206 | −0.1013059996516502 |
| chr6 | 10206489 | 10206489 | B-Naive_4053 | −0.1002819170456438 |
| chr12 | 28917645 | 28917645 | B-Naive_988 | −0.0858756167291658 |
| chr22 | 21016471 | 21016471 | B-Naive_2982 | −0.0853398727694994 |
| chr13 | 65163179 | 65163179 | B-Naive_1235 | −0.0822556875580517 |
| chr13 | 92032969 | 92032969 | B-Naive_1268 | −0.0818623227866115 |
| chr15 | 62890660 | 62890660 | B-Naive_1519 | −0.0817575062774486 |
| chr17 | 6553748 | 6553748 | B-Naive_1815 | −0.0813094755306748 |
| chr5 | 54858380 | 54858380 | B-Naive_3827 | −0.0744019021012716 |
| chr14 | 75827257 | 75827257 | B-Naive_1388 | −0.0721198946038491 |
| chr1 | 14919825 | 14919825 | B-Naive_41 | −0.0715337200332542 |
| chr22 | 26723098 | 26723098 | B-Naive_2997 | −0.0682059885990981 |
| chr2 | 221576550 | 221576550 | B-Naive_2662 | −0.0644491936549687 |
| chr7 | 96416241 | 96416241 | B-Naive_4532 | −0.0610599547081695 |
| chr9 | 19238243 | 19238243 | B-Naive_4945 | −0.0516328658111293 |
| chr8 | 900591 | 900591 | B-Naive_4645 | −0.0496290305860663 |
| chr4 | 115008165 | 115008165 | B-Naive_3607 | 0.0506968257691038 |
| chr4 | 15243792 | 15243792 | B-Naive_3442 | 0.0512132977118125 |
| chr19 | 1004326 | 1004326 | B-Naive_2135 | 0.0571655705224997 |
| chr3 | 109172089 | 109172089 | B-Naive_3236 | 0.0610444240330376 |
| chr9 | 71023872 | 71023872 | B-Naive_5002 | 0.0615982753468455 |
| chr4 | 25869170 | 25869170 | B-Naive_3459 | 0.0634276285985973 |
| chr18 | 48987821 | 48987821 | B-Naive_2060 | 0.0656939620239701 |
| chr6 | 162078810 | 162078810 | B-Naive_4298 | 0.0706766490943914 |
| chr4 | 23615136 | 23615136 | B-Naive_3454 | 0.0707532889961584 |
| chr1 | 234378125 | 234378125 | B-Naive_370 | 0.0724761182783984 |
| chr4 | 37512578 | 37512578 | B-Naive_3475 | 0.0734557033025143 |
| chr9 | 12341832 | 12341832 | B-Naive_4937 | 0.0738284932862416 |
| chr8 | 62257398 | 62257398 | B-Naive_4769 | 0.0750689530394614 |
| chr9 | 61658168 | 61658168 | B-Naive_4987 | 0.0800193270503164 |
| chr12 | 67784736 | 67784736 | B-Naive_1045 | 0.0800261742056717 |
| chr12 | 34796338 | 34796338 | B-Naive_1002 | 0.0816674423329223 |
| chr8 | 122435647 | 122435647 | B-Naive_4853 | 0.0824887401206305 |
| chr18 | 53614442 | 53614442 | B-Naive_2074 | 0.0840040338158314 |
| chr5 | 62304568 | 62304568 | B-Naive_3848 | 0.0860219911006442 |
| chr17 | 27940561 | 27940561 | B-Naive_1863 | 0.0900096421282354 |
| chr5 | 151924356 | 151924356 | B-Naive_3968 | 0.0926360164974363 |
| chr1 | 168207980 | 168207980 | B-Naive_263 | 0.0992529471445711 |
| chr2 | 149075916 | 149075916 | B-Naive_2553 | 0.1000324850592382 |
| chr13 | 42455894 | 42455894 | B-Naive_1189 | 0.100931549897628 |
| chr9 | 25595809 | 25595809 | B-Naive_4959 | 0.1035759360649139 |
| chr19 | 18680958 | 18680958 | B-Naive_2202 | 0.1047376592139852 |
| chr8 | 19656840 | 19656840 | B-Naive_4696 | 0.1066163734065877 |
| chr17 | 62618039 | 62618039 | B-Naive_1934 | 0.1067722506397058 |
| chr1 | 842367 | 842367 | B-Naive_0 | 0.1068909078411023 |
| chr7 | 51616512 | 51616512 | B-Naive_4454 | 0.1071311787079689 |
| chr22 | 48660364 | 48660364 | B-Naive_3052 | 0.1206147116768385 |
| chr4 | 140480071 | 140480071 | B-Naive_3633 | 0.1256747070351996 |
| chr6 | 68932106 | 68932106 | B-Naive_4181 | 0.1287143955855828 |
| chr5 | 63165168 | 63165168 | B-Naive_3851 | 0.1303924702811324 |
| chr7 | 3054031 | 3054031 | B-Naive_4353 | 0.1311691212712237 |
| chrY | 11516034 | 11516034 | B-Naive_5356 | 0.1352349404362135 |
| chr2 | 24938517 | 24938517 | B-Naive_2350 | 0.1370167428736367 |
| chr22 | 19084180 | 19084180 | B-Naive_2976 | 0.144400599918357 |
| chrX | 43016783 | 43016783 | B-Naive_5187 | 0.1485675365532111 |
| chr15 | 55204654 | 55204654 | B-Naive_1505 | 0.1503578777479437 |
| chr17 | 49441654 | 49441654 | B-Naive_1908 | 0.1538009928645101 |
| chr6 | 48468943 | 48468943 | B-Naive_4153 | 0.1646163313867866 |
| chr7 | 154100602 | 154100602 | B-Naive_4622 | 0.167964512673233 |
| chr8 | 20175075 | 20175075 | B-Naive_4698 | 0.1703116939416256 |
| chr17 | 60762630 | 60762630 | B-Naive_1930 | 0.1739876038961848 |
| chr19 | 39859122 | 39859122 | B-Naive_2245 | 0.1751445023531181 |
| chr2 | 190366547 | 190366547 | B-Naive_2622 | 0.1934975606889072 |
| chr9 | 111686239 | 111686239 | B-Naive_5055 | 0.2048717071994511 |
| chrY | 15158609 | 15158609 | B-Naive_5358 | 0.2343171538260372 |
| chr20 | 9893184 | 9893184 | B-Naive_2752 | 0.2367222965246935 |
| chr4 | 28977588 | 28977588 | B-Naive_3466 | 0.2538977860039731 |
| chr7 | 147312503 | 147312503 | B-Naive_4609 | 0.2697029721611722 |
| chr2 | 123634768 | 123634768 | B-Naive_2510 | 0.2737898960685332 |
| chr17 | 80918469 | 80918469 | B-Naive_1981 | 0.2804413320819313 |
| chr12 | 1345219 | 1345219 | B-Naive_914 | 0.3256283874359206 |
| chr6 | 159201769 | 159201769 | B-Naive_4290 | 0.3573934465808278 |

MRSA Monocyte

| | | | | |
|---|---|---|---|---|
| chr5 | 161161554 | 161161554 | Monocyte_11512 | −0.1745133133724374 |
| chr12 | 32672893 | 32672893 | Monocyte_2892 | −0.150706739840215 |
| chr13 | 47975770 | 47975782 | Monocyte_3489 | −0.1310754380493574 |
| chr18 | 50830330 | 50830330 | Monocyte_6168 | −0.1139856102464796 |
| chr1 | 179922892 | 179922892 | Monocyte_798 | −0.1137069264415606 |
| chr20 | 62757709 | 62757709 | Monocyte_8389 | −0.1070439760098795 |
| chr2 | 152922388 | 152922388 | Monocyte_7524 | −0.1044899636800415 |
| chr1 | 29013153 | 29013153 | Monocyte_245 | −0.1026047710938669 |
| chr10 | 50329590 | 50329590 | Monocyte_1468 | −0.100916788859776 |
| chr1 | 169940598 | 169940598 | Monocyte_756 | −0.0974688605400853 |
| chr22 | 50686869 | 50686869 | Monocyte_8929 | −0.0944942379821338 |
| chr4 | 118671407 | 118671407 | Monocyte_10445 | −0.0920797007842258 |
| chr12 | 52201629 | 52201629 | Monocyte_2965 | −0.091294145375792 |
| chr2 | 165667611 | 165667611 | Monocyte_7568 | −0.0908136345891081 |
| chr10 | 57491334 | 57491334 | Monocyte_1503 | −0.0903118888882971 |
| chr16 | 4397770 | 4397770 | Monocyte_4776 | −0.0888646363007948 |
| chr12 | 40341366 | 40341366 | Monocyte_2917 | −0.0870959140948915 |
| chr2 | 191075724 | 191075724 | Monocyte_7689 | −0.0845780222046922 |
| chr7 | 74115616 | 74115616 | Monocyte_12947 | −0.084337588126639 |
| chr6 | 44096433 | 44096433 | Monocyte_12002 | −0.0813265842056735 |
| chr5 | 8779430 | 8779443 | Monocyte_10874 | −0.0797929475565287 |
| chr4 | 176802207 | 176802207 | Monocyte_10700 | −0.0783397770159307 |
| chr11 | 86627869 | 86627869 | Monocyte_2406 | −0.0767368627412892 |
| chr5 | 172528615 | 172528615 | Monocyte_11572 | −0.0764560345999454 |
| chr12 | 31576404 | 31576404 | Monocyte_2886 | −0.0763486066583429 |
| chr1 | 15213859 | 15213910 | Monocyte_136 | −0.0735314669168384 |
| chr11 | 60067466 | 60067466 | Monocyte_2260 | −0.0728479548266767 |
| chr16 | 25657956 | 25657956 | Monocyte_4949 | −0.0696219531293276 |
| chr3 | 175855967 | 175855967 | Monocyte_9777 | −0.0695290429260847 |
| chr12 | 31447465 | 31447465 | Monocyte_2883 | −0.0694771183572971 |
| chr16 | 17762586 | 17762586 | Monocyte_4904 | −0.0687311120727278 |
| chr13 | 37089036 | 37089036 | Monocyte_3439 | −0.0666505420000683 |
| chr10 | 48703201 | 48703201 | Monocyte_1458 | −0.0663317061047753 |
| chr12 | 47710962 | 47710962 | Monocyte_2942 | −0.0645913986466835 |
| chr2 | 106537874 | 106537874 | Monocyte_7306 | −0.0644380575672026 |
| chr14 | 52818836 | 52818836 | Monocyte_3949 | −0.0638680069860591 |
| chr4 | 143289008 | 143289008 | Monocyte_10537 | −0.0625180290795022 |
| chr1 | 46018497 | 46018497 | Monocyte_317 | −0.0623668921357729 |
| chr10 | 12132568 | 12132568 | Monocyte_1259 | −0.060922021415317 |
| chrX | 17565087 | 17565140 | Monocyte_14782 | −0.0604561491771514 |
| chr6 | 166202334 | 166202504 | Monocyte_12463 | −0.059875524589817 |
| chr20 | 36922126 | 36922126 | Monocyte_8208 | −0.0579460083480278 |
| chr22 | 26786054 | 26786054 | Monocyte_8706 | −0.0568691719921439 |
| chr17 | 39058644 | 39058644 | Monocyte_5581 | −0.0561220667855688 |
| chr6 | 163344752 | 163344752 | Monocyte_12433 | −0.0547833218792344 |
| chrX | 85768796 | 85768796 | Monocyte_15027 | 0.0560249700235651 |
| chr9 | 111154930 | 111154930 | Monocyte_14520 | 0.0572921200228032 |
| chr9 | 78483913 | 78483913 | Monocyte_14367 | 0.0575229163300669 |
| chr20 | 62044851 | 62044851 | Monocyte_8376 | 0.0576907691329502 |
| chr10 | 51396261 | 51396261 | Monocyte_1472 | 0.0580782511158534 |
| chr3 | 127072252 | 127072252 | Monocyte_9559 | 0.0608838612898952 |
| chr12 | 29965642 | 29965642 | Monocyte_2877 | 0.0617055970210854 |
| chr13 | 34254011 | 34254011 | Monocyte_3436 | 0.0619883290012792 |
| chr3 | 113122663 | 113122663 | Monocyte_9485 | 0.0628503413330045 |
| chr15 | 92932891 | 92932891 | Monocyte_4669 | 0.0653562302627932 |
| chr7 | 25017849 | 25017849 | Monocyte_12697 | 0.0656186114585004 |
| chr19 | 28114210 | 28114210 | Monocyte_6554 | 0.0678952536956605 |
| chr4 | 26255618 | 26255618 | Monocyte_10062 | 0.0681151263518076 |
| chr1 | 113679894 | 113679894 | Monocyte_583 | 0.068551262316434 |
| chr7 | 136069662 | 136069662 | Monocyte_13182 | 0.0737879589162129 |
| chrX | 107979417 | 107979417 | Monocyte_15107 | 0.0747814363184015 |
| chr7 | 158126196 | 158126196 | Monocyte_13342 | 0.0748901953163068 |
| chr19 | 35090030 | 35090030 | Monocyte_6587 | 0.0756892062944771 |
| chr3 | 70784211 | 70784211 | Monocyte_9333 | 0.0779052149857798 |
| chr7 | 124205935 | 124205935 | Monocyte_13113 | 0.0781828564495937 |
| chrY | 4703387 | 4703387 | Monocyte_15314 | 0.078765989664739 |
| chr4 | 54304295 | 54304295 | Monocyte_10197 | 0.0794607489482235 |
| chr9 | 6499994 | 6499994 | Monocyte_14147 | 0.0818241238415136 |
| chr22 | 18944139 | 18944139 | Monocyte_8643 | 0.0842059717693594 |
| chr12 | 18650611 | 18650611 | Monocyte_2825 | 0.0859934391772752 |
| chr3 | 186488542 | 186488542 | Monocyte_9832 | 0.0875037998263362 |
| chr4 | 128070409 | 128070409 | Monocyte_10486 | 0.0886660372913199 |
| chr7 | 145241570 | 145241570 | Monocyte_13250 | 0.0920709910775197 |
| chr4 | 189553841 | 189553841 | Monocyte_10761 | 0.0934138502983706 |
| chrX | 108281996 | 108281996 | Monocyte_15109 | 0.0947330030589061 |
| chr7 | 104560163 | 104560163 | Monocyte_13053 | 0.098415595267331 |
| chr22 | 21083483 | 21083483 | Monocyte_8656 | 0.0991712552132743 |
| chr10 | 87160134 | 87160134 | Monocyte_1647 | 0.0999052054437168 |
| chrY | 11552011 | 11552011 | Monocyte_15319 | 0.100439128465068 |
| chr20 | 30293700 | 30293700 | Monocyte_8169 | 0.101272991221075 |
| chrX | 148301231 | 148301231 | Monocyte_15268 | 0.1024511520531766 |
| chr7 | 34803267 | 34803267 | Monocyte_12747 | 0.1045806209060729 |
| chr13 | 101413986 | 101413986 | Monocyte_3705 | 0.1064180823183455 |
| chr1 | 238739779 | 238739779 | Monocyte_1110 | 0.1075203782834284 |
| chrY | 10174869 | 10174869 | Monocyte_15317 | 0.1118956369056031 |
| chr15 | 90551943 | 90551943 | Monocyte_4654 | 0.1139080780387982 |
| chr4 | 97920507 | 97920507 | Monocyte_10372 | 0.1193253234588087 |
| chr3 | 9865647 | 9865647 | Monocyte_8987 | 0.1219432659304293 |
| chr1 | 179851733 | 179851733 | Monocyte_794 | 0.1244358418170671 |
| chr1 | 219043136 | 219043136 | Monocyte_979 | 0.1297760937249432 |
| chr14 | 18440417 | 18440417 | Monocyte_3785 | 0.1373849589718343 |
| chrY | 11729014 | 11729014 | Monocyte_15320 | 0.1455971003799059 |
| chr10 | 89998097 | 89998097 | Monocyte_1658 | 0.1462981311593861 |
| chr10 | 40928787 | 40928787 | Monocyte_1411 | 0.1619703500230118 |
| chrY | 19034686 | 19034686 | Monocyte_15329 | 0.1682173200428589 |
| chrY | 15036900 | 15036900 | Monocyte_15326 | 0.1753408576109027 |
| chr3 | 157729116 | 157729116 | Monocyte_9708 | 0.1831708814655633 |
| chr6 | 24957215 | 24957215 | Monocyte_11838 | 0.1857942269156539 |
| chr3 | 94795624 | 94795624 | Monocyte_9413 | 0.1987727446561799 |
| chrY | 14139681 | 14139681 | Monocyte_15323 | 0.2315094180994428 |

MRSA NK-cell1

| | | | | |
|---|---|---|---|---|
| chr2 | 219578607 | 219578607 | NK-cell1_3327 | −0.3196910163712663 |
| chr3 | 196155916 | 196155916 | NK-cell1_4199 | −0.2989237913965357 |
| chr18 | 25364405 | 25364405 | NK-cell1_2561 | −0.2820953637559257 |
| chr8 | 11491451 | 11491451 | NK-cell1_5703 | −0.2131628270708033 |
| chr9 | 133914054 | 133914054 | NK-cell1_6246 | −0.2035738948228062 |
| chr2 | 194645972 | 194645972 | NK-cell1_3267 | −0.1815120635349069 |
| chr2 | 102620402 | 102620407 | NK-cell1_3072 | −0.1792833724620249 |
| chr1 | 63471396 | 63471396 | NK-cell1_178 | −0.1650950168330998 |
| chr3 | 114249127 | 114249127 | NK-cell1_4044 | −0.1627738488531636 |
| chr6 | 82411392 | 82411392 | NK-cell1_5184 | −0.1587792962942664 |
| chr4 | 29289814 | 29289814 | NK-cell1_4286 | −0.1531822264037039 |
| chr19 | 10123767 | 10123767 | NK-cell1_2704 | −0.1529473142174018 |
| chr10 | 13760211 | 13760211 | NK-cell1_575 | −0.1488512872083104 |
| chr13 | 66830043 | 66830043 | NK-cell1_1616 | −0.1475708234002744 |
| chr3 | 11506462 | 11506462 | NK-cell1_3851 | −0.1430584968974297 |
| chr1 | 85505110 | 85505110 | NK-cell1_225 | −0.13388743739966 |
| chr20 | 29304689 | 29304689 | NK-cell1_3491 | −0.1286228220879413 |
| chr18 | 1451267 | 1451267 | NK-cell1_2521 | −0.1267454530710455 |
| chr6 | 37766027 | 37766027 | NK-cell1_5091 | −0.1261837577617294 |
| chr17 | 68849264 | 68849264 | NK-cell1_2464 | −0.1170147301392183 |
| chr21 | 45947105 | 45947105 | NK-cell1_3696 | −0.1143514001448475 |
| chr1 | 95388197 | 95388197 | NK-cell1_245 | −0.1107461950284471 |
| chr9 | 93238131 | 93238131 | NK-cell1_6142 | −0.105947914075876 |
| chr15 | 89443352 | 89443352 | NK-cell1_2035 | −0.1050184258756313 |
| chr11 | 113087382 | 113087382 | NK-cell1_1173 | −0.1038375647686996 |
| chr6 | 87082384 | 87082384 | NK-cell1_5192 | −0.1036543812464209 |
| chr5 | 95766787 | 95766787 | NK-cell1_4813 | −0.1036257941554037 |
| chr13 | 90690333 | 90690333 | NK-cell1_1662 | −0.1032011737527657 |
| chr3 | 46660689 | 46660689 | NK-cell1_3914 | −0.1004446109925404 |
| chr2 | 201979352 | 201979352 | NK-cell1_3284 | −0.0989492001727238 |
| chr1 | 91937462 | 91937462 | NK-cell1_242 | −0.0976188496338418 |
| chr20 | 59677044 | 59677044 | NK-cell1_3589 | −0.0965304940125441 |
| chr12 | 102364494 | 102364494 | NK-cell1_1438 | −0.0948821279040705 |
| chr5 | 103928975 | 103928975 | NK-cell1_4827 | −0.0811788283679118 |
| chr6 | 42863290 | 42863290 | NK-cell1_5103 | −0.0808295382061388 |
| chr16 | 54432150 | 54432150 | NK-cell1_2186 | −0.0779906509497939 |
| chr13 | 66417332 | 66417332 | NK-cell1_1615 | −0.076471074805296 |
| chr5 | 36301317 | 36301317 | NK-cell1_4708 | −0.0764364404090611 |
| chr2 | 128879907 | 128879907 | NK-cell1_3138 | −0.0751536382501583 |
| chr10 | 132908914 | 132908914 | NK-cell1_872 | −0.0738710828666126 |
| chr1 | 101189664 | 101189664 | NK-cell1_251 | −0.0733077128562014 |
| chr4 | 154640817 | 154640817 | NK-cell1_4515 | −0.0670405677628217 |
| chr14 | 82987840 | 82987840 | NK-cell1_1845 | −0.0615614417883441 |
| chr9 | 135574626 | 135574626 | NK-cell1_6253 | −0.0599498131594607 |
| chr11 | 7482074 | 7482074 | NK-cell1_909 | −0.0572602492775831 |
| chr16 | 86083818 | 86083818 | NK-cell1_2254 | −0.0548867663313995 |
| chr10 | 14688718 | 14688718 | NK-cell1_579 | 0.0547477216544724 |
| chr8 | 25346547 | 25346547 | NK-cell1_5731 | 0.0551611920734571 |
| chr1 | 207298438 | 207298438 | NK-cell1_419 | 0.0573119810888458 |
| chr20 | 2421049 | 2421049 | NK-cell1_3420 | 0.0654963604015876 |
| chr5 | 170061315 | 170061315 | NK-cell1_4949 | 0.0662789260186687 |
| chr9 | 122941953 | 122941953 | NK-cell1_6204 | 0.0666236615841331 |
| chr16 | 68806796 | 68806796 | NK-cell1_2211 | 0.0676210671769465 |
| chr11 | 1443476 | 1443476 | NK-cell1_884 | 0.0688803872914327 |
| chr3 | 168727134 | 168727134 | NK-cell1_4140 | 0.0689466865894684 |
| chr16 | 15144029 | 15144029 | NK-cell1_2113 | 0.0695884075130002 |
| chr5 | 54285913 | 54285913 | NK-cell1_4735 | 0.073537957876446 |
| chrX | 82433310 | 82433310 | NK-cell1_6402 | 0.0741253176219116 |
| chr4 | 89925602 | 89925602 | NK-cell1_4393 | 0.0805477738172907 |
| chr4 | 2333532 | 2333532 | NK-cell1_4212 | 0.0811580320944752 |
| chr1 | 237684982 | 237684982 | NK-cell1_511 | 0.0824173433774952 |
| chr1 | 3323396 | 3323396 | NK-cell1_12 | 0.0831014618672925 |
| chr2 | 151511541 | 151511541 | NK-cell1_3180 | 0.0835517441806586 |
| chr1 | 6105214 | 6105214 | NK-cell1_26 | 0.0838347287512268 |
| chr12 | 42111582 | 42111582 | NK-cell1_1335 | 0.0838392325129016 |
| chr10 | 122295644 | 122295644 | NK-cell1_815 | 0.086150523782511 |
| chr2 | 42097413 | 42097413 | NK-cell1_2945 | 0.0869667923812173 |
| chr4 | 142845387 | 142845387 | NK-cell1_4491 | 0.0888727423638345 |
| chr15 | 51483712 | 51483712 | NK-cell1_1954 | 0.091925014128707 |
| chr16 | 66587551 | 66587551 | NK-cell1_2207 | 0.0976415320993837 |
| chr8 | 130631432 | 130631432 | NK-cell1_5935 | 0.1026628082852791 |
| chrX | 39328747 | 39328747 | NK-cell1_6334 | 0.1052423228831398 |
| chr19 | 58538021 | 58538021 | NK-cell1_2838 | 0.109121705997355 |
| chr5 | 170790122 | 170790122 | NK-cell1_4952 | 0.1093458790295459 |
| chrX | 39923031 | 39923031 | NK-cell1_6336 | 0.1098072548001858 |
| chr15 | 69594664 | 69594664 | NK-cell1_1991 | 0.1103484492040529 |
| chr15 | 77974027 | 77974027 | NK-cell1_2006 | 0.1116492917683346 |
| chrX | 115719392 | 115719398 | NK-cell1_6443 | 0.1124854042162576 |
| chr13 | 84787731 | 84787731 | NK-cell1_1651 | 0.114222856655595 |
| chr2 | 227390213 | 227390213 | NK-cell1_3353 | 0.1149823966773799 |
| chr2 | 199306371 | 199306371 | NK-cell1_3276 | 0.121832339146042 |
| chr22 | 42882214 | 42882214 | NK-cell1_3781 | 0.1253991859144084 |
| chr6 | 170177939 | 170177939 | NK-cell1_5356 | 0.1293148166184266 |
| chr9 | 138015226 | 138015226 | NK-cell1_6267 | 0.1310027050866895 |
| chr2 | 190249508 | 190249508 | NK-cell1_3260 | 0.1395752806512813 |
| chrX | 121966051 | 121966051 | NK-cell1_6454 | 0.1427748073656593 |
| chr7 | 101821549 | 101821549 | NK-cell1_5563 | 0.1509571718115018 |
| chr14 | 56496677 | 56496677 | NK-cell1_1784 | 0.1527961805777102 |
| chr1 | 152629513 | 152629513 | NK-cell1_306 | 0.1535161710986156 |
| chr14 | 94759608 | 94759608 | NK-cell1_1866 | 0.156750173737289 |
| chr9 | 124141236 | 124141236 | NK-cell1_6208 | 0.1633182042767091 |
| chrX | 62757213 | 62757213 | NK-cell1_6376 | 0.1643076240718775 |
| chr1 | 214317412 | 214317412 | NK-cell1_442 | 0.169587979141158 |
| chr12 | 110861114 | 110861114 | NK-cell1_1456 | 0.1733828960260987 |
| chr19 | 22700737 | 22700737 | NK-cell1_2729 | 0.1772221820647485 |
| chr6 | 56729561 | 56729561 | NK-cell1_5149 | 0.1867484183399251 |
| chr6 | 22927902 | 22927902 | NK-cell1_5053 | 0.2063500393189467 |
| chr2 | 68653171 | 68653171 | NK-cell1_3010 | 0.2095666613910457 |
| chrX | 152655570 | 152655570 | NK-cell1_6516 | 0.2327679596977246 |
| chr1 | 78005870 | 78005870 | NK-cell1_205 | 0.2439938201932086 |

MRSA_NK-cell2

| | | | | |
|---|---|---|---|---|
| chr4 | 56228328 | 56228328 | NK-cell2_6267 | −0.2032014868165767 |
| chr4 | 110226585 | 110226585 | NK-cell2_6403 | −0.2004144402910033 |
| chr20 | 62757709 | 62757709 | NK-cell2_5063 | −0.1951899046598685 |
| chrX | 87617787 | 87617787 | NK-cell2_9397 | −0.1928300087833981 |
| chr2 | 240905980 | 240905980 | NK-cell2_4805 | −0.1779670497388649 |
| chr7 | 31782154 | 31782154 | NK-cell2_7893 | −0.1753458934615355 |
| chr1 | 246810549 | 246810549 | NK-cell2_705 | −0.16125464031234 |
| chr9 | 94484252 | 94484252 | NK-cell2_9009 | −0.157553328337567 |
| chr17 | 59321435 | 59321435 | NK-cell2_3481 | −0.1573331315245784 |
| chr5 | 55630474 | 55630474 | NK-cell2_6849 | −0.1494305463460246 |
| chr11 | 120516489 | 120516489 | NK-cell2_1568 | −0.1372962073121924 |
| chr5 | 55660148 | 55660148 | NK-cell2_6850 | −0.1361904522054937 |
| chr1 | 111308091 | 111308091 | NK-cell2_347 | −0.1320563341482697 |
| chr3 | 34225910 | 34225910 | NK-cell2_5558 | −0.1265663490805758 |
| chr11 | 14013268 | 14013268 | NK-cell2_1255 | −0.1263714181416162 |
| chr6 | 6714811 | 6714811 | NK-cell2_7276 | −0.1220401694632414 |
| chr10 | 5329023 | 5329023 | NK-cell2_737 | −0.1203584039289598 |
| chr11 | 27054236 | 27054236 | NK-cell2_1293 | −0.1139665267397348 |
| chr16 | 50133292 | 50133292 | NK-cell2_3108 | −0.1122783069712895 |
| chr10 | 12150318 | 12150318 | NK-cell2_768 | −0.1111321785781916 |
| chr2 | 123392908 | 123392908 | NK-cell2_4476 | −0.110603425392643 |
| chr8 | 119834348 | 119834348 | NK-cell2_8707 | −0.1080065851546445 |
| chr10 | 12149934 | 12149934 | NK-cell2_767 | −0.1067247224475761 |
| chr17 | 6549534 | 6549534 | NK-cell2_3305 | −0.1052404885581786 |
| chr1 | 93920699 | 93920699 | NK-cell2_293 | −0.1032315471478789 |
| chr20 | 55551081 | 55551081 | NK-cell2_5014 | −0.1019839391707432 |
| chr18 | 54942493 | 54942493 | NK-cell2_3746 | −0.1007292721730675 |
| chr11 | 10825834 | 10825834 | NK-cell2_1238 | −0.0992281637020406 |
| chr2 | 241665673 | 241665673 | NK-cell2_4812 | −0.0904523979164241 |
| chr1 | 154462459 | 154462459 | NK-cell2_403 | −0.0887524682542566 |
| chr16 | 89654535 | 89654535 | NK-cell2_3275 | −0.0885933581790479 |
| chr5 | 55661853 | 55661853 | NK-cell2_6851 | −0.0870821122147495 |
| chr6 | 51329155 | 51329155 | NK-cell2_7431 | −0.0869141608128999 |
| chr10 | 126159172 | 126159172 | NK-cell2_1116 | −0.0842443116750083 |
| chr3 | 71128549 | 71128549 | NK-cell2_5669 | −0.0831423884455719 |
| chr13 | 113790143 | 113790143 | NK-cell2_2374 | −0.0821300777577445 |
| chr14 | 39686487 | 39686487 | NK-cell2_2447 | −0.0817508230300646 |
| chr15 | 72825480 | 72825480 | NK-cell2_2833 | −0.0767160969884592 |
| chr1 | 106749670 | 106749670 | NK-cell2_328 | −0.0764296190854789 |
| chr6 | 20199882 | 20199882 | NK-cell2_7320 | −0.0747598704212906 |
| chr13 | 99765751 | 99765751 | NK-cell2_2314 | −0.0705855339429477 |
| chr7 | 106626107 | 106626107 | NK-cell2_8104 | −0.0702452953083963 |
| chr9 | 76892727 | 76892727 | NK-cell2_8952 | −0.0701075761578441 |
| chr5 | 154229454 | 154229454 | NK-cell2_7133 | −0.068673069547813 |
| chr9 | 86426361 | 86426361 | NK-cell2_8971 | −0.0681032000007671 |
| chr8 | 120002429 | 120002429 | NK-cell2_8708 | −0.0671408229447211 |
| chr8 | 2528136 | 2528136 | NK-ccll2_8325 | −0.0666783054487542 |
| chr3 | 62012781 | 62012781 | NK-cell2_5641 | −0.0663557533526074 |
| chr21 | 43700239 | 43700239 | NK-cell2_5196 | −0.0647000882174159 |
| chrX | 141695494 | 141695494 | NK-cell2_9489 | 0.0569453088818747 |
| chr17 | 53038057 | 53038057 | NK-cell2_3454 | 0.0584148766694326 |
| chrX | 35065442 | 35065442 | NK-cell2_9275 | 0.0595197943674122 |
| chr10 | 99956141 | 99956141 | NK-cell2_1023 | 0.0611635287672573 |
| chr7 | 32087186 | 32087186 | NK-cell2_7896 | 0.0629602574931429 |
| chr11 | 117504745 | 117504745 | NK-cell2_1553 | 0.06328942467335 |
| chr11 | 9608006 | 9608006 | NK-cell2_1236 | 0.0643412707536295 |
| chr1 | 1645089 | 1645089 | NK-cell2_0 | 0.0658003508742925 |
| chr8 | 20901257 | 20901257 | NK-cell2_8432 | 0.0661556047371632 |
| chr18 | 37289534 | 37289534 | NK-cell2_3683 | 0.068168228499348 |
| chr5 | 733917 | 733917 | NK-cell2_6645 | 0.0684138835989934 |
| chrY | 9975281 | 9975281 | NK-cell2_9533 | 0.0713926522577049 |
| chr15 | 22036359 | 22036359 | NK-cell2_2688 | 0.0714467708819137 |
| chr5 | 6837100 | 6837100 | NK-cell2_6697 | 0.0719066886605799 |
| chr5 | 176034575 | 176034575 | NK-cell2_7218 | 0.0720350540921236 |
| chrX | 8345296 | 8345296 | NK-cell2_9202 | 0.0734804940460135 |
| chr4 | 26114657 | 26114657 | NK-cell2_6169 | 0.0753808162761622 |
| chr3 | 21009694 | 21009694 | NK-cell2_5495 | 0.0791901763528456 |
| chr9 | 69079756 | 69079756 | NK-cell2_8935 | 0.0796991228240167 |
| chrY | 19378895 | 19378895 | NK-cell2_9540 | 0.0818037410059209 |
| chr10 | 124051190 | 124051190 | NK-cell2_1111 | 0.0829945291578173 |
| chr3 | 59253416 | 59253416 | NK-cell2_5633 | 0.0895048583667983 |
| chr20 | 44907814 | 44907814 | NK-cell2_4969 | 0.0936703249852071 |
| chr5 | 67202022 | 67202022 | NK-cell2_6889 | 0.0944663372421897 |
| chr3 | 159234526 | 159234526 | NK-cell2_5902 | 0.0979333352192345 |
| chr5 | 13759546 | 13759546 | NK-cell2_6718 | 0.1027329827781859 |
| chr11 | 84029556 | 84029556 | NK-cell2_1461 | 0.1043603666456441 |
| chr16 | 64002185 | 64002185 | NK-cell2_3148 | 0.1149531871841455 |
| chrX | 87822060 | 87822060 | NK-cell2_9398 | 0.1160884750753742 |
| chr4 | 37509206 | 37509206 | NK-cell2_6208 | 0.1182575198412735 |
| chr19 | 5317561 | 5317561 | NK-cell2_3861 | 0.1263640076251874 |
| chr5 | 148015210 | 148015210 | NK-cell2_7107 | 0.1332564057528084 |
| chr2 | 219780266 | 219780266 | NK-cell2_4717 | 0.1333355611237281 |
| chr8 | 86451366 | 86451366 | NK-cell2_8623 | 0.1344110214772504 |
| chr4 | 37509488 | 37509488 | NK-cell2_6209 | 0.1363398210812488 |
| chrX | 33041908 | 33041908 | NK-cell2_9266 | 0.1403992828679788 |
| chr1 | 17421931 | 17421931 | NK-cell2_85 | 0.140543584607409 |
| chr4 | 98619858 | 98619858 | NK-ccll2_6367 | 0.1433892970146363 |
| chr13 | 69602652 | 69602652 | NK-cell2_2236 | 0.1439699614673547 |
| chrY | 21455682 | 21455682 | NK-cell2_9541 | 0.1479969825360025 |
| chr2 | 70823922 | 70823922 | NK-cell2_4297 | 0.1484701428980365 |
| chr8 | 12173371 | 12173371 | NK-cell2_8389 | 0.1608326511474594 |
| chr8 | 3007532 | 3007608 | NK-cell2_8332 | 0.1637694830827371 |
| chr19 | 18680958 | 18680958 | NK-cell2_3917 | 0.1701821150467656 |
| chr9 | 115041309 | 115041309 | NK-cell2_9079 | 0.18183277766283 |
| chrY | 5134738 | 5134738 | NK-cell2_9527 | 0.1986379478679154 |
| chrX | 101057939 | 101057939 | NK-cell2_9427 | 0.2262165583628206 |
| chr20 | 58982186 | 58982186 | NK-cell2_5036 | 0.2579557467085724 |
| chr4 | 189553841 | 189553841 | NK-cell2_6632 | 0.2710291012020165 |
| chr7 | 5138916 | 5138916 | NK-cell2_7823 | 0.3084462301744547 |
| chr10 | 80397646 | 80397646 | NK-cell2_979 | 0.3519823368015436 |

MRSA Tc-Mem

| | | | | |
|---|---|---|---|---|
| chr1 | 106749670 | 106749670 | Tc-Mem_878 | −0.1512584780583244 |
| chr4 | 118671407 | 118671407 | Tc-Mem_15251 | −0.1501134116063405 |
| chr5 | 142294189 | 142294189 | Tc-Mem_16804 | −0.1292536735345875 |
| chr17 | 48116605 | 48116605 | Tc-Mem_8064 | −0.1274570819638354 |
| chr13 | 64196996 | 64196996 | Tc-Mem_5137 | −0.118628340439525 |
| chr14 | 94320734 | 94320734 | Tc-Mem_6036 | −0.1160959784883187 |
| chr18 | 49053896 | 49053896 | Tc-Mem_8774 | −0.1048863679732451 |
| chr17 | 68849264 | 68849264 | Tc-Mem_8280 | −0.0960398534078249 |
| chr5 | 149141106 | 149141106 | Tc-Mem_16870 | −0.0923018703459842 |
| chr2 | 87662286 | 87662286 | Tc-Mem_10407 | −0.0896361819200156 |
| chr6 | 41227702 | 41227702 | Tc-Mem_17557 | −0.0883022000203358 |
| chr10 | 8466264 | 8466264 | Tc-Mem_1763 | −0.0865183083031703 |
| chr6 | 61707748 | 61707748 | Tc-Mem_17703 | −0.0845966872332229 |
| chr5 | 143104882 | 143105040 | Tc-Mem_16814 | −0.0843983573892184 |
| chr11 | 117974352 | 117974352 | Tc-Mem_3677 | −0.0841602745787735 |
| chr6 | 105091339 | 105091339 | Tc-Mem_17945 | −0.0837248927186258 |
| chr11 | 19694386 | 19694386 | Tc-Mem_2999 | −0.0828472837294585 |
| chr2 | 102726774 | 102726774 | Tc-Mem_10504 | −0.0787622072693496 |
| chr6 | 105129811 | 105129811 | Tc-Mem_17949 | −0.0786992079706982 |
| chr13 | 64192607 | 64192607 | Tc-Mem_5136 | −0.078493056516056 |
| chr1 | 216829849 | 216829849 | Tc-Mem_1437 | −0.0781337528162119 |
| chr4 | 146410986 | 146410986 | Tc-Mem_15444 | −0.0768773855701306 |
| chr2 | 196685704 | 196685704 | Tc-Mem_11159 | −0.0751999121109375 |
| chr9 | 99989899 | 99989899 | Tc-Mem_21272 | −0.0745451029964481 |
| chr7 | 75432139 | 75432139 | Tc-Mem_18970 | −0.0733713422815024 |
| chr5 | 72884820 | 72884820 | Tc-Mem_16381 | −0.0728283183571761 |
| chr5 | 30890537 | 30890537 | Tc-Mem_16092 | −0.0712274540505202 |
| chr12 | 117158124 | 117158124 | Tc-Mem_4640 | −0.0699903370224598 |
| chr18 | 48597260 | 48597260 | Tc-Mem_8763 | −0.0685805820598374 |
| chr1 | 27654802 | 27654802 | Tc-Mem_357 | −0.0684671571389358 |
| chr4 | 158521457 | 158521457 | Tc-Mem_15548 | −0.0671592498318638 |
| chr16 | 89654535 | 89654535 | Tc-Mem_7613 | −0.066901903722791 |
| chr9 | 100077426 | 100077426 | Tc-Mem_21274 | −0.0666517545181208 |
| chr1 | 57526126 | 57526126 | Tc-Mem_603 | −0.0649888585749558 |
| chr13 | 112651289 | 112651289 | Tc-Mem_5482 | −0.0648470130109643 |
| chr17 | 31146334 | 31146334 | Tc-Mem_7922 | −0.064099162728621 |
| chr1 | 245658120 | 245658120 | Tc-Mem_1674 | −0.0639950979389145 |
| chr19 | 21030805 | 21030805 | Tc-Mem_9330 | −0.0639462913670388 |
| chr16 | 7000339 | 7000339 | Tc-Mem_6925 | −0.0631571706268553 |
| chr2 | 208237789 | 208237789 | Tc-Mem_11239 | −0.0622457051153502 |
| chr10 | 117827902 | 117827902 | Tc-Mem_2584 | 0.0605717378246922 |
| chr19 | 49012674 | 49012674 | Tc-Mem_9555 | −0.0591128285358857 |
| chr10 | 52154355 | 52154355 | Tc-Mem_2098 | −0.0579270845116784 |
| chr9 | 5648131 | 5648131 | Tc-Mem_20747 | −0.0567085016880316 |
| chr5 | 1818516 | 1818516 | Tc-Mem_15847 | −0.0565472200383377 |
| chr7 | 136344724 | 136344724 | Tc-Mem_19338 | 0.0557988547436831 |
| chr6 | 124838736 | 124838736 | Tc-Mem_18035 | 0.0560916978997345 |
| chr11 | 18305777 | 18305777 | Tc-Mem_2987 | 0.0561658609229973 |
| chrY | 3674942 | 3674942 | Tc-Mem_22353 | 0.0568457263143622 |
| chr3 | 91037719 | 91037719 | Tc-Mem_13734 | 0.0573364432292318 |
| chr5 | 180969597 | 180969597 | Tc-Mem_17165 | 0.0573601334150133 |
| chr11 | 63778363 | 63778363 | Tc-Mem_3295 | 0.0578774034193149 |
| chr4 | 104464355 | 104464355 | Tc-Mem_15175 | 0.0595590493663402 |
| chr17 | 60766118 | 60766118 | Tc-Mem_8191 | 0.0604393500084681 |
| chr17 | 67776542 | 67776542 | Tc-Mem_8264 | 0.0604468023469231 |
| chrY | 8718441 | 8718441 | Tc-Mem_22357 | 0.060816273643535 |
| chr5 | 70378684 | 70378684 | Tc-Mem_16367 | 0.0618888303788197 |
| chr15 | 43451430 | 43451430 | Tc-Mem_6368 | 0.0639440023241743 |
| chr16 | 30735870 | 30735870 | Tc-Mem_7138 | 0.0669208715524067 |
| chr6 | 167168037 | 167168037 | Tc-Mem_18324 | 0.0671059659269452 |
| chr22 | 18714856 | 18714856 | Tc-Mem_12616 | 0.0675648359812474 |
| chr12 | 55325496 | 55325496 | Tc-Mem_4233 | 0.0680250130668426 |
| chr1 | 169917825 | 169917825 | Tc-Mem_1130 | 0.0692317351299407 |
| chr10 | 39108220 | 39108220 | Tc-Mem_2003 | 0.0693454407284919 |
| chr11 | 113337464 | 113337464 | Tc-Mem_3625 | 0.0696734139362242 |
| chr12 | 43305427 | 43305427 | Tc-Mem_4158 | 0.0702472707707679 |
| chr5 | 160663695 | 160663695 | Tc-Mem_16967 | 0.0728942126687457 |
| chr2 | 204746742 | 204746742 | Tc-Mem_11210 | 0.07364069267957 |
| chrX | 69150568 | 69150568 | Tc-Mem_21967 | 0.0739144900486607 |
| chrY | 22804844 | 22804844 | Tc-Mem_22371 | 0.0745912658764022 |
| chr13 | 77621398 | 77621398 | Tc-Mem_5215 | 0.0781409535240451 |
| chrX | 44790553 | 44790553 | Tc-Mem_21880 | 0.0789416496858671 |
| chrY | 20036822 | 20036822 | Tc-Mem_22368 | 0.079269397956079 |
| chr12 | 36578440 | 36578440 | Tc-Mem_4112 | 0.0808043749186446 |
| chr1 | 174015624 | 174015624 | Tc-Mem_1144 | 0.0816372891232699 |
| chr22 | 15950933 | 15950933 | Tc-Mem_12591 | 0.0833944903398292 |
| chr8 | 65621719 | 65621719 | Tc-Mem_20153 | 0.0836180813255082 |
| chrX | 45775174 | 45775174 | Tc-Mem_21884 | 0.0837241769391408 |
| chr12 | 119292456 | 119292456 | Tc-Mem_4664 | 0.0857195092295484 |
| chr6 | 56173354 | 56173354 | Tc-Mem_17672 | 0.0916184526941892 |
| chr1 | 60944023 | 60944023 | Tc-Mem_621 | 0.0929409326073633 |
| chr3 | 545732 | 545732 | Tc-Mem_13035 | 0.0953231991458816 |
| chr6 | 29635804 | 29635804 | Tc-Mem_17414 | 0.0980014906515434 |
| chrY | 22294978 | 22294978 | Tc-Mem_22370 | 0.1033256799649887 |
| chr3 | 127072252 | 127072252 | Tc-Mem_13937 | 0.104977956513121 |
| chr8 | 92785622 | 92785622 | Tc-Mem_20308 | 0.1063044144276331 |
| chrY | 19434842 | 19434842 | Tc-Mem_22367 | 0.108155353732028 |
| chr5 | 175045265 | 175045265 | Tc-Mem_17110 | 0.1101348321205818 |
| chr20 | 28199186 | 28199186 | Tc-Mem_11820 | 0.11095956636618 |
| chr6 | 59760693 | 59760693 | Tc-Mem_17687 | 0.1128532322691306 |
| chr6 | 4148288 | 4148288 | Tc-Mem_17211 | 0.1136332942469999 |
| chr22 | 12049700 | 12049700 | Tc-Mem_12586 | 0.1190024962361521 |
| chrY | 10101398 | 10101398 | Tc-Mem_22360 | 0.1393243159479847 |
| chr1 | 143450602 | 143450602 | Tc-Mem_980 | 0.1393626755242891 |
| chr4 | 9162529 | 9162529 | Tc-Mem_14530 | 0.162501674508307 |
| chr7 | 61461655 | 61461655 | Tc-Mem_18845 | 0.1695393383179066 |
| chr15 | 56316524 | 56316524 | Tc-Mem_6446 | 0.1776619676631391 |
| chr6 | 158100565 | 158100565 | Tc-Mem_18226 | 0.1850919691169961 |
| chr11 | 89960109 | 89960109 | Tc-Mem_3477 | 0.1946173637354295 |
| chr16 | 37229392 | 37229392 | Tc-Mem_7183 | 0.2008752976343301 |

MRSA_Tc-Naïve

| | | | | |
|---|---|---|---|---|
| chr21 | 13279399 | 13279399 | Tc-Naive_10130 | −0.204898954600475 |
| chr12 | 23032362 | 23032362 | Tc-Naive_3403 | −0.1551144344035439 |
| chr8 | 7038171 | 7038171 | Tc-Naive_16363 | −0.1385591777348692 |
| chr4 | 7477416 | 7477426 | Tc-Naive_11994 | −0.1284014427437114 |
| chr17 | 62507188 | 62507188 | Tc-Naive_6902 | −0.1229638439631243 |
| chr10 | 12148322 | 12148322 | Tc-Naive_1543 | −0.1151017413064421 |
| chr12 | 26639618 | 26639618 | Tc-Naive_3425 | −0.1113187680198287 |
| chr8 | 38646813 | 38646813 | Tc-Naive_16565 | −0.1080121026707454 |
| chr2 | 87657712 | 87657712 | Tc-Naive_8676 | −0.104285741412657 |
| chr10 | 28956415 | 28956415 | Tc-Naive_1656 | −0.101358632205714 |
| chr21 | 13423301 | 13423301 | Tc-Naive_10134 | −0.0987172277794169 |
| chr2 | 170953261 | 170953261 | Tc-Naive_9134 | −0.0985831327364744 |
| chr14 | 39698417 | 39698417 | Tc-Naive_4729 | −0.0985285217732728 |
| chr3 | 21083541 | 21083541 | Tc-Naive_10911 | −0.0918189690593913 |
| chr1 | 241816403 | 241816403 | Tc-Naive_1396 | −0.0885340183518504 |
| chr13 | 43967593 | 43967593 | Tc-Naive_4204 | −0.0878243514593928 |
| chr4 | 38750130 | 38750130 | Tc-Naive_12198 | −0.0815757705834049 |
| chr2 | 140968626 | 140968626 | Tc-Naive_8997 | −0.0806283563978112 |
| chr6 | 41231363 | 41231363 | Tc-Naive_14596 | −0.0806176268061999 |
| chr10 | 103284206 | 103284206 | Tc-Naive_2107 | −0.0797703922396424 |
| chr4 | 108399799 | 108399799 | Tc-Naive_12592 | −0.0794111199767202 |
| chr2 | 191965522 | 191965522 | Tc-Naive_9268 | −0.0789435502800967 |
| chr14 | 100152154 | 100152154 | Tc-Naive_5108 | −0.0768243926927162 |
| chr16 | 12245188 | 12245188 | Tc-Naive_5836 | −0.0758066584017864 |
| chr18 | 70969220 | 70969220 | Tc-Naive_7501 | −0.0743516543246507 |
| chr22 | 26693361 | 26693361 | Tc-Naive_10506 | −0.0733188440974107 |
| chr11 | 133287928 | 133287928 | Tc-Naive_3199 | −0.0732118086487533 |
| chr14 | 100126621 | 100126621 | Tc-Naive_5106 | −0.0693861400092312 |
| chr6 | 105936013 | 105936013 | Tc-Naive_14924 | −0.0687564852995505 |
| chrX | 142426815 | 142426815 | Tc-Naive_18451 | −0.0677567703058546 |
| chr14 | 76084003 | 76084003 | Tc-Naive_4962 | −0.0676000515412415 |
| chr12 | 52826381 | 52826381 | Tc-Naive_3580 | −0.0674201372752533 |
| chr6 | 168899279 | 168899279 | Tc-Naive_15249 | −0.0664941860839602 |
| chr12 | 131466787 | 131466787 | Tc-Naive_4027 | −0.0662277366738764 |
| chr11 | 99443168 | 99443168 | Tc-Naive_2999 | −0.064441838837025 |
| chr8 | 5521290 | 5521290 | Tc-Naive_16345 | −0.0636774340711602 |
| chr2 | 146078483 | 146078483 | Tc-Naive_9012 | −0.0635078051282131 |
| chr2 | 28424798 | 28424798 | Tc-Naive_8293 | −0.0629770602112653 |
| chr7 | 132293403 | 132293403 | Tc-Naive_16064 | −0.0629015676566735 |
| chr2 | 77220575 | 77220575 | Tc-Naive_8605 | −0.0626524811368771 |
| chr6 | 167686794 | 167686794 | Tc-Naive_15232 | −0.0622470665051794 |
| chr1 | 79594651 | 79594651 | Tc-Naive_602 | −0.0615518392838554 |
| chr17 | 62637128 | 62637128 | Tc-Naive_6908 | 0.0617790451535657 |
| chr19 | 17683632 | 17683632 | Tc-Naive_7805 | 0.061966188077021 |
| chr5 | 178253673 | 178253673 | Tc-Naive_14181 | 0.0625912333048745 |
| chr4 | 59755382 | 59755382 | Tc-Naive_12324 | 0.063170581393361 |
| chr7 | 147607269 | 147607269 | Tc-Naive_16153 | 0.06317897020437 |
| chrY | 21836431 | 21836431 | Tc-Naive_18535 | 0.0640427511683288 |
| chr2 | 178596494 | 178596494 | Tc-Naive_9193 | 0.0647078541059359 |
| chr10 | 9027052 | 9027052 | Tc-Naive_1521 | 0.0651840232992215 |
| chr9 | 111272906 | 111272906 | Tc-Naive_17712 | 0.0667185790118649 |
| chrX | 107979417 | 107979417 | Tc-Naive_18322 | 0.0672461521395475 |
| chrY | 17579580 | 17579580 | Tc-Naive_18534 | 0.0701043168769814 |
| chr4 | 113322999 | 113322999 | Tc-Naive_12610 | 0.0716507678037503 |
| chr5 | 118646957 | 118646957 | Tc-Naive_13776 | 0.0717082185948193 |
| chr10 | 68895946 | 68895946 | Tc-Naive_1886 | 0.0721157820419065 |
| chr11 | 18305777 | 18305777 | Tc-Naive_2563 | 0.0721549458893256 |
| chrX | 109073176 | 109073176 | Tc-Naive_18328 | 0.0742221012119943 |
| chrY | 11956080 | 11956080 | Tc-Naive_18530 | 0.0752470797267469 |
| chrY | 11954040 | 11954067 | Tc-Naive_18529 | 0.0761015733171956 |
| chr2 | 149448274 | 149448274 | Tc-Naive_9022 | 0.0779563623125486 |
| chr19 | 9350640 | 9350640 | Tc-Naive_7688 | 0.0783207622793222 |
| chr1 | 113634062 | 113634062 | Tc-Naive_776 | 0.0784393336021989 |
| chr13 | 83704664 | 83704664 | Tc-Naive_4423 | 0.0821995797562649 |
| chr10 | 19661436 | 19661436 | Tc-Naive_1609 | 0.0844733001718944 |
| chrX | 102023020 | 102023020 | Tc-Naive_18298 | 0.0844952830149132 |
| chr4 | 1781540 | 1781540 | Tc-Naive_11934 | 0.0863915824449623 |
| chr22 | 29437650 | 29437650 | Tc-Naive_10536 | 0.0865671122208498 |
| chr3 | 178118061 | 178118061 | Tc-Naive_11765 | 0.0883610415554764 |
| chrY | 9982982 | 9982982 | Tc-Naive_18527 | 0.0906862151771239 |
| chr16 | 36759939 | 36759939 | Tc-Naive_5997 | 0.0907374878380754 |
| chr7 | 36258821 | 36258821 | Tc-Naive_15529 | 0.0920337650980406 |
| chr22 | 21977447 | 21977447 | Tc-Naive_10456 | 0.0936179049169761 |
| chrY | 15716477 | 15716477 | Tc-Naive_18532 | 0.0954164851848126 |
| chrY | 9225432 | 9225432 | Tc-Naive_18525 | 0.0959766570449002 |
| chrX | 102598043 | 102598043 | Tc-Naive_18299 | 0.0964613837351636 |
| chr11 | 111774100 | 111774100 | Tc-Naive_3059 | 0.0973438770327929 |
| chr21 | 8653904 | 8653904 | Tc-Naive_10118 | 0.1006656881686059 |
| chr16 | 11510045 | 11510045 | Tc-Naive_5827 | 0.1009321359212547 |
| chr3 | 157377227 | 157377227 | Tc-Naive_11644 | 0.1021318769686816 |
| chr12 | 127314923 | 127314923 | Tc-Naive_3962 | 0.1021406678675498 |
| chr7 | 5139806 | 5139806 | Tc-Naive_15340 | 0.1032414934074476 |
| chr3 | 21036192 | 21036192 | Tc-Naive_10909 | 0.1057709239309055 |
| chrX | 101440149 | 101440149 | Tc-Naive_18291 | 0.1060120765806019 |
| chr11 | 3804005 | 3804005 | Tc-Naive_2442 | 0.1061348259021479 |
| chr17 | 53444428 | 53444428 | Tc-Naive_6844 | 0.1081969112830267 |
| chr12 | 29791736 | 29791736 | Tc-Naive_3439 | 0.1140019292441346 |
| chr14 | 20426991 | 20426991 | Tc-Naive_4644 | 0.114005393978386 |
| chr18 | 54236495 | 54236495 | Tc-Naive_7409 | 0.1178291727565781 |
| chrY | 2844530 | 2844530 | Tc-Naive_18520 | 0.1206424062805243 |
| chr9 | 40955980 | 40955980 | Tc-Naive_17419 | 0.1265702936898016 |
| chr11 | 6346126 | 6346126 | Tc-Naive_2473 | 0.1322233088688198 |
| chr17 | 80918469 | 80918469 | Tc-Naive_7077 | 0.1406816291624307 |
| chr20 | 63172364 | 63172364 | Tc-Naive_10092 | 0.1453352231197601 |
| chr1 | 241562943 | 241562943 | Tc-Naive_1391 | 0.1556817216380886 |
| chr12 | 96380170 | 96380170 | Tc-Naive_3772 | 0.1716234904121475 |
| chr15 | 42015868 | 42015868 | Tc-Naive_5307 | 0.1721616562996831 |
| chrY | 5440408 | 5440408 | Tc-Naive_18522 | 0.1725451301947027 |
| chr7 | 20014750 | 20014750 | Tc-Naive_15419 | 0.1833739596097031 |
| chr2 | 90246012 | 90246012 | Tc-Naive_8690 | 0.1890538068719571 |

MRSA Th-Mem

| | | | | |
|---|---|---|---|---|
| chr8 | 9060832 | 9060832 | Th-Mem_21138 | −0.1357816374956447 |
| chr4 | 159740545 | 159740545 | Th-Mem_16642 | −0.1329537366651506 |
| chr1 | 69413426 | 69413426 | Th-Mem_697 | −0.125592578306191 |
| chr10 | 50329590 | 50329590 | Th-Mem_2272 | −0.1125031382689963 |
| chr8 | 5291675 | 5291675 | Th-Mem_21093 | −0.1115787335180992 |
| chr7 | 147958126 | 147958126 | Th-Mem_20879 | −0.1019895906409716 |
| chr14 | 57402118 | 57402118 | Th-Mem_6377 | −0.0940659825992289 |
| chr16 | 89654535 | 89654535 | Th-Mem_8263 | −0.0924500410657555 |
| chrX | 87236044 | 87236044 | Th-Mem_23855 | −0.0894597780851834 |
| chr17 | 51312514 | 51312514 | Th-Mem_8696 | −0.0882081911587197 |
| chr5 | 1820426 | 1820426 | Th-Mem_16972 | 0.0865982821863342 |
| chr1 | 1735884 | 1735884 | Th-Mem_12 | −0.0861492138770329 |
| chr2 | 64278670 | 64278670 | Th-Mem_10870 | −0.0809650328520018 |
| chr8 | 15129200 | 15129208 | Th-Mem_21209 | −0.0805636632416732 |
| chr4 | 71254140 | 71254140 | Th-Mem_15962 | −0.0802926775515404 |
| chr3 | 155869107 | 155869107 | Th-Mem_14984 | −0.0802818964639263 |
| chr1 | 200943752 | 200943752 | Th-Mem_1427 | −0.079882834080098 |
| chr1 | 69377687 | 69377687 | Th-Mem_694 | −0.0788637158518435 |
| chr16 | 4661392 | 4661392 | Th-Mem_7495 | −0.078471446493559 |
| chr4 | 55170855 | 55171164 | Th-Mem_15830 | −0.0783644155161939 |
| chr1 | 54959802 | 54959802 | Th-Mem_570 | −0.0779387446148412 |
| chr14 | 100277218 | 100277218 | Th-Mem_6710 | −0.0725720312476906 |
| chr3 | 43161910 | 43161910 | Th-Mem_14100 | −0.0699347359759434 |
| chr10 | 97032381 | 97032381 | Th-Mem_2646 | −0.069415520710318 |
| chr1 | 63255654 | 63255654 | Th-Mem_633 | −0.0687495863947791 |
| chr5 | 1891686 | 1891686 | Th-Mem_16978 | −0.0655723966187949 |
| chr2 | 226467477 | 226467477 | Th-Mem_12130 | −0.0654322523850947 |
| chr4 | 33356300 | 33356300 | Th-Mem_15667 | 0.0647773954914375 |
| chr14 | 100126621 | 100126621 | Th-Mem_6706 | −0.0643736930424117 |
| chr4 | 55171728 | 55171728 | Th-Mem_15832 | −0.063393126714265 |
| chr1 | 87731334 | 87731334 | Th-Mem_834 | −0.0623648330460881 |
| chr2 | 198712655 | 198712655 | Th-Mem_11896 | −0.0596395834540479 |
| chr16 | 23821801 | 23821801 | Th-Mem_7707 | −0.0591429478952064 |
| chr10 | 106738252 | 106738252 | Th-Mem_2732 | −0.0588305342974167 |
| chr5 | 74551218 | 74551218 | Th-Mem_17570 | −0.0582799087194774 |
| chr6 | 89078362 | 89078362 | Th-Mem_19116 | −0.0576542798328975 |
| chr15 | 68736822 | 68736822 | Th-Mem_7148 | −0.0552258570965095 |
| chr16 | 272935 | 272935 | Th-Mem_7449 | −0.0548376413265548 |
| chr7 | 129200293 | 129200293 | Th-Mem_20746 | −0.0545147788086204 |
| chr10 | 76586282 | 76586282 | Th-Mem_2484 | −0.0533930548796487 |
| chr1 | 63468599 | 63468599 | Th-Mem_637 | −0.0522412190638321 |
| chr1 | 69419693 | 69419693 | Th-Mem_698 | −0.0521080724210095 |
| chr8 | 6163397 | 6163397 | Th-Mem_21108 | −0.0511444540052417 |
| chr1 | 110063942 | 110064052 | Th-Mem_969 | −0.0508030247665289 |
| chr2 | 113811759 | 113811759 | Th-Mem_11247 | −0.0504954033734424 |
| chr14 | 75782761 | 75782761 | Th-Mem_6510 | −0.0503221157289449 |
| chr7 | 797770 | 797770 | Th-Mem_19735 | −0.0492376507990823 |
| chr17 | 47340744 | 47340744 | Th-Mem_8665 | 0.0514228969851596 |
| chr14 | 27938043 | 27938043 | Th-Mem_6148 | 0.0518539966282059 |
| chr17 | 68813786 | 68813786 | Th-Mem_8858 | 0.0528068954821922 |
| chr10 | 80932213 | 80932213 | Th-Mem_2531 | 0.0528766023194207 |
| chr5 | 9873904 | 9873904 | Th-Mem_17103 | 0.0535027017067041 |
| chr1 | 53173716 | 53173716 | Th-Mem_555 | 0.0535788507438791 |
| chr10 | 6188810 | 6188810 | Th-Mem_1896 | 0.0545739463055237 |
| chr10 | 70206907 | 70206907 | Th-Mem_2433 | 0.0568169277752099 |
| chr4 | 189623367 | 189623367 | Th-Mem_16922 | 0.0578459307856527 |
| chr2 | 21219762 | 21219762 | Th-Mem_10527 | 0.058621036808867 |
| chr1 | 174306123 | 174306123 | Th-Mem_1250 | 0.058970507957569 |
| chr6 | 149008353 | 149008353 | Th-Mem_19465 | 0.0618373868923478 |
| chr17 | 47313106 | 47313106 | Th-Mem_8663 | 0.0636157857284202 |
| chr20 | 5792562 | 5792562 | Th-Mem_12382 | 0.0641015005982458 |
| chr4 | 50696012 | 50696012 | Th-Mem_15804 | 0.0648186182649955 |
| chr8 | 7331915 | 7331915 | Th-Mem_21128 | 0.0654078495671935 |
| chr2 | 83407114 | 83407114 | Th-Mem_11037 | 0.0691036734056914 |
| chr5 | 49624277 | 49624277 | Th-Mem_17394 | 0.0722968648847435 |
| chr8 | 6724708 | 6724708 | Th-Mem_21119 | 0.0731053075137284 |
| chr6 | 58727852 | 58727852 | Th-Mem_18913 | 0.0778276523100235 |
| chr5 | 19549689 | 19549689 | Th-Mem_17185 | 0.0794621515505371 |
| chr19 | 11752591 | 11752591 | Th-Mem_9883 | 0.079621439038256 |
| chr11 | 52662176 | 52662176 | Th-Mem_3489 | 0.0841554515957027 |
| chr9 | 110676415 | 110676415 | Th-Mem_23046 | 0.0856125925718042 |
| chr3 | 115608604 | 115608604 | Th-Mem_14645 | 0.0875501795488843 |
| chrY | 11899749 | 11899749 | Th-Mem_24257 | 0.0934475840964948 |
| chr19 | 16851058 | 16851058 | Th-Mem_9960 | 0.0954794268473029 |
| chr4 | 153267434 | 153267434 | Th-Mem_16580 | 0.0958820318419412 |
| chr3 | 1672789 | 1672789 | Th-Mem_13731 | 0.0977909593054714 |
| chr7 | 3054031 | 3054031 | Th-Mem_19766 | 0.1033045753451217 |
| chr11 | 5696983 | 5696983 | Th-Mem_3133 | 0.1052881719129094 |
| chr16 | 27188785 | 27188785 | Th-Mem_7740 | 0.108106483307693 |
| chr10 | 53898027 | 53898027 | Th-Mem_2294 | 0.1223554415397785 |
| chr15 | 30368177 | 30368177 | Th-Mem_6878 | 0.1235815889440332 |
| chr5 | 180969597 | 180969597 | Th-Mem_18330 | 0.1247734870273118 |
| chr11 | 18305777 | 18305777 | Th-Mem_3233 | 0.1264754546320606 |
| chr7 | 29211101 | 29211101 | Th-Mem_19991 | 0.129338927651103 |
| chr2 | 42097413 | 42097413 | Th-Mem_10710 | 0.1319651906385613 |
| chrX | 132277327 | 132277327 | Th-Mem_24084 | 0.1392547082605148 |
| chr22 | 11971528 | 11971528 | Th-Mem_13319 | 0.1408478419262621 |
| chrY | 6653419 | 6653419 | Th-Mem_24251 | 0.1422884680807056 |
| chrY | 17081685 | 17081685 | Th-Mem_24262 | 0.1485245113098281 |
| chr7 | 36535069 | 36535069 | Th-Mem_20066 | 0.1534317534976117 |
| chr2 | 124140258 | 124140258 | Th-Mem_11357 | 0.1546335187062657 |
| chrY | 11303134 | 11303134 | Th-Mem_24254 | 0.1552177987195499 |
| chr7 | 60685042 | 60685042 | Th-Mem_20260 | 0.1571910864867273 |
| chrY | 12490871 | 12490871 | Th-Mem_24258 | 0.1669591146918343 |
| chr17 | 62640058 | 62640058 | Th-Mem_8791 | 0.1789637636809243 |
| chr10 | 40738785 | 40738785 | Th-Mem_2184 | 0.1844610155042828 |
| chr2 | 198669753 | 198669753 | Th-Mem_11894 | 0.1903087103077009 |
| chr4 | 107486644 | 107486644 | Th-Mem_16243 | 0.220858459639579 |
| chrY | 6081318 | 6081318 | Th-Mem_24249 | 0.2211072374834545 |
| chrY | 16178251 | 16178251 | Th-Mem_24261 | 0.2253875291408639 |

MRSA Th-Naïve

| | | | | |
|---|---|---|---|---|
| chr8 | 7037468 | 7037468 | Th-Naive_19049 | −0.1459117894723466 |
| chr10 | 19469769 | 19469769 | Th-Naive_1898 | −0.1263908417478832 |
| chr12 | 17795210 | 17795210 | Th-Naive_3955 | −0.1239943101793382 |
| chr4 | 158521457 | 158521457 | Th-Naive_14915 | −0.1224522988883092 |
| chr8 | 13885764 | 13885764 | Th-Naive_19109 | −0.1188184503915586 |
| chr11 | 93261867 | 93261867 | Th-Naive_3407 | −0.1108043914318978 |
| chr9 | 129558212 | 129558212 | Th-Naive_20796 | −0.1060360000899282 |
| chr4 | 101274290 | 101274290 | Th-Naive_14531 | −0.085012917735616 |
| chr7 | 140366052 | 140366052 | Th-Naive_18757 | −0.0829831935557895 |
| chr20 | 62757709 | 62757709 | Th-Naive_11716 | −0.080916478926496 |
| chr20 | 4589917 | 4589917 | Th-Naive_11164 | −0.0806134944538709 |
| chr21 | 17120160 | 17120160 | Th-Naive_11794 | −0.0804203694448049 |
| chr15 | 27897780 | 27897780 | Th-Naive_6103 | −0.0802739341807335 |
| chr17 | 59699152 | 59699152 | Th-Naive_7872 | −0.0755283269211331 |
| chr4 | 170166524 | 170166524 | Th-Naive_14992 | −0.0742941862920735 |
| chr11 | 39369137 | 39369137 | Th-Naive_3105 | −0.0702740332385722 |
| chr7 | 137180402 | 137180402 | Th-Naive_18737 | −0.0702283357963249 |
| chr11 | 35011943 | 35011943 | Th-Naive_3074 | −0.0691333714851217 |
| chr4 | 26694111 | 26694111 | Th-Naive_14049 | −0.0690168400265136 |
| chr7 | 145336954 | 145336954 | Th-Naive_18797 | −0.0678689896408463 |
| chr17 | 27386423 | 27386423 | Th-Naive_7616 | −0.0664905646554095 |
| chr6 | 55864928 | 55864928 | Th-Naive_17064 | −0.0653450369436841 |
| chr11 | 75146300 | 75146300 | Th-Naive_3315 | −0.0618919654050702 |
| chr4 | 140633794 | 140633794 | Th-Naive_14797 | −0.0618823281671579 |
| chr15 | 27903375 | 27903375 | Th-Naive_6104 | −0.0614834115551464 |
| chr1 | 72588248 | 72588248 | Th-Naive_661 | −0.0606262136024213 |
| chr7 | 37313255 | 37313255 | Th-Naive_18086 | −0.0590281871254243 |
| chr5 | 55444184 | 55444184 | Th-Naive_15639 | −0.0580041780029182 |
| chr16 | 89654535 | 89654535 | Th-Naive_7384 | −0.0576815114409299 |
| chr21 | 44330364 | 44330364 | Th-Naive_12024 | −0.0572488113091637 |
| chr3 | 130224670 | 130224670 | Th-Naive_13331 | −0.056807925810487 |
| chr6 | 55463274 | 55463274 | Th-Naive_17063 | −0.0556147771452854 |
| chr13 | 75203725 | 75203725 | Th-Naive_5098 | −0.0553985242125982 |
| chr17 | 34600711 | 34600711 | Th-Naive_7664 | −0.0553784214809518 |
| chr8 | 87221754 | 87221754 | Th-Naive_19604 | −0.0548730871870607 |
| chr7 | 131305094 | 131305094 | Th-Naive_18703 | −0.0539872521632423 |
| chr3 | 21067585 | 21067585 | Th-Naive_12613 | 0.0548180566936004 |
| chr1 | 174141977 | 174141977 | Th-Naive_1141 | 0.0557424025799883 |
| chr22 | 22996044 | 22996044 | Th-Naive_12137 | 0.0559954202914651 |
| chr9 | 111762845 | 111762845 | Th-Naive_20648 | 0.056499310708284 |
| chr4 | 101367620 | 101367620 | Th-Naive_14532 | 0.0568575331199839 |
| chr20 | 50284807 | 50284807 | Th-Naive_11564 | 0.0571888757647184 |
| chr10 | 67628221 | 67628221 | Th-Naive_2230 | 0.0581438190910782 |
| chr4 | 61074471 | 61074471 | Th-Naive_14280 | 0.0586845471657517 |
| chrX | 107979417 | 107979417 | Th-Naive_21385 | 0.0588256729808061 |
| chr6 | 18986731 | 18986911 | Th-Naive_16723 | 0.0598044589943688 |
| chr17 | 47313106 | 47313106 | Th-Naive_7746 | 0.0598443012640622 |
| chr10 | 79280622 | 79280622 | Th-Naive_2314 | 0.0598823936692313 |
| chr20 | 30202063 | 30202063 | Th-Naive_11404 | 0.0614939922281576 |
| chr8 | 79335696 | 79335696 | Th-Naive_19556 | 0.0621459834188139 |
| chr7 | 54673097 | 54673182 | Th-Naive_18232 | 0.0631379880363416 |
| chr6 | 7609803 | 7609803 | Th-Naive_16620 | 0.0632912780174509 |
| chr2 | 14799879 | 14799879 | Th-Naive_9434 | 0.0647683809353844 |
| chr14 | 21163858 | 21163858 | Th-Naive_5405 | 0.0658129239575469 |
| chr18 | 76038397 | 76038397 | Th-Naive_8672 | 0.0662125218431677 |
| chr2 | 131756568 | 131756568 | Th-Naive_10315 | 0.0663544164118224 |
| chr13 | 31053240 | 31053240 | Th-Naive_4821 | 0.0666557585843963 |
| chr12 | 128987196 | 128987196 | Th-Naive_4666 | 0.0674026026058278 |
| chrX | 129154267 | 129154267 | Th-Naive_21495 | 0.0684987675755997 |
| chr7 | 38344677 | 38344677 | Th-Naive_18093 | 0.0685570751310806 |
| chr22 | 21485696 | 21485696 | Th-Naive_12121 | 0.0686242842145297 |
| chrY | 3273319 | 3273319 | Th-Naive_21621 | 0.0700863420760015 |
| chr22 | 22972761 | 22972761 | Th-Naive_12134 | 0.0753334422043965 |
| chr7 | 61225708 | 61225708 | Th-Naive_18260 | 0.0757018521541831 |
| chr15 | 67788759 | 67788759 | Th-Naive_6390 | 0.0797127737004604 |
| chr22 | 49292357 | 49292357 | Th-Naive_12405 | 0.0800060869216326 |
| chr15 | 40677024 | 40677024 | Th-Naive_6175 | 0.0800379383197354 |
| chr8 | 40003397 | 40003397 | Th-Naive_19306 | 0.0800385380422106 |
| chr2 | 106413094 | 106413094 | Th-Naive_10118 | 0.0812146646625621 |
| chr5 | 30817601 | 30817601 | Th-Naive_15463 | 0.0834443287026786 |
| chr4 | 103165924 | 103165924 | Th-Naive_14557 | 0.0834984186708803 |
| chr5 | 3804412 | 3804412 | Th-Naive_15246 | 0.0857537397612014 |
| chr4 | 64735891 | 64735891 | Th-Naive_14297 | 0.0871861934735298 |
| chr4 | 183464123 | 183464123 | Th-Naive_15075 | 0.0895159699890484 |
| chr14 | 99552084 | 99552084 | Th-Naive_5947 | 0.0934302562948046 |
| chr4 | 130122026 | 130122026 | Th-Naive_14723 | 0.0941234360251329 |
| chr8 | 74397395 | 74397395 | Th-Naive_19532 | 0.0969132448586934 |
| chr6 | 15758084 | 15758084 | Th-Naive_16696 | 0.097902786410928 |
| chr2 | 177657691 | 177657691 | Th-Naive_10613 | 0.0981919246730601 |
| chr9 | 66776365 | 66776365 | Th-Naive_20308 | 0.1020235411502234 |
| chrX | 147046481 | 147046481 | Th-Naive_21579 | 0.1057189321255455 |
| chr8 | 31708687 | 31708687 | Th-Naive_19237 | 0.1068721452503842 |
| chr2 | 87654341 | 87654341 | Th-Naive_10011 | 0.1094043567878824 |
| chr18 | 7884902 | 7884902 | Th-Naive_8194 | 0.110413412919204 |
| chr9 | 43051518 | 43051518 | Th-Naive_20297 | 0.1106841093673228 |
| chr18 | 7652823 | 7652823 | Th-Naive_8191 | 0.1107835779166859 |
| chr1 | 159608951 | 159608951 | Th-Naive_1037 | 0.1111447044595522 |
| chrX | 44562376 | 44562376 | Th-Naive_21132 | 0.1129144511028015 |
| chr16 | 60055195 | 60055195 | Th-Naive_7109 | 0.1131916510058738 |
| chr16 | 38103802 | 38103802 | Th-Naive_6985 | 0.1171870288623461 |
| chr17 | 77216253 | 77216253 | Th-Naive_8032 | 0.1207845022009917 |
| chr3 | 21036192 | 21036192 | Th-Naive_12612 | 0.1224451327567748 |
| chr5 | 142330613 | 142330613 | Th-Naive_16180 | 0.1486560156857946 |
| chr4 | 81786713 | 81786713 | Th-Naive_14402 | 0.1515351701681184 |
| chr12 | 106442075 | 106442075 | Th-Naive_4476 | 0.1572226568941558 |
| chrX | 114704758 | 114704758 | Th-Naive_21417 | 0.1629769742805345 |
| chr16 | 37229392 | 37229392 | Th-Naive_6983 | 0.1720442754431506 |
| chr11 | 6346126 | 6346126 | Th-Naive_2858 | 0.1834378287740692 |
| chr15 | 42015868 | 42015868 | Th-Naive_6184 | 0.2168135823751006 |
| chr4 | 87068081 | 87068081 | Th-Naive_14425 | 0.2712488816883985 |

BA B-Mem

| | | | | |
|---|---|---|---|---|
| chr6 | 73802964 | 73802965 | B-Mem_6163 | −0.2283957096026279 |
| chr14 | 90467233 | 90467233 | B-Mem_2165 | −0.2030894937241492 |
| chr11 | 103497071 | 103497071 | B-Mem_1308 | −0.2021652608878442 |
| chr3 | 59334675 | 59334676 | B-Mem_4761 | −0.1852144069085415 |
| chr1 | 89780176 | 89780176 | B-Mem_276 | −0.1776366739639492 |
| chr10 | 2280019 | 2280019 | B-Mem_645 | −0.175064054903808 |
| chr1 | 242398802 | 242398803 | B-Mem_613 | −0.1639971565464631 |
| chr6 | 12033479 | 12033479 | B-Mem_6016 | −0.1627111269970084 |
| chr13 | 86746170 | 86746170 | B-Mem_1931 | −0.1552557408117346 |
| chr7 | 28358203 | 28358203 | B-Mem_6480 | −0.1411978197246404 |
| chr22 | 34305386 | 34305386 | B-Mem_4536 | −0.1398103467126233 |
| chr20 | 46855741 | 46855741 | B-Mem_4250 | −0.1371321427694607 |
| chr2 | 98374198 | 98374198 | B-Mem_3762 | −0.13564971010077 |
| chr1 | 223124894 | 223124894 | B-Mem_529 | −0.1338028442416104 |
| chrX | 23884217 | 23884217 | B-Mem_7548 | −0.1305845941960707 |
| chr11 | 70274678 | 70274678 | B-Mem_1233 | −0.1275608324698802 |
| chr8 | 35159735 | 35159735 | B-Mem_6905 | −0.1255265214643461 |
| chr6 | 142098817 | 142098817 | B-Mem_6302 | −0.1239267660539116 |
| chr1 | 224148253 | 224148253 | B-Mem_543 | −0.1181586154744448 |
| chr13 | 27600203 | 27600203 | B-Mem_1814 | −0.1178311672237022 |
| chr18 | 7760683 | 7760683 | B-Mem_3049 | −0.1148797400028455 |
| chr1 | 181313050 | 181313050 | B-Mem_438 | −0.1135596976741774 |
| chr4 | 57706075 | 57706075 | B-Mem_5228 | −0.1132330299157094 |
| chr2 | 194645972 | 194645972 | B-Mem_3991 | −0.1130270192961715 |
| chr4 | 170659897 | 170659897 | B-Mem_5459 | −0.110419960954212 |
| chr14 | 63138010 | 63138010 | B-Mem_2095 | −0.1098717438892863 |
| chr11 | 102958394 | 102958394 | B-Mem_1307 | −0.1061917460883332 |
| chr17 | 55023838 | 55023838 | B-Mem_2918 | −0.1055323248632449 |
| chr6 | 138109034 | 138109034 | B-Mem_6291 | −0.1029381731592764 |
| chr3 | 59922555 | 59922555 | B-Mem_4765 | −0.1016685363776916 |
| chr1 | 152204027 | 152204027 | B-Mem_362 | −0.0979569388471365 |
| chr9 | 71658925 | 71658925 | B-Mem_7262 | −0.0970321374283545 |
| chr5 | 31790767 | 31790767 | B-Mem_5622 | −0.0932706672087575 |
| chr18 | 65108091 | 65108091 | B-Mem_3190 | −0.0881109584246932 |
| chr1 | 27356860 | 27356860 | B-Mem_122 | −0.0879625954522244 |
| chr10 | 126044817 | 126044817 | B-Mem_982 | −0.0867808981208309 |
| chr20 | 53549223 | 53549223 | B-Mem_4270 | −0.0841059416847453 |
| chr1 | 44717144 | 44717144 | B-Mem_175 | −0.0811656803355662 |
| chr1 | 60610044 | 60610044 | B-Mem_214 | −0.080319468186741 |
| chr6 | 111917726 | 111917727 | B-Mem_6238 | −0.0765909733274403 |
| chr1 | 18613887 | 18613887 | B-Mem_84 | −0.0763337033920045 |
| chr4 | 8554864 | 8554864 | B-Mem_5120 | −0.0722074717238387 |
| chr1 | 157339448 | 157339448 | B-Mem_374 | −0.0711355475933754 |
| chr10 | 16454313 | 16454313 | B-Mem_695 | −0.0667077344213908 |
| chr1 | 104554420 | 104554420 | B-Mem_309 | −0.0639869206385076 |
| chr3 | 11606467 | 11606467 | B-Mem_4655 | −0.0616774405831731 |
| chr9 | 35097564 | 35097564 | B-Mem_7236 | 0.0647202646791766 |
| chr8 | 131902808 | 131902808 | B-Mem_7110 | 0.0647561289959215 |
| chr1 | 224174148 | 224174148 | B-Mem_545 | 0.0676482369407441 |
| chr16 | 75425761 | 75425761 | B-Mem_2657 | 0.0676784190510444 |
| chr5 | 74937572 | 74937572 | B-Mem_5702 | 0.0679877379492628 |
| chr20 | 56767240 | 56767241 | B-Mem_4284 | 0.0699830546990528 |
| chr10 | 69079717 | 69079717 | B-Mem_830 | 0.0773680399856888 |
| chr20 | 27476554 | 27476554 | B-Mem_4189 | 0.0780425404308161 |
| chr11 | 70887146 | 70887146 | B-Mem_1237 | 0.0786999314561501 |
| chr14 | 22949516 | 22949516 | B-Mem_2019 | 0.0790992448975665 |
| chr6 | 4020423 | 4020423 | B-Mem_5986 | 0.0794178525455957 |
| chr14 | 63761909 | 63761910 | B-Mem_2098 | 0.0802427218936756 |
| chr17 | 75706172 | 75706172 | B-Mem_2975 | 0.0806465990568646 |
| chr4 | 75626583 | 75626583 | B-Mem_5256 | 0.0809093618562385 |
| chr2 | 23641709 | 23641709 | B-Mem_3584 | 0.0838174988420379 |
| chr2 | 194654636 | 194654636 | B-Mem_3992 | 0.0844696398436668 |
| chr3 | 112081370 | 112081370 | B-Mem_4891 | 0.0886448832110273 |
| chr20 | 54067335 | 54067335 | B-Mem_4274 | 0.0955521949549734 |
| chr15 | 63876169 | 63876169 | B-Mem_2342 | 0.0967058150633226 |
| chr13 | 27605492 | 27605492 | B-Mem_1815 | 0.0972377048937463 |
| chr4 | 159548897 | 159548898 | B-Mem_5423 | 0.0977799942748357 |
| chr16 | 33991709 | 33991709 | B-Mem_2562 | 0.1011936590964484 |
| chr21 | 29076475 | 29076476 | B-Mem_4372 | 0.1014789400315759 |
| chr13 | 32969149 | 32969149 | B-Mem_1825 | 0.1021804389477964 |
| chr2 | 57195206 | 57195206 | B-Mem_3669 | 0.1028411623422528 |
| chrX | 19541826 | 19541826 | B-Mem_7538 | 0.1055059905757838 |
| chr11 | 134691345 | 134691345 | B-Mem_1428 | 0.1161113080166762 |
| chr4 | 127705896 | 127705896 | B-Mem_5368 | 0.1166508210195596 |
| chr11 | 43162275 | 43162275 | B-Mem_1145 | 0.1194389845560008 |
| chr7 | 27252577 | 27252577 | B-Mem_6476 | 0.1214871056498325 |
| chr2 | 198768146 | 198768146 | B-Mem_4000 | 0.1234182404193053 |
| chr6 | 92774422 | 92774422 | B-Mem_6198 | 0.1241434821519959 |
| chr6 | 15659576 | 15659576 | B-Mem_6026 | 0.1308437007109232 |
| chr2 | 219751097 | 219751097 | B-Mem_4049 | 0.1316914773330559 |
| chr2 | 109099023 | 109099023 | B-Mem_3797 | 0.1419346201420216 |
| chr4 | 74420868 | 74420869 | B-Mem_5255 | 0.1421572678606877 |
| chrY | 12074965 | 12074965 | B-Mem_7819 | 0.1631816559620538 |
| chr4 | 181401090 | 181401090 | B-Mem_5479 | 0.1657742584599645 |
| chr14 | 68599594 | 68599594 | B-Mem_2107 | 0.1668198404397327 |
| chr21 | 29027788 | 29027788 | B-Mem_4371 | 0.1702290996070042 |
| chr8 | 15833228 | 15833228 | B-Mem_6843 | 0.1749681897403905 |
| chr2 | 173703921 | 173703921 | B-Mem_3949 | 0.1833400023521677 |
| chrX | 18703997 | 18703997 | B-Mem_7536 | 0.184500770397731 |
| chr11 | 84526903 | 84526903 | B-Mem_1275 | 0.1984267184551097 |
| chr14 | 54860818 | 54860818 | B-Mem_2075 | 0.2115391711116829 |
| chr3 | 1024121 | 1024121 | B-Mem_4626 | 0.2136855105107569 |
| chr7 | 154118532 | 154118532 | B-Mem_6772 | 0.2157609478344008 |
| chr22 | 35449550 | 35449550 | B-Mem_4539 | 0.2189020382495668 |
| chrX | 125596173 | 125596173 | B-Mem_7749 | 0.2281841344851008 |
| chr9 | 63477983 | 63477983 | B-Mem_7251 | 0.2360802185560665 |
| chr2 | 108549325 | 108549325 | B-Mem_3796 | 0.2363155351882347 |
| chr9 | 33213088 | 33213088 | B-Mem_7227 | 0.2661487109271673 |
| chr18 | 18754998 | 18754998 | B-Mem_3069 | 0.3082947731068414 |
| chr13 | 89932740 | 89932740 | B-Mem_1938 | 0.3138581303050221 |

BA B-Naive

| | | | | |
|---|---|---|---|---|
| chr2 | 198741635 | 198741635 | B-Naive_1957 | −0.6384211764184814 |
| chr5 | 170894954 | 170894954 | B-Naive_2875 | −0.3802927523111113 |
| chr12 | 125316762 | 125316762 | B-Naive_834 | −0.3394425015961829 |
| chr3 | 131706371 | 131706371 | B-Naive_2420 | −0.3360997723652841 |
| chr4 | 28190045 | 28190045 | B-Naive_2546 | −0.3312769795502114 |
| chr8 | 6850018 | 6850018 | B-Naive_3334 | −0.3132813468359776 |
| chr2 | 22269929 | 22269929 | B-Naive_1768 | −0.2664002207964385 |
| chr2 | 102908474 | 102908474 | B-Naive_1858 | −0.2612411775143239 |
| chr14 | 98586580 | 98586580 | B-Naive_1049 | −0.253254318652791 |
| chr5 | 181266020 | 181266020 | B-Naive_2896 | −0.2512155341756552 |
| chr5 | 112167444 | 112167444 | B-Naive_2817 | −0.2438257296588547 |
| chr12 | 62866114 | 62866114 | B-Naive_768 | −0.2197491193234354 |
| chr1 | 44720383 | 44720384 | B-Naive_85 | −0.2017754953626114 |
| chr4 | 170684337 | 170684337 | B-Naive_2689 | −0.1939940239909361 |
| chr1 | 25082452 | 25082453 | B-Naive_46 | −0.1823525909863695 |
| chr1 | 70749338 | 70749338 | B-Naive_116 | −0.1785234162103664 |
| chr18 | 24358258 | 24358258 | B-Naive_1526 | −0.1722272522192944 |
| chr17 | 55101975 | 55101975 | B-Naive_1439 | −0.1681877333473698 |
| chr22 | 26723098 | 26723099 | B-Naive_2212 | −0.1435710228359689 |
| chr12 | 128992392 | 128992392 | B-Naive_842 | −0.1368776877150257 |
| chr13 | 62221017 | 62221018 | B-Naive_905 | −0.1255344570098707 |
| chr19 | 55404692 | 55404692 | B-Naive_1723 | 0.1243816008532072 |
| chr5 | 159152758 | 159152758 | B-Naive_2859 | −0.1217270789925119 |
| chr9 | 88868166 | 88868166 | B-Naive_3588 | −0.1202376739590769 |
| chr17 | 81629785 | 81629785 | B-Naive_1493 | −0.1185987241256577 |
| chr14 | 52205640 | 52205640 | B-Naive_992 | −0.1063111121397947 |
| chr16 | 77639700 | 77639701 | B-Naive_1301 | −0.1053703026779974 |
| chr18 | 10842608 | 10842608 | B-Naive_1510 | −0.0960214720713407 |
| chr6 | 2393315 | 2393315 | B-Naive_2899 | −0.089247824142101 |
| chr4 | 170657949 | 170657949 | B-Naive_2687 | −0.0879539652152161 |
| chr3 | 12374340 | 12374340 | B-Naive_2303 | −0.0821855897477096 |
| chr18 | 61687383 | 61687383 | B-Naive_1574 | −0.0781607485387977 |
| chr11 | 67396661 | 67396661 | B-Naive_581 | −0.0761679245269008 |
| chr18 | 53782981 | 53782981 | B-Naive_1566 | −0.0713294305111866 |
| chr13 | 39811058 | 39811068 | B-Naive_886 | −0.0668571805637548 |
| chr18 | 33347811 | 33347811 | B-Naive_1538 | −0.0656317519283483 |
| chr4 | 168114817 | 168114818 | B-Naive_2682 | −0.0636110715526309 |
| chr1 | 67886027 | 67886027 | B-Naive_114 | −0.0592027508287278 |
| chr1 | 25082027 | 25082027 | B-Naive_45 | −0.0589005908233852 |
| chr11 | 128136608 | 128136608 | B-Naive_675 | −0.0539818497201545 |
| chr4 | 66145406 | 66145406 | B-Naive_2590 | −0.0520056113635483 |
| chr10 | 52798056 | 52798056 | B-Naive_356 | −0.0471199167519372 |
| chr18 | 12066447 | 12066447 | B-Naive_1514 | −0.04595981591034 |
| chr1 | 233462263 | 233462263 | B-Naive_255 | −0.0398455835290859 |
| chr10 | 78862599 | 78862599 | B-Naive_398 | −0.0387940083899227 |
| chr15 | 41582256 | 41582256 | B-Naive_1101 | −0.0374926752209703 |
| chr3 | 7896180 | 7896180 | B-Naive_2289 | −0.0265190139675729 |
| chr22 | 41278517 | 41278517 | B-Naive_2240 | −0.0258602646674696 |
| chr11 | 104157161 | 104157161 | B-Naive_637 | −0.0185809367790771 |
| chr3 | 83598055 | 83598055 | B-Naive_2381 | −0.0065025358823686 |
| chr19 | 12624514 | 12624514 | B-Naive_1630 | 0.0042203138896191 |
| chr19 | 11826100 | 11826100 | B-Naive_1628 | 0.0057001973525908 |
| chr11 | 70887146 | 70887146 | B-Naive_599 | 0.0121296414104611 |
| chr2 | 224007666 | 224007666 | B-Naive_1995 | 0.0136931962425454 |
| chr22 | 48020686 | 48020686 | B-Naive_2261 | 0.0166678776363089 |
| chrX | 120757333 | 120757333 | B-Naive_3869 | 0.0167691511114432 |
| chr11 | 34703628 | 34703628 | B-Naive_527 | 0.0168515774975641 |
| chr15 | 80428928 | 80428928 | B-Naive_1156 | 0.0171334203169167 |
| chr4 | 24481095 | 24481095 | B-Naive_2542 | 0.0180793447919439 |
| chr1 | 236642085 | 236642085 | B-Naive_265 | 0.0240684085974822 |
| chr8 | 15833228 | 15833228 | B-Naive_3357 | 0.0264731260238 |
| chr9 | 94857661 | 94857661 | B-Naive_3598 | 0.0315834079431823 |
| chr8 | 50066146 | 50066146 | B-Naive_3396 | 0.0414031670627693 |
| chrY | 11778236 | 11778236 | B-Naive_3940 | 0.0468324562971079 |
| chr11 | 67962120 | 67962120 | B-Naive_583 | 0.0548278688589996 |
| chrX | 45332171 | 45332171 | B-Naive_3735 | 0.0700770639357502 |
| chr16 | 33959160 | 33959160 | B-Naive_1244 | 0.0708203054703548 |
| chr11 | 66321161 | 66321161 | B-Naive_579 | 0.077826446726097 |
| chr12 | 13361281 | 13361281 | B-Naive_697 | 0.0903330297033359 |
| chr18 | 58646524 | 58646524 | B-Naive_1572 | 0.0952169349216745 |
| chr4 | 161526053 | 161526053 | B-Naive_2671 | 0.105179663364719 |
| chr7 | 103158954 | 103158954 | B-Naive_3245 | 0.1126004907443714 |
| chr11 | 1649719 | 1649719 | B-Naive_486 | 0.1355508768279008 |
| chr15 | 42070623 | 42070624 | B-Naive_1103 | 0.1390614032657619 |
| chr1 | 58639715 | 58639715 | B-Naive_104 | 0.1423576171107719 |
| chr2 | 5089267 | 5089267 | B-Naive_1738 | 0.1443123257290809 |
| chr9 | 77682850 | 77682850 | B-Naive_3577 | 0.1481667703658694 |
| chrY | 16659827 | 16659827 | B-Naive_3943 | 0.1532251035459639 |
| chr9 | 129256642 | 129256642 | B-Naive_3641 | 0.1645941552756736 |
| chr3 | 99662114 | 99662114 | B-Naive_2395 | 0.1758830046083036 |
| chr12 | 86497918 | 86497918 | B-Naive_788 | 0.188720778974107 |
| chr10 | 125506490 | 125506491 | B-Naive_464 | 0.1954695990960346 |
| chr11 | 33439602 | 33439602 | B-Naive_525 | 0.1958011607179785 |
| chr7 | 43985743 | 43985743 | B-Naive_3178 | 0.2107331050672098 |
| chr7 | 129979435 | 129979435 | B-Naive_3278 | 0.2166713503303536 |
| chr14 | 19159159 | 19159159 | B-Naive_966 | 0.2199525018058354 |
| chr7 | 168484 | 168484 | B-Naive_3104 | 0.2292313777860042 |
| chr16 | 68277963 | 68277963 | B-Naive_1291 | 0.2374289524378901 |
| chr12 | 3047510 | 3047510 | B-Naive_685 | 0.261252730862697 |
| chr6 | 164576088 | 164576089 | B-Naive_3077 | 0.2841653135753259 |
| chr8 | 50230522 | 50230523 | B-Naive_3400 | 0.2845191615686344 |
| chr8 | 7538869 | 7538869 | B-Naive_3336 | 0.2853492429156177 |
| chrY | 56826846 | 56826846 | B-Naive_3944 | 0.2982932358131941 |
| chr15 | 49546444 | 49546444 | B-Naive_1114 | 0.3057005756289088 |
| chrX | 14565734 | 14565734 | B-Naive_3692 | 0.3424850906985831 |
| chrY | 9921400 | 9921400 | B-Naive_3937 | 0.4261353920247306 |
| chrX | 98149487 | 98149487 | B-Naive_3833 | 0.4569613357892091 |
| chr4 | 90054037 | 90054037 | B-Naive_2610 | 0.4872432429409412 |
| chr1 | 34814939 | 34814939 | B-Naive_68 | 0.5508562806036634 |
| chrY | 5640241 | 5640241 | B-Naive_3935 | 0.5559037820767669 |

BA Monocyte

| | | | | |
|---|---|---|---|---|
| chr12 | 74757060 | 74757060 | Monocyte_1845 | −0.2878246853916625 |
| chr7 | 95292178 | 95292179 | Monocyte_7703 | −0.2338490170374841 |
| chr9 | 90352298 | 90352298 | Monocyte_8560 | −0.1911085463287029 |
| chr7 | 142397856 | 142397856 | Monocyte_7843 | −0.1814720906968036 |
| chr15 | 43339157 | 43339157 | Monocyte_2654 | −0.1742538814619023 |
| chr3 | 163781805 | 163781805 | Monocyte_5830 | −0.1635653549476313 |
| chr7 | 40333358 | 40333358 | Monocyte_7567 | −0.1626415297856606 |
| chr2 | 105824545 | 105824546 | Monocyte_4432 | −0.1616496816318454 |
| chr18 | 10842608 | 10842609 | Monocyte_3560 | −0.158574528145583 |
| chr1 | 171235441 | 171235441 | Monocyte_431 | −0.1518496112937901 |
| chr3 | 163723049 | 163723049 | Monocyte_5826 | −0.1511435029451924 |
| chr12 | 32035358 | 32035359 | Monocyte_1718 | −0.143067092062721 |
| chr13 | 97780136 | 97780137 | Monocyte_2251 | −0.1402199655322296 |
| chrX | 13290039 | 13290039 | Monocyte_8812 | −0.1335848977605516 |
| chr5 | 126817054 | 126817054 | Monocyte_6742 | −0.1306084875782989 |
| chr6 | 76865760 | 76865760 | Monocyte_7169 | −0.1304041104523973 |
| chr21 | 17044336 | 17044336 | Monocyte_5065 | −0.1297392955942673 |
| chr1 | 62930671 | 62930672 | Monocyte_242 | −0.1185651489099044 |
| chr15 | 42799914 | 42799914 | Monocyte_2646 | −0.1177930591451145 |
| chr18 | 61687383 | 61687384 | Monocyte_3693 | −0.1173992465681086 |
| chr9 | 20231165 | 20231165 | Monocyte_8438 | −0.1166275982172799 |
| chr19 | 14952424 | 14952424 | Monocyte_3881 | −0.1161100707208004 |
| chr17 | 39127540 | 39127540 | Monocyte_3332 | −0.1138529026612266 |
| chr1 | 116024675 | 116024676 | Monocyte_355 | −0.111940604051256 |
| chr13 | 36775709 | 36775709 | Monocyte_2110 | −0.110599314165504 |
| chr1 | 120285669 | 120285669 | Monocyte_1549 | −0.1092014178634572 |
| chr11 | 11748318 | 11748318 | Monocyte_1195 | −0.1076934945215068 |
| chr6 | 21107048 | 21107048 | Monocyte_7004 | −0.1056349295555497 |
| chr20 | 56774275 | 56774276 | Monocyte_4987 | −0.1050161239308454 |
| chr3 | 36420554 | 36420555 | Monocyte_5513 | −0.0982695568029807 |
| chr4 | 57706075 | 57706075 | Monocyte_6103 | −0.0963120379898127 |
| chr12 | 32037602 | 32037603 | Monocyte_1719 | −0.0954340585617216 |
| chr16 | 11303249 | 11303249 | Monocyte_2901 | −0.0944406865216849 |
| chr12 | 53692352 | 53692352 | Monocyte_1794 | −0.0939571271745654 |
| chr4 | 114115674 | 114115674 | Monocyte_6241 | −0.0932886041093078 |
| chr1 | 179548005 | 179548006 | Monocyte_462 | −0.0926368694617512 |
| chr16 | 24705574 | 24705574 | Monocyte_2948 | −0.0894757989639132 |
| chr8 | 3467471 | 3467472 | Monocyte_7935 | −0.086382981134818 |
| chrX | 49581795 | 49581795 | Monocyte_8930 | −0.0860638180839164 |
| chr19 | 47468122 | 47468122 | Monocyte_4048 | −0.085212523029277 |
| chr8 | 40604156 | 40604156 | Monocyte_8077 | −0.0837710427476736 |
| chr14 | 89958331 | 89958331 | Monocyte_2494 | −0.0827629515017409 |
| chr8 | 19164481 | 19164481 | Monocyte_8014 | −0.0807438583828261 |
| chr5 | 36533754 | 36533755 | Monocyte_6567 | −0.0803559335755844 |
| chr4 | 56119778 | 56119778 | Monocyte_6095 | −0.0802720637116017 |
| chr4 | 69124322 | 69124323 | Monocyte_6135 | −0.0794403155620521 |
| chr15 | 45456408 | 45456408 | Monocyte_2666 | −0.0790080284011459 |
| chr16 | 58660620 | 58660620 | Monocyte_3048 | −0.0756492265574325 |
| chr2 | 231248549 | 231248549 | Monocyte_4739 | −0.0734958302908259 |
| chr3 | 163734750 | 163734751 | Monocyte_5828 | −0.0727015292816613 |
| chr16 | 59558412 | 59558412 | Monocyte_3049 | −0.0716015780897913 |
| chr9 | 129400880 | 129400880 | Monocyte_8703 | −0.0676421861479806 |
| chr2 | 70825449 | 70825449 | Monocyte_4339 | 0.0699214172390332 |
| chr2 | 23640686 | 23640687 | Monocyte_4225 | 0.0722057056043661 |
| chrX | 9791968 | 9791968 | Monocyte_8796 | 0.0734474556230323 |
| chr1 | 166240542 | 166240542 | Monocyte_415 | 0.0740296310496317 |
| chr14 | 76601694 | 76601694 | Monocyte_2463 | 0.0758713829671288 |
| chr7 | 80977388 | 80977388 | Monocyte_7678 | 0.076099814162012 |
| chr1 | 11263844 | 11263844 | Monocyte_66 | 0.0763474564789885 |
| chr15 | 85028757 | 85028757 | Monocyte_2781 | 0.0802892264291651 |
| chr17 | 12759328 | 12759328 | Monocyte_3253 | 0.0820526366677764 |
| chr13 | 73207743 | 73207743 | Monocyte_2189 | 0.0849986669932856 |
| chr5 | 97216045 | 97216045 | Monocyte_6677 | 0.0852357843713451 |
| chr6 | 39237581 | 39237581 | Monocyte_7081 | 0.0902928105752848 |
| chr1 | 217951657 | 217951657 | Monocyte_551 | 0.0927944409474384 |
| chr7 | 106636053 | 106636053 | Monocyte_7751 | 0.0941921303964675 |
| chr2 | 42470436 | 42470436 | Monocyte_4272 | 0.0970035551746628 |
| chr20 | 53648481 | 53648481 | Monocyte_4973 | 0.0982405079917148 |
| chr10 | 131744888 | 131744888 | Monocyte_1124 | 0.1009507408434645 |
| chrY | 9138267 | 9138267 | Monocyte_9264 | 0.1020193886568536 |
| chrX | 53751592 | 53751592 | Monocyte_8949 | 0.104603997201496 |
| chr4 | 150526693 | 150526693 | Monocyte_6332 | 0.1066161185374578 |
| chr14 | 24488334 | 24488334 | Monocyte_2332 | 0.1067129868895544 |
| chr12 | 85758165 | 85758165 | Monocyte_1872 | 0.1071343013161067 |
| chr12 | 38991538 | 38991538 | Monocyte_1740 | 0.1118660284522391 |
| chr9 | 61616797 | 61616797 | Monocyte_8499 | 0.1160304068422863 |
| chr18 | 69799289 | 69799289 | Monocyte_3716 | 0.1187969489884724 |
| chrX | 66057104 | 66057104 | Monocyte_8975 | 0.1238000826869205 |
| chr18 | 79919519 | 79919519 | Monocyte_3765 | 0.1249171987878637 |
| chrX | 135470048 | 135470048 | Monocyte_9178 | 0.1263856317835253 |
| chr13 | 28525864 | 28525864 | Monocyte_2084 | 0.1268267605279465 |
| chr7 | 114802982 | 114802982 | Monocyte_7779 | 0.1291677967012166 |
| chr14 | 75289215 | 75289215 | Monocyte_2458 | 0.1337229229210663 |
| chr11 | 86934112 | 86934112 | Monocyte_1448 | 0.1346638329933771 |
| chr8 | 49479839 | 49479839 | Monocyte_8108 | 0.1386095773974572 |
| chr10 | 46118350 | 46118350 | Monocyte_828 | 0.1406389899528426 |
| chr21 | 41453808 | 41453809 | Monocyte_5160 | 0.1430362778197252 |
| chr9 | 28895880 | 28895880 | Monocyte_8461 | 0.1455319350564421 |
| chr7 | 32081931 | 32081932 | Monocyte_7544 | 0.1465963382533613 |
| chrY | 9002289 | 9002289 | Monocyte_9263 | 0.1471145964425273 |
| chr8 | 15833228 | 15833229 | Monocyte_8000 | 0.1696402121267848 |
| chr13 | 89298442 | 89298442 | Monocyte_2230 | 0.2074390457604398 |
| chr6 | 168075891 | 168075891 | Monocyte_7412 | 0.211061012935664 |
| chr3 | 80866318 | 80866318 | Monocyte_5647 | 0.225661269593019 |
| chr11 | 1649719 | 1649719 | Monocyte_1154 | 0.235336674029071 |
| chrY | 19841787 | 19841787 | Monocyte_9278 | 0.2870446640150154 |
| chrY | 14359490 | 14359490 | Monocyte_9272 | 0.3580488053748154 |
| chrY | 7449643 | 7449643 | Monocyte_9262 | 0.3657835020393599 |
| chr17 | 22888617 | 22888617 | Monocyte_3288 | 0.4392265060501383 |
| chr7 | 145241570 | 145241570 | Monocyte_7848 | 0.4788348881089256 |

BA NK-cell1

| | | | | |
|---|---|---|---|---|
| chr9 | 24351772 | 24351772 | NK-cell1_8420 | −0.2630671669589844 |
| chr4 | 146947470 | 146947470 | NK-cell1_6190 | −0.1944819946408479 |
| chr7 | 80095717 | 80095717 | NK-cell1_7631 | −0.1879702364275075 |
| chr6 | 19913948 | 19913949 | NK-cell1_6948 | −0.1751174197155695 |
| chr1 | 102553839 | 102553839 | NK-cell1_352 | 0.1646445246872307 |
| chr2 | 74336609 | 74336609 | NK-cell1_4088 | −0.1625251400579457 |
| chr12 | 104600986 | 104600986 | NK-cell1_1979 | −0.1572005808189405 |
| chr7 | 36088177 | 36088177 | NK-cell1_7489 | −0.1495551555146644 |
| chr2 | 3415237 | 3415237 | NK-cell1_3906 | −0.144478618558518 |
| chr1 | 207925710 | 207925710 | NK-cell1_597 | −0.143595603283986 |
| chr10 | 93081340 | 93081340 | NK-cell1_1039 | −0.1313691524783782 |
| chrX | 126134432 | 126134432 | NK-cell1_8927 | −0.129491933423715 |
| chr4 | 96251107 | 96251107 | NK-cell1_6041 | −0.1267585409316597 |
| chr8 | 86493430 | 86493431 | NK-cell1_8173 | −0.1169319031275418 |
| chr15 | 95584973 | 95584973 | NK-cell1_2810 | −0.1158258682170267 |
| chr11 | 127689953 | 127689953 | NK-cell1_1626 | 0.1127502145040772 |
| chr8 | 17411757 | 17411757 | NK-cell1_7958 | −0.1100742341107678 |
| chr18 | 24265893 | 24265893 | NK-cell1_3483 | −0.1094083213280951 |
| chr2 | 28293739 | 28293739 | NK-cell1_3975 | −0.1078306693516247 |
| chr12 | 12013292 | 12013292 | NK-cell1_1722 | −0.1054637599462623 |
| chr16 | 78837251 | 78837251 | NK-cell1_3053 | −0.1032894089523769 |
| chr12 | 109684876 | 109684876 | NK-cell1_1993 | −0.1007556342861911 |
| chr7 | 37365116 | 37365117 | NK-cell1_7492 | −0.0990503597173678 |
| chr6 | 46834632 | 46834632 | NK-cell1_7035 | −0.0990001983001498 |
| chr12 | 4067233 | 4067233 | NK-cell1_1694 | −0.0972360767065702 |
| chr12 | 16757779 | 16757779 | NK-cell1_1734 | −0.0939191284195378 |
| chr4 | 169449272 | 169449272 | NK-cell1_6254 | −0.091009765877723 |
| chr2 | 155304121 | 155304121 | NK-cell1_4358 | −0.0905661439019997 |
| chr12 | 50495201 | 50495201 | NK-cell1_1835 | −0.0890530905710786 |
| chr20 | 40467179 | 40467179 | NK-cell1_4781 | −0.0873139737424982 |
| chr18 | 58296337 | 58296337 | NK-cell1_3594 | −0.0832178154923618 |
| chr1 | 162073559 | 162073559 | NK-cell1_468 | −0.082358918149561 |
| chr8 | 92741469 | 92741469 | NK-cell1_8197 | −0.0789385517108435 |
| chr7 | 53630973 | 53630973 | NK-cell1_7559 | −0.076999982716642 |
| chr12 | 118475190 | 118475190 | NK-cell1_2029 | −0.0769845811500117 |
| chr2 | 11355508 | 11355519 | NK-cell1_3932 | −0.0747424333723114 |
| chr7 | 89268471 | 89268471 | NK-cell1_7665 | −0.0738775050761261 |
| chr20 | 32798008 | 32798008 | NK-cell1_4747 | −0.0736823965711533 |
| chr12 | 22157753 | 22157753 | NK-cell1_1756 | −0.072416797863363 |
| chr1 | 234496233 | 234496233 | NK-cell1_681 | −0.0716586023335129 |
| chr19 | 30119993 | 30119993 | NK-cell1_3769 | −0.0710680398389592 |
| chr12 | 52599134 | 52599134 | NK-cell1_1846 | −0.0709950760467846 |
| chr14 | 94443827 | 94443827 | NK-cell1_2543 | −0.0695835308189739 |
| chr9 | 6370971 | 6370971 | NK-cell1_8367 | −0.0681773180252304 |
| chr2 | 241014228 | 241014228 | NK-cell1_4629 | −0.0674395920382179 |
| chr15 | 55909216 | 55909216 | NK-cell1_2690 | −0.0632187299747328 |
| chr11 | 130207330 | 130207330 | NK-cell1_1639 | −0.0631535357352006 |
| chr20 | 41712090 | 41712090 | NK-cell1_4786 | −0.0600128613262477 |
| chr7 | 134992806 | 134992806 | NK-cell1_7792 | −0.0598432987569616 |
| chr1 | 9685837 | 9685837 | NK-cell1_50 | 0.0611372976792548 |
| chr19 | 670387 | 670388 | NK-cell1_3674 | 0.0650132520498686 |
| chr4 | 82621463 | 82621463 | NK-cell1_6002 | 0.0655208092507764 |
| chr13 | 80091845 | 80091845 | NK-cell1_2261 | 0.0678356774030789 |
| chrX | 35693975 | 35693975 | NK-cell1_8779 | 0.0697794165145924 |
| chrX | 11208306 | 11208306 | NK-cell1_8719 | 0.0715271013522988 |
| chr4 | 99073303 | 99073303 | NK-cell1_6051 | 0.0736424374840359 |
| chr7 | 13980679 | 13980679 | NK-cell1_7412 | 0.0742130307360031 |
| chr1 | 32024418 | 32024418 | NK-cell1_152 | 0.0757116362158582 |
| chr2 | 158887877 | 158887877 | NK-cell1_4374 | 0.07810816779961 |
| chr5 | 140857752 | 140857752 | NK-cell1_6718 | 0.0795110410328017 |
| chr9 | 130158298 | 130158298 | NK-cell1_8643 | 0.0798511125338307 |
| chr11 | 133549542 | 133549542 | NK-cell1_1662 | 0.0816848118647743 |
| chr21 | 44772818 | 44772818 | NK-cell1_5004 | 0.081772797208649 |
| chr14 | 28210578 | 28210578 | NK-cell1_2381 | 0.0841036604222437 |
| chrX | 41013113 | 41013113 | NK-cell1_8789 | 0.0859081546423422 |
| chr7 | 127773224 | 127773224 | NK-cell1_7779 | 0.0924355383364307 |
| chr10 | 62088129 | 62088129 | NK-cell1_948 | 0.0935081778371137 |
| chr14 | 86813089 | 86813089 | NK-cell1_2519 | 0.0947653203925922 |
| chr8 | 73002718 | 73002718 | NK-cell1_8130 | 0.0961507957464054 |
| chr22 | 17827344 | 17827345 | NK-cell1_5020 | 0.0971182427159612 |
| chr6 | 53189537 | 53189537 | NK-cell1_7055 | 0.0986469013900183 |
| chr17 | 2124338 | 2124338 | NK-cell1_3109 | 0.1007426185760344 |
| chr2 | 71561650 | 71561650 | NK-cell1_4076 | 0.1008079991533792 |
| chr3 | 123935786 | 123935786 | NK-cell1_5522 | 0.101915476999733 |
| chr15 | 60877370 | 60877370 | NK-cell1_2704 | 0.1057057504104236 |
| chr22 | 41401753 | 41401753 | NK-cell1_5098 | 0.1079942140448065 |
| chrX | 137653898 | 137653898 | NK-cell1_8960 | 0.1109935009478569 |
| chr12 | 121898580 | 121898581 | NK-cell1_2041 | 0.1119338399128798 |
| chr10 | 87539952 | 87539952 | NK-cell1_1025 | 0.1167231000710136 |
| chr12 | 81034498 | 81034498 | NK-cell1_1921 | 0.1170226108014198 |
| chr2 | 200455807 | 200455807 | NK-cell1_4476 | 0.1170435230034931 |
| chr1 | 112418001 | 112418001 | NK-cell1_390 | 0.1177529343157856 |
| chr3 | 3935379 | 3935379 | NK-cell1_5174 | 0.121154982124912 |
| chr4 | 174248059 | 174248059 | NK-cell1_6272 | 0.1259518168408385 |
| chr14 | 98124253 | 98124253 | NK-cell1_2554 | 0.1280261085033186 |
| chr7 | 27911141 | 27911141 | NK-cell1_7457 | 0.1288724943024998 |
| chr10 | 28139340 | 28139340 | NK-cell1_844 | 0.1323027524741271 |
| chrX | 9687642 | 9687642 | NK-cell1_8712 | 0.1387253503742855 |
| chr19 | 48622051 | 48622051 | NK-cell1_3847 | 0.1577385650333399 |
| chr15 | 57163122 | 57163123 | NK-cell1_2693 | 0.1593423824057946 |
| chr18 | 12765382 | 12765382 | NK-cell1_3466 | 0.1605609914080771 |
| chr7 | 53454705 | 53454705 | NK-cell1_7557 | 0.1655799303958533 |
| chr6 | 90629049 | 90629049 | NK-cell1_7153 | 0.1754876503628217 |
| chr8 | 122863298 | 122863298 | NK-cell1_8293 | 0.2066343825270021 |
| chrX | 21988387 | 21988388 | NK-cell1_8746 | 0.2121998953000349 |
| chr21 | 28866538 | 28866538 | NK-cell1_4946 | 0.2152503816443217 |
| chr10 | 95132299 | 95132299 | NK-cell1_1046 | 0.2258799274125558 |
| chr4 | 3938793 | 3938793 | NK-cell1_5766 | 0.2288283560305685 |
| chr2 | 10407018 | 10407018 | NK-cell1_3927 | 0.2884940707302449 |
| chr10 | 52563867 | 52563867 | NK-cell1_919 | 0.3101871575671975 |

BA NK-cell2

| | | | | |
|---|---|---|---|---|
| chr17 | 22876334 | 22876334 | NK-cell2_1976 | −0.6808304830760891 |
| chr2 | 240367188 | 240367188 | NK-cell2_2867 | −0.4745944298740069 |
| chr22 | 45220567 | 45220567 | NK-cell2_3195 | −0.4532361822417316 |
| chr8 | 14681999 | 14681999 | NK-cell2_4792 | −0.4172422478629509 |
| chr17 | 31648665 | 31648665 | NK-cell2_1989 | −0.329710337321271 |
| chr5 | 175300037 | 175300038 | NK-cell2_4169 | −0.3019730860139884 |
| chr15 | 58680795 | 58680795 | NK-cell2_1627 | −0.230669642546084 |
| chr4 | 159554384 | 159554384 | NK-cell2_3826 | −0.2286837582812198 |
| chr8 | 110035417 | 110035417 | NK-cell2_4967 | −0.226488831775134 |
| chr16 | 81497055 | 81497055 | NK-cell2_1882 | −0.2085665300098467 |
| chr1 | 188361426 | 188361426 | NK-cell2_304 | −0.1909508129844365 |
| chr20 | 999234 | 999234 | NK-cell2_2874 | −0.1791617892859978 |
| chr2 | 105839931 | 105839931 | NK-cell2_2623 | −0.1779401781260592 |
| chr15 | 80202118 | 80202118 | NK-cell2_1668 | −0.1771884293224609 |
| chr3 | 56400947 | 56400947 | NK-cell2_3325 | −0.1763854405282445 |
| chr11 | 121094978 | 121094978 | NK-cell2_943 | −0.1754529474219016 |
| chr3 | 7891741 | 7891741 | NK-cell2_3233 | −0.167438142406634 |
| chr20 | 32750015 | 32750015 | NK-cell2_2939 | −0.1638603325133438 |
| chr1 | 232339128 | 232339128 | NK-cell2_387 | −0.1573837070343074 |
| chr8 | 83874715 | 83874715 | NK-cell2_4936 | −0.1562702143801652 |
| chr4 | 166515430 | 166515430 | NK-cell2_3842 | −0.1494259911786121 |
| chr3 | 171200648 | 171200648 | NK-cell2_3483 | −0.1336159076779391 |
| chr20 | 50483272 | 50483272 | NK-cell2_2973 | −0.131190190653515 |
| chr2 | 229229186 | 229229186 | NK-cell2_2825 | −0.1299794772373889 |
| chr11 | 104085549 | 104085549 | NK-cell2_900 | −0.1287872836892559 |
| chr12 | 101915989 | 101915990 | NK-cell2_1152 | −0.1249416769149483 |
| chr4 | 164954206 | 164954207 | NK-cell2_3835 | −0.1200059247351273 |
| chr16 | 51216001 | 51216001 | NK-cell2_1811 | −0.1043227053049029 |
| chr12 | 101890203 | 101890204 | NK-cell2_1150 | −0.1034079659655723 |
| chr18 | 33327200 | 33327200 | NK-cell2_2179 | −0.1028849253492718 |
| chr8 | 3325193 | 3325193 | NK-cell2_4766 | −0.1001646916965367 |
| chr12 | 62867090 | 62867090 | NK-cell2_1105 | −0.098017597730182 |
| chr1 | 104024568 | 104024568 | NK-cell2_199 | −0.0937872116396083 |
| chr8 | 26547737 | 26547737 | NK-cell2_4829 | −0.0845620222792294 |
| chr16 | 86165578 | 86165579 | NK-cell2_1896 | −0.0818026141086841 |
| chr8 | 1470040 | 1470040 | NK-cell2_4759 | −0.0752399806497258 |
| chr17 | 14664673 | 14664699 | NK-cell2_1967 | −0.0647827578392468 |
| chr6 | 164743838 | 164743839 | NK-cell2_4453 | −0.0522534613144826 |
| chr19 | 13247539 | 13247539 | NK-cell2_2317 | −0.0427200510601722 |
| chr16 | 51220308 | 51220308 | NK-cell2_1812 | −0.04226775085455 |
| chr2 | 120859682 | 120859683 | NK-cell2_2646 | −0.0398939302746564 |
| chr21 | 41470927 | 41470927 | NK-cell2_3081 | 0.0382039860371209 |
| chr16 | 86347816 | 86347816 | NK-cell2_1899 | 0.0388658936177195 |
| chr2 | 70823922 | 70823922 | NK-cell2_2564 | 0.0466963037735455 |
| chr22 | 19653775 | 19653775 | NK-cell2_3114 | 0.0479563770074662 |
| chr13 | 76095198 | 76095198 | NK-cell2_1307 | 0.0485776777817794 |
| chrX | 66153496 | 66153496 | NK-cell2_5383 | 0.0488958329061039 |
| chr4 | 113002455 | 113002455 | NK-cell2_3760 | 0.051031829371996 |
| chr10 | 97533569 | 97533569 | NK-cell2_590 | 0.0539682823100621 |
| chr12 | 131290276 | 131290276 | NK-cell2_1207 | 0.055865286186038 |
| chr20 | 54891561 | 54891561 | NK-cell2_2987 | 0.058785932858599 |
| chr10 | 125506490 | 125506491 | NK-cell2_660 | 0.0589835458826684 |
| chr1 | 236637817 | 236637817 | NK-cell2_397 | 0.0609835635950766 |
| chr13 | 44901578 | 44901578 | NK-cell2_1262 | 0.061267736393192 |
| chr10 | 28971737 | 28971737 | NK-cell2_479 | 0.0614013998316187 |
| chr1 | 176752180 | 176752180 | NK-cell2_284 | 0.0620021508538711 |
| chr3 | 189332516 | 189332517 | NK-cell2_3508 | 0.0623640612269476 |
| chr5 | 1188169 | 1188170 | NK-cell2_3903 | 0.0645310819744857 |
| chr19 | 49435931 | 49435932 | NK-cell2_2423 | 0.0742058008317423 |
| chr22 | 11441508 | 11441508 | NK-cell2_3099 | 0.0747876860936772 |
| chr11 | 58865782 | 58865782 | NK-cell2_812 | 0.0750016807164525 |
| chr8 | 69249600 | 69249600 | NK-cell2_4916 | 0.0781651781063828 |
| chr8 | 139737802 | 139737802 | NK-cell2_5011 | 0.0787446078892149 |
| chr13 | 30079365 | 30079365 | NK-cell2_1243 | 0.0801780318141705 |
| chr1 | 41825179 | 41825179 | NK-cell2_113 | 0.0856250982504213 |
| chr10 | 29300687 | 29300688 | NK-cell2_480 | 0.0861560423094914 |
| chr3 | 52835247 | 52835248 | NK-cell2_3310 | 0.0925358662213278 |
| chr17 | 5797079 | 5797079 | NK-cell2_1941 | 0.096123751062494 |
| chr7 | 21860647 | 21860647 | NK-cell2_4508 | 0.096405581268331 |
| chr16 | 20928283 | 20928283 | NK-cell2_1776 | 0.0972736144908464 |
| chr14 | 100758584 | 100758584 | NK-cell2_1531 | 0.0977397564313287 |
| chr6 | 34161723 | 34161723 | NK-cell2_4259 | 0.1003759159987469 |
| chr9 | 3234232 | 3234232 | NK-cell2_5043 | 0.1068353846541444 |
| chr4 | 32276512 | 32276512 | NK-cell2_3630 | 0.1075996999684194 |
| chrY | 15455766 | 15455772 | NK-cell2_5595 | 0.1164521535509432 |
| chr1 | 16058679 | 16058679 | NK-cell2_50 | 0.1219339174987612 |
| chr12 | 130750171 | 130750171 | NK-cell2_1205 | 0.1222965334563441 |
| chr11 | 67314875 | 67314875 | NK-cell2_837 | 0.1233703841806289 |
| chr6 | 71231331 | 71231331 | NK-cell2_4305 | 0.1249360273502232 |
| chr18 | 75802052 | 75802052 | NK-ccll2_2260 | 0.1283392136541755 |
| chr16 | 10822636 | 10822636 | NK-cell2_1750 | 0.1302616916307992 |
| chr2 | 132182309 | 132182309 | NK-cell2_2670 | 0.1413440590331827 |
| chr4 | 3931212 | 3931213 | NK-cell2_3554 | 0.1528072937148267 |
| chr4 | 26645890 | 26645890 | NK-cell2_3616 | 0.1599636637564954 |
| chr8 | 3194680 | 3194680 | NK-cell2_4763 | 0.1653024144249416 |
| chr13 | 73416708 | 73416708 | NK-cell2_1304 | 0.1704552032401918 |
| chrX | 118645419 | 118645420 | NK-cell2_5483 | 0.1894324617908414 |
| chr6 | 909437 | 909437 | NK-cell2_4186 | 0.1922162193880383 |
| chr10 | 30710822 | 30710822 | NK-cell2_487 | 0.1922693545613909 |
| chr19 | 16852446 | 16852447 | NK-cell2_2335 | 0.1980842834228997 |
| chr1 | 48800914 | 48800915 | NK-cell2_130 | 0.2012181666835834 |
| chr4 | 3930950 | 3930950 | NK-cell2_3553 | 0.2084792833042462 |
| chr20 | 52539668 | 52539668 | NK-cell2_2979 | 0.2229340078734731 |
| chr11 | 71655831 | 71655831 | NK-cell2_857 | 0.2306832731141132 |
| chr1 | 76788156 | 76788156 | NK-cell2_161 | 0.2620271833389856 |
| chrY | 26520989 | 26520989 | NK-cell2_5596 | 0.3063935028403274 |
| chr1 | 174763313 | 174763314 | NK-cell2_280 | 0.3107183263708449 |
| chr2 | 175616275 | 175616275 | NK-cell2_2729 | 0.3251818176798424 |
| chr1 | 34814939 | 34814939 | NK-cell2_103 | 0.4368284771166382 |
| chr6 | 168075917 | 168075917 | NK-cell2_4462 | 0.6774469957068235 |

BA Tc-Mem

| | | | | |
|---|---|---|---|---|
| chr8 | 142978129 | 142978129 | Tc-Mem_12314 | −0.175491699526238 |
| chr1 | 210376689 | 210376689 | Tc-Mem_810 | −0.171938972332803 |
| chr19 | 47246687 | 47246687 | Tc-Mem_5936 | −0.1460701918436905 |
| chr19 | 42990933 | 42990934 | Tc-Mem_5893 | −0.1381272170799014 |
| chr17 | 9643233 | 9643233 | Tc-Mem_4748 | −0.1369923367118536 |
| chr5 | 5811087 | 5811087 | Tc-Mem_9521 | −0.135402424487295 |
| chr5 | 162600385 | 162600385 | Tc-Mem_10097 | −0.1328679535247315 |
| chr8 | 40604156 | 40604156 | Tc-Mem_11881 | −0.1326005456533095 |
| chr2 | 30392016 | 30392016 | Tc-Mem_6216 | −0.12979289060388 |
| chr22 | 18050668 | 18050669 | Tc-Mem_7614 | −0.1286116906589862 |
| chr21 | 31088697 | 31088697 | Tc-Mem_7460 | −0.1280975291192934 |
| chr8 | 14709561 | 14709561 | Tc-Mem_11749 | −0.119958422954438 |
| chr8 | 52031712 | 52031712 | Tc-Mem_11946 | −0.1184715711093776 |
| chr2 | 37680292 | 37680442 | Tc-Mem_6244 | −0.1174655822039225 |
| chr19 | 14963971 | 14963971 | Tc-Mem_5684 | −0.1152977008620456 |
| chr9 | 112118570 | 112118570 | Tc-Mem_12735 | −0.1120620948209883 |
| chr13 | 84397692 | 84397692 | Tc-Mem_3202 | −0.1091742500563369 |
| chr12 | 130165182 | 130165182 | Tc-Mem_2917 | −0.1089311195615209 |
| chr10 | 54295841 | 54295841 | Tc-Mem_1262 | −0.1064194238771321 |
| chr21 | 40134686 | 40134686 | Tc-Mem_7540 | −0.1053503017803169 |
| chr15 | 92004333 | 92004334 | Tc-Mem_4086 | −0.1017268391430395 |
| chr21 | 40302149 | 40302149 | Tc-Mem_7544 | −0.1009329802736063 |
| chr1 | 112895654 | 112895655 | Tc-Mem_509 | −0.0988391312977244 |
| chr4 | 55969247 | 55969248 | Tc-Mem_8952 | −0.0962411850360841 |
| chr13 | 78919807 | 78919808 | Tc-Mem_3194 | −0.0951000105690259 |
| chr1 | 236136705 | 236136705 | Tc-Mem_944 | −0.0941477021281968 |
| chr2 | 198712655 | 198712655 | Tc-Mem_6815 | 0.0884300365289722 |
| chr1 | 186537480 | 186537480 | Tc-Mem_721 | −0.0879890596720725 |
| chr1 | 233749225 | 233749225 | Tc-Mem_931 | −0.0857156463724687 |
| chrX | 149788561 | 149788562 | Tc-Mem_13335 | −0.085259554368952 |
| chr17 | 50534060 | 50534060 | Tc-Mem_4960 | −0.0833230936217735 |
| chr10 | 16500184 | 16500184 | Tc-Mem_1108 | −0.082908226183621 |
| chr19 | 38042051 | 38042052 | Tc-Mem_5848 | −0.0824398801543516 |
| chr2 | 198728176 | 198728176 | Tc-Mem_6818 | −0.0819132676431381 |
| chr17 | 81631701 | 81631701 | Tc-Mem_5167 | −0.0816423548971414 |
| chr18 | 73977134 | 73977135 | Tc-Mem_5501 | 0.0787790327221021 |
| chr1 | 222919307 | 222919307 | Tc-Mem_864 | −0.0786649707591951 |
| chr4 | 168160849 | 168160850 | Tc-Mem_9335 | −0.0786350415937889 |
| chr9 | 20233570 | 20233570 | Tc-Mem_12414 | −0.07577422268264 |
| chr14 | 102983549 | 102983549 | Tc-Mem_3724 | −0.0744866312793364 |
| chr12 | 102042151 | 102042151 | Tc-Mem_2754 | −0.0737566882562974 |
| chr10 | 16457682 | 16457682 | Tc-Mem_1107 | −0.0699769670919772 |
| chrY | 19910354 | 19910354 | Tc-Mem_13391 | 0.0703331483674532 |
| chrX | 79656859 | 79656859 | Tc-Mem_13146 | 0.0710057067583745 |
| chr17 | 6156149 | 6156149 | Tc-Mem_4714 | 0.0727033357550897 |
| chr18 | 12585096 | 12585096 | Tc-Mem_5224 | 0.0762396030491491 |
| chr6 | 155106267 | 155106267 | Tc-Mem_10814 | 0.0777962627649731 |
| chr22 | 24415698 | 24415698 | Tc-Mem_7648 | 0.0800950492878659 |
| chr9 | 126471733 | 126471733 | Tc-Mem_12804 | 0.0802606896247214 |
| chr1 | 92351289 | 92351289 | Tc-Mem_443 | 0.0811171854067169 |
| chr9 | 62607814 | 62607814 | Tc-Mem_12514 | 0.0854991798884879 |
| chr6 | 54065659 | 54065659 | Tc-Mem_10478 | 0.0860979816211958 |
| chr18 | 74181531 | 74181531 | Tc-Mem_5503 | 0.0868144817613589 |
| chrY | 9661261 | 9661261 | Tc-Mem_13363 | 0.0917342527667038 |
| chrY | 10032712 | 10032712 | Tc-Mem_13364 | 0.0953907281517 |
| chr6 | 165013190 | 165013190 | Tc-Mem_10860 | 0.0957333456587169 |
| chrY | 19111179 | 19111179 | Tc-Mem_13389 | 0.0965439897973246 |
| chrY | 11985791 | 11985791 | Tc-Mem_13375 | 0.0977582070403787 |
| chrY | 19630039 | 19630039 | Tc-Mem_13390 | 0.0983379128202344 |
| chr18 | 15024315 | 15024315 | Tc-Mem_5240 | 0.0987530315590243 |
| chr3 | 145333912 | 145333912 | Tc-Mem_8437 | 0.1023858646789129 |
| chrY | 14253076 | 14253076 | Tc-Mem_13380 | 0.1028374683477505 |
| chrY | 22724994 | 22724994 | Tc-Mem_13392 | 0.1075075849680372 |
| chr10 | 735897 | 735897 | Tc-Mem_1018 | 0.1128770097520264 |
| chr19 | 16852446 | 16852447 | Tc-Mem_5708 | 0.1133183125916817 |
| chr2 | 109980284 | 109980284 | Tc-Mem_6529 | 0.1168044649976735 |
| chr17 | 3572690 | 3572690 | Tc-Mem_4692 | 0.1186648564053108 |
| chrY | 11103316 | 11103316 | Tc-Mem_13369 | 0.1196625626169759 |
| chr1 | 56291619 | 56291620 | Tc-Mem_340 | 0.1208671408205668 |
| chr20 | 62333907 | 62333907 | Tc-Mem_7335 | 0.1221132065510325 |
| chr20 | 28645214 | 28645214 | Tc-Mem_7143 | 0.1221336885248681 |
| chr22 | 41726922 | 41726922 | Tc-Mem_7755 | 0.1236176865220659 |
| chrY | 8785103 | 8785103 | Tc-Mem_13360 | 0.124388476584645 |
| chrY | 14679300 | 14679300 | Tc-Mem_13381 | 0.1252638874807601 |
| chr7 | 28851404 | 28851404 | Tc-Mem_11067 | 0.1269813563151062 |
| chr19 | 27271248 | 27271248 | Tc-Mem_5762 | 0.1341989584040544 |
| chrY | 11743579 | 11743579 | Tc-Mem_13373 | 0.1356858926288193 |
| chr4 | 3931212 | 3931213 | Tc-Mem_8703 | 0.1369519164283332 |
| chr15 | 64408359 | 64408359 | Tc-Mem_3957 | 0.1391819615471274 |
| chr4 | 148007220 | 148007221 | Tc-Mem_9254 | 0.1559642070253249 |
| chr8 | 37491600 | 37491600 | Tc-Mem_11859 | 0.1565956806338044 |
| chr2 | 1159055 | 1159055 | Tc-Mem_6049 | 0.1566047878880121 |
| chr5 | 137968146 | 137968146 | Tc-Mem_9987 | 0.156723456434269 |
| chr12 | 8307182 | 8307182 | Tc-Mem_2365 | 0.160468090482698 |
| chr8 | 840630 | 840630 | Tc-Mem_11655 | 0.1606411075689684 |
| chr19 | 3763576 | 3763576 | Tc-Mem_5596 | 0.1631930335502822 |
| chr2 | 183925889 | 183925889 | Tc-Mem_6767 | 0.1654914232237196 |
| chrY | 12549594 | 12549594 | Tc-Mem_13378 | 0.1679900523873032 |
| chrY | 19105220 | 19105220 | Tc-Mem_13388 | 0.1781362127033539 |
| chrY | 10653307 | 10653307 | Tc-Mem_13366 | 0.1883702832342795 |
| chr4 | 145054642 | 145054642 | Tc-Mem_9241 | 0.1952072947952545 |
| chrY | 18408420 | 18408420 | Tc-Mem_13387 | 0.2063947908437374 |
| chrY | 11940881 | 11940881 | Tc-Mem_13374 | 0.2177343370613205 |
| chrY | 12355648 | 12355648 | Tc-Mem_13377 | 0.2248512148953909 |
| chr8 | 68675512 | 68675512 | Tc-Mem_12010 | 0.2274384815395558 |
| chr11 | 64354939 | 64354939 | Tc-Mem_1968 | 0.2286974826453501 |
| chr13 | 44182647 | 44182647 | Tc-Mem_3081 | 0.2325716813409446 |
| chr2 | 227196608 | 227196608 | Tc-Mem_6933 | 0.251628791991319 |
| chrY | 12980642 | 12980642 | Tc-Mem_13379 | 0.2521022131450451 |
| chrY | 15996350 | 15996350 | Tc-Mem_13386 | 0.267997299797668 |

| | | | |
|---|---|---|---|
| Chromosome | Start Position | End position | Weight |

OP B-Mem

| | | | |
|---|---|---|---|
| chr1 | 2313783 | 2313783 | 0.0063130281344816 |
| chr1 | 2326884 | 2326885 | −0.010819390389768 |
| chr1 | 2469095 | 2469096 | 0.0045586632561654 |
| chr1 | 3359809 | 3359810 | 0.0102252658215654 |
| chr1 | 3620627 | 3620627 | −0.0004472618663428 |
| chr1 | 3846325 | 3846326 | −0.0184484966692343 |
| chr1 | 3852000 | 3852001 | −0.0315538983612923 |
| chr1 | 3861674 | 3861674 | −0.0027147805538855 |
| chr1 | 4527909 | 4527909 | −0.0002977481422921 |
| chr1 | 7852375 | 7852375 | −0.0021643235312901 |
| chr1 | 8933633 | 8933634 | 0.0064369852690344 |
| chr1 | 8937002 | 8937002 | 0.0156805624663496 |
| chr1 | 10677505 | 10677505 | −0.0109502164795473 |
| chr1 | 13879637 | 13879674 | 0.006923435491554 |
| chr1 | 20670846 | 20670846 | 0.0139532564779222 |
| chr1 | 20729086 | 20729086 | −0.0016702836979702 |
| chr1 | 21559542 | 21559542 | 0.004795054821116 |
| chr1 | 22511357 | 22511357 | 0.0090243606927105 |
| chr1 | 22539846 | 22539846 | −0.0120796149773882 |
| chr1 | 24237083 | 24237084 | 0.0015914774210153 |
| chr1 | 24237395 | 24237396 | 0.0086387141372298 |
| chr1 | 24239831 | 24239831 | 0.0148220690806663 |
| chr1 | 24240858 | 24240858 | 0.0038836016601936 |
| chr1 | 24241173 | 24241174 | 0.0108219885122128 |
| chr1 | 24255003 | 24255003 | 0.0046242696881793 |
| chr1 | 24296062 | 24296062 | 0.0048059456364577 |
| chr1 | 24311080 | 24311080 | 0.0045718803874271 |
| chr1 | 25082452 | 25082452 | −0.0054768615166848 |
| chr1 | 25794587 | 25794587 | 0.0141124294548173 |
| chr1 | 25828709 | 25828709 | 0.0114355113089059 |
| chr1 | 25867019 | 25867019 | 0.0030143172582695 |
| chr1 | 25874673 | 25874673 | 0.004801949819323 |
| chr1 | 26178265 | 26178265 | 0.0004830102433428 |
| chr1 | 26215289 | 26215289 | −0.0053911017839551 |
| chr1 | 29430850 | 29430850 | −0.007368470103631 |
| chr1 | 30673641 | 30673641 | −0.008090136277281 |
| chr1 | 35207726 | 35207727 | 0.0086002558705126 |
| chr1 | 35287754 | 35287755 | 0.0076315138136557 |
| chr1 | 35782697 | 35782697 | 0.0063108047767326 |
| chr1 | 36658801 | 36658801 | −0.0001754665432637 |
| chr1 | 37037277 | 37037307 | 0.0067345285212452 |
| chr1 | 37775440 | 37775440 | −0.0074313599098632 |
| chr1 | 44811473 | 44811473 | 0.0075339827317999 |
| chr1 | 46648893 | 46648893 | 0.0108751780074282 |
| chr1 | 47213407 | 47213408 | −0.0180667574742857 |
| chr1 | 54297379 | 54297380 | 0.0019564431470533 |
| chr1 | 54343083 | 54343083 | 0.000753853824723 |
| chr1 | 54432657 | 54432657 | 0.0033641408779675 |
| chr1 | 56648790 | 56648790 | 0.0058920216656275 |
| chr1 | 56712116 | 56712116 | 0.0028817766723085 |
| chr1 | 57276260 | 57276261 | 0.0003916934208645 |
| chr1 | 58139183 | 58139183 | 8.455511441636654e−06 |
| chr1 | 58400849 | 58400849 | −0.0135299178133138 |
| chr1 | 58406851 | 58406851 | −0.0027523657478119 |
| chr1 | 58808693 | 58808694 | 0.0031802597679046 |
| chr1 | 58918606 | 58918606 | 0.0030279095070972 |
| chr1 | 60211179 | 60211179 | 0.0023132440549011 |
| chr1 | 61305972 | 61305972 | 0.0038505808801605 |
| chr1 | 61591897 | 61591897 | 0.0120921181075595 |
| chr1 | 61602602 | 61602602 | 0.0127033189949443 |
| chr1 | 61625267 | 61625268 | −0.0117454994289574 |
| chr1 | 61638217 | 61638217 | 0.015404255639113 |
| chr1 | 61976116 | 61976117 | −0.0043665123986641 |
| chr1 | 62218488 | 62218489 | 0.0056187255243873 |
| chr1 | 62260202 | 62260202 | 0.0092005915166261 |
| chr1 | 63020156 | 63020157 | 0.0128321687495741 |
| chr1 | 64305330 | 64305331 | −0.011874355608589 |
| chr1 | 64414807 | 64414808 | 0.0120177623970092 |
| chr1 | 67208601 | 67208601 | −0.0002819543529285 |
| chr1 | 67581417 | 67581417 | 0.0126871641116174 |
| chr1 | 68881444 | 68881444 | 0.0113213349296867 |
| chr1 | 69988557 | 69988557 | 0.0045141773345729 |
| chr1 | 76623835 | 76623835 | 0.0165370943534209 |
| chr1 | 81397405 | 81397405 | 0.0010512166885934 |
| chr1 | 81493457 | 81493457 | 0.0023986649628331 |
| chr1 | 83251324 | 83251324 | 0.0029529798533949 |
| chr1 | 83770989 | 83770989 | 0.0038424565000423 |
| chr1 | 85047495 | 85047495 | −0.0069697901059885 |
| chr1 | 85279883 | 85279884 | −0.0062804966535798 |
| chr1 | 86357515 | 86357515 | −0.0075351421070475 |
| chr1 | 86964076 | 86964076 | 0.0097625494581361 |
| chr1 | 87102096 | 87102096 | 0.0028391211541659 |
| chr1 | 87622308 | 87622308 | 0.0217088244908429 |
| chr1 | 87653517 | 87653518 | 0.0153262036645922 |
| chr1 | 87654011 | 87654011 | 0.0248787624094704 |
| chr1 | 87659840 | 87659840 | −0.0060185460504759 |
| chr1 | 88642175 | 88642175 | 0.0072500317396565 |
| chr1 | 88952235 | 88952235 | 0.0006643458160068 |
| chr1 | 91227373 | 91227373 | 0.0008419034611744 |
| chr1 | 93104810 | 93104810 | 0.0046033836324521 |
| chr1 | 93949142 | 93949142 | 0.0052863563862637 |
| chr1 | 95520897 | 95520897 | 0.0011026755744005 |
| chr1 | 99626106 | 99626107 | 0.0107415647905562 |
| chr1 | 104680151 | 104680152 | −0.0008030546664051 |
| chr1 | 107468968 | 107468969 | 0.0098523874114729 |
| chr1 | 109389286 | 109389286 | 0.006954964095112 |
| chr1 | 109558757 | 109558757 | 0.0133840567276344 |
| chr1 | 110330187 | 110330187 | −0.0055010361839594 |
| chr1 | 110410727 | 110410727 | 0.0009545445483603 |
| chr1 | 110571326 | 110571326 | 0.0069227314756383 |

| | | | | |
|---|---|---|---|---|
| Chromosome | Start Position | End position | Cell ID | Weight |

BA Tc-Naive

| | | | | |
|---|---|---|---|---|
| chr9 | 118656180 | 118656180 | Tc-Naive_7963 | −0.339904710308958 |
| chr2 | 160073688 | 160073688 | Tc-Naive_4230 | −0.2920738473669165 |
| chr17 | 1955847 | 1955854 | Tc-Naive_2987 | −0.2870355784334801 |
| chr11 | 52760308 | 52760308 | Tc-Naive_1248 | −0.2778406559618482 |
| chr14 | 85398348 | 85398348 | Tc-Naive_2328 | −0.2566494602825004 |
| chr14 | 98799972 | 98799972 | Tc-Naive_2372 | −0.2109194808243925 |
| chr4 | 69088433 | 69088433 | Tc-Naive_5676 | −0.2029318248395739 |
| chr5 | 64276849 | 64276849 | Tc-Naive_6121 | −0.1965104169219183 |
| chr15 | 58680795 | 58680795 | Tc-Naive_2526 | −0.1952333996206149 |
| chr4 | 69057122 | 69057123 | Tc-Naive_5673 | −0.1765738932232771 |
| chr7 | 101329526 | 101329527 | Tc-Naive_7092 | −0.1556005699431586 |
| chr19 | 58067210 | 58067211 | Tc-Naive_3840 | −0.1554318365727715 |
| chr4 | 68773525 | 68773525 | Tc-Naive_5667 | −0.1513317207486275 |
| chr7 | 93422818 | 93422818 | Tc-Naive_7072 | −0.1498044886871529 |
| chr9 | 10124920 | 10124920 | Tc-Naive_7758 | −0.1318143181945662 |
| chr13 | 32764063 | 32764063 | Tc-Naive_1974 | −0.119446789016526 |
| chr12 | 12888136 | 12888174 | Tc-Naive_1558 | −0.1157845468133375 |
| chr10 | 17304544 | 17304544 | Tc-Naive_717 | −0.1138221993356799 |
| chr2 | 2509486 | 2509486 | Tc-Naive_3856 | −0.1125698908685707 |
| chr1 | 3757588 | 3757588 | Tc-Naive_24 | −0.1115219373130726 |
| chr16 | 65843682 | 65843682 | Tc-Naive_2865 | −0.1110184605216818 |
| chrX | 69559027 | 69559027 | Tc-Naive_8281 | −0.1089699219987785 |
| chr4 | 68741224 | 68741225 | Tc-Naive_5664 | −0.1079024600209719 |
| chr10 | 92975164 | 92975164 | Tc-Naive_925 | −0.10693630669828 |
| chr18 | 38398552 | 38398553 | Tc-Naive_3392 | −0.1062532447626567 |
| chr22 | 26723098 | 26723099 | Tc-Naive_4844 | −0.1049033441256781 |
| chr1 | 233703508 | 233703508 | Tc-Naive_583 | −0.1010675764161093 |
| chr17 | 81624518 | 81624519 | Tc-Naive_3302 | −0.0999538353592039 |
| chr4 | 56431830 | 56431831 | Tc-Naive_5626 | −0.0989884924896843 |
| chr12 | 101897248 | 101897248 | Tc-Naive_1809 | −0.0977271542078127 |
| chr18 | 54932428 | 54932428 | Tc-Naive_3449 | −0.0961671676544332 |
| chr4 | 187013161 | 187013161 | Tc-Naive_5974 | −0.0957636837945881 |
| chr2 | 193616863 | 193616863 | Tc-Naive_4292 | −0.0952181004055425 |
| chr3 | 186138192 | 186138193 | Tc-Naive_5411 | 0.0918959040027379 |
| chr1 | 188356343 | 188356343 | Tc-Naive_446 | −0.0885833721408572 |
| chr2 | 233850587 | 233850587 | Tc-Naive_4396 | −0.0885113385864448 |
| chr10 | 77611573 | 77611573 | Tc-Naive_886 | −0.0876961228404451 |
| chr19 | 39773635 | 39773635 | Tc-Naive_3748 | −0.08179383789554 |
| chr2 | 105824545 | 105824546 | Tc-Naive_4098 | −0.0801277188428553 |
| chr11 | 20032213 | 20032213 | Tc-Naive_1158 | 0.0760422883320764 |
| chr20 | 51063215 | 51063215 | Tc-Naive_4591 | −0.0744167257654382 |
| chr5 | 178455627 | 178455627 | Tc-Naive_6351 | −0.0720101440826758 |
| chr5 | 56076289 | 56076289 | Tc-Naive_6107 | −0.0709171596951259 |
| chr21 | 39120932 | 39120933 | Tc-Naive_4746 | −0.0686996024361894 |
| chr3 | 56453901 | 56453901 | Tc-Naive_5140 | −0.0668344810397379 |
| chr18 | 12350506 | 12350506 | Tc-Naive_3350 | 0.066528867665703 |
| chr8 | 50227161 | 50227162 | Tc-Naive_7468 | 0.0670613562401729 |
| chr4 | 9432773 | 9432774 | Tc-Naive_5516 | 0.0684597667794532 |
| chr18 | 12634111 | 12634117 | Tc-Naive_3353 | 0.0684993881948014 |
| chr8 | 143780790 | 143780790 | Tc-Naive_7718 | 0.0722231359187839 |
| chr2 | 220056843 | 220056843 | Tc-Naive_4344 | 0.0725519542442147 |
| chr12 | 38740818 | 38740818 | Tc-Naive_1645 | 0.0740463955359173 |
| chr2 | 102730635 | 102730635 | Tc-Naive_4091 | 0.0764939915170001 |
| chr1 | 82473854 | 82473854 | Tc-Naive_235 | 0.0766881222506611 |
| chr8 | 49481496 | 49481496 | Tc-Naive_7461 | 0.0817084670736108 |
| chr16 | 32084761 | 32084761 | Tc-Naive_2779 | 0.0843450992860984 |
| chr3 | 141191418 | 141191418 | Tc-Naive_5318 | 0.0863089357237354 |
| chr3 | 185371737 | 185371737 | Tc-Naive_5407 | 0.0883772186651 |
| chr11 | 113744391 | 113744391 | Tc-Naive_1431 | 0.0885862487672207 |
| chr4 | 170565179 | 170565180 | Tc-Naive_5922 | 0.0896274020483331 |
| chr2 | 23640686 | 23640687 | Tc-Naive_3925 | 0.0907900941814112 |
| chr8 | 91609709 | 91609710 | Tc-Naive_7559 | 0.0916147676723256 |
| chr10 | 8806823 | 8806823 | Tc-Naive_682 | 0.0952314683797617 |
| chr18 | 27898393 | 27898393 | Tc-Naive_3371 | 0.0953811470881384 |
| chr3 | 195714250 | 195714250 | Tc-Naive_5440 | 0.0975187693821845 |
| chr1 | 1837221 | 1837221 | Tc-Naive_8 | 0.101604523858742 |
| chr1 | 55328231 | 55328231 | Tc-Naive_186 | 0.1071476432928682 |
| chr6 | 76327341 | 76327341 | Tc-Naive_6576 | 0.1086758098395269 |
| chr2 | 6977077 | 6977077 | Tc-Naive_3871 | 0.1104705026188988 |
| chr14 | 79883259 | 79883259 | Tc-Naive_2319 | 0.1108589710447625 |
| chr2 | 36279546 | 36279546 | Tc-Naive_3977 | 0.1112865330330232 |
| chr16 | 13983994 | 13983994 | Tc-Naive_2738 | 0.112524518323921 |
| chr4 | 103552099 | 103552099 | Tc-Naive_5751 | 0.1166967381932331 |
| chr1 | 196464253 | 196464253 | Tc-Naive_463 | 0.1176474463918307 |
| chr5 | 32462647 | 32462648 | Tc-Naive_6070 | 0.1184044932922564 |
| chr4 | 67967462 | 67967462 | Tc-Naive_5661 | 0.1310686663326544 |
| chr10 | 737536 | 737536 | Tc-Naive_639 | 0.1313897590024822 |
| chr14 | 86412630 | 86412630 | Tc-Naive_2332 | 0.1334651583811986 |
| chrX | 66112972 | 66112972 | Tc-Naive_8259 | 0.1340271308219382 |
| chr12 | 3395784 | 3395784 | Tc-Naive_1535 | 0.1407451543446137 |
| chr20 | 45384448 | 45384448 | Tc-Naive_4564 | 0.1429614832935862 |
| chr2 | 219824863 | 219824864 | Tc-Naive_4342 | 0.1455665715881496 |
| chr11 | 6661980 | 6661981 | Tc-Naive_1122 | 0.1457256237079523 |
| chr2 | 266907 | 266907 | Tc-Naive_3844 | 0.150530010995434 |
| chr2 | 72016288 | 72016288 | Tc-Naive_4035 | 0.1568492398528088 |
| chr19 | 16852446 | 16852447 | Tc-Naive_3638 | 0.1678000374987561 |
| chr10 | 116735426 | 116735426 | Tc-Naive_993 | 0.1774770703194533 |
| chrY | 10174739 | 10174739 | Tc-Naive_8566 | 0.1780302700794452 |
| chr22 | 29171868 | 29171868 | Tc-Naive_4851 | 0.1823989086809066 |
| chr10 | 11753239 | 11753239 | Tc-Naive_692 | 0.1832176207814918 |
| chr4 | 179566613 | 179566613 | Tc-Naive_5949 | 0.1849187538407278 |
| chr16 | 11316179 | 11316180 | Tc-Naive_2723 | 0.1869136249601629 |
| chr4 | 9172453 | 9172453 | Tc-Naive_5515 | 0.1995182621174872 |
| chrY | 11323488 | 11323488 | Tc-Naive_8567 | 0.2055243732177388 |
| chr12 | 25562778 | 25562778 | Tc-Naive_1593 | 0.2073611620546679 |
| chrY | 11742426 | 11742426 | Tc-Naive_8568 | 0.2077094803340105 |
| chr10 | 48923525 | 48923525 | Tc-Naive_808 | 0.230850476918727 |
| chr1 | 57569287 | 57569287 | Tc-Naive_191 | 0.2556981620316366 |
| chr4 | 161526054 | 161526054 | Tc-Naive_5891 | 0.259198975058713 |
| chr16 | 16827484 | 16827484 | Tc-Naive_2741 | 0.5508916855359677 |

BA Th-Mem

| | | | | |
|---|---|---|---|---|
| chr16 | 51216000 | 51216001 | Th-Mem_3535 | −0.2458750950881285 |
| chr2 | 44754714 | 44754714 | Th-Mem_5029 | −0.2226111894863905 |
| chr19 | 7669624 | 7669624 | Th-Mem_4511 | −0.2021061937945345 |
| chr21 | 28149937 | 28149967 | Th-Mem_5951 | −0.1952527260042301 |
| chr7 | 112533287 | 112533287 | Th-Mem_8979 | −0.1848841380218278 |
| chr22 | 17318397 | 17318639 | Th-Mem_6057 | −0.1680890997342137 |
| chr10 | 112241256 | 112241256 | Th-Mem_1275 | −0.1631677362224464 |
| chr4 | 29350233 | 29350233 | Th-Mem_7018 | −0.16313243663518 |
| chr10 | 77621261 | 77621262 | Th-Mem_1133 | −0.1543171910438318 |
| chr9 | 118705591 | 118705591 | Th-Mem_10026 | −0.1498656430262666 |
| chr3 | 100565163 | 100565163 | Th-Mem_6581 | −0.1379504701402399 |
| chr4 | 69093465 | 69093465 | Th-Mem_7139 | −0.1350730913430501 |
| chr14 | 102887846 | 102887846 | Th-Mem_2998 | 0.1320585290278925 |
| chr12 | 51928137 | 51928137 | Th-Mem_2053 | −0.1309670427539776 |
| chr13 | 31022635 | 31022636 | Th-Mem_2436 | −0.1290674568759864 |
| chr9 | 129274745 | 129274746 | Th-Mem_10082 | −0.1211537002864475 |
| chr4 | 69087695 | 69087695 | Th-Mem_7138 | −0.1164392243692887 |
| chrX | 13351808 | 13351808 | Th-Mem_10208 | −0.1159904948180395 |
| chr20 | 54916088 | 54916088 | Th-Mem_5826 | −0.1158626218341425 |
| chr4 | 164954206 | 164954207 | Th-Mem_7385 | −0.1130727271476347 |
| chr12 | 119494929 | 119494930 | Th-Mem_2307 | −0.1130037386749458 |
| chr19 | 40966872 | 40966872 | Th-Mem_4697 | −0.1120213995469718 |
| chr11 | 112145790 | 112145790 | Th-Mem_1805 | −0.106445657264804 |
| chr19 | 38054462 | 38054463 | Th-Mem_4685 | −0.1034021517111538 |
| chr9 | 333428 | 333428 | Th-Mem_9707 | −0.1004646683256513 |
| chr7 | 126878510 | 126878510 | Th-Mem_9027 | −0.0995240242931273 |
| chr13 | 70084519 | 70084519 | Th-Mem_2566 | −0.0950646456662843 |
| chr5 | 13831171 | 13831171 | Th-Mem_7541 | −0.0935283884650153 |
| chr19 | 54397463 | 54397463 | Th-Mem_4790 | −0.0912248896921219 |
| chr3 | 119505805 | 119505805 | Th-Mem_6636 | −0.0912218266601778 |
| chr3 | 189953159 | 189953160 | Th-Mem_6855 | −0.0880362989606446 |
| chr17 | 37625074 | 37625074 | Th-Mem_3916 | −0.0873921056965628 |
| chr11 | 2263778 | 2263778 | Th-Mem_1432 | −0.0839965768641566 |
| chr10 | 98103470 | 98103470 | Th-Mem_1211 | −0.0837852179220988 |
| chr15 | 22376079 | 22376079 | Th-Mem_3041 | −0.0829767043992049 |
| chr12 | 84868873 | 84868873 | Th-Mem_2174 | −0.0828675905287018 |
| chr8 | 73135600 | 73135600 | Th-Mem_9425 | −0.0818876401208078 |
| chr2 | 227027903 | 227027903 | Th-Mem_5525 | −0.0789839561844477 |
| chr9 | 6706393 | 6706393 | Th-Mem_9720 | −0.076411962693289 |
| chr8 | 54392497 | 54392498 | Th-Mem_9370 | −0.0748299090774394 |
| chr7 | 82366275 | 82366275 | Th-Mem_8871 | −0.0729507079981164 |
| chr16 | 385814 | 385814 | Th-Mem_3369 | −0.0714029021881466 |
| chr9 | 17494735 | 17494735 | Th-Mem_9755 | −0.0710451238108682 |
| chr21 | 25981252 | 25981252 | Th-Mem_5942 | −0.0707303603012681 |
| chrX | 55386238 | 55386238 | Th-Mem_10344 | 0.0688084984488381 |
| chr11 | 96196558 | 96196558 | Th-Mem_1765 | 0.0690260435737884 |
| chr1 | 204390635 | 204390635 | Th-Mem_656 | 0.0747561236298197 |
| chr1 | 223486293 | 223486293 | Th-Mem_724 | 0.0753018232430965 |
| chrX | 76547744 | 76547744 | Th-Mem_10399 | 0.0757987720521905 |
| chr22 | 29677580 | 29677580 | Th-Mem_6132 | 0.0764408654843258 |
| chr10 | 3343935 | 3343935 | Th-Mem_856 | 0.0779342276036171 |
| chr4 | 170563201 | 170563201 | Th-Mem_7409 | 0.0853404756210384 |
| chr13 | 24448003 | 24448003 | Th-Mem_2404 | 0.0856946922653272 |
| chr10 | 133208303 | 133208303 | Th-Mem_1398 | 0.0857497950630141 |
| chr17 | 46266004 | 46266004 | Th-Mem_3957 | 0.0859858286079862 |
| chrX | 93155315 | 93155315 | Th-Mem_10453 | 0.0862463686958722 |
| chr13 | 102970070 | 102970070 | Th-Mem_2662 | 0.091157265875671 |
| chr22 | 12174860 | 12174860 | Th-Mem_6055 | 0.0918656920854255 |
| chr10 | 21916748 | 21916748 | Th-Mem_933 | 0.0920681681522036 |
| chr17 | 82897472 | 82897472 | Th-Mem_4151 | 0.0964190536151164 |
| chr12 | 121375575 | 121375575 | Th-Mem_2318 | 0.097082949519622 |
| chr5 | 177953047 | 177953047 | Th-Mem_7964 | 0.0978800345244158 |
| chr4 | 147935802 | 147935802 | Th-Mem_7343 | 0.0989243950318569 |
| chr18 | 312695 | 312695 | Th-Mem_4154 | 0.0994221368208313 |
| chr8 | 13406151 | 13406151 | Th-Mem_9208 | 0.1032719880391315 |
| chr10 | 1123112 | 1123112 | Th-Mem_836 | 0.1077269041146934 |
| chr22 | 26466187 | 26466187 | Th-Mem_6115 | 0.1080812546118403 |
| chr10 | 125506490 | 125506490 | Th-Mem_1344 | 0.1087471054738579 |
| chr16 | 79468262 | 79468262 | Th-Mem_3646 | 0.1091411458057322 |
| chr8 | 6308638 | 6308639 | Th-Mem_9176 | 0.1092506025518753 |
| chr12 | 71624881 | 71624882 | Th-Mem_2135 | 0.1138631331339612 |
| chr6 | 20442077 | 20442077 | Th-Mem_8090 | 0.1138683571733146 |
| chr22 | 48013855 | 48013855 | Th-Mem_6237 | 0.1141949876866827 |
| chr4 | 119154870 | 119154870 | Th-Mem_7270 | 0.1171108185180741 |
| chr7 | 103158954 | 103158954 | Th-Mem_8943 | 0.1194390973832814 |
| chr6 | 128980887 | 128980887 | Th-Mem_8387 | 0.1212512637370206 |
| chr4 | 60440508 | 60440508 | Th-Mem_7110 | 0.1232189358383216 |
| chr8 | 51860670 | 51860670 | Th-Mem_9355 | 0.1281186843827669 |
| chr2 | 115560480 | 115560480 | Th-Mem_5229 | 0.1446578941832814 |
| chr10 | 40738785 | 40738785 | Th-Mem_1000 | 0.1497169370400921 |
| chrX | 64804640 | 64804688 | Th-Mem_10363 | 0.1524836608610342 |
| chr7 | 102553711 | 102553711 | Th-Mem_8941 | 0.1543354215519723 |
| chr15 | 61820093 | 61820093 | Th-Mem_3208 | 0.1547928453447161 |
| chr14 | 70621039 | 70621039 | Th-Mem_2863 | 0.1564313221833858 |
| chr1 | 76788156 | 76788156 | Th-Mem_330 | 0.1589001069943569 |
| chr15 | 71593199 | 71593199 | Th-Mem_3254 | 0.1617894117907329 |
| chrY | 6812087 | 6812087 | Th-Mem_10646 | 0.1634619556970255 |
| chrY | 4886474 | 4886474 | Th-Mem_10644 | 0.1732884620894456 |
| chr20 | 45392403 | 45392403 | Th-Mem_5771 | 0.1782138584332714 |
| chr2 | 140775013 | 140775013 | Th-Mem_5297 | 0.2145386342749689 |
| chrY | 16835801 | 16835801 | Th-Mem_10656 | 0.215792022547644 |
| chr2 | 233119625 | 233119625 | Th-Mem_5542 | 0.2205201728684367 |
| chr11 | 67715441 | 67715441 | Th-Mem_1658 | 0.2224567663622853 |
| chrX | 154471021 | 154471021 | Th-Mem_10638 | 0.2375646078208962 |
| chr19 | 5035888 | 5035888 | Th-Mem_4500 | 0.2401955870341497 |
| chr8 | 15833228 | 15833229 | Th-Mem_9219 | 0.2585577650489027 |
| chr11 | 24977466 | 24977466 | Th-Mem_1512 | 0.2888850394750704 |
| chr1 | 851429 | 851429 | Th-Mem_{—} | 0.3096906918231176 |
| chr1 | 21430355 | 21430355 | Th-Mem_142 | 0.3540247428502615 |
| chr7 | 145241570 | 145241570 | Th-Mem_9071 | 0.3736990074805627 |

BA Th-Naive

| | | | | |
|---|---|---|---|---|
| chr11 | 14107407 | 14107407 | Th-Naive_1101 | −0.3440010739715115 |
| chr12 | 27327806 | 27327806 | Th-Naive_1526 | −0.3419634904964836 |
| chr6 | 102027959 | 102027959 | Th-Naive_6647 | −0.2785300180269802 |
| chr1 | 25082452 | 25082453 | Th-Naive_144 | −0.2567754462477963 |
| chr10 | 88054656 | 88054656 | Th-Naive_879 | −0.2430790780022514 |
| chr9 | 20248823 | 20248824 | Th-Naive_7678 | −0.2089062258177356 |
| chr1 | 233653754 | 233653754 | Th-Naive_610 | −0.1875143945418072 |
| chr4 | 149791770 | 149791770 | Th-Naive_5839 | −0.1601753430039492 |
| chr6 | 73709701 | 73709701 | Th-Naive_6595 | −0.1553330834391134 |
| chr4 | 57752277 | 57752278 | Th-Naive_5653 | −0.148251553094779 |
| chr20 | 5043691 | 5043691 | Th-Naive_4405 | −0.1441732436233228 |
| chr6 | 168510939 | 168510939 | Th-Naive_6818 | −0.1418241473492809 |
| chr4 | 57708238 | 57708238 | Th-Naive_5652 | −0.1404661356290591 |
| chr8 | 65056882 | 65056882 | Th-Naive_7436 | −0.1305671523550511 |
| chr10 | 118853239 | 118853239 | Th-Naive_962 | −0.1293760480683587 |
| chr19 | 28830017 | 28830018 | Th-Naive_3591 | 0.1178651378811967 |
| chr12 | 32382165 | 32382165 | Th-Naive_1546 | −0.1158175857104832 |
| chr10 | 122573099 | 122573099 | Th-Naive_978 | −0.1154453340155517 |
| chr10 | 65139426 | 65139426 | Th-Naive_824 | −0.1147610087835362 |
| chr4 | 57707184 | 57707184 | Th-Naive_5651 | −0.114032918648653 |
| chr12 | 48020275 | 48020446 | Th-Naive_1591 | −0.0984734090834491 |
| chr12 | 101915989 | 101915989 | Th-Naive_1727 | −0.097069332426396 |
| chr4 | 71971458 | 71971458 | Th-Naive_5691 | −0.0966979047603976 |
| chr3 | 7088557 | 7088557 | Th-Naive_4967 | −0.0952021209223306 |
| chr21 | 19697885 | 19697886 | Th-Naive_4642 | −0.0939042070083017 |
| chr20 | 63684240 | 63684240 | Th-Naive_4610 | −0.093747298233908 |
| chr10 | 77621261 | 77621261 | Th-Naive_855 | −0.0930135737277638 |
| chr6 | 155642262 | 155642262 | Th-Naive_6756 | −0.0926054564762463 |
| chr12 | 53263682 | 53263683 | Th-Naive_1610 | −0.0913864542577833 |
| chr7 | 97432619 | 97432619 | Th-Naive_7062 | −0.091218383814247 |
| chr8 | 50209850 | 50209850 | Th-Naive_7381 | −0.0867150320936073 |
| chr3 | 56400948 | 56400948 | Th-Naive_5119 | −0.0861752608379593 |
| chr15 | 23485781 | 23485781 | Th-Naive_2348 | −0.0860155192656556 |
| chrX | 128115714 | 128115714 | Th-Naive_8356 | −0.0830449588700249 |
| chr18 | 33327200 | 33327200 | Th-Naive_3318 | −0.0822357943844288 |
| chr17 | 81631701 | 81631701 | Th-Naive_3230 | −0.0816213987593784 |
| chr12 | 80645776 | 80645776 | Th-Naive_1679 | −0.0802289460948578 |
| chr12 | 17313785 | 17313785 | Th-Naive_1508 | −0.0798101353778459 |
| chr4 | 112489271 | 112489271 | Th-Naive_5769 | −0.0791690202706895 |
| chr3 | 36781925 | 36781926 | Th-Naive_5058 | −0.0788185374556632 |
| chr17 | 4591539 | 4591540 | Th-Naive_2958 | −0.0768308008153349 |
| chr6 | 32883063 | 32883063 | Th-Naive_6504 | −0.0721809151102934 |
| chr5 | 175301962 | 175301962 | Th-Naive_6371 | −0.0717903892937037 |
| chr17 | 81013989 | 81013990 | Th-Naive_3215 | −0.0713236501035855 |
| chr7 | 109714465 | 109714465 | Th-Naive_7095 | −0.0712845493984485 |
| chr1 | 233749225 | 233749225 | Th-Naive_611 | −0.0710490999278679 |
| chr3 | 163734750 | 163734750 | Th-Naive_5376 | −0.0708704141147977 |
| chr2 | 198724906 | 198724906 | Th-Naive_4227 | −0.0702372863971832 |
| chr14 | 98707396 | 98707396 | Th-Naive_2282 | −0.0695262318273286 |
| chr12 | 40394577 | 40394578 | Th-Naive_1572 | −0.0693691032764368 |
| chr9 | 20231165 | 20231166 | Th-Naive_7677 | −0.0691843470696817 |
| chr7 | 130389969 | 130389969 | Th-Naive_7138 | −0.065504581698464 |
| chr15 | 33257830 | 33257830 | Th-Naive_2380 | 0.0647826082664001 |
| chr8 | 126294357 | 126294357 | Th-Naive_7546 | 0.0667103713414882 |
| chr15 | 64408359 | 64408359 | Th-Naive_2469 | 0.0717501470793713 |
| chrX | 89808981 | 89808981 | Th-Naive_8251 | 0.0748850671361714 |
| chr4 | 65996170 | 65996171 | Th-Naive_5672 | 0.0762684028808774 |
| chr13 | 28527262 | 28527262 | Th-Naive_1906 | 0.0785460607870038 |
| chr20 | 641955 | 641955 | Th-Naive_4380 | 0.0807692504459173 |
| chr9 | 63365879 | 63365879 | Th-Naive_7731 | 0.0884461555040797 |
| chr2 | 22323758 | 22323758 | Th-Naive_3835 | 0.0884667891831034 |
| chrY | 12335592 | 12335592 | Th-Naive_8458 | 0.0894305253432752 |
| chr13 | 74310490 | 74310490 | Th-Naive_1983 | 0.0961207781080644 |
| chr3 | 35214371 | 35214371 | Th-Naive_5052 | 0.1005058700476869 |
| chr5 | 177953047 | 177953047 | Th-Naive_6388 | 0.1007352542500938 |
| chr4 | 9611287 | 9611287 | Th-Naive_5531 | 0.1025335840288313 |
| chr9 | 135661915 | 135661916 | Th-Naive_7975 | 0.1039919957363975 |
| chr4 | 72594407 | 72594407 | Th-Naive_5693 | 0.104489555178782 |
| chr3 | 8923092 | 8923092 | Th-Naive_4975 | 0.1080935191813998 |
| chr7 | 150430024 | 150430024 | Th-Naive_7178 | 0.1093090921820635 |
| chrX | 66287262 | 66287262 | Th-Naive_8172 | 0.1150701997400534 |
| chr14 | 76601694 | 76601694 | Th-Naive_2216 | 0.12310958271968 |
| chr4 | 94647741 | 94647741 | Th-Naive_5735 | 0.1316955208280674 |
| chr5 | 81874512 | 81874512 | Th-Naive_6190 | 0.1406019853498072 |
| chr8 | 15833228 | 15833229 | Th-Naive_7268 | 0.1433026259390443 |
| chrX | 62282974 | 62282974 | Th-Naive_8155 | 0.1443245647502319 |
| chr6 | 49063532 | 49063532 | Th-Naive_6548 | 0.1497375605715614 |
| chr7 | 48203181 | 48203181 | Th-Naive_6957 | 0.1517676103511603 |
| chr8 | 138620223 | 138620223 | Th-Naive_7581 | 0.1551608799692273 |
| chrX | 108836577 | 108836577 | Th-Naive_8307 | 0.1682359949982285 |
| chr9 | 24050556 | 24050556 | Th-Naive_7684 | 0.1756618891257859 |
| chr14 | 18918663 | 18918663 | Th-Naive_2089 | 0.1809102976786986 |
| chrY | 11455449 | 11455449 | Th-Naive_8454 | 0.1857351742636238 |
| chrX | 93155315 | 93155315 | Th-Naive_8255 | 0.1876492229514546 |
| chr19 | 15170575 | 15170575 | Th-Naive_3537 | 0.190568160605077 |
| chr13 | 28525864 | 28525864 | Th-Naive_1905 | 0.1917946919373715 |
| chrY | 10813686 | 10813686 | Th-Naive_8452 | 0.1971451338970885 |
| chr2 | 233119625 | 233119625 | Th-Naive_4326 | 0.1996955638076731 |
| chr16 | 7557797 | 7557797 | Th-Naive_2649 | 0.2009767394104656 |
| chrX | 155568118 | 155568118 | Th-Naive_8447 | 0.2022674733347382 |
| chr10 | 54550518 | 54550519 | Th-Naive_791 | 0.208366458091442 |
| chr9 | 3234232 | 3234232 | Th-Naive_7626 | 0.2208608367187939 |
| chrY | 16848558 | 16848558 | Th-Naive_8460 | 0.2307386137292244 |
| chrX | 66936261 | 66936261 | Th-Naive_8178 | 0.2378694165992045 |
| chr9 | 4523382 | 4523382 | Th-Naive_7630 | 0.2600502303490069 |
| chr12 | 97227806 | 97227806 | Th-Naive_1719 | 0.262415332640325 |
| chrY | 4593644 | 4593644 | Th-Naive_8448 | 0.3260769758753131 |
| chr9 | 130055263 | 130055263 | Th-Naive_7937 | 0.3593676992284619 |
| chr1 | 178605692 | 178605692 | Th-Naive_462 | 0.3846310159261332 |
| chr2 | 176814043 | 176814043 | Th-Naive_4176 | 0.4635655255693352 |

| | | | |
|---|---|---|---|
| Chromosome | Start Position | End position | Weight |

OP B-Naive

| | | | |
|---|---|---|---|
| chr1 | 2044669 | 2044669 | −0.0099941094673024 |
| chr1 | 3296026 | 3296026 | 0.0076428975049124 |
| chr1 | 3326786 | 3326786 | −0.0091165522865008 |
| chr1 | 4683644 | 4683644 | −0.0021527697549846 |
| chr1 | 8933633 | 8933634 | 0.008975685012949 |
| chr1 | 8937002 | 8937003 | 0.0127618928010053 |
| chr1 | 11502219 | 11502220 | 0.0093569893884577 |
| chr1 | 12163393 | 12163424 | −0.011256554392096 |
| chr1 | 13821019 | 13821019 | −0.0108038374783377 |
| chr1 | 14248916 | 14248917 | 0.012527364369762 |
| chr1 | 14404229 | 14404229 | 0.0004513783865521 |
| chr1 | 14499938 | 14499938 | 0.000487059357637 |
| chr1 | 15321332 | 15321332 | 0.0016343747568581 |
| chr1 | 16639363 | 16639363 | −0.0108376247841251 |
| chr1 | 22290537 | 22290537 | −0.0046142532214872 |
| chr1 | 24238593 | 24238627 | 0.0025657227938649 |
| chr1 | 24296063 | 24296063 | 0.004080079104335 |
| chr1 | 24546684 | 24546685 | −0.0127541090326721 |
| chr1 | 25135493 | 25135493 | 0.0056604408114175 |
| chr1 | 26297835 | 26297836 | −0.0015292988970829 |
| chr1 | 26307815 | 26307816 | −0.0209219275775861 |
| chr1 | 36399960 | 36399960 | −0.0087930339822816 |
| chr1 | 36966981 | 36966981 | −0.0052971880192018 |
| chr1 | 36977798 | 36977798 | −0.0004996092276979 |
| chr1 | 40885801 | 40885801 | 0.0199864315738295 |
| chr1 | 41946575 | 41946575 | 0.0138703207360855 |
| chr1 | 44540559 | 44540560 | −0.0094501056822028 |
| chr1 | 46355830 | 46355830 | −0.0195045021462978 |
| chr1 | 48060666 | 48060667 | 0.0087680357511526 |
| chr1 | 48730662 | 48730662 | 0.0108327834054861 |
| chr1 | 49574750 | 49574750 | −0.0067385965712449 |
| chr1 | 54422327 | 54422488 | 0.0011913815959101 |
| chr1 | 54434006 | 54434006 | 0.0088730406975975 |
| chr1 | 54582567 | 54582567 | 0.0011984438133281 |
| chr1 | 54622735 | 54622736 | 0.0059066029562082 |
| chr1 | 55006691 | 55006691 | −0.0014144161305399 |
| chr1 | 57240069 | 57240070 | 0.0015357980053922 |
| chr1 | 57571012 | 57571013 | −0.0293261631934358 |
| chr1 | 57766389 | 57766389 | 0.0005774920080316 |
| chr1 | 57819354 | 57819355 | 0.0022538686244391 |
| chr1 | 58368353 | 58368354 | 0.0114918731621092 |
| chr1 | 58422124 | 58422125 | −0.0087404473535251 |
| chr1 | 58454516 | 58454516 | −0.0128962256030755 |
| chr1 | 58468969 | 58468969 | −0.0282452185720128 |
| chr1 | 58545331 | 58545331 | 0.0001012474108644 |
| chr1 | 59756708 | 59756708 | −0.0037945933849715 |
| chr1 | 59764559 | 59764559 | 0.0120260888722405 |
| chr1 | 61007261 | 61007262 | −0.0021745862160237 |
| chr1 | 61296689 | 61296689 | 0.0146898782825286 |
| chr1 | 61592525 | 61592526 | 0.0032357254281352 |
| chr1 | 61620204 | 61620204 | 0.0127079087375068 |
| chr1 | 61913979 | 61913979 | −0.0002486760976123 |
| chr1 | 61936877 | 61936877 | 0.0043535008978876 |
| chr1 | 62139852 | 62139853 | 0.0058514256952798 |
| chr1 | 62141784 | 62141785 | 0.0138028945495457 |
| chr1 | 62184372 | 62184372 | 0.003709093317473 |
| chr1 | 63020156 | 63020156 | 0.0076368563313189 |
| chr1 | 63061286 | 63061287 | 0.0126330902564328 |
| chr1 | 63613936 | 63613936 | −0.0156417102324478 |
| chr1 | 63630190 | 63630190 | −0.010401200261348 |
| chr1 | 65608269 | 65608269 | 0.0119729971481458 |
| chr1 | 66677371 | 66677372 | 0.0012617350913031 |
| chr1 | 67133974 | 67133975 | 0.0022745367813571 |
| chr1 | 67526938 | 67526939 | −0.0073418173422699 |
| chr1 | 67707581 | 67707581 | 0.0021121055365224 |
| chr1 | 68781256 | 68781257 | −0.0182767506493353 |
| chr1 | 68822995 | 68822995 | 0.0023367898655128 |
| chr1 | 68836461 | 68836461 | 0.0122411075415991 |
| chr1 | 69312504 | 69312504 | −0.0099931502073048 |
| chr1 | 70709934 | 70709934 | 0.0195598718032816 |
| chr1 | 70755194 | 70755195 | 0.0037877905223813 |
| chr1 | 71233083 | 71233084 | 0.0132492017797166 |
| chr1 | 71500474 | 71500474 | 0.0042865608898505 |
| chr1 | 72306132 | 72306132 | 0.0140974900134976 |
| chr1 | 73534560 | 73534560 | −0.0130484452565505 |
| chr1 | 73574252 | 73574252 | −0.011498205889165 |
| chr1 | 74766039 | 74766040 | 0.0119280239042668 |
| chr1 | 79717956 | 79717956 | −0.023962595942876 |
| chr1 | 80696526 | 80696526 | −0.0054252426294396 |
| chr1 | 87617491 | 87617491 | −0.0034405999062782 |
| chr1 | 87659840 | 87659840 | −0.0019907114719156 |
| chr1 | 94130626 | 94130626 | 0.0027544171862339 |
| chr1 | 94885328 | 94885328 | −0.0052571989832285 |
| chr1 | 94909189 | 94909189 | 0.0004182961008762 |
| chr1 | 95316446 | 95316446 | −0.006653536953319 |
| chr1 | 95334299 | 95334300 | −0.0183462625934085 |
| chr1 | 95938906 | 95938907 | −0.0205684713713563 |
| chr1 | 95960701 | 95960701 | −0.0116485314395448 |
| chr1 | 96029041 | 96029042 | −0.005216032750967 |
| chr1 | 99201715 | 99201716 | 0.0058734311839696 |
| chr1 | 111289293 | 111289293 | 0.0026672248405237 |
| chr1 | 114537895 | 114537895 | −0.0150877217797255 |
| chr1 | 115539246 | 115539247 | −0.0221808822727619 |
| chr1 | 115739969 | 115739969 | −0.0026553950513482 |
| chr1 | 116009949 | 116009949 | −0.0011929109733866 |
| chr1 | 116175618 | 116175619 | −0.0007704581363802 |
| chr1 | 125069921 | 125069921 | 0.0063394112237037 |
| chr1 | 125174096 | 125174096 | −0.0141137545332636 |
| chr1 | 147716557 | 147716557 | −0.0059408506748997 |
| chr1 | 150359038 | 150359038 | −0.0122283867485793 |

OP Monocyte

| | | | |
|---|---|---|---|
| chr1 | 1378865 | 1378866 | −0.005949971512489 |
| chr1 | 1380513 | 1380514 | −0.0065431231214835 |
| chr1 | 2363405 | 2363406 | −0.0019252469957931 |
| chr1 | 3394648 | 3394649 | −0.0044497982989952 |
| chr1 | 3400191 | 3400192 | −0.0024466021461204 |
| chr1 | 3542116 | 3542116 | 0.0032532861230635 |
| chr1 | 3812606 | 3812606 | −0.0007772325752452 |
| chr1 | 3841184 | 3841185 | −0.0036398779297279 |
| chr1 | 4018388 | 4018388 | −0.0060135388952328 |
| chr1 | 4287614 | 4287614 | −0.0052949258913315 |
| chr1 | 5676731 | 5676731 | −0.0023950083911139 |
| chr1 | 7460069 | 7460069 | −0.0035839972975409 |
| chr1 | 7803233 | 7803234 | −0.003057697220755 |
| chr1 | 7881157 | 7881158 | −0.0024414533955599 |
| chr1 | 7905886 | 7905886 | 0.00254993336416 |
| chr1 | 8890661 | 8890662 | −0.0030230838688138 |
| chr1 | 8930797 | 8930798 | 0.0095220919353401 |
| chr1 | 8933633 | 8933634 | 0.00267719057003 |
| chr1 | 8937002 | 8937003 | 0.0084943478785265 |
| chr1 | 11612551 | 11612552 | −0.0039384859128211 |
| chr1 | 12530720 | 12530720 | −0.0112978133318493 |
| chr1 | 12715702 | 12715702 | 0.0005351392349679 |
| chr1 | 13508515 | 13508516 | −0.0014025152134696 |
| chr1 | 13515137 | 13515137 | −0.0057454044193963 |
| chr1 | 13581294 | 13581294 | 0.0024525100203711 |
| chr1 | 13822683 | 13823107 | −0.0006833889967451 |
| chr1 | 13824244 | 13824244 | −0.0078496335797758 |
| chr1 | 15542788 | 15542789 | −0.0026216616142293 |
| chr1 | 15935708 | 15935708 | 0.001666885822893 |
| chr1 | 20380920 | 20380920 | −0.0003677484971047 |
| chr1 | 21342867 | 21342868 | −0.002943766295914 |
| chr1 | 21548896 | 21548896 | 0.0007449651128095 |
| chr1 | 21565986 | 21565986 | 0.0002425388355272 |
| chr1 | 23250757 | 23250757 | 0.0016011327593315 |
| chr1 | 24057607 | 24057607 | 0.000642432559089 |
| chr1 | 24120723 | 24120724 | −0.0024748539973054 |
| chr1 | 24208947 | 24208948 | −0.0101784237768277 |
| chr1 | 24229195 | 24229195 | 0.0035719845497005 |
| chr1 | 24237083 | 24237084 | 0.0084164371846172 |
| chr1 | 24237395 | 24237396 | 0.0041447647122474 |
| chr1 | 24238593 | 24238627 | 0.0021565292072098 |
| chr1 | 24239830 | 24239831 | 0.0076823794338672 |
| chr1 | 24241173 | 24241174 | 0.008505145894462 |
| chr1 | 24890854 | 24890855 | −0.0123649903186782 |
| chr1 | 26201372 | 26201372 | −0.0027033673654748 |
| chr1 | 29293463 | 29293463 | 0.0041063654282447 |
| chr1 | 29348421 | 29348422 | 0.0064120781755504 |
| chr1 | 30021743 | 30021744 | 0.0005276762324012 |
| chr1 | 30035045 | 30035045 | 0.0053548763914558 |
| chr1 | 30676801 | 30676801 | −0.001327636534184 |
| chr1 | 31555815 | 31555816 | −0.0076998451088443 |
| chr1 | 33309748 | 33309748 | 0.0055203608936326 |
| chr1 | 34250992 | 34250992 | 0.0070635783246381 |
| chr1 | 37361156 | 37361156 | 0.0013787840533406 |
| chr1 | 37767728 | 37767728 | −0.0008400283906593 |
| chr1 | 37773070 | 37773071 | −0.0100567522993174 |
| chr1 | 37828954 | 37828954 | 0.0031257360605832 |
| chr1 | 44541092 | 44541093 | −0.0046471997316042 |
| chr1 | 44590089 | 44590090 | −0.0039262364411087 |
| chr1 | 44983214 | 44983214 | −0.0023697474457611 |
| chr1 | 47268572 | 47268713 | −0.0008360325821799 |
| chr1 | 47509927 | 47509928 | −0.0004869944684535 |
| chr1 | 47525076 | 47525077 | −0.0002471446732771 |
| chr1 | 49280999 | 49280999 | 0.0054239476411724 |
| chr1 | 49348753 | 49348753 | −0.0030157462688732 |
| chr1 | 49472982 | 49472983 | 0.0015574356041122 |
| chr1 | 49934925 | 49934925 | −0.0049468783199034 |
| chr1 | 49991165 | 49991166 | 0.0014051013668111 |
| chr1 | 50055448 | 50055448 | 0.0023939066768065 |
| chr1 | 52062937 | 52062937 | −0.00180532352836 |
| chr1 | 52660483 | 52660484 | 0.0003889674017376 |
| chr1 | 53139341 | 53139342 | 0.0022215603403465 |
| chr1 | 53145933 | 53145934 | −0.0006719942248254 |
| chr1 | 53168391 | 53168391 | −0.0055683927815965 |
| chr1 | 53465173 | 53465173 | −0.0003093871227223 |
| chr1 | 53555470 | 53555470 | −0.0004332544293785 |
| chr1 | 53598264 | 53598265 | 0.0006684147778766 |
| chr1 | 54422071 | 54422072 | 0.001764638072095 |
| chr1 | 54614894 | 54614895 | 0.0071981776204687 |
| chr1 | 55006691 | 55006691 | −0.0031631678464793 |
| chr1 | 55038976 | 55038976 | −0.0012066779065985 |
| chr1 | 56474932 | 56474932 | −0.0006841086459396 |
| chr1 | 56608876 | 56609013 | −0.0021773174938091 |
| chr1 | 56702362 | 56702363 | 0.0090240126308492 |
| chr1 | 56801004 | 56801004 | 0.000503172754441 |
| chr1 | 57059440 | 57059440 | −0.0057909374762161 |
| chr1 | 57239532 | 57239533 | 0.0002833829977984 |
| chr1 | 57276260 | 57276261 | 0.0006382047232351 |
| chr1 | 57291852 | 57291853 | 0.0070754801550075 |
| chr1 | 57939451 | 57939451 | 0.0014481528538583 |
| chr1 | 58136195 | 58136195 | 0.0052384139069821 |
| chr1 | 58337102 | 58337103 | −0.0075607741325404 |
| chr1 | 58368353 | 58368354 | 0.0034113966751785 |
| chr1 | 58393524 | 58393525 | −0.0008253864317201 |
| chr1 | 58400849 | 58400849 | −0.0102929949571948 |
| chr1 | 58423825 | 58423825 | −0.0047865160846678 |
| chr1 | 58450974 | 58450975 | 0.0061064465702617 |
| chr1 | 58454516 | 58454516 | −0.0068593033761622 |
| chr1 | 58464771 | 58464772 | −0.0055819856236343 |
| chr1 | 58468204 | 58468205 | −0.0133531906112464 |

OP NK-cell1

| | | | |
|---|---|---|---|
| chr1 | 974040 | 974040 | −0.0007796899819288 |
| chr1 | 996997 | 996997 | 0.0149762329128717 |
| chr1 | 1124701 | 1124701 | −0.0116152522130214 |
| chr1 | 1380513 | 1380513 | −0.0090073126553904 |
| chr1 | 1558346 | 1558346 | −0.007733369065935 |
| chr1 | 2305183 | 2305183 | −0.0008000027706893 |
| chr1 | 2342136 | 2342136 | −0.0130724178814836 |
| chr1 | 2843354 | 2843354 | 0.0099243235385837 |
| chr1 | 3326787 | 3326787 | −0.0125266542545915 |
| chr1 | 4197160 | 4197160 | 0.0126801420090311 |
| chr1 | 5454551 | 5454551 | −0.0050573492597648 |
| chr1 | 6133404 | 6133404 | −0.0254226068112546 |
| chr1 | 7284774 | 7284774 | 0.0045404386504974 |
| chr1 | 7686404 | 7686404 | 0.0069885890546542 |
| chr1 | 7857765 | 7857765 | −0.0013677003400825 |
| chr1 | 9378117 | 9378117 | 0.0006419715813197 |
| chr1 | 10808935 | 10808935 | −0.0004368855491988 |
| chr1 | 11430920 | 11430920 | −0.0068990870486868 |
| chr1 | 12330140 | 12330140 | 0.0025011142784985 |
| chr1 | 13121744 | 13121780 | 0.0016226475883131 |
| chr1 | 13821019 | 13821019 | −0.0123385423309074 |
| chr1 | 14260459 | 14260459 | 0.0217448603352177 |
| chr1 | 14503258 | 14503258 | 0.0167609028034191 |
| chr1 | 15020397 | 15020397 | 0.0020131461391844 |
| chr1 | 15129340 | 15129340 | 0.0011929941168046 |
| chr1 | 15581748 | 15581748 | −0.0011913798227959 |
| chr1 | 16227677 | 16227677 | −0.0034857474466896 |
| chr1 | 16252575 | 16252575 | −0.0152141561209194 |
| chr1 | 17220053 | 17220053 | 0.0087621876429578 |
| chr1 | 17579562 | 17579562 | −0.010877019106219 |
| chr1 | 17632542 | 17632542 | −0.0023723948756406 |
| chr1 | 18803701 | 18803701 | −0.0059469041256177 |
| chr1 | 19634741 | 19634741 | −0.0029809775989339 |
| chr1 | 20550216 | 20550216 | 0.010052560034239 |
| chr1 | 20625500 | 20625500 | −0.0102711932259871 |
| chr1 | 20715488 | 20715488 | −0.0109170140649939 |
| chr1 | 20968282 | 20968282 | −0.0016046153255204 |
| chr1 | 21570470 | 21570470 | 0.0001784599589831 |
| chr1 | 24051477 | 24051477 | 0.00920008090661 |
| chr1 | 24120724 | 24120724 | −0.0037821337328066 |
| chr1 | 24546684 | 24546684 | −0.0320871971462663 |
| chr1 | 25072983 | 25072983 | −0.0061632944238221 |
| chr1 | 25074945 | 25074945 | 0.0108854915979959 |
| chr1 | 25082452 | 25082452 | −0.0103959221993875 |
| chr1 | 26245485 | 26245485 | −0.000950779383105 |
| chr1 | 26282815 | 26282815 | −0.022255118446043 |
| chr1 | 29386191 | 29386191 | 0.0020831828047215 |
| chr1 | 31508385 | 31508385 | 0.0069005851665607 |
| chr1 | 34100618 | 34100618 | 0.0063220781500629 |
| chr1 | 34765362 | 34765362 | 0.0011273084380889 |
| chr1 | 36187284 | 36187284 | 0.0029023653235652 |
| chr1 | 41783714 | 41783714 | 0.0028143545236749 |
| chr1 | 46439693 | 46439693 | 0.0003392100329843 |
| chr1 | 46480178 | 46480178 | 0.0014400548522299 |
| chr1 | 47385772 | 47385772 | 0.0282623125445392 |
| chr1 | 51458554 | 51458555 | −0.0212351798301829 |
| chr1 | 52834292 | 52834292 | 0.0004535734112988 |
| chr1 | 53103308 | 53103308 | −0.0185014120957993 |
| chr1 | 53598567 | 53598567 | −0.0068662966457178 |
| chr1 | 54426809 | 54426809 | 0.0063119883226535 |
| chr1 | 55006691 | 55006691 | −0.0092289341052511 |
| chr1 | 56295415 | 56295415 | 0.0006990355433572 |
| chr1 | 58412597 | 58412598 | −0.0141537770615209 |
| chr1 | 58958962 | 58958962 | −0.0064249212201759 |
| chr1 | 59171766 | 59171766 | 0.0023059371056106 |
| chr1 | 60147409 | 60147409 | 0.0023262139875063 |
| chr1 | 61930930 | 61930931 | 0.0102003693700568 |
| chr1 | 62140457 | 62140457 | 0.0055995756832371 |
| chr1 | 64414808 | 64414808 | 0.0106159549086216 |
| chr1 | 64916563 | 64916564 | −0.0197076671072583 |
| chr1 | 68755622 | 68755622 | −0.0380245795874258 |
| chr1 | 68871912 | 68871912 | 0.0037204794132356 |
| chr1 | 74452785 | 74452785 | 0.0061272447020648 |
| chr1 | 74752068 | 74752068 | 0.0018492917141145 |
| chr1 | 75473112 | 75473112 | −0.0028441148001714 |
| chr1 | 76034293 | 76034293 | −0.0036547138685166 |
| chr1 | 79570002 | 79570002 | −0.0003710599022794 |
| chr1 | 80274412 | 80274412 | 4.590644459120372e−05 |
| chr1 | 83582179 | 83582179 | −0.0130037371869411 |
| chr1 | 86353162 | 86353162 | −0.0229305688686396 |
| chr1 | 86661444 | 86661444 | 0.002481364969492 |
| chr1 | 87076635 | 87076635 | 0.0008959716592474 |
| chr1 | 90725713 | 90725713 | −0.0151407252176722 |
| chr1 | 92663053 | 92663053 | 0.0045103183046154 |
| chr1 | 93004470 | 93004470 | 0.0064182896996829 |
| chr1 | 93198047 | 93198047 | 0.0052423166361579 |
| chr1 | 95098776 | 95098776 | 0.0056374728548913 |
| chr1 | 96634226 | 96634226 | −0.0001008933887325 |
| chr1 | 99754263 | 99754263 | −0.0024372313213143 |
| chr1 | 100643171 | 100643171 | 0.0028387529011639 |
| chr1 | 102235000 | 102235000 | −0.0016379060671081 |
| chr1 | 106081024 | 106081024 | −0.0070898625261843 |
| chr1 | 106968895 | 106968895 | 0.0013781979974688 |
| chr1 | 107502968 | 107502968 | 0.0019933688887628 |
| chr1 | 107651997 | 107651997 | 0.0056305029467831 |
| chr1 | 107701992 | 107701992 | −0.0098876211131514 |
| chr1 | 109401271 | 109401271 | 0.0034718285426105 |
| chr1 | 109822198 | 109822198 | −0.0098296102549889 |
| chr1 | 110901756 | 110901756 | 0.0040895656087774 |
| chr1 | 111077613 | 111077613 | 0.0075505570345147 |

OP NK-cell2

| chr1 | 3865267 | 3865267 | −0.0111224446553096 |
|---|---|---|---|
| chr1 | 8883188 | 8883189 | −0.0109084473787405 |
| chr1 | 8883777 | 8883777 | 0.0032855227107395 |
| chr1 | 8922847 | 8922847 | −0.0221275611501655 |
| chr1 | 8930797 | 8930797 | 0.019518647901717 |
| chr1 | 8980148 | 8980148 | −0.0018180486902911 |
| chr1 | 10766201 | 10766201 | 0.0066327454717493 |
| chr1 | 11379551 | 11379551 | −1.4963603665185902e−05 |
| chr1 | 16252575 | 16252575 | −0.0249249284758076 |
| chr1 | 16633495 | 16633495 | −0.0102030859911021 |
| chr1 | 21342867 | 21342867 | −0.0158537913194297 |
| chr1 | 21720782 | 21720782 | 0.0046485453131747 |
| chr1 | 21787978 | 21787979 | 0.0005182471366458 |
| chr1 | 23250756 | 23250757 | 0.0086074282494096 |
| chr1 | 24207677 | 24207677 | −0.0017944437174158 |
| chr1 | 24229195 | 24229195 | 0.0129380221538584 |
| chr1 | 24237083 | 24237084 | 0.0012868340933772 |
| chr1 | 24237396 | 24237396 | 0.0006151297257502 |
| chr1 | 24237793 | 24237794 | 0.0101983322031287 |
| chr1 | 24241173 | 24241173 | 0.0029675880392197 |
| chr1 | 24283444 | 24283444 | −0.0103902565301628 |
| chr1 | 24890854 | 24890855 | −0.0245769873097777 |
| chr1 | 24996555 | 24996556 | −0.0203932855784612 |
| chr1 | 25082452 | 25082453 | −0.0085367537946823 |
| chr1 | 25882531 | 25882531 | 0.0066559453666911 |
| chr1 | 25883674 | 25883674 | 0.0099514369957463 |
| chr1 | 25888826 | 25888826 | 0.000429122131894 |
| chr1 | 26245485 | 26245486 | −0.0085421350347659 |
| chr1 | 26296502 | 26296503 | 0.0217416172528059 |
| chr1 | 26308409 | 26308409 | −0.0011745072466497 |
| chr1 | 26835179 | 26835179 | 0.0006334004778824 |
| chr1 | 27210610 | 27210610 | −0.0226971476525902 |
| chr1 | 29190145 | 29190146 | 0.0172386111485732 |
| chr1 | 29294948 | 29294948 | 0.0023385506390457 |
| chr1 | 29303609 | 29303609 | 0.0156248581717954 |
| chr1 | 30046938 | 30046938 | −0.0027167847199384 |
| chr1 | 30660395 | 30660395 | −0.0205689651920001 |
| chr1 | 30673641 | 30673641 | −0.0018023501233726 |
| chr1 | 31555815 | 31555816 | −0.0128714053562011 |
| chr1 | 32619320 | 32619320 | 0.0111844377302549 |
| chr1 | 34492464 | 34492464 | 0.0028754049595644 |
| chr1 | 34632443 | 34632443 | −0.0214684551806814 |
| chr1 | 34698478 | 34698478 | −0.0049586892808871 |
| chr1 | 35212537 | 35212537 | 0.0041603645495395 |
| chr1 | 35580542 | 35580543 | 0.00512000194703 |
| chr1 | 36024744 | 36024745 | 0.0122566094996022 |
| chr1 | 36182324 | 36182325 | 0.0104932769146351 |
| chr1 | 36399959 | 36399960 | −0.0111554805969924 |
| chr1 | 39547209 | 39547209 | 0.001287887107604 |
| chr1 | 47509927 | 47509928 | −0.0017604155854259 |
| chr1 | 48283884 | 48283884 | −0.0027880149236036 |
| chr1 | 49916734 | 49916734 | −0.0199727254708425 |
| chr1 | 51490980 | 51490980 | 0.0086692193621274 |
| chr1 | 54711638 | 54711638 | −0.00359803637304 |
| chr1 | 56474932 | 56474932 | −0.0107198303507456 |
| chr1 | 56609013 | 56609013 | −0.0020100650670336 |
| chr1 | 57588963 | 57588963 | 0.0007161139826759 |
| chr1 | 57829039 | 57829039 | 0.012448642977687 |
| chr1 | 58454516 | 58454516 | −0.0330370579398874 |
| chr1 | 58958962 | 58958962 | −0.0064697339204169 |
| chr1 | 59765622 | 59765623 | 0.0026911000098057 |
| chr1 | 60870857 | 60870857 | −0.0221935034695211 |
| chr1 | 61296689 | 61296689 | 0.0162103252041492 |
| chr1 | 61591897 | 61591897 | 0.0052872149889241 |
| chr1 | 61602602 | 61602602 | 0.0185461420977371 |
| chr1 | 61608883 | 61609144 | −0.0071385312425558 |
| chr1 | 61929987 | 61929988 | 0.0001132815318157 |
| chr1 | 62190502 | 62190503 | 0.0090570312889056 |
| chr1 | 62224909 | 62224909 | 0.0046985633458867 |
| chr1 | 62711695 | 62711695 | −0.0040991221583868 |
| chr1 | 63071166 | 63071166 | 0.0011249950971765 |
| chr1 | 64414808 | 64414808 | 0.0084007805175826 |
| chr1 | 65121131 | 65121132 | −0.0221681109164682 |
| chr1 | 68181637 | 68181638 | 0.0065972537584106 |
| chr1 | 68366465 | 68366478 | 0.0024773299464106 |
| chr1 | 68386310 | 68386310 | 0.0003702401043167 |
| chr1 | 68390957 | 68390958 | 0.0086676366092889 |
| chr1 | 68781256 | 68781257 | −0.0183037762515085 |
| chr1 | 68895693 | 68895694 | 0.0048892660322156 |
| chr1 | 69219804 | 69219804 | 0.0020874507961397 |
| chr1 | 69306375 | 69306375 | −0.0022120735413171 |
| chr1 | 69566708 | 69566708 | −0.0131465203709263 |
| chr1 | 74727806 | 74727806 | 0.0092593954621693 |
| chr1 | 79605216 | 79605216 | 0.0044760408231365 |
| chr1 | 80696525 | 80696525 | −0.0105320455445011 |
| chr1 | 81397405 | 81397405 | 0.0050211186903934 |
| chr1 | 83441944 | 83441945 | 0.014936675265164 |
| chr1 | 84982382 | 84982382 | 0.0086476422941775 |
| chr1 | 86314758 | 86314759 | −0.0020565810259517 |
| chr1 | 87619596 | 87619597 | 0.0089945653417328 |
| chr1 | 91319329 | 91319329 | −0.0032315620173093 |
| chr1 | 95334299 | 95334300 | −0.0035750054642677 |
| chr1 | 95952458 | 95952458 | −0.0049489464466334 |
| chr1 | 96166656 | 96166656 | 0.0030144083067356 |
| chr1 | 98949444 | 98949444 | −0.0007240468886569 |
| chr1 | 99232735 | 99232790 | 0.0058107140905266 |
| chr1 | 99508352 | 99508352 | −0.003150019765182 |
| chr1 | 102164973 | 102164973 | 0.0167350939912526 |
| chr1 | 102235000 | 102235000 | −0.0010390601063983 |
| chr1 | 104077966 | 104077966 | 0.0176682990168231 |

OP Tc-Mem

| | | | |
|---|---|---|---|
| chr1 | 607497 | 607497 | 0.0046302591011464 |
| chr1 | 1053551 | 1053551 | 0.0024233950794016 |
| chr1 | 1378865 | 1378866 | −0.0024977656518669 |
| chr1 | 1379663 | 1379664 | −0.002131980322971 |
| chr1 | 1380513 | 1380514 | −0.0055118988595216 |
| chr1 | 2051265 | 2051265 | −0.0017959263056052 |
| chr1 | 2314194 | 2314195 | 0.0012173654410395 |
| chr1 | 2326884 | 2326884 | −0.0051923236711123 |
| chr1 | 2469095 | 2469096 | 0.0002335034914326 |
| chr1 | 3327466 | 3327466 | 0.0019152365601969 |
| chr1 | 3396100 | 3396101 | −0.0040539563413091 |
| chr1 | 3836174 | 3836175 | −0.0015928362545455 |
| chr1 | 3841184 | 3841185 | −0.0054736422046158 |
| chr1 | 3849114 | 3849114 | −0.0035452930777678 |
| chr1 | 3852698 | 3852698 | −0.0068684643780145 |
| chr1 | 3861674 | 3861675 | −0.0019310081920921 |
| chr1 | 3865267 | 3865267 | −0.001475520227192 |
| chr1 | 3933574 | 3933574 | 0.0031896141503079 |
| chr1 | 4018387 | 4018388 | −0.0082246091344137 |
| chr1 | 5244963 | 5244963 | −0.0008194163927709 |
| chr1 | 6012213 | 6012213 | 0.0032447600633465 |
| chr1 | 6204785 | 6204786 | −0.0036396471168218 |
| chr1 | 6928279 | 6928279 | −0.0007089931414603 |
| chr1 | 7309585 | 7309586 | 0.0054897526376412 |
| chr1 | 7795434 | 7795434 | −0.0020119686863284 |
| chr1 | 7803233 | 7803233 | −0.0061076574887254 |
| chr1 | 7857764 | 7857765 | −0.0068992459983862 |
| chr1 | 7863501 | 7863502 | −0.0025278994157717 |
| chr1 | 8930797 | 8930798 | 0.0057448064834952 |
| chr1 | 8937002 | 8937003 | 0.0049452530469132 |
| chr1 | 9478219 | 9478219 | 0.0025076562957286 |
| chr1 | 9632075 | 9632075 | −0.0054102332366282 |
| chr1 | 10484489 | 10484490 | −0.0060107976077587 |
| chr1 | 10638287 | 10638287 | −0.0014873454542198 |
| chr1 | 11107854 | 11107854 | 0.003095494749804 |
| chr1 | 11157608 | 11157609 | 0.0014572522949965 |
| chr1 | 11163254 | 11163254 | 0.0020226302505892 |
| chr1 | 11502219 | 11502220 | 0.0027445460218412 |
| chr1 | 11519811 | 11519811 | −0.0010716636297582 |
| chr1 | 11622806 | 11622806 | −0.0038647427078427 |
| chr1 | 11916475 | 11916475 | 0.0037688943892497 |
| chr1 | 12078735 | 12078735 | −0.0010968350765262 |
| chr1 | 13375016 | 13375017 | 0.0044193552280094 |
| chr1 | 13493080 | 13493080 | −0.0008549866036033 |
| chr1 | 13508516 | 13508516 | −0.0030553232883805 |
| chr1 | 13581294 | 13581294 | 0.0020713016373721 |
| chr1 | 13822683 | 13823106 | −0.0011779560215348 |
| chr1 | 13824244 | 13824244 | −0.0004488138938967 |
| chr1 | 13957965 | 13957976 | 0.003054949623534 |
| chr1 | 14248916 | 14248916 | 0.0008212244632079 |
| chr1 | 14260459 | 14260459 | 0.0037117752073075 |
| chr1 | 15344986 | 15344987 | 0.0040086211931706 |
| chr1 | 15542788 | 15542789 | −0.0026505482379784 |
| chr1 | 15776690 | 15776690 | 0.0024008454744252 |
| chr1 | 16225011 | 16225011 | −0.0064345243354836 |
| chr1 | 17420102 | 17420102 | −0.0016536276234102 |
| chr1 | 17719448 | 17719448 | 0.0014224849785133 |
| chr1 | 17952815 | 17952815 | −0.0012393886191422 |
| chr1 | 18866665 | 18866665 | 0.0020627060438245 |
| chr1 | 19202621 | 19202621 | −0.0009449893410978 |
| chr1 | 19642050 | 19642050 | 0.0006213362491574 |
| chr1 | 21342867 | 21342868 | −0.0043512076635324 |
| chr1 | 21471014 | 21471015 | 0.0058967928191352 |
| chr1 | 23177242 | 23177242 | −0.0013565976062824 |
| chr1 | 23250756 | 23250757 | 0.0064504140602869 |
| chr1 | 23954616 | 23954616 | −0.0003982646529673 |
| chr1 | 24140765 | 24140773 | 0.0026164864105483 |
| chr1 | 24208947 | 24208948 | −0.0060425256365279 |
| chr1 | 24229195 | 24229195 | 0.0088229292449531 |
| chr1 | 24229461 | 24229462 | 0.0026967113130243 |
| chr1 | 24237083 | 24237084 | 0.0035798632985482 |
| chr1 | 24238593 | 24238626 | 0.0001754682263735 |
| chr1 | 24239830 | 24239831 | 0.0066075770692619 |
| chr1 | 24241173 | 24241174 | 0.0035261054298408 |
| chr1 | 24269207 | 24269208 | −0.0065275935893854 |
| chr1 | 24294310 | 24294311 | 0.0007366513194461 |
| chr1 | 24349300 | 24349301 | −0.0031044162007155 |
| chr1 | 24386232 | 24386233 | −0.0021327270385167 |
| chr1 | 24499613 | 24499614 | −0.0052517430313324 |
| chr1 | 24514277 | 24514277 | −0.0014988023459081 |
| chr1 | 24973147 | 24973147 | 0.0030277951457136 |
| chr1 | 24977973 | 24977974 | 0.006705629125822 |
| chr1 | 25075427 | 25075428 | −0.0075327232976724 |
| chr1 | 25075526 | 25075527 | 0.0003441316418744 |
| chr1 | 25124437 | 25124438 | 0.0026502615849351 |
| chr1 | 25141908 | 25141908 | 0.000198125502382 |
| chr1 | 26239173 | 26239174 | −0.0061903511533397 |
| chr1 | 26245485 | 26245486 | −0.0073331385166671 |
| chr1 | 26252732 | 26252733 | −0.0006372559697611 |
| chr1 | 26294355 | 26294356 | −0.004524774388821 |
| chr1 | 26600362 | 26600362 | 0.0016210779429592 |
| chr1 | 26641478 | 26641478 | 0.0004462608801417 |
| chr1 | 26724827 | 26724827 | −0.0002557136016977 |
| chr1 | 27127947 | 27127947 | −0.0057195111023175 |
| chr1 | 27133228 | 27133229 | 0.001662289153968 |
| chr1 | 27178128 | 27178128 | −0.0027532660242692 |
| chr1 | 28245576 | 28245576 | 0.0009451568442779 |
| chr1 | 29275807 | 29275807 | 0.0009785719656645 |
| chr1 | 29294948 | 29294949 | 0.0025057548812603 |
| chr1 | 29931256 | 29931256 | 0.0010247646211324 |

OP Tc-Naïve

| chr1 | 1053551 | 1053551 | 0.0043392107135015 |
|---|---|---|---|
| chr1 | 1378865 | 1378866 | −0.0020056037942228 |
| chr1 | 1454315 | 1454315 | 0.0110014541565488 |
| chr1 | 2340806 | 2340807 | −0.0031106150713389 |
| chr1 | 2430227 | 2430227 | −0.0014663947975536 |
| chr1 | 3296026 | 3296026 | 0.0037378173072489 |
| chr1 | 3326786 | 3326787 | −0.0031204998278111 |
| chr1 | 3836174 | 3836175 | −0.0036109675225787 |
| chr1 | 3849115 | 3849115 | −0.0032768527903998 |
| chr1 | 3854235 | 3854236 | −0.0046972826059808 |
| chr1 | 4760591 | 4760591 | 0.0002008364577981 |
| chr1 | 7309585 | 7309586 | 0.0003848679981719 |
| chr1 | 7632852 | 7632852 | −0.004068980137524 |
| chr1 | 8933633 | 8933634 | 0.0072048366867648 |
| chr1 | 8937002 | 8937003 | 0.0062476535412157 |
| chr1 | 8940568 | 8940568 | 0.0017132488389552 |
| chr1 | 8947986 | 8947987 | 0.0051602288030073 |
| chr1 | 10583772 | 10583772 | −0.0005429537215855 |
| chr1 | 11379551 | 11379552 | −0.0054921473335412 |
| chr1 | 12530720 | 12530721 | −0.0053118948029159 |
| chr1 | 12534263 | 12534263 | −0.002098146164372 |
| chr1 | 12600476 | 12600477 | −0.0015807591225723 |
| chr1 | 12838849 | 12838849 | 0.0043637035680726 |
| chr1 | 13375016 | 13375017 | 0.0066755408823982 |
| chr1 | 13508515 | 13508516 | −0.0054149325980834 |
| chr1 | 13821019 | 13821019 | −0.0096936114036171 |
| chr1 | 14392437 | 14392437 | −0.0020017504649975 |
| chr1 | 14866244 | 14866244 | 0.0024827462051771 |
| chr1 | 21253308 | 21253309 | 0.0007023880016764 |
| chr1 | 21342867 | 21342868 | −0.0116400510442683 |
| chr1 | 21524602 | 21524603 | 0.0046031182860698 |
| chr1 | 22151000 | 22151000 | −0.0031635759193468 |
| chr1 | 23241966 | 23241967 | 0.0015019363137212 |
| chr1 | 23262834 | 23262835 | 0.0015431766095598 |
| chr1 | 24208947 | 24208948 | −0.0038338818980272 |
| chr1 | 24229461 | 24229462 | 0.0054773009150334 |
| chr1 | 24237083 | 24237084 | 0.0053292781819653 |
| chr1 | 24237395 | 24237396 | 0.0066130626869111 |
| chr1 | 24237793 | 24237794 | 0.0073290473661922 |
| chr1 | 24239830 | 24239831 | 0.0059112706197252 |
| chr1 | 24240857 | 24240858 | 9.86873558949556e−05 |
| chr1 | 24241173 | 24241174 | 0.0038920723856216 |
| chr1 | 24296062 | 24296062 | 0.0039390276886462 |
| chr1 | 24499613 | 24499614 | −0.0015215060620854 |
| chr1 | 24977971 | 24977973 | 0.0123111147379421 |
| chr1 | 25072983 | 25072984 | −0.00451192184184 |
| chr1 | 25075427 | 25075428 | −0.0007767199832434 |
| chr1 | 25075526 | 25075527 | 0.0011657222138245 |
| chr1 | 25161378 | 25161378 | 0.0017297882885483 |
| chr1 | 25867019 | 25867020 | 0.0018908082222769 |
| chr1 | 26252732 | 26252733 | −0.0074977141924913 |
| chr1 | 26297835 | 26297836 | −0.0056449437068709 |
| chr1 | 26875466 | 26875467 | 0.0006285078224245 |
| chr1 | 27041195 | 27041196 | 0.0017599736119952 |
| chr1 | 27194387 | 27194388 | −0.0006972344220687 |
| chr1 | 27210609 | 27210610 | −0.0072192375959401 |
| chr1 | 28228914 | 28228914 | −0.0012174055259354 |
| chr1 | 29293463 | 29293463 | 0.0002130312637486 |
| chr1 | 30021743 | 30021744 | 0.0058190708935735 |
| chr1 | 31555815 | 31555816 | −0.0056651511130189 |
| chr1 | 34462720 | 34462720 | −0.0004590807799807 |
| chr1 | 35239657 | 35239657 | 0.0051504445169373 |
| chr1 | 35377428 | 35377428 | 0.004536244317452 |
| chr1 | 35691720 | 35691720 | 0.0129298397282153 |
| chr1 | 35909510 | 35909510 | 0.0074862718299673 |
| chr1 | 36172604 | 36172605 | 0.0025174873263356 |
| chr1 | 44590089 | 44590090 | −0.0001823612584307 |
| chr1 | 47425689 | 47425689 | −0.0055771818990332 |
| chr1 | 47487290 | 47487291 | −0.0002263620576373 |
| chr1 | 47803737 | 47803737 | 0.0010889088356188 |
| chr1 | 48493808 | 48493808 | 0.0046558588115151 |
| chr1 | 48678174 | 48678174 | −0.0003950285611769 |
| chr1 | 49250663 | 49250663 | 0.0035736447611162 |
| chr1 | 49435212 | 49435212 | −0.0018160358056036 |
| chr1 | 49531802 | 49532003 | −0.0068484250635417 |
| chr1 | 49985187 | 49985188 | 0.0046390614431954 |
| chr1 | 49991165 | 49991165 | 0.0053322228885343 |
| chr1 | 50388792 | 50388792 | 0.0020749271103428 |
| chr1 | 52659927 | 52659928 | −0.0021930409010982 |
| chr1 | 52660483 | 52660483 | 0.0006470198299922 |
| chr1 | 53141332 | 53141333 | 9.256092401378748e−05 |
| chr1 | 53145472 | 53145473 | −0.0053179880499224 |
| chr1 | 54422071 | 54422072 | 0.0011486870529994 |
| chr1 | 54426808 | 54426809 | 0.006033763460942 |
| chr1 | 54434005 | 54434006 | 0.000226516366024 |
| chr1 | 54467067 | 54467068 | −0.002091993766605 |
| chr1 | 54614894 | 54614895 | 0.0007423080698754 |
| chr1 | 55006691 | 55006692 | −0.0018746555719919 |
| chr1 | 55034267 | 55034267 | −0.0006923427065877 |
| chr1 | 56391046 | 56391047 | 0.0013940367369757 |
| chr1 | 56621544 | 56621544 | −8.876394372213327e−05 |
| chr1 | 56676788 | 56676789 | 0.0110759941469018 |
| chr1 | 56682948 | 56682948 | 0.0008838589050717 |
| chr1 | 56687111 | 56687112 | 0.0051728955163049 |
| chr1 | 56690353 | 56690354 | 0.0031637959047822 |
| chr1 | 56712635 | 56712635 | 0.0023920867928371 |
| chr1 | 57219847 | 57219847 | 0.0017116993242819 |
| chr1 | 57224005 | 57224006 | 0.0006285568388172 |
| chr1 | 57239532 | 57239533 | 0.0020276758881899 |
| chr1 | 57291852 | 57291853 | 0.0041099731933589 |

OP Th-Mem

| chr1 | 1123889 | 1123889 | 0.0049556421546121 |
|---|---|---|---|
| chr1 | 1167769 | 1167770 | −0.000288536737625 |
| chr1 | 1602666 | 1602666 | −0.0009560231295022 |
| chr1 | 2035575 | 2035575 | −0.0084736067664424 |
| chr1 | 2238792 | 2238793 | 0.002740993199041 |
| chr1 | 2313782 | 2313783 | 0.0021371010781524 |
| chr1 | 2337212 | 2337212 | −0.0020421713749692 |
| chr1 | 2338570 | 2338570 | −0.0035462658781454 |
| chr1 | 2357881 | 2357881 | −0.0021374136520405 |
| chr1 | 2697621 | 2697621 | 0.0135983585589761 |
| chr1 | 2981311 | 2981311 | 0.0044115981177187 |
| chr1 | 3326786 | 3326786 | −0.0071373711851618 |
| chr1 | 3396100 | 3396101 | −0.0043362055663314 |
| chr1 | 3400191 | 3400192 | −0.004938398331183 |
| chr1 | 3838480 | 3838481 | −0.0011615733822301 |
| chr1 | 3841184 | 3841185 | −0.0055304532326858 |
| chr1 | 3846325 | 3846326 | −0.0020542956471183 |
| chr1 | 4018387 | 4018388 | −0.0010149241100591 |
| chr1 | 4171391 | 4171391 | −0.0013634306607985 |
| chr1 | 4218739 | 4218739 | 0.0004051882910751 |
| chr1 | 5224784 | 5224784 | 0.0009980600172338 |
| chr1 | 6120435 | 6120435 | −0.0050790349170518 |
| chr1 | 7309586 | 7309586 | 0.0003307039998687 |
| chr1 | 7827544 | 7827545 | 0.0055892275812283 |
| chr1 | 7850335 | 7850379 | −0.0009932687602249 |
| chr1 | 8931242 | 8931243 | 0.0065101233580351 |
| chr1 | 8933633 | 8933634 | 0.0069411153028088 |
| chr1 | 8937002 | 8937002 | 0.0086462888468652 |
| chr1 | 8947986 | 8947987 | 0.0001472268904437 |
| chr1 | 9099678 | 9099678 | 0.0054790247786925 |
| chr1 | 9343942 | 9343942 | −0.0036785292235347 |
| chr1 | 10897925 | 10897927 | −0.0024246964914511 |
| chr1 | 12530720 | 12530720 | −0.0009657134508876 |
| chr1 | 12625095 | 12625096 | −0.0073771634122215 |
| chr1 | 13821019 | 13821019 | −0.0064445338469895 |
| chr1 | 13823106 | 13823107 | −0.0028884125692945 |
| chr1 | 13825721 | 13825722 | −0.0036199309816194 |
| chr1 | 14828942 | 14828942 | −0.0044590615586391 |
| chr1 | 15588050 | 15588051 | −0.0031527320952479 |
| chr1 | 18078982 | 18078982 | 0.0063533501922729 |
| chr1 | 21342868 | 21342868 | −0.0045147434014775 |
| chr1 | 21347404 | 21347404 | −0.0114191593029304 |
| chr1 | 21430355 | 21430356 | 0.0164066043344033 |
| chr1 | 21566487 | 21566487 | 0.0023276710144964 |
| chr1 | 21787978 | 21787979 | 0.0029130603107635 |
| chr1 | 22511356 | 22511356 | 0.0027093161110197 |
| chr1 | 23250756 | 23250757 | 0.0044053265437557 |
| chr1 | 24237083 | 24237084 | 0.0010062104003236 |
| chr1 | 24349300 | 24349301 | −0.0087111266412038 |
| chr1 | 24956634 | 24956671 | 0.0014060936551431 |
| chr1 | 25075427 | 25075428 | −0.0064342122208894 |
| chr1 | 25588225 | 25588225 | 0.0039270159502219 |
| chr1 | 25828709 | 25828709 | 0.0052702969267648 |
| chr1 | 25841294 | 25841294 | −0.0057675735529316 |
| chr1 | 26239173 | 26239174 | −0.0168939796005249 |
| chr1 | 26243824 | 26243824 | −0.008365551938754 |
| chr1 | 26295104 | 26295105 | −0.007972145036461 |
| chr1 | 26325286 | 26325286 | −0.0039461298323175 |
| chr1 | 28181469 | 28181469 | −0.0012144305504954 |
| chr1 | 29348207 | 29348207 | −0.009839438655511 |
| chr1 | 30673077 | 30673077 | −0.0074739533990822 |
| chr1 | 35207726 | 35207726 | 0.0039172834399834 |
| chr1 | 35226308 | 35226309 | 0.0004902146640356 |
| chr1 | 35278121 | 35278122 | 0.0021154283687053 |
| chr1 | 35308681 | 35308682 | 0.0033715503067358 |
| chr1 | 35601159 | 35601160 | 0.0061905475689117 |
| chr1 | 35719865 | 35719865 | 9.552172530727772e−05 |
| chr1 | 36117324 | 36117325 | −0.0065696186027633 |
| chr1 | 37819884 | 37819884 | 0.0004873678016934 |
| chr1 | 38968893 | 38968894 | 0.0005170019257349 |
| chr1 | 40414189 | 40414189 | −0.0001757613225859 |
| chr1 | 43291690 | 43291690 | 0.0002776282476398 |
| chr1 | 44068110 | 44068110 | 0.0017755796486168 |
| chr1 | 46201118 | 46201119 | 0.0022431817609779 |
| chr1 | 46661111 | 46661112 | 0.0041200254268625 |
| chr1 | 46701175 | 46701175 | 0.0021895061277094 |
| chr1 | 47247325 | 47247326 | −0.0035034718232037 |
| chr1 | 48224558 | 48224558 | 0.0058412660701807 |
| chr1 | 48739256 | 48739257 | −0.0067865002221998 |
| chr1 | 48758911 | 48758911 | −0.0060720138724383 |
| chr1 | 49472982 | 49472983 | 3.1427219553552974e−06 |
| chr1 | 50055448 | 50055449 | 0.001318706669803 |
| chr1 | 52660842 | 52660842 | 0.0006854517356754 |
| chr1 | 52884235 | 52884235 | −0.0006071690087131 |
| chr1 | 53498712 | 53498713 | 0.0053700456871449 |
| chr1 | 53592558 | 53592559 | 0.0004912991512304 |
| chr1 | 54308470 | 54308470 | 0.0021953579787695 |
| chr1 | 54343084 | 54343084 | 0.0028305803555844 |
| chr1 | 54419766 | 54419767 | 0.0062181622464779 |
| chr1 | 54422327 | 54422488 | 0.0019897831735086 |
| chr1 | 54426808 | 54426809 | 0.0029336279418188 |
| chr1 | 54436816 | 54436817 | 0.0001956546218734 |
| chr1 | 54441494 | 54441494 | 0.0005152348853973 |
| chr1 | 54454432 | 54454433 | 0.0007080013641072 |
| chr1 | 54479189 | 54479190 | −0.0020099233590103 |
| chr1 | 54485098 | 54485098 | −0.0024050970413241 |
| chr1 | 54582015 | 54582016 | 9.441348243516776e−05 |
| chr1 | 54582567 | 54582568 | 0.0009667224829836 |
| chr1 | 55038976 | 55038976 | −0.0050935217086893 |
| chr1 | 55107193 | 55107193 | 0.0005028477986071 |

OP Th-Naïve

| chr1 | 1381268 | 1381294 | −0.0052077202894677 |
|---|---|---|---|
| chr1 | 2340788 | 2340806 | −0.0004602425261361 |
| chr1 | 3093405 | 3093405 | −0.0023577855450379 |
| chr1 | 3294036 | 3294037 | 0.0004287593173436 |
| chr1 | 3326786 | 3326786 | −0.0016778103458403 |
| chr1 | 3398795 | 3398796 | −0.0058449535955074 |
| chr1 | 3812606 | 3812606 | −0.0021040798645598 |
| chr1 | 3836175 | 3836175 | −0.0007157572936677 |
| chr1 | 3841184 | 3841185 | −0.0064564426444946 |
| chr1 | 3846325 | 3846326 | −0.0086886653990786 |
| chr1 | 3852698 | 3852699 | −0.0088115728873674 |
| chr1 | 3861674 | 3861675 | −0.0036396528836979 |
| chr1 | 4171390 | 4171391 | −0.0008164777679121 |
| chr1 | 4427540 | 4427540 | 0.0005690248054155 |
| chr1 | 5725075 | 5725076 | 0.0020956769012144 |
| chr1 | 6168722 | 6168722 | 0.0008727607439972 |
| chr1 | 7623969 | 7623970 | −0.0018944851549297 |
| chr1 | 7803233 | 7803234 | −0.0030778984253225 |
| chr1 | 7866341 | 7866341 | −0.0037889200038957 |
| chr1 | 8089265 | 8089265 | −0.009427212525591 |
| chr1 | 8885004 | 8885005 | 0.0016487801497383 |
| chr1 | 8930797 | 8930798 | 0.0055113606055298 |
| chr1 | 8933633 | 8933633 | 0.0010199956447766 |
| chr1 | 8937002 | 8937003 | 0.0059638145458755 |
| chr1 | 9314315 | 9314316 | −8.337958027779992e−05 |
| chr1 | 9319047 | 9319047 | −0.0001396044923434 |
| chr1 | 9632075 | 9632075 | −0.0032000910874358 |
| chr1 | 11382741 | 11382741 | −0.0032038322431663 |
| chr1 | 11601007 | 11601007 | −0.0050922911804752 |
| chr1 | 13359224 | 13359224 | 0.0049840123713786 |
| chr1 | 13529642 | 13529642 | −0.0017266155862432 |
| chr1 | 13821019 | 13821020 | −0.0095715083563472 |
| chr1 | 13824244 | 13824244 | −0.0081353385101044 |
| chr1 | 13825721 | 13825722 | −0.0054159370513002 |
| chr1 | 14121402 | 14121403 | 0.0024859165899597 |
| chr1 | 14769499 | 14769500 | −0.0027746724268237 |
| chr1 | 15542788 | 15542789 | −0.0013069096616824 |
| chr1 | 15588050 | 15588051 | −0.001234790676313 |
| chr1 | 16056840 | 16056840 | 0.0195769272801681 |
| chr1 | 16615987 | 16615987 | −0.0001538247769203 |
| chr1 | 18025893 | 18025893 | 0.0022372288894495 |
| chr1 | 21347404 | 21347404 | −0.0035738575654152 |
| chr1 | 21558702 | 21558702 | 0.0052131597286081 |
| chr1 | 21559542 | 21559543 | 0.0052629410800471 |
| chr1 | 22070706 | 22070706 | −0.0014780861378436 |
| chr1 | 22174195 | 22174195 | −0.000953011404184 |
| chr1 | 23004003 | 23004003 | −0.007323637083993 |
| chr1 | 23261096 | 23261097 | 0.0019462542565852 |
| chr1 | 24229462 | 24229462 | 0.0052656801187042 |
| chr1 | 24234608 | 24234608 | 0.0004975928465172 |
| chr1 | 24237083 | 24237084 | 0.0036315689788038 |
| chr1 | 24237395 | 24237396 | 0.0004428241476365 |
| chr1 | 24237793 | 24237794 | 0.0068547555556271 |
| chr1 | 24239830 | 24239831 | 0.0053860890314964 |
| chr1 | 24240857 | 24240858 | 5.690556791081639e−05 |
| chr1 | 24241173 | 24241174 | 0.003653066273287 |
| chr1 | 24269207 | 24269208 | −0.0068069549115578 |
| chr1 | 24311080 | 24311080 | 0.000763201467406 |
| chr1 | 24499613 | 24499614 | −0.0046274332673084 |
| chr1 | 24514277 | 24514278 | −0.0050353606068036 |
| chr1 | 25074945 | 25074946 | 0.0028423425945557 |
| chr1 | 25075526 | 25075527 | 0.0002795878722692 |
| chr1 | 25082452 | 25082453 | −0.0079712145479506 |
| chr1 | 25813428 | 25813429 | 0.0014294225602211 |
| chr1 | 26294355 | 26294356 | −0.0046267767601898 |
| chr1 | 26325285 | 26325286 | −0.0042403594030789 |
| chr1 | 27194387 | 27194388 | −0.0006243105381965 |
| chr1 | 27210609 | 27210610 | −0.0010003348472316 |
| chr1 | 29136547 | 29136547 | −0.0025776159067871 |
| chr1 | 29348484 | 29348484 | −0.0023622726099761 |
| chr1 | 31225619 | 31225724 | −0.0012128639124124 |
| chr1 | 33365128 | 33365129 | −0.0003075962985247 |
| chr1 | 33851139 | 33851140 | −0.0018167396107923 |
| chr1 | 34795565 | 34795565 | −0.0010820204439083 |
| chr1 | 36117324 | 36117325 | −0.0021842182926402 |
| chr1 | 37828954 | 37828954 | 0.0006957920483154 |
| chr1 | 39700348 | 39700349 | 0.0022953870863728 |
| chr1 | 42889353 | 42889354 | −0.0048118581704214 |
| chr1 | 44590089 | 44590090 | −0.002228991738412 |
| chr1 | 47247325 | 47247326 | −0.0017723464067823 |
| chr1 | 47771074 | 47771074 | 0.0101851693463872 |
| chr1 | 48368307 | 48368308 | 0.003665581965561 |
| chr1 | 48493808 | 48493808 | 0.0009846817538152 |
| chr1 | 48730662 | 48730663 | 0.0043210813371514 |
| chr1 | 49123199 | 49123200 | −0.0042332637318122 |
| chr1 | 49197494 | 49197495 | 0.0044358809137542 |
| chr1 | 49363924 | 49363924 | −0.0059840469715058 |
| chr1 | 49574749 | 49574750 | −0.0050577254576252 |
| chr1 | 49756239 | 49756240 | −0.000942604819155 |
| chr1 | 53135245 | 53135246 | 0.0004863561120892 |
| chr1 | 53145472 | 53145472 | −0.0061278913800772 |
| chr1 | 53168391 | 53168391 | −0.0029068611916557 |
| chr1 | 53598567 | 53598567 | −0.0005495135066333 |
| chr1 | 54293446 | 54293447 | 0.0021581639104536 |
| chr1 | 54342106 | 54342107 | 0.0029646360037557 |
| chr1 | 54422327 | 54422488 | 0.0046171318135285 |
| chr1 | 54426808 | 54426809 | 0.0013823466222175 |
| chr1 | 54428514 | 54428515 | 0.0030353893907921 |
| chr1 | 54434005 | 54434006 | 0.0025093012898075 |
| chr1 | 54438636 | 54438637 | 0.0045396574778217 |

| | | | | |
|---|---|---|---|---|
| Chromo- | Start | End | | |
| some | Position | position | Cell ID | Weight |

COVID B-Mem

| | | | | |
|---|---|---|---|---|
| chr6 | 30023761 | 30023761 | B-Mem_10799 | −0.1392994870826677 |
| chr9 | 12372416 | 12372416 | B-Mem_13098 | −0.1373868261889206 |
| chr11 | 98664589 | 98664589 | B-Mem_2293 | −0.1344305421483205 |
| chr2 | 170106283 | 170106283 | B-Mem_6984 | −0.1247159948532541 |
| chr9 | 90749599 | 90749599 | B-Mem_13335 | −0.116866327186048 |
| chr12 | 74866841 | 74866841 | B-Mem_2806 | −0.112657791073207 |
| chr14 | 46163750 | 46163750 | B-Mem_3683 | −0.1092780675996854 |
| chr8 | 34063096 | 34063096 | B-Mem_12537 | −0.1029270673601296 |
| chr6 | 112911684 | 112911684 | B-Mem_11171 | −0.1002829435049926 |
| chr22 | 29464882 | 29464882 | B-Mem_8047 | −0.0991681832439522 |
| chr11 | 3541979 | 3541979 | B-Mem_1895 | −0.0988412442293212 |
| chr9 | 105807606 | 105807606 | B-Mem_13420 | −0.097072343167716 |
| chr7 | 99390429 | 99390429 | B-Mem_11953 | −0.0948905906492052 |
| chr2 | 234431838 | 234431838 | B-Mem_7232 | −0.0943070479119024 |
| chr1 | 205382451 | 205382451 | B-Mem_905 | −0.0934105544493282 |
| chr22 | 50396323 | 50396323 | B-Mem_8250 | −0.0913134825367558 |
| chr5 | 137510019 | 137510019 | B-Mem_10394 | −0.0903183663904688 |
| chr5 | 3679669 | 3679669 | B-Mem_9914 | −0.0898176380343156 |
| chr7 | 133583400 | 133583400 | B-Mem_12113 | −0.0891157453171294 |
| chr15 | 27845179 | 27845179 | B-Mem_4058 | −0.0882082922330038 |
| chr10 | 132366593 | 132366593 | B-Mem_1836 | −0.0860546221857273 |
| chr20 | 40463008 | 40463008 | B-Mem_7526 | −0.0858505619055846 |
| chr3 | 136504145 | 136504145 | B-Mem_8887 | −0.0854760799822999 |
| chr5 | 50471136 | 50471136 | B-Mem_10084 | −0.0830521454544391 |
| chr9 | 93129566 | 93129566 | B-Mem_13346 | −0.0824710040478491 |
| chrX | 24283048 | 24283048 | B-Mem_13710 | −0.0821034523407427 |
| chr1 | 211071576 | 211071576 | B-Mem_940 | −0.0820181226936901 |
| chr9 | 102984890 | 102984890 | B-Mem_13398 | −0.0811658129514439 |
| chr17 | 65722882 | 65722882 | B-Mem_5278 | −0.0794335791934922 |
| chr4 | 107010622 | 107010622 | B-Mem_9566 | −0.0792550994584525 |
| chr5 | 90496010 | 90496010 | B-Mem_10223 | −0.0777583239534737 |
| chr9 | 65063503 | 65063503 | B-Mem_13237 | −0.077475463752973 |
| chr6 | 108728546 | 108728546 | B-Mem_11155 | −0.0769188921224446 |
| chr1 | 245920129 | 245920129 | B-Mem_1124 | −0.076830400891973 |
| chr14 | 51770830 | 51770830 | B-Mem_3718 | −0.0746691212408539 |
| chrX | 131907974 | 131907974 | B-Mem_14062 | −0.0743978859462723 |
| chr9 | 24251477 | 24251477 | B-Mem_13142 | −0.0742002219529354 |
| chr5 | 157532965 | 157532965 | B-Mem_10474 | −0.0725550527354431 |
| chr4 | 80908506 | 80908506 | B-Mem_9492 | −0.071332919111642 |
| chr17 | 52145024 | 52145024 | B-Mem_5211 | −0.0712786041776584 |
| chr12 | 29628806 | 29628807 | B-Mem_2620 | −0.0697250068864072 |
| chr21 | 22384095 | 22384095 | B-Mem_7801 | −0.0693004971326342 |
| chr11 | 129142230 | 129142230 | B-Mem_2433 | −0.0681702020827562 |
| chr9 | 94824121 | 94824121 | B-Mem_13360 | −0.066691012938651 |
| chr1 | 209795340 | 209795340 | B-Mem_933 | −0.0661309674725921 |
| chr9 | 60772292 | 60772292 | B-Mem_13231 | −0.0656900057068121 |
| chr16 | 68545771 | 68545771 | B-Mem_4767 | −0.0630922020902428 |
| chr11 | 97062298 | 97062298 | B-Mem_2288 | −0.0630753151260299 |
| chr2 | 52018163 | 52018164 | B-Mem_6482 | −0.0613184627263556 |
| chr13 | 25615389 | 25615389 | B-Mem_3156 | −0.0601205153969966 |
| chr17 | 37806127 | 37806127 | B-Mem_5128 | −0.0600311378032889 |
| chr11 | 4039314 | 4039314 | B-Mem_1898 | −0.0598410756853789 |
| chr17 | 78851225 | 78851225 | B-Mem_5362 | −0.0595330372203239 |
| chrX | 91148295 | 91148295 | B-Mem_13943 | −0.0580942318884171 |
| chr17 | 37605592 | 37605592 | B-Mem_5125 | −0.0564605166272383 |
| chr6 | 100656814 | 100656814 | B-Mem_11131 | −0.0552205717130573 |
| chr16 | 85175092 | 85175092 | B-Mem_4867 | −0.053395958349035 |
| chr5 | 118830338 | 118830338 | B-Mem_10323 | −0.0527443012630291 |
| chr10 | 53600476 | 53600476 | B-Mem_1455 | 0.0519459349848855 |
| chr10 | 109879221 | 109879221 | B-Mem_1683 | 0.0567305171605299 |
| chr15 | 67035052 | 67035052 | B-Mem_4255 | 0.0570101124137504 |
| chr11 | 47768742 | 47768742 | B-Mem_2077 | 0.0578825528395336 |
| chr18 | 3992448 | 3992449 | B-Mem_5447 | 0.0617419733111073 |
| chr11 | 1409568 | 1409568 | B-Mem_1865 | 0.0622500251267187 |
| chr4 | 154481347 | 154481347 | B-Mem_9715 | 0.0633545879047385 |
| chr8 | 13710634 | 13710634 | B-Mem_12407 | 0.0636669181460451 |
| chr17 | 64180186 | 64180186 | B-Mem_5266 | 0.0641545613027253 |
| chr2 | 20554191 | 20554191 | B-Mem_6337 | 0.0653645260245843 |
| chr8 | 43288897 | 43288897 | B-Mem_12588 | 0.0658597458950935 |
| chr15 | 64768028 | 64768028 | B-Mem_4241 | 0.0663795586333893 |
| chr14 | 41491211 | 41491211 | B-Mem_3662 | 0.0664460807110981 |
| chr1 | 101576525 | 101576525 | B-Mem_552 | 0.0667676935231647 |
| chr19 | 14788288 | 14788288 | B-Mem_5971 | 0.0671296451970214 |
| chr2 | 138462010 | 138462010 | B-Mem_6872 | 0.0677053111066948 |
| chr17 | 75613030 | 75613030 | B-Mem_5333 | 0.0687736563934404 |
| chrX | 101348697 | 101348697 | B-Mem_13968 | 0.0715285116534457 |
| chr8 | 124910924 | 124910924 | B-Mem_12924 | 0.0734474785028955 |
| chr6 | 2803482 | 2803483 | B-Mem_10644 | 0.0745651987870181 |
| chr4 | 89877756 | 89877756 | B-Mem_9530 | 0.0748609425472328 |
| chr1 | 235542620 | 235542620 | B-Mem_1076 | 0.0751704563491886 |
| chr6 | 52459697 | 52459697 | B-Mem_10926 | 0.0756553181490753 |
| chr11 | 40692163 | 40692163 | B-Mem_2041 | 0.0773223558119669 |
| chr6 | 24352134 | 24352135 | B-Mem_10766 | 0.0783058244138817 |
| chr1 | 50251383 | 50251384 | B-Mem_366 | 0.0786036984953501 |
| chr14 | 60089505 | 60089505 | B-Mem_3755 | 0.0823294034218884 |
| chr19 | 42891345 | 42891345 | B-Mem_6116 | 0.0849440727973346 |
| chr1 | 235000190 | 235000190 | B-Mem_1074 | 0.0865392365134393 |
| chr7 | 139954458 | 139954458 | B-Mem_12147 | 0.0922322621257661 |
| chr3 | 121922845 | 121922846 | B-Mem_8810 | 0.0933848264284328 |
| chr5 | 81137691 | 81137691 | B-Mem_10194 | 0.093430397360952 |
| chr10 | 129569289 | 129569289 | B-Mem_1814 | 0.0947075443044113 |
| chr6 | 136832836 | 136832837 | B-Mem_11258 | 0.0953350858377475 |
| chr17 | 68849264 | 68849264 | B-Mem_5297 | 0.0958348965307001 |
| chr9 | 43418954 | 43418954 | B-Mem_13228 | 0.0981268208026665 |
| chr2 | 31029795 | 31029795 | B-Mem_6398 | 0.101540291264275 |
| chr2 | 9189869 | 9189869 | B-Mem_6288 | 0.109349517212206 |
| chr10 | 115038461 | 115038461 | B-Mem_1714 | 0.1114813319712488 |
| chr2 | 11517056 | 11517056 | B-Mem_6299 | 0.1152435070053346 |
| chr1 | 110081481 | 110081482 | B-Mem_584 | 0.1315095659529359 |
| chr15 | 63111117 | 63111117 | B-Mem_4232 | 0.2140242236492502 |

COVID B-Naive

| | | | | |
|---|---|---|---|---|
| chr22 | 20902721 | 20902721 | B-Naive_4422 | −0.2877934161972891 |
| chr3 | 122779231 | 122779231 | B-Naive_4887 | −0.261348254173791 |
| chr9 | 98875361 | 98875361 | B-Naive_7362 | −0.1922010186825035 |
| chr2 | 35804908 | 35804908 | B-Naive_3606 | −0.1832358721542585 |
| chr9 | 102890635 | 102890635 | B-Naive_7373 | −0.1747323302126147 |
| chr19 | 51998378 | 51998378 | B-Naive_3440 | −0.1623468935927322 |
| chr1 | 41946575 | 41946575 | B-Naive_206 | −0.1558480814922102 |
| chr22 | 20832576 | 20832576 | B-Naive_4418 | −0.1553426733607572 |
| chr10 | 37920711 | 37920711 | B-Naive_728 | −0.153286816649526 |
| chrX | 151424986 | 151424986 | B-Naive_7859 | −0.1528266610509691 |
| chr3 | 160740228 | 160740228 | B-Naive_4971 | −0.1496172524923133 |
| chr2 | 3102118 | 3102139 | B-Naive_3503 | 0.1450598531258979 |
| chr22 | 34156425 | 34156425 | B-Naive_4460 | −0.1419003754055298 |
| chr19 | 51981395 | 51981396 | B-Naive_3439 | −0.1374078681567543 |
| chr12 | 98744989 | 98744990 | B-Naive_1627 | −0.1362100786820613 |
| chr19 | 13690527 | 13690527 | B-Naive_3294 | −0.1316312076998965 |
| chr2 | 165220106 | 165220106 | B-Naive_3882 | −0.1275482251263311 |
| chr2 | 212165058 | 212165058 | B-Naive_3970 | −0.1262016646781269 |
| chr21 | 42774065 | 42774065 | B-Naive_4366 | −0.1255668970360904 |
| chr14 | 45902154 | 45902154 | B-Naive_2037 | −0.1237170666342424 |
| chr2 | 127846114 | 127846114 | B-Naive_3809 | −0.1165448000556804 |
| chr1 | 42568433 | 42568433 | B-Naive_209 | −0.1116997928163383 |
| chr21 | 44754070 | 44754070 | B-Naive_4379 | −0.1111373271478577 |
| chr6 | 152994560 | 152994560 | B-Naive_6259 | −0.1101229451328568 |
| chr11 | 27043298 | 27043298 | B-Naive_1061 | −0.1086115020500392 |
| chr7 | 104334635 | 104334635 | B-Naive_6653 | −0.1040775154389615 |
| chr16 | 6076334 | 6076334 | B-Naive_2455 | −0.1033713233379922 |
| chr9 | 31374959 | 31374959 | B-Naive_7244 | −0.101155567908378 |
| chr2 | 159198421 | 159198421 | B-Naive_3865 | −0.1000698846603254 |
| chr3 | 33997460 | 33997460 | B-Naive_4661 | −0.0980212270360906 |
| chr3 | 153542973 | 153542974 | B-Naive_4960 | −0.0976885578970944 |
| chr16 | 86758529 | 86758529 | B-Naive_2695 | −0.0904624050038623 |
| chr8 | 71392263 | 71392263 | B-Naive_7014 | −0.0897520290722891 |
| chr14 | 99552084 | 99552084 | B-Naive_2158 | −0.0884142078040714 |
| chr17 | 49024627 | 49024627 | B-Naive_2879 | −0.0879961019110196 |
| chr17 | 47461752 | 47461752 | B-Naive_2872 | −0.0836356014514469 |
| chr22 | 16152904 | 16152904 | B-Naive_4397 | −0.0823562878820997 |
| chr6 | 46778263 | 46778263 | B-Naive_6029 | −0.0820399321591225 |
| chr1 | 191751998 | 191751998 | B-Naive_455 | −0.0816194459155655 |
| chr20 | 51657730 | 51657730 | B-Naive_4208 | −0.0797247537674399 |
| chr16 | 64905241 | 64905241 | B-Naive_2592 | −0.0793717612095408 |
| chr2 | 240893148 | 240893148 | B-Naive_4059 | −0.0788280132474206 |
| chr15 | 30835636 | 30835637 | B-Naive_2237 | −0.0776430167090589 |
| chr2 | 70846073 | 70846073 | B-Naive_3689 | −0.0766512259883483 |
| chr13 | 102903005 | 102903005 | B-Naive_1935 | −0.0743199540801262 |
| chr1 | 176890787 | 176890788 | B-Naive_426 | −0.0739159048426133 |
| chr21 | 44750052 | 44750052 | B-Naive_4377 | −0.0737054518354833 |
| chr6 | 10957877 | 10957877 | B-Naive_5902 | −0.0733217964165356 |
| chr9 | 2936863 | 2936863 | B-Naive_7176 | −0.0710535859623394 |
| chr3 | 109273559 | 109273559 | B-Naive_4846 | −0.07103280300646 |
| chr17 | 64136539 | 64136540 | B-Naive_2920 | −0.0702580573426636 |
| chr19 | 46431527 | 46431527 | B-Naive_3410 | −0.0692221668451729 |
| chr1 | 6150712 | 6150712 | B-Naive_48 | −0.0683443238544823 |
| chr2 | 3108507 | 3108508 | B-Naive_3504 | −0.0680539371274029 |
| chr2 | 43438039 | 43438040 | B-Naive_3627 | −0.064090662747345 |
| chr11 | 132404397 | 132404397 | B-Naive_1348 | −0.0635823238116751 |
| chr4 | 56280599 | 56280599 | B-Naive_5176 | −0.0617420667339445 |
| chr3 | 13005019 | 13005019 | B-Naive_4603 | −0.0605699677112985 |
| chr4 | 10512451 | 10512451 | B-Naive_5072 | 0.0591761806580374 |
| chr15 | 64081623 | 64081623 | B-Naive_2308 | 0.0608634424457209 |
| chr1 | 11207226 | 11207227 | B-Naive_61 | 0.062048113370395 |
| chr12 | 30901046 | 30901046 | B-Naive_1474 | 0.0627522776808388 |
| chr6 | 7360904 | 7360904 | B-Naive_5890 | 0.0669156660297826 |
| chr3 | 128638010 | 128638010 | B-Naive_4908 | 0.0672865172970557 |
| chr3 | 128645766 | 128645766 | B-Naive_4911 | 0.0686916866338971 |
| chr1 | 189060598 | 189060598 | B-Naive_445 | 0.0692292879344156 |
| chr3 | 30154295 | 30154295 | B-Naive_4654 | 0.0735195513745176 |
| chr22 | 20686018 | 20686019 | B-Naive_4416 | 0.0741825391428922 |
| chr8 | 25993624 | 25993625 | B-Naive_6907 | 0.0743257868150635 |
| chr5 | 4018650 | 4018650 | B-Naive_5457 | 0.0744426085719791 |
| chr1 | 234175010 | 234175010 | B-Naive_563 | 0.0758400484791581 |
| chr3 | 109489311 | 109489311 | B-Naive_4848 | 0.0765023218054251 |
| chr21 | 46368217 | 46368217 | B-Naive_4390 | 0.0792900331287063 |
| chr4 | 183032030 | 183032031 | B-Naive_5391 | 0.0810791269912244 |
| chr4 | 57772516 | 57772516 | B-Naive_5182 | 0.0862834369716301 |
| chr11 | 129305504 | 129305505 | B-Naive_1333 | 0.0931295673162182 |
| chr13 | 41967804 | 41967804 | B-Naive_1822 | 0.0938666973965474 |
| chr2 | 134060717 | 134060717 | B-Naive_3831 | 0.0962814120711409 |
| chr2 | 31030320 | 31030321 | B-Naive_3594 | 0.1036236403150332 |
| chr14 | 93999067 | 93999067 | B-Naive_2136 | 0.1074270993401569 |
| chr2 | 31025558 | 31025558 | B-Naive_3593 | 0.1100137023995296 |
| chr20 | 15490644 | 15490645 | B-Naive_4106 | 0.1151768191290325 |
| chr2 | 208027470 | 208027471 | B-Naive_3962 | 0.1174379655835215 |
| chr7 | 121107753 | 121107753 | B-Naive_6691 | 0.1182209016806399 |
| chr12 | 11544900 | 11544900 | B-Naive_1423 | 0.1190059083655925 |
| chr7 | 1287470 | 1287470 | B-Naive_6354 | 0.125366524957357 |
| chr20 | 42649021 | 42649021 | B-Naive_4171 | 0.137363145836913 |
| chr4 | 59844779 | 59844779 | B-Naive_5185 | 0.1403811040823548 |
| chr6 | 168636579 | 168636580 | B-Naive_6326 | 0.143461979636656 |
| chr12 | 95690405 | 95690405 | B-Naive_1616 | 0.1437755438882935 |
| chr16 | 19469210 | 19469210 | B-Naive_2493 | 0.1445008168811548 |
| chr6 | 18603577 | 18603577 | B-Naive_5926 | 0.1567722556639148 |
| chr8 | 3500417 | 3500417 | B-Naive_6837 | 0.1650623414031674 |
| chr2 | 74897171 | 74897172 | B-Naive_3704 | 0.1704965757209169 |
| chr7 | 133960602 | 133960602 | B-Naive_6733 | 0.1705665232590975 |
| chr15 | 50326615 | 50326616 | B-Naive_2265 | 0.1889852905598539 |
| chr12 | 103763840 | 103763840 | B-Naive_1638 | 0.1948683686243329 |
| chr1 | 189061911 | 189061912 | B-Naive_446 | 0.1980785973702322 |
| chr1 | 167535829 | 167535829 | B-Naive_412 | 0.2013176730516203 |
| chr6 | 22181845 | 22181846 | B-Naive_5937 | 0.2356491239928066 |

COVID Monocyte

| | | | | |
|---|---|---|---|---|
| chr8 | 76921910 | 76921910 | Monocyte_37851 | −0.0719068379860225 |
| chr17 | 34569261 | 34569261 | Monocyte_14937 | −0.061747564338397 |
| chr12 | 98759974 | 98759975 | Monocyte_8311 | −0.0604665047793051 |
| chr14 | 67611467 | 67611467 | Monocyte_10990 | −0.0598839991455684 |
| chr13 | 95584361 | 95584362 | Monocyte_9911 | −0.0595800350111991 |
| chr18 | 1900346 | 1900346 | Monocyte_15781 | −0.0578452695310945 |
| chr16 | 70624001 | 70624001 | Monocyte_13977 | −0.0518671275082 |
| chr3 | 153787253 | 153787253 | Monocyte_26099 | −0.0510914985889472 |
| chr3 | 187086268 | 187086268 | Monocyte_26498 | −0.0505616878607944 |
| chr3 | 156511228 | 156511229 | Monocyte_26126 | −0.0502899956316597 |
| chr5 | 80652592 | 80652592 | Monocyte_30296 | −0.0500218886196018 |
| chr12 | 29524008 | 29524008 | Monocyte_7474 | −0.0499832118209227 |
| chrX | 8937154 | 8937154 | Monocyte_40632 | −0.0499431054074085 |
| chrX | 121313287 | 121313287 | Monocyte_41815 | −0.0499012659853813 |
| chr10 | 16421311 | 16421311 | Monocyte_3280 | −0.0496873193721995 |
| chr11 | 92846163 | 92846163 | Monocyte_6438 | −0.0491054992633877 |
| chr16 | 82546247 | 82546247 | Monocyte_14167 | −0.0488457461292004 |
| chr11 | 126367385 | 126367385 | Monocyte_6906 | −0.0488156770633815 |
| chr8 | 125636996 | 125636996 | Monocyte_38403 | −0.0488026858795609 |
| chr8 | 33633899 | 33633899 | Monocyte_37223 | −0.047415500327851 |
| chr5 | 109799131 | 109799132 | Monocyte_30569 | −0.0462836032775833 |
| chr8 | 130927209 | 130927209 | Monocyte_38471 | −0.0460091281542623 |
| chr15 | 85166979 | 85166979 | Monocyte_12521 | −0.0456498588594072 |
| chr10 | 32372896 | 32372896 | Monocyte_3542 | −0.0456101403765179 |
| chr13 | 55967257 | 55967257 | Monocyte_9515 | 0.0454050339612302 |
| chr3 | 136394926 | 136394926 | Monocyte_25899 | −0.0449328039683799 |
| chr10 | 96823052 | 96823052 | Monocyte_4432 | −0.0439302010818638 |
| chr5 | 115985427 | 115985427 | Monocyte_30641 | −0.0426565917791634 |
| chr5 | 86463689 | 86463690 | Monocyte_30344 | −0.0422011723987467 |
| chr18 | 24403363 | 24403363 | Monocyte_16096 | −0.0421331987859663 |
| chr11 | 63069078 | 63069078 | Monocyte_6008 | −0.041972390061569 |
| chrX | 3955209 | 3955209 | Monocyte_40582 | −0.0416039141980532 |
| chr12 | 107677551 | 107677551 | Monocyte_8432 | −0.0412567615505054 |
| chrX | 37466306 | 37466306 | Monocyte_40976 | −0.0408930679249745 |
| chrY | 14336836 | 14336836 | Monocyte_42354 | −0.040597974233948 |
| chr4 | 74392437 | 74392437 | Monocyte_27781 | −0.0401858095689453 |
| chr17 | 47678443 | 47678444 | Monocyte_15120 | −0.0396263731734606 |
| chr22 | 21885015 | 21885015 | Monocyte_23369 | −0.0396038041879051 |
| chrX | 100701696 | 100701696 | Monocyte_41592 | −0.039566616551773 |
| chr9 | 79863919 | 79863920 | Monocyte_39571 | −0.0395357520667965 |
| chr10 | 2744956 | 2744956 | Monocyte_3049 | −0.0390930051841695 |
| chr12 | 76297236 | 76297237 | Monocyte_8039 | −0.0390792036913596 |
| chr3 | 28219885 | 28219885 | Monocyte_24412 | −0.0389622348051487 |
| chr6 | 71249677 | 71249677 | Monocyte_32669 | −0.0389471858431749 |
| chr10 | 61184773 | 61184773 | Monocyte_3977 | −0.038808274313198 |
| chr9 | 31377064 | 31377065 | Monocyte_39275 | −0.0384001841166979 |
| chr1 | 31504628 | 31504628 | Monocyte_658 | −0.0382830021485581 |
| chr6 | 30474638 | 30474638 | Monocyte_32108 | −0.0376062584934438 |
| chr8 | 14688631 | 14688631 | Monocyte_36892 | −0.0376013526781393 |
| chr17 | 47634853 | 47634853 | Monocyte_15117 | −0.0375809840644455 |
| chrX | 112078296 | 112078296 | Monocyte_41700 | −0.0372803546167218 |
| chr13 | 73333635 | 73333635 | Monocyte_9694 | −0.0371085053624499 |
| chr1 | 188894497 | 188894497 | Monocyte_2248 | −0.0369695376388493 |
| chr6 | 23287402 | 23287402 | Monocyte_31986 | −0.0366606094338155 |
| chrX | 39155537 | 39155537 | Monocyte_41001 | −0.0362713467514188 |
| chr15 | 22991129 | 22991130 | Monocyte_11689 | −0.0362424157573687 |
| chr4 | 88227634 | 88227634 | Monocyte_27898 | −0.0356795917162049 |
| chr2 | 230301219 | 230301220 | Monocyte_21179 | −0.0355785859014425 |
| chrX | 152698254 | 152698254 | Monocyte_42209 | −0.0354771870158033 |
| chr4 | 650916 | 650916 | Monocyte_26693 | −0.0351242459908457 |
| chr21 | 36171914 | 36172029 | Monocyte_23040 | 0.0354547676350895 |
| chr2 | 100141984 | 100141984 | Monocyte_19547 | 0.035740379445565 |
| chr5 | 10743307 | 10743307 | Monocyte_29491 | 0.0357468505030738 |
| chr10 | 17989929 | 17989935 | Monocyte_3299 | 0.0358040352532701 |
| chr13 | 35502556 | 35502556 | Monocyte_9278 | 0.0365924207285891 |
| chr9 | 127531340 | 127531341 | Monocyte_40269 | 0.0366739537770598 |
| chr16 | 19461335 | 19461335 | Monocyte_13288 | 0.0371296568066117 |
| chr5 | 107366807 | 107366807 | Monocyte_30560 | 0.0372895272138758 |
| chr3 | 171747111 | 171747119 | Monocyte_26288 | 0.0374569211023543 |
| chr17 | 47513800 | 47513800 | Monocyte_15116 | 0.0377601257805398 |
| chr14 | 94389494 | 94389494 | Monocyte_11366 | 0.0382700420435099 |
| chr7 | 136226485 | 136226485 | Monocyte_36091 | 0.038321341793212 |
| chr12 | 132311788 | 132311788 | Monocyte_8986 | 0.038836059821554 |
| chr13 | 103939841 | 103939842 | Monocyte_10027 | 0.0388447190876464 |
| chr6 | 134715364 | 134715364 | Monocyte_33521 | 0.039000054781215 |
| chr5 | 55134899 | 55134899 | Monocyte_30083 | 0.0390088894279431 |
| chr9 | 125964064 | 125964065 | Monocyte_40233 | 0.0390786287426428 |
| chr16 | 2943814 | 2943814 | Monocyte_12950 | 0.0392835296006915 |
| chr3 | 131104553 | 131104554 | Monocyte_25840 | 0.04018345011214 |
| chr17 | 69062812 | 69062812 | Monocyte_15454 | 0.0404964990969717 |
| chr18 | 34483496 | 34483496 | Monocyte_16281 | 0.0413532125667138 |
| chr3 | 53613799 | 53613799 | Monocyte_24715 | 0.0415065550975966 |
| chr13 | 114082438 | 114082438 | Monocyte_10279 | 0.0420380525246375 |
| chr6 | 2810072 | 2810072 | Monocyte_31692 | 0.0420626861225544 |
| chr4 | 165458207 | 165458207 | Monocyte_28743 | 0.0421109424892023 |
| chr7 | 147063008 | 147063009 | Monocyte_36241 | 0.0425321962167273 |
| chrX | 142075762 | 142075762 | Monocyte_42045 | 0.0426767376980561 |
| chr1 | 100217356 | 100217356 | Monocyte_1521 | 0.0427381209591644 |
| chr8 | 22967484 | 22967484 | Monocyte_37041 | 0.0446957535318136 |
| chr5 | 31929973 | 31929973 | Monocyte_29843 | 0.0454037971367228 |
| chr9 | 19811916 | 19811916 | Monocyte_39080 | 0.0469668121969595 |
| chr2 | 173315660 | 173315660 | Monocyte_20514 | 0.0485840991290847 |
| chr10 | 88054125 | 88054125 | Monocyte_4330 | 0.049786264372663 |
| chr7 | 152504181 | 152504182 | Monocyte_36326 | 0.0512561103264603 |
| chr3 | 128639789 | 128639790 | Monocyte_25799 | 0.0531079315436056 |
| chr3 | 140835254 | 140835274 | Monocyte_25962 | 0.0532463616136819 |
| chr7 | 152507405 | 152507405 | Monocyte_36327 | 0.0553732751948417 |
| chr12 | 117761481 | 117761482 | Monocyte_8636 | 0.0574997632318477 |
| chr6 | 14917121 | 14917121 | Monocyte_31855 | 0.0593627679574284 |
| chr4 | 143264276 | 143264277 | Monocyte_28487 | 0.0657695995321639 |

COVID_NK-cell1

| | | | | |
|---|---|---|---|---|
| chr7 | 19197755 | 19197755 | NK-cell1_7476 | −0.236237119108191 |
| chrX | 119657597 | 119657597 | NK-cell1_8930 | −0.2200899139851938 |
| chr3 | 138575263 | 138575263 | NK-cell1_5656 | −0.2086645208170542 |
| chr1 | 53417074 | 53417074 | NK-cell1_266 | −0.2057149412692501 |
| chr9 | 95830970 | 95830970 | NK-cell1_8527 | −0.203929587600747 |
| chr17 | 57791915 | 57791915 | NK-cell1_3385 | −0.1976294861463343 |
| chr10 | 86237307 | 86237307 | NK-cell1_990 | −0.1924604981261976 |
| chr1 | 43562763 | 43562763 | NK-cell1_232 | −0.1900429486296996 |
| chr7 | 2581366 | 2581366 | NK-cell1_7422 | −0.1720079206544297 |
| chr10 | 29455605 | 29455605 | NK-cell1_812 | −0.1646432729764905 |
| chr21 | 19057818 | 19057818 | NK-cell1_5003 | −0.1596068415672792 |
| chr11 | 28641646 | 28641646 | NK-cell1_1297 | −0.1456644976378916 |
| chr15 | 55994753 | 55994753 | NK-cell1_2700 | −0.1430651107937401 |
| chr4 | 8849081 | 8849081 | NK-cell1_5868 | −0.1322790596396983 |
| chr9 | 133125235 | 133125235 | NK-cell1_8663 | −0.1276780043506827 |
| chr18 | 68876222 | 68876222 | NK-cell1_3697 | −0.1262639548948822 |
| chr5 | 43938323 | 43938323 | NK-cell1_6504 | −0.1256660450697769 |
| chr9 | 115880357 | 115880357 | NK-cell1_8592 | −0.1192546858096652 |
| chr2 | 60168392 | 60168392 | NK-cell1_4202 | −0.1101516373400899 |
| chr4 | 61216181 | 61216181 | NK-cell1_6013 | −0.1094595916962365 |
| chr10 | 90296521 | 90296521 | NK-cell1_998 | −0.1090402877194437 |
| chr16 | 7219608 | 7219608 | NK-cell1_2902 | −0.1056524139066182 |
| chr4 | 8524835 | 8524835 | NK-cell1_5864 | −0.103840837290355 |
| chr21 | 33643895 | 33643895 | NK-cell1_5049 | −0.0988059551625711 |
| chr3 | 30133986 | 30133986 | NK-cell1_5370 | −0.0967761006574431 |
| chr17 | 1680121 | 1680121 | NK-cell1_3206 | −0.0929737612113296 |
| chr18 | 54499056 | 54499056 | NK-cell1_3645 | −0.0914107860576971 |
| chr1 | 42555904 | 42555904 | NK-cell1_228 | −0.0911085579419185 |
| chr16 | 17827610 | 17827610 | NK-cell1_2955 | −0.0898705587627292 |
| chr16 | 77322348 | 77322348 | NK-cell1_3117 | −0.0895768730149023 |
| chr21 | 35229840 | 35229840 | NK-cell1_5054 | −0.0864614605805064 |
| chr21 | 35488687 | 35488687 | NK-cell1_5055 | −0.0833863597362081 |
| chr8 | 13522101 | 13522101 | NK-cell1_7949 | −0.0820867255862742 |
| chr18 | 8808189 | 8808189 | NK-cell1_3537 | −0.0817724265646025 |
| chr1 | 36485488 | 36485488 | NK-cell1_198 | −0.0795884653371855 |
| chr14 | 59308915 | 59308915 | NK-cell1_2409 | −0.0782271380091832 |
| chr18 | 58805108 | 58805108 | NK-cell1_3664 | −0.0749505034696192 |
| chr2 | 115697907 | 115697907 | NK-cell1_4344 | −0.074453292597264 |
| chr5 | 21342135 | 21342135 | NK-cell1_6456 | −0.0733648494605421 |
| chr1 | 158547352 | 158547352 | NK-cell1_456 | −0.0730627345945487 |
| chr1 | 238803562 | 238803562 | NK-cell1_687 | −0.0715872320288578 |
| chr22 | 38396630 | 38396630 | NK-cell1_5200 | −0.0704964459440945 |
| chr9 | 7681970 | 7681970 | NK-cell1_8350 | −0.0695308227562913 |
| chr6 | 113993050 | 113993050 | NK-cell1_7220 | −0.0679421254662579 |
| chr1 | 86952398 | 86952398 | NK-cell1_354 | −0.0675849376331279 |
| chr5 | 179735121 | 179735121 | NK-cell1_6903 | −0.0671265756496058 |
| chr2 | 140011618 | 140011618 | NK-cell1_4436 | −0.066433032345762 |
| chr5 | 7787125 | 7787125 | NK-cell1_6409 | −0.0651924692729771 |
| chr11 | 123139945 | 123139945 | NK-cell1_1586 | −0.0648034815911804 |
| chr7 | 43110729 | 43110729 | NK-cell1_7543 | −0.0642841423399493 |
| chr6 | 167737194 | 167737194 | NK-cell1_7388 | −0.0642342769531194 |
| chr7 | 139011006 | 139011006 | NK-cell1_7808 | −0.0627318540261393 |
| chr12 | 115914798 | 115914798 | NK-cell1_1972 | −0.0626481399149711 |
| chr19 | 34901590 | 34901590 | NK-cell1_3891 | −0.0608553368926749 |
| chr12 | 123532582 | 123532582 | NK-cell1_2002 | −0.05935178327628 |
| chr18 | 40862586 | 40862586 | NK-cell1_3609 | 0.0600150611015343 |
| chr17 | 80902427 | 80902427 | NK-cell1_3489 | 0.0614493860397635 |
| chr5 | 25180651 | 25180651 | NK-cell1_6462 | 0.0615419185554313 |
| chr3 | 167429027 | 167429027 | NK-cell1_5716 | 0.0619177374252625 |
| chr7 | 141260363 | 141260363 | NK-cell1_7815 | 0.0619669437795784 |
| chr2 | 207973097 | 207973097 | NK-cell1_4590 | 0.0646918397545388 |
| chr8 | 129182103 | 129182103 | NK-cell1_8275 | 0.0656152263870004 |
| chr16 | 3013049 | 3013049 | NK-cell1_2882 | 0.0703099573323612 |
| chr17 | 76797512 | 76797512 | NK-cell1_3462 | 0.0737275172171716 |
| chr6 | 47733035 | 47733035 | NK-cell1_7073 | 0.0739663398348317 |
| chr8 | 96150420 | 96150420 | NK-cell1_8207 | 0.0752417945657494 |
| chr2 | 101226814 | 101226814 | NK-cell1_4303 | 0.0770342783672541 |
| chr4 | 151504820 | 151504820 | NK-cell1_6240 | 0.0782873446693298 |
| chr12 | 114730025 | 114730025 | NK-cell1_1966 | 0.078855655883234 |
| chr17 | 47513356 | 47513356 | NK-cell1_3349 | 0.0812128698124672 |
| chr4 | 5031251 | 5031251 | NK-cell1_5841 | 0.0813292204768218 |
| chr1 | 164681178 | 164681178 | NK-cell1_474 | 0.0816868417553489 |
| chr10 | 15239130 | 15239130 | NK-cell1_776 | 0.0832369492789254 |
| chr7 | 1294469 | 1294469 | NK-cell1_7415 | 0.0838822850088201 |
| chr11 | 130409640 | 130409640 | NK-cell1_1611 | 0.0843624350480168 |
| chr7 | 156607783 | 156607783 | NK-cell1_7875 | 0.0857223040947343 |
| chr1 | 213979774 | 213979774 | NK-cell1_608 | 0.0887207544529023 |
| chr4 | 109190002 | 109190002 | NK-cell1_6130 | 0.0905206085360474 |
| chr1 | 24907384 | 24907384 | NK-cell1_143 | 0.090643350201207 |
| chr18 | 3806713 | 3806713 | NK-cell1_3516 | 0.0906874720412401 |
| chr20 | 17883588 | 17883588 | NK-cell1_4787 | 0.0910892241162524 |
| chr13 | 69398217 | 69398217 | NK-cell1_2190 | 0.1012512908785298 |
| chr13 | 63931918 | 63931918 | NK-cell1_2182 | 0.1036973092194044 |
| chr1 | 100865428 | 100865428 | NK-cell1_385 | 0.1048608264607182 |
| chr7 | 42493455 | 42493455 | NK-cell1_7542 | 0.1149443826171915 |
| chr16 | 17452019 | 17452019 | NK-cell1_2952 | 0.1167409751684358 |
| chr22 | 36972055 | 36972055 | NK-cell1_5192 | 0.1167682821051298 |
| chr9 | 9890962 | 9890962 | NK-cell1_8357 | 0.1221952329767264 |
| chr1 | 86182849 | 86182849 | NK-cell1_351 | 0.1270220991405147 |
| chr11 | 131021075 | 131021075 | NK-cell1_1620 | 0.1292025710512144 |
| chr12 | 17544568 | 17544568 | NK-cell1_1711 | 0.140761681288258 |
| chr3 | 162776544 | 162776544 | NK-cell1_5710 | 0.1466918961415636 |
| chr3 | 115841607 | 115841607 | NK-cell1_5596 | 0.1512297448612496 |
| chr1 | 178733076 | 178733076 | NK-cell1_514 | 0.1563306199321013 |
| chr2 | 43863916 | 43863916 | NK-cell1_4157 | 0.1748728054832004 |
| chr6 | 1704585 | 1704585 | NK-cell1_6917 | 0.1864147445669837 |
| chr8 | 66318701 | 66318701 | NK-cell1_8128 | 0.2096573256871329 |
| chr3 | 82290607 | 82290607 | NK-cell1_5521 | 0.21083529905622 |
| chr4 | 126752962 | 126752962 | NK-cell1_6184 | 0.2746051994405931 |
| chr1 | 173322072 | 173322072 | NK-cell1_496 | 0.2747256030559264 |

COVID NK-cell2

| | | | | |
|---|---|---|---|---|
| chr6 | 118677470 | 118677470 | NK-cell2_8825 | −0.1864635651009766 |
| chr19 | 35090003 | 35090003 | NK-cell2_4693 | −0.1592636970317286 |
| chr10 | 61376139 | 61376139 | NK-cell2_1068 | −0.1571486889772102 |
| chr11 | 4555166 | 4555166 | NK-cell2_1451 | −0.1539217299083337 |
| chrX | 121659913 | 121659913 | NK-cell2_10913 | −0.149917130898496 |
| chr1 | 196038309 | 196038309 | NK-cell2_618 | −0.1445499103647022 |
| chr6 | 61323024 | 61323024 | NK-cell2_8592 | −0.128209502543698 |
| chr12 | 48644120 | 48644121 | NK-cell2_2122 | −0.1191355116941974 |
| chr6 | 40538064 | 40538064 | NK-cell2_8507 | −0.1167262240330018 |
| chrX | 110312888 | 110312888 | NK-cell2_10895 | −0.1163015881325527 |
| chr18 | 30077922 | 30077923 | NK-cell2_4315 | −0.1038541767500135 |
| chr4 | 156394892 | 156394892 | NK-cell2_7554 | −0.1018780839662694 |
| chr2 | 1422358 | 1422358 | NK-cell2_4849 | −0.0999503114896567 |
| chr15 | 90109988 | 90109988 | NK-cell2_3402 | −0.0954853349685601 |
| chr10 | 109270681 | 109270681 | NK-cell2_1254 | −0.0954072232072304 |
| chr6 | 60832316 | 60832316 | NK-cell2_8586 | −0.0843927372303963 |
| chr12 | 56492077 | 56492078 | NK-cell2_2156 | −0.0832296384614402 |
| chr10 | 101427395 | 101427395 | NK-cell2_1210 | −0.0824702761865329 |
| chr16 | 18382839 | 18382854 | NK-cell2_3549 | −0.0778532176211119 |
| chr17 | 78217718 | 78217718 | NK-cell2_4150 | −0.07315437992225 |
| chr7 | 25430616 | 25430617 | NK-cell2_9125 | −0.0723082545924761 |
| chrX | 62523389 | 62523389 | NK-cell2_10786 | −0.0720528923443197 |
| chr18 | 30105426 | 30105426 | NK-cell2_4317 | −0.069434527944801 |
| chrX | 106847909 | 106847909 | NK-cell2_10883 | −0.0690549861735777 |
| chr8 | 131322287 | 131322287 | NK-cell2_10113 | −0.0645028573992736 |
| chr21 | 37838014 | 37838014 | NK-cell2_6085 | −0.0623357412718322 |
| chr9 | 110691878 | 110691878 | NK-cell2_10496 | −0.0615562839302543 |
| chr9 | 95615631 | 95615631 | NK-cell2_10452 | −0.0613810302400181 |
| chr12 | 9285110 | 9285110 | NK-cell2_1979 | −0.060534729060902 |
| chr19 | 45664227 | 45664227 | NK-cell2_4756 | 0.0575438355255228 |
| chr13 | 20922396 | 20922396 | NK-cell2_2468 | 0.0595968382027279 |
| chr8 | 23217806 | 23217806 | NK-cell2_9750 | 0.0597371383717926 |
| chr15 | 23962738 | 23962738 | NK-cell2_3166 | 0.0600699691210932 |
| chr22 | 45186978 | 45186978 | NK-cell2_6294 | 0.0601101075482777 |
| chr8 | 105099729 | 105099729 | NK-cell2_10029 | 0.0621288428532219 |
| chr9 | 32114183 | 32114183 | NK-cell2_10324 | 0.0622410837922846 |
| chr17 | 47513800 | 47513800 | NK-cell2_4013 | 0.0632477004933346 |
| chr12 | 39147408 | 39147408 | NK-cell2_2101 | 0.0634260679597597 |
| chr18 | 9757084 | 9757084 | NK-cell2_4230 | 0.0640894834959761 |
| chr2 | 111052538 | 111052538 | NK-cell2_5232 | 0.0641530091510168 |
| chr13 | 45039532 | 45039532 | NK-cell2_2550 | 0.0645083614771614 |
| chr5 | 2703564 | 2703565 | NK-cell2_7718 | 0.06571503330496 |
| chr10 | 112953262 | 112953262 | NK-cell2_1271 | 0.066811250359134 |
| chr10 | 109263251 | 109263251 | NK-cell2_1253 | 0.0681571247388531 |
| chr14 | 72838426 | 72838426 | NK-cell2_2993 | 0.0687296021073698 |
| chr6 | 16362157 | 16362158 | NK-cell2_8399 | 0.0694474101223821 |
| chr17 | 81204704 | 81204704 | NK-cell2_4180 | 0.0700571897487521 |
| chr7 | 121157358 | 121157358 | NK-cell2_9469 | 0.071676583920712 |
| chr2 | 200392480 | 200392480 | NK-cell2_5534 | 0.071748173546414 |
| chr12 | 14777120 | 14777120 | NK-cell2_1997 | 0.0742668059029441 |
| chr13 | 30040543 | 30040543 | NK-cell2_2514 | 0.0758153384150449 |
| chr11 | 30879980 | 30879980 | NK-cell2_1519 | 0.0763128054986496 |
| chr16 | 68487371 | 68487371 | NK-cell2_3708 | 0.0765277590726815 |
| chr6 | 103563141 | 103563366 | NK-cell2_8765 | 0.0789495998874562 |
| chr6 | 46369183 | 46369183 | NK-cell2_8534 | 0.0794602692048222 |
| chr5 | 163535424 | 163535431 | NK-cell2_8236 | 0.0797221407529417 |
| chr15 | 70412484 | 70412484 | NK-cell2_3341 | 0.0802205642014649 |
| chr19 | 22270271 | 22270271 | NK-cell2_4639 | 0.0814936053174025 |
| chr9 | 31888785 | 31888785 | NK-cell2_10318 | 0.0821154964955528 |
| chr17 | 41839052 | 41839052 | NK-cell2_3986 | 0.0824169717358166 |
| chr15 | 68763576 | 68763576 | NK-cell2_3334 | 0.0835416025121426 |
| chr16 | 12820981 | 12820981 | NK-cell2_3534 | 0.0865366565413649 |
| chr20 | 42649020 | 42649021 | NK-cell2_5841 | 0.0875255650578102 |
| chr17 | 2848663 | 2848663 | NK-cell2_3842 | 0.0878474721165705 |
| chr10 | 109239081 | 109239081 | NK-cell2_1249 | 0.0881859053216161 |
| chr16 | 26776811 | 26776811 | NK-cell2_3587 | 0.0898386799050495 |
| chr7 | 133960601 | 133960601 | NK-cell2_9516 | 0.0915618285253727 |
| chr5 | 144889950 | 144889951 | NK-cell2_8159 | 0.0932201299220176 |
| chr2 | 38644925 | 38644925 | NK-cell2_4997 | 0.0983809073931675 |
| chr10 | 69154887 | 69154887 | NK-cell2_1091 | 0.0986689143937239 |
| chr6 | 63225571 | 63225572 | NK-cell2_8606 | 0.0999365055171885 |
| chr6 | 103624773 | 103624773 | NK-cell2_8767 | 0.100205243793382 |
| chr11 | 132251876 | 132251876 | NK-cell2_1912 | 0.1007691323157971 |
| chr15 | 29033834 | 29033834 | NK-cell2_3193 | 0.1014622482160743 |
| chrY | 19366371 | 19366371 | NK-cell2_11034 | 0.1031795927316947 |
| chr10 | 8405953 | 8405953 | NK-cell2_846 | 0.1032057811887964 |
| chr12 | 75800125 | 75800125 | NK-cell2_2210 | 0.1068006050939152 |
| chrX | 44480538 | 44480538 | NK-cell2_10737 | 0.1105707604497547 |
| chr7 | 112649013 | 112649013 | NK-cell2_9443 | 0.110802545076186 |
| chr10 | 101555420 | 101555421 | NK-cell2_1211 | 0.1117386361885396 |
| chr9 | 31981547 | 31981548 | NK-cell2_10319 | 0.1163684303989727 |
| chr6 | 168428695 | 168428695 | NK-cell2_9017 | 0.1210755439770417 |
| chr8 | 6239014 | 6239014 | NK-cell2_9679 | 0.121742839653414 |
| chr19 | 54727757 | 54727757 | NK-cell2_4820 | 0.1292564408659017 |
| chr6 | 137661481 | 137661481 | NK-cell2_8882 | 0.130657312639854 |
| chr18 | 79733052 | 79733052 | NK-cell2_4499 | 0.1323495833703535 |
| chr10 | 1592953 | 1592953 | NK-cell2_815 | 0.133586235755272 |
| chr15 | 98170375 | 98170375 | NK-cell2_3443 | 0.1368975735377847 |
| chr22 | 17880621 | 17880622 | NK-cell2_6162 | 0.1373091308741905 |
| chr5 | 148067981 | 148067981 | NK-cell2_8170 | 0.1386524795432258 |
| chr17 | 64328010 | 64328010 | NK-cell2_4082 | 0.1438557288378476 |
| chr3 | 119464857 | 119464857 | NK-cell2_6750 | 0.1644583268985389 |
| chr8 | 69524980 | 69524980 | NK-cell2_9914 | 0.1663507931466625 |
| chr4 | 143331949 | 143331949 | NK-cell2_7509 | 0.1699191433959195 |
| chr17 | 3740374 | 3740374 | NK-cell2_3847 | 0.1734131599496934 |
| chr1 | 34268464 | 34268464 | NK-cell2_212 | 0.1886696681684545 |
| chr4 | 56119778 | 56119778 | NK-cell2_7220 | 0.1958638486331329 |
| chr9 | 76149081 | 76149082 | NK-cell2_10387 | 0.2257861828616603 |
| chr12 | 93961611 | 93961612 | NK-cell2_2268 | 0.2459452182242004 |
| chr12 | 93962644 | 93962858 | NK-cell2_2269 | 0.2753285407182314 |

COVID Tc-Mem

| | | | | |
|---|---|---|---|---|
| chr17 | 47397632 | 47397632 | Tc-Mem_8085 | −0.1868201394687548 |
| chr16 | 1788836 | 1788836 | Tc-Mem_6889 | −0.1451408130394685 |
| chrY | 8414166 | 8414166 | Tc-Mem_22057 | −0.1308807617921988 |
| chr8 | 1562966 | 1562966 | Tc-Mem_19410 | −0.123392147782086 |
| chr11 | 22371987 | 22371988 | Tc-Mem_3045 | −0.1182978134387802 |
| chr22 | 21847875 | 21847876 | Tc-Mem_12503 | −0.1181560252024995 |
| chr12 | 35845793 | 35845793 | Tc-Mem_4188 | −0.108405495770419 |
| chr4 | 69422393 | 69422393 | Tc-Mem_14728 | −0.1075294492606411 |
| chrX | 87332296 | 87332296 | Tc-Mem_21803 | −0.1072275721192548 |
| chr6 | 57409330 | 57409331 | Tc-Mem_17273 | −0.1039040886859132 |
| chr17 | 47469474 | 47469475 | Tc-Mem_8086 | −0.1010432618798051 |
| chr19 | 51930214 | 51930215 | Tc-Mem_9688 | −0.1000295285888668 |
| chr10 | 28781924 | 28781924 | Tc-Mem_1977 | −0.0987329789453944 |
| chr11 | 65986893 | 65986894 | Tc-Mem_3337 | −0.094727600791349 |
| chr16 | 82546247 | 82546248 | Tc-Mem_7497 | −0.0936117496450065 |
| chr11 | 4556538 | 4556539 | Tc-Mem_2903 | −0.0936012276758232 |
| chr6 | 118517618 | 118517619 | Tc-Mem_17665 | −0.0929657859210425 |
| chr17 | 929225 | 929225 | Tc-Mem_7660 | −0.0917869112441034 |
| chr4 | 63150630 | 63150630 | Tc-Mem_14697 | −0.0864372829413406 |
| chr16 | 81111347 | 81111347 | Tc-Mem_7474 | −0.0853996077889144 |
| chr17 | 66554254 | 66554255 | Tc-Mem_8243 | −0.0850985175138783 |
| chr17 | 47494310 | 47494311 | Tc-Mem_8087 | −0.0840039954073851 |
| chrY | 14430250 | 14430250 | Tc-Mem_22076 | −0.0831147680839917 |
| chr2 | 166391733 | 166391733 | Tc-Mem_10890 | −0.0824095574420173 |
| chr14 | 39063864 | 39063865 | Tc-Mem_5649 | −0.0819979040423197 |
| chr2 | 130495532 | 130495532 | Tc-Mem_10699 | −0.0813804088464373 |
| chrY | 19304133 | 19304133 | Tc-Mem_22085 | −0.0807089951717761 |
| chr14 | 38935727 | 38935728 | Tc-Mem_5645 | −0.0804956611831006 |
| chr6 | 25514419 | 25514420 | Tc-Mem_16996 | −0.080054498145528 |
| chr1 | 10224209 | 10224210 | Tc-Mem_148 | −0.0780201990838814 |
| chr11 | 126373016 | 126373017 | Tc-Mem_3787 | −0.0767051635003473 |
| chr4 | 21105026 | 21105026 | Tc-Mem_14405 | −0.076218827419369 |
| chr5 | 122611214 | 122611214 | Tc-Mem_16270 | −0.0752968107303614 |
| chr17 | 47862380 | 47862380 | Tc-Mem_8099 | −0.0733454272373848 |
| chr2 | 232900718 | 232900718 | Tc-Mem_11338 | −0.0733290252055677 |
| chr7 | 153086877 | 153086878 | Tc-Mem_19297 | −0.0729290112724818 |
| chr15 | 64961852 | 64961852 | Tc-Mem_6550 | −0.0725875300305343 |
| chr8 | 80140379 | 80140380 | Tc-Mem_20005 | −0.0725580643795549 |
| chr13 | 100486874 | 100486874 | Tc-Mem_5354 | −0.0723961879290523 |
| chrY | 17154219 | 17154219 | Tc-Mem_22084 | −0.0713483581070282 |
| chrX | 79392414 | 79392414 | Tc-Mem_21781 | −0.0704974500429326 |
| chr14 | 39004090 | 39004090 | Tc-Mem_5648 | −0.0689166907645602 |
| chr10 | 15395159 | 15395160 | Tc-Mem_1875 | −0.0667451852467268 |
| chr21 | 36187680 | 36187680 | Tc-Mem_12289 | −0.0667154929686536 |
| chr21 | 23392100 | 23392101 | Tc-Mem_12188 | −0.0663725022286552 |
| chr1 | 212854802 | 212854802 | Tc-Mem_1439 | −0.0634489020464496 |
| chr9 | 37200105 | 37200106 | Tc-Mem_20794 | −0.0633557013582264 |
| chr19 | 24893750 | 24893750 | Tc-Mem_9406 | −0.0632167730844189 |
| chr8 | 38934191 | 38934191 | Tc-Mem_19744 | −0.0630886061374842 |
| chr5 | 110901689 | 110901689 | Tc-Mem_16204 | −0.0628705037030693 |
| chr15 | 91463770 | 91463771 | Tc-Mem_6762 | −0.061657855246253 |
| chr14 | 67634586 | 67634586 | Tc-Mem_5851 | −0.0614662369889231 |
| chr5 | 9381987 | 9381989 | Tc-Mem_15644 | −0.0603448626464843 |
| chrX | 122077797 | 122077798 | Tc-Mem_21918 | −0.0602797334947203 |
| chr1 | 177104018 | 177104018 | Tc-Mem_1197 | −0.0577625976662183 |
| chrY | 21853439 | 21853439 | Tc-Mem_22090 | −0.0572278510846478 |
| chr17 | 42329510 | 42329510 | Tc-Mem_8037 | −0.0571545949566577 |
| chr10 | 24780936 | 24780936 | Tc-Mem_1954 | 0.0559676762854093 |
| chr14 | 96785604 | 96785605 | Tc-Mem_6085 | 0.0564681543147027 |
| chr4 | 47957933 | 47957933 | Tc-Mem_14597 | 0.0566671657704056 |
| chr18 | 79733052 | 79733053 | Tc-Mem_9070 | 0.0567256473653068 |
| chr9 | 81644669 | 81644669 | Tc-Mem_20951 | 0.0575829223338341 |
| chr9 | 127439809 | 127439810 | Tc-Mem_21316 | 0.0587834163052457 |
| chr10 | 8440347 | 8440348 | Tc-Mem_1812 | 0.0589003554041603 |
| chr16 | 60094159 | 60094159 | Tc-Mem_7296 | 0.0595214790218429 |
| chr17 | 47594139 | 47594140 | Tc-Mem_8090 | 0.0595330959734436 |
| chr12 | 50230767 | 50230767 | Tc-Mem_4247 | 0.059538713293889 |
| chr18 | 44199863 | 44199863 | Tc-Mem_8786 | 0.0614514295215324 |
| chr4 | 112505816 | 112505816 | Tc-Mem_14983 | 0.0617169901552522 |
| chr6 | 134715364 | 134715364 | Tc-Mem_17757 | 0.065058613594161 |
| chrX | 9960819 | 9960819 | Tc-Mem_21539 | 0.0650655529386944 |
| chr11 | 62334351 | 62334351 | Tc-Mem_3301 | 0.0666706635368534 |
| chr7 | 63527592 | 63527592 | Tc-Mem_18643 | 0.0668720736171734 |
| chr5 | 163643112 | 163643113 | Tc-Mem_16566 | 0.0674804241784749 |
| chr1 | 50324208 | 50324208 | Tc-Mem_553 | 0.0691178701116245 |
| chr12 | 90063090 | 90063090 | Tc-Mem_4476 | 0.071904129903622 |
| chr6 | 147547520 | 147547521 | Tc-Mem_17852 | 0.0775788539175134 |
| chr6 | 136789983 | 136789984 | Tc-Mem_17773 | 0.0777352441227496 |
| chr22 | 36708828 | 36708829 | Tc-Mem_12630 | 0.0787904964014958 |
| chr18 | 79731280 | 79731281 | Tc-Mem_9069 | 0.0801250246187783 |
| chr7 | 152626037 | 152626042 | Tc-Mem_19293 | 0.0826424749210652 |
| chr2 | 3221066 | 3221066 | Tc-Mem_9838 | 0.0840669775264539 |
| chr4 | 143264276 | 143264277 | Tc-Mem_15185 | 0.0887371988342537 |
| chr17 | 68840380 | 68840380 | Tc-Mem_8263 | 0.0922142620176027 |
| chr17 | 69606447 | 69606447 | Tc-Mem_8271 | 0.092793219052317 |
| chr8 | 102720548 | 102720549 | Tc-Mem_20165 | 0.0936124165666802 |
| chr10 | 89590617 | 89590617 | Tc-Mem_2413 | 0.0964832257735484 |
| chr19 | 20974239 | 20974240 | Tc-Mem_9377 | 0.0974421946854174 |
| chr13 | 104673555 | 104673556 | Tc-Mem_5382 | 0.0994638869751461 |
| chr10 | 57821677 | 57821678 | Tc-Mem_2187 | 0.1041907269189002 |
| chr11 | 44589112 | 44589113 | Tc-Mem_3202 | 0.1047736537257387 |
| chr3 | 128638009 | 128638010 | Tc-Mem_13692 | 0.1056873304708263 |
| chr1 | 36234579 | 36234580 | Tc-Mem_416 | 0.1086528919196011 |
| chr1 | 189061911 | 189061912 | Tc-Mem_1269 | 0.1093897251091922 |
| chr6 | 100843386 | 100843386 | Tc-Mem_17549 | 0.1106825860714678 |
| chr4 | 74361248 | 74361249 | Tc-Mem_14761 | 0.1131776993296445 |
| chr1 | 54242874 | 54242875 | Tc-Mem_572 | 0.1142499135451818 |
| chr14 | 101720202 | 101720203 | Tc-Mem_6155 | 0.1190875761939619 |
| chr9 | 71759278 | 71759279 | Tc-Mem_20877 | 0.1196737884788606 |
| chr17 | 68849264 | 68849264 | Tc-Mem_8265 | 0.1801525069519577 |

COVID Tc-Naive

| | | | | |
|---|---|---|---|---|
| chr6 | 61323024 | 61323024 | Tc-Naive_22832 | −0.1249879109798197 |
| chr5 | 86380486 | 86380486 | Tc-Naive_21278 | −0.1000791346646576 |
| chr11 | 80687497 | 80687497 | Tc-Naive_4560 | −0.0978160294134216 |
| chr2 | 122409013 | 122409013 | Tc-Naive_14190 | −0.0953748510684563 |
| chr13 | 27521163 | 27521163 | Tc-Naive_6639 | −0.0940671569850411 |
| chr19 | 52755763 | 52755763 | Tc-Naive_12983 | −0.0860212273268575 |
| chr11 | 126372477 | 126372477 | Tc-Naive_4993 | −0.0841473985030743 |
| chr17 | 47689405 | 47689405 | Tc-Naive_10829 | −0.083811218787477 |
| chrX | 32959155 | 32959155 | Tc-Naive_28143 | −0.0813206310080053 |
| chr20 | 57608373 | 57608373 | Tc-Naive_ 15997 | −0.0798629112871601 |
| chr5 | 147249678 | 147249679 | Tc-Naive_21738 | −0.078317036589119 |
| chr14 | 42079066 | 42079066 | Tc-Naive_7636 | −0.075058400219337 |
| chr3 | 114484820 | 114484820 | Tc-Naive_18202 | −0.0707235664962524 |
| chr22 | 21885015 | 21885015 | Tc-Naive_16670 | −0.0649343463756053 |
| chr9 | 24093658 | 24093658 | Tc-Naive_27009 | −0.0646732779663963 |
| chr8 | 19780326 | 19780326 | Tc-Naive_25594 | −0.0624511567776966 |
| chr9 | 87524951 | 87524951 | Tc-Naive_27355 | −0.0614752748531088 |
| chr2 | 169348289 | 169348289 | Tc-Naive_14574 | −0.0602246484288057 |
| chr12 | 75996342 | 75996342 | Tc-Naive_5903 | −0.0593736139216961 |
| chr4 | 117993304 | 117993304 | Tc-Naive_19971 | −0.0583454669269423 |
| chr5 | 40496173 | 40496174 | Tc-Naive_20966 | −0.0582776142171352 |
| chr11 | 104452165 | 104452165 | Tc-Naive_4722 | −0.0574889824192117 |
| chr12 | 16049451 | 16049451 | Tc-Naive_5340 | −0.0573398782714766 |
| chr14 | 92209347 | 92209362 | Tc-Naive_8138 | −0.0560207218403659 |
| chr6 | 162250169 | 162250169 | Tc-Naive_23675 | 0.0559855286449917 |
| chr16 | 68777834 | 68777834 | Tc-Naive_9894 | −0.0546206107709269 |
| chr9 | 37200105 | 37200106 | Tc-Naive_27123 | −0.0545422366524543 |
| chr9 | 19271058 | 19271058 | Tc-Naive_26961 | −0.0527709307545515 |
| chr11 | 132404396 | 132404397 | Tc-Naive_5082 | −0.0524776388375366 |
| chr9 | 88948390 | 88948390 | Tc-Naive_27376 | −0.0524282726088884 |
| chrY | 15261263 | 15261263 | Tc-Naive_28741 | −0.0522281144399289 |
| chr21 | 22420174 | 22420174 | Tc-Naive_16222 | −0.052099408074241 |
| chr5 | 125501188 | 125501188 | Tc-Naive_21543 | −0.0514262101004023 |
| chr18 | 58537745 | 58537745 | Tc-Naive_11969 | −0.0512296578895793 |
| chr12 | 119643628 | 119643629 | Tc-Naive_6295 | −0.0506118752887073 |
| chr11 | 92839337 | 92839338 | Tc-Naive_4648 | −0.0503756934095854 |
| chr10 | 3275172 | 3275173 | Tc-Naive_2369 | −0.0502644014298186 |
| chr17 | 47642450 | 47642450 | Tc-Naive_10828 | −0.0500801410126731 |
| chr11 | 128151197 | 128151197 | Tc-Naive_5022 | −0.049929473724169 |
| chr5 | 125506067 | 125506068 | Tc-Naive_21544 | −0.0498617947429114 |
| chr13 | 105159532 | 105159533 | Tc-Naive_7264 | −0.0494204541643707 |
| chr12 | 124890180 | 124890181 | Tc-Naive_6398 | −0.0493841229505771 |
| chr14 | 101895400 | 101895400 | Tc-Naive_8304 | −0.049091363915212 |
| chr8 | 80107147 | 80107147 | Tc-Naive_26165 | −0.0490038954302931 |
| chr7 | 68189677 | 68189677 | Tc-Naive_24492 | −0.0488105539394844 |
| chr1 | 3706760 | 3706760 | Tc-Naive_102 | −0.0484387630522916 |
| chr9 | 138097920 | 138097920 | Tc-Naive_28002 | −0.0484255777838236 |
| chr2 | 234597515 | 234597515 | Tc-Naive_15147 | −0.0463545419954331 |
| chr2 | 166589670 | 166589670 | Tc-Naive_14556 | −0.0463118799725237 |
| chr15 | 100004199 | 100004200 | Tc-Naive_9198 | −0.0461242082973726 |
| chr14 | 26063927 | 26063928 | Tc-Naive_7492 | −0.0456990849956776 |
| chr12 | 115590977 | 115590978 | Tc-Naive_6244 | −0.0454477891578438 |
| chr3 | 101921396 | 101921396 | Tc-Naive_18094 | −0.0451713123827296 |
| chr5 | 155648822 | 155648822 | Tc-Naive_21834 | −0.0448349154416665 |
| chr22 | 21904094 | 21904095 | Tc-Naive_16672 | −0.044208817341195 |
| chr6 | 134190010 | 134190011 | Tc-Naive_23386 | −0.043453826454888 |
| chr5 | 83204222 | 83204222 | Tc-Naive_21258 | −0.0433332637959674 |
| chr3 | 329476 | 329476 | Tc-Naive_17155 | 0.0437093433397587 |
| chr4 | 49312885 | 49312885 | Tc-Naive_19475 | 0.0449412129364634 |
| chrX | 151021798 | 151021798 | Tc-Naive_28694 | 0.0450690207499092 |
| chr1 | 177262928 | 177262929 | Tc-Naive_1636 | 0.045114999041533 |
| chr16 | 87343430 | 87343430 | Tc-Naive_10185 | 0.0456640265532777 |
| chr15 | 63115379 | 63115380 | Tc-Naive_8800 | 0.0457007809753636 |
| chr12 | 127670763 | 127670763 | Tc-Naive_6428 | 0.0460445216724684 |
| chr16 | 84522351 | 84522351 | Tc-Naive_10106 | 0.0479244574921994 |
| chr16 | 68640884 | 68640885 | Tc-Naive_9885 | 0.0481092471659304 |
| chr5 | 4023616 | 4023617 | Tc-Naive_20634 | 0.0483560799375237 |
| chr4 | 186230988 | 186230988 | Tc-Naive_20505 | 0.0495342776815496 |
| chr15 | 63127803 | 63127803 | Tc-Naive_8801 | 0.0495809341674121 |
| chr10 | 24773017 | 24773017 | Tc-Naive_2621 | 0.0501342385689953 |
| chr5 | 127815311 | 127815312 | Tc-Naive_21555 | 0.0504301521279458 |
| chr1 | 34249690 | 34249691 | Tc-Naive_615 | 0.0513252805702253 |
| chr8 | 36912840 | 36912840 | Tc-Naive_25818 | 0.0519007919971785 |
| chr17 | 47549028 | 47549029 | Tc-Naive_10824 | 0.0526224665826897 |
| chr13 | 103635462 | 103635462 | Tc-Naive_7246 | 0.0527222081590587 |
| chr2 | 177028004 | 177028005 | Tc-Naive_14652 | 0.0527497474209013 |
| chr16 | 68414785 | 68414785 | Tc-Naive_9880 | 0.0528474467173035 |
| chr4 | 74361248 | 74361249 | Tc-Naive_19621 | 0.0533759170657654 |
| chr13 | 62896989 | 62896990 | Tc-Naive_6934 | 0.0536901226052045 |
| chr17 | 69588456 | 69588456 | Tc-Naive_11078 | 0.0539158443709617 |
| chr1 | 156161136 | 156161136 | Tc-Naive_1434 | 0.0542044743226403 |
| chr2 | 107419012 | 107419013 | Tc-Naive_14040 | 0.0552962798744752 |
| chr9 | 76149081 | 76149082 | Tc-Naive_27268 | 0.0567627463036829 |
| chr12 | 22369875 | 22369875 | Tc-Naive_5388 | 0.0571707572069899 |
| chr5 | 4025924 | 4025925 | Tc-Naive_20635 | 0.0586950084209243 |
| chr9 | 22625342 | 22625343 | Tc-Naive_26997 | 0.0592454475218536 |
| chr7 | 5385069 | 5385070 | Tc-Naive_23925 | 0.0606465461182943 |
| chr2 | 241468803 | 241468804 | Tc-Naive_15271 | 0.0633218740591241 |
| chr8 | 21844791 | 21844792 | Tc-Naive_25630 | 0.0649097099050478 |
| chr4 | 143264276 | 143264276 | Tc-Naive_20140 | 0.0670215574072726 |
| chr11 | 44589112 | 44589113 | Tc-Naive_4184 | 0.0677151768422832 |
| chr13 | 104666554 | 104666555 | Tc-Naive_7256 | 0.0692411482656756 |
| chr11 | 39760955 | 39760955 | Tc-Naive_4142 | 0.0726099134908895 |
| chr10 | 55504150 | 55504150 | Tc-Naive_2928 | 0.0783326341839647 |
| chr13 | 112662423 | 112662424 | Tc-Naive_7362 | 0.083562304968127 |
| chr2 | 241401818 | 241401818 | Tc-Naive_15270 | 0.0869541101140625 |
| chr19 | 34586884 | 34586884 | Tc-Naive_12722 | 0.0938501414127512 |
| chr21 | 13959711 | 13959712 | Tc-Naive_16160 | 0.0962512217752424 |
| chr16 | 81393163 | 81393163 | Tc-Naive_10044 | 0.1157881308162059 |
| chr8 | 23259603 | 23259603 | Tc-Naive_25662 | 0.1199059363282215 |

COVID Th-Mem

| | | | | |
|---|---|---|---|---|
| chr13 | 105138763 | 105138764 | Th-Mem_4806 | −0.1445060214016352 |
| chr11 | 65980222 | 65980223 | Th-Mem_2905 | −0.1435089178175073 |
| chrY | 12203509 | 12203509 | Th-Mem_19800 | −0.1307177073739917 |
| chr11 | 65989693 | 65989693 | Th-Mem_2908 | −0.1230617544518655 |
| chr7 | 95642626 | 95642627 | Th-Mem_16840 | −0.122397672741096 |
| chr1 | 145452559 | 145452559 | Th-Mem_880 | −0.1220090978734337 |
| chr5 | 178825170 | 178825170 | Th-Mem_14882 | −0.120002270968762 |
| chr1 | 112423969 | 112423969 | Th-Mem_821 | −0.1146106606606733 |
| chr11 | 22716522 | 22716522 | Th-Mem_2656 | −0.1101938453820953 |
| chr8 | 111352352 | 111352352 | Th-Mem_18100 | −0.109950061638453 |
| chrY | 16008459 | 16008459 | Th-Mem_19810 | −0.1017951942165973 |
| chr1 | 236415548 | 236415549 | Th-Mem_1423 | −0.1014491036447259 |
| chr9 | 7682930 | 7682930 | Th-Mem_18406 | −0.1004246671744059 |
| chr4 | 53098500 | 53098501 | Th-Mem_13030 | −0.0990568383953213 |
| chrX | 152654676 | 152654677 | Th-Mem_19760 | −0.0986098046645791 |
| chr1 | 236423833 | 236423833 | Th-Mem_1426 | −0.0974440082076911 |
| chr5 | 80609593 | 80609593 | Th-Mem_14278 | −0.0970588572250285 |
| chr11 | 33818360 | 33818360 | Th-Mem_2713 | −0.0944594617001664 |
| chr12 | 85602999 | 85603000 | Th-Mem_3877 | −0.0937214744965666 |
| chr3 | 15879591 | 15879592 | Th-Mem_11504 | −0.0929364466758942 |
| chr13 | 49412928 | 49412929 | Th-Mem_4521 | −0.0904549213440496 |
| chr3 | 13144411 | 13144411 | Th-Mem_11480 | −0.0844415867158092 |
| chr11 | 92846162 | 92846163 | Th-Mem_3071 | −0.0841577549888235 |
| chr2 | 48652763 | 48652764 | Th-Mem_9075 | −0.0799602620299075 |
| chr10 | 57711825 | 57711825 | Th-Mem_1959 | −0.0793642919903778 |
| chr1 | 1620608 | 1620609 | Th-Mem_17 | −0.0775191115837492 |
| chrX | 122077797 | 122077798 | Th-Mem_19631 | −0.0753709591693785 |
| chr5 | 178828681 | 178828681 | Th-Mem_14885 | −0.0743754359027268 |
| chr2 | 3450887 | 3450888 | Th-Mem_8766 | −0.0743648983075816 |
| chr16 | 53788475 | 53788476 | Th-Mem_6489 | −0.0743057825223863 |
| chr10 | 11925663 | 11925664 | Th-Mem_1630 | −0.0742659182503553 |
| chr11 | 81829068 | 81829068 | Th-Mem_3020 | −0.0731729686942514 |
| chr21 | 17507932 | 17507933 | Th-Mem_10745 | −0.0726141022813361 |
| chr12 | 85758166 | 85758166 | Th-Mem_3878 | −0.0708349363542231 |
| chr16 | 68799986 | 68799986 | Th-Mem_6632 | −0.0702523662800503 |
| chr15 | 59716987 | 59716987 | Th-Mem_5810 | −0.0687509542701161 |
| chr5 | 37485934 | 37485934 | Th-Mem_14076 | −0.0683444810584179 |
| chr1 | 201659387 | 201659387 | Th-Mem_1187 | −0.0682518913393977 |
| chr7 | 155848611 | 155848611 | Th-Mem_17260 | −0.0673851481065963 |
| chr19 | 24893750 | 24893750 | Th-Mem_8431 | −0.0652038606734502 |
| chr8 | 73010881 | 73010881 | Th-Mem_17880 | −0.0646028948544288 |
| chr8 | 51896048 | 51896049 | Th-Mem_17763 | −0.0640994270297063 |
| chr7 | 113476743 | 113476744 | Th-Mem_16956 | −0.0640869480494379 |
| chr1 | 115917733 | 115917733 | Th-Mem_847 | −0.0631793006542456 |
| chr5 | 162002269 | 162002270 | Th-Mem_14760 | −0.0614173931981646 |
| chr1 | 115917244 | 115917244 | Th-Mem_846 | −0.060189710793657 |
| chr14 | 66013955 | 66013956 | Th-Mem_5240 | −0.0595776136646645 |
| chr2 | 158878568 | 158878569 | Th-Mem_9677 | −0.0590207272700483 |
| chr6 | 31323287 | 31323288 | Th-Mem_15202 | −0.0586878655441388 |
| chr11 | 128230224 | 128230225 | Th-Mem_3306 | −0.057858185728339 |
| chr14 | 101890138 | 101890139 | Th-Mem_5489 | −0.0577114766988398 |
| chr8 | 1190892 | 1190893 | Th-Mem_17325 | −0.0572809367563174 |
| chr17 | 57597368 | 57597369 | Th-Mem_7316 | −0.0559351257330824 |
| chr13 | 54321707 | 54321707 | Th-Mem_4557 | −0.0555292185887588 |
| chr12 | 132461042 | 132461043 | Th-Mem_4282 | −0.0550329976538782 |
| chr1 | 245202406 | 245202406 | Th-Mem_1482 | −0.0544221194268032 |
| chr9 | 121583819 | 121583820 | Th-Mem_18986 | 0.0546431940437897 |
| chr4 | 143289008 | 143289009 | Th-Mem_13487 | 0.0548592286878624 |
| chr14 | 100344546 | 100344547 | Th-Mem_5468 | 0.0557451899597974 |
| chr17 | 35609636 | 35609637 | Th-Mem_7158 | 0.0566356295367436 |
| chr17 | 35706053 | 35706054 | Th-Mem_7161 | 0.0569155395454424 |
| chr17 | 68849264 | 68849265 | Th-Mem_7403 | 0.0571796478038547 |
| chr5 | 162218464 | 162218464 | Th-Mem_14761 | 0.0576910869034729 |
| chr6 | 124195806 | 124195806 | Th-Mem_15810 | 0.0594849298556358 |
| chr12 | 112540399 | 112540399 | Th-Mem_4051 | 0.0639660335273025 |
| chr22 | 48020686 | 48020687 | Th-Mem_11334 | 0.0646255198657518 |
| chr14 | 100353343 | 100353344 | Th-Mem_5469 | 0.0649620476687256 |
| chr8 | 6258267 | 6258268 | Th-Mem_17400 | 0.0663487698416822 |
| chr4 | 112525982 | 112525982 | Th-Mem_13341 | 0.068093075055803 |
| chr3 | 122752983 | 122752984 | Th-Mem_12115 | 0.0687942611486585 |
| chr5 | 2655002 | 2655002 | Th-Mem_13854 | 0.0691706266513078 |
| chr2 | 226291928 | 226291947 | Th-Mem_10041 | 0.0692944070715103 |
| chr16 | 2139228 | 2139228 | Th-Mem_6155 | 0.0694052820541391 |
| chr4 | 74377846 | 74377846 | Th-Mem_13159 | 0.0712572562068439 |
| chr10 | 89595177 | 89595177 | Th-Mem_2158 | 0.0712681701097173 |
| chr3 | 163734750 | 163734751 | Th-Mem_12390 | 0.07434791152879 |
| chr3 | 128667199 | 128667200 | Th-Mem_12176 | 0.0745071811230106 |
| chr13 | 104673556 | 104673556 | Th-Mem_4802 | 0.0749488120719894 |
| chrX | 131280005 | 131280006 | Th-Mem_19662 | 0.0758531531163402 |
| chr22 | 48037067 | 48037068 | Th-Mem_11335 | 0.0769662893921228 |
| chr8 | 6247259 | 6247259 | Th-Mem_17399 | 0.0773906719567305 |
| chr10 | 57821677 | 57821678 | Th-Mem_1960 | 0.0774457258443155 |
| chr13 | 46809115 | 46809115 | Th-Mem_4505 | 0.0783535945825813 |
| chrX | 4258369 | 4258369 | Th-Mem_19182 | 0.0808977710443555 |
| chr22 | 45158316 | 45158316 | Th-Mem_11285 | 0.0822337811968085 |
| chr8 | 104339303 | 104339304 | Th-Mem_18065 | 0.0833980719914791 |
| chr1 | 92802591 | 92802591 | Th-Mem_712 | 0.0862972314789019 |
| chr16 | 27959732 | 27959733 | Th-Mem_6396 | 0.0908014136637977 |
| chr17 | 69606447 | 69606447 | Th-Mem_7409 | 0.1034913319823397 |
| chr6 | 45182346 | 45182346 | Th-Mem_15341 | 0.1039277881054896 |
| chr14 | 45700617 | 45700618 | Th-Mem_5087 | 0.1058533298463458 |
| chr16 | 19516455 | 19516456 | Th-Mem_6337 | 0.1065669215615562 |
| chr6 | 24585318 | 24585319 | Th-Mem_15126 | 0.1090444555335036 |
| chr14 | 47107045 | 47107045 | Th-Mem_5096 | 0.1118242953877253 |
| chr10 | 10069856 | 10069856 | Th-Mem_1612 | 0.1187406290964427 |
| chr18 | 5102193 | 5102194 | Th-Mem_7619 | 0.1188939129669129 |
| chr8 | 105108221 | 105108221 | Th-Mem_18073 | 0.1194941071678583 |
| chr13 | 112662423 | 112662424 | Th-Mem_4886 | 0.1230128791401108 |
| chr1 | 110081481 | 110081482 | Th-Mem_807 | 0.1245303080140642 |
| chr3 | 128632912 | 128632913 | Th-Mem_12170 | 0.1351052675252737 |

COVID Th-Naïve

| | | | | |
|---|---|---|---|---|
| chr19 | 52919403 | 52919403 | Th-Naive_19952 | −0.1236727790588722 |
| chr5 | 99009492 | 99009492 | Th-Naive_33596 | −0.1100819429116341 |
| chr6 | 30474638 | 30474638 | Th-Naive_35600 | −0.0924363515040922 |
| chr11 | 65986893 | 65986894 | Th-Naive_6839 | −0.0859789876740504 |
| chr6 | 61323024 | 61323024 | Th-Naive_36110 | −0.0792043313659323 |
| chr2 | 212165058 | 212165058 | Th-Naive_23213 | −0.0753624975855183 |
| chr6 | 115009404 | 115009404 | Th-Naive_36758 | −0.0742999336711804 |
| chr2 | 167163864 | 167163865 | Th-Naive_22584 | −0.0692980717783009 |
| chr1 | 143250684 | 143250684 | Th-Naive_2072 | −0.0683452903501267 |
| chr18 | 53823493 | 53823493 | Th-Naive_18439 | −0.0661247576196879 |
| chr19 | 24893750 | 24893751 | Th-Naive_19440 | −0.0648328529578183 |
| chr11 | 92839337 | 92839338 | Th-Naive_7241 | −0.0641890937949979 |
| chr9 | 37200105 | 37200106 | Th-Naive_43047 | −0.0615731676424247 |
| chr22 | 48708563 | 48708563 | Th-Naive_26468 | −0.058577228596574 |
| chr12 | 121119424 | 121119424 | Th-Naive_9864 | −0.0565061605608122 |
| chr9 | 37130435 | 37130436 | Th-Naive_43042 | −0.0561705824643623 |
| chr19 | 53437352 | 53437352 | Th-Naive_19960 | −0.0556504901973642 |
| chr7 | 81973727 | 81973727 | Th-Naive_39022 | −0.0552854753813003 |
| chr1 | 43552145 | 43552145 | Th-Naive_971 | −0.0543496447923168 |
| chr14 | 38957201 | 38957201 | Th-Naive_11920 | −0.0538865414474777 |
| chr17 | 47642449 | 47642450 | Th-Naive_16675 | −0.0537269536443985 |
| chr9 | 37330070 | 37330071 | Th-Naive_43051 | −0.0537215417712981 |
| chr17 | 47361586 | 47361586 | Th-Naive_16668 | −0.0530949073114707 |
| chr19 | 51998379 | 51998379 | Th-Naive_19937 | −0.052951085182754 |
| chr10 | 34454458 | 34454458 | Th-Naive_4207 | −0.0529323461053629 |
| chr18 | 64911329 | 64911329 | Th-Naive_18647 | −0.0527155040543102 |
| chr11 | 119032047 | 119032047 | Th-Naive_7664 | −0.0517339340822358 |
| chr2 | 122139576 | 122139576 | Th-Naive_21921 | −0.050857937404084 |
| chr9 | 37093874 | 37093874 | Th-Naive_43039 | −0.0490482415495024 |
| chr3 | 107303670 | 107303671 | Th-Naive_28181 | −0.0485449557079537 |
| chr20 | 62268087 | 62268087 | Th-Naive_25000 | −0.0480830177721695 |
| chr1 | 236411448 | 236411448 | Th-Naive_3379 | −0.0472312704888127 |
| chr11 | 87220881 | 87220882 | Th-Naive_7170 | −0.0471536314615808 |
| chrX | 122077797 | 122077798 | Th-Naive_45042 | −0.0464992758344447 |
| chr18 | 30117950 | 30117951 | Th-Naive_18016 | −0.0464208992721249 |
| chr11 | 126367385 | 126367385 | Th-Naive_7830 | −0.0460193649576722 |
| chr10 | 60202893 | 60202894 | Th-Naive_4595 | −0.0453608459606888 |
| chr1 | 236646253 | 236646253 | Th-Naive_3393 | −0.0451731308323745 |
| chr10 | 109270681 | 109270682 | Th-Naive_5317 | −0.0445902972960301 |
| chr4 | 63076162 | 63076163 | Th-Naive_30444 | −0.0442757857554091 |
| chr11 | 51231107 | 51231107 | Th-Naive_6644 | −0.0432264308916628 |
| chr8 | 16661347 | 16661347 | Th-Naive_40505 | −0.0430740031445122 |
| chr3 | 182675768 | 182675769 | Th-Naive_29297 | −0.0430583107497766 |
| chr12 | 33234759 | 33234759 | Th-Naive_8660 | −0.0427912301978264 |
| chr14 | 51014853 | 51014853 | Th-Naive_12103 | −0.042347310081127 |
| chr9 | 20719867 | 20719868 | Th-Naive_42838 | −0.0422520164483314 |
| chr18 | 36148020 | 36148020 | Th-Naive_18114 | −0.042250209136931 |
| chr18 | 30207297 | 30207298 | Th-Naive_18038 | −0.0417839008428898 |
| chr4 | 43734713 | 43734713 | Th-Naive_30269 | −0.0417808054751817 |
| chrX | 51514490 | 51514490 | Th-Naive_44683 | −0.0416137786621304 |
| chr4 | 6412915 | 6412915 | Th-Naive_29688 | −0.041285585137552 |
| chr18 | 61650994 | 61650995 | Th-Naive_18592 | −0.0409205662925354 |
| chr4 | 40930464 | 40930464 | Th-Naive_30218 | −0.0407406055563223 |
| chr2 | 148590885 | 148590885 | Th-Naive_22342 | −0.0406706471696788 |
| chr12 | 115590977 | 115590978 | Th-Naive_9759 | −0.0405137517197794 |
| chr13 | 33152401 | 33152401 | Th-Naive_10407 | −0.0402503348659061 |
| chr5 | 80689949 | 80689950 | Th-Naive_33360 | −0.0402307662809257 |
| chr1 | 115917244 | 115917244 | Th-Naive_1991 | −0.039848168017544 |
| chrX | 16501058 | 16501058 | Th-Naive_44485 | −0.0392813385244293 |
| chr15 | 50129497 | 50129497 | Th-Naive_13476 | −0.0392452693081284 |
| chr1 | 112425327 | 112425328 | Th-Naive_1938 | −0.0391588810536968 |
| chr16 | 15247366 | 15247366 | Th-Naive_14694 | −0.0384538461640507 |
| chr11 | 37352937 | 37352937 | Th-Naive_6462 | 0.0379709253314146 |
| chr11 | 10425131 | 10425131 | Th-Naive_6070 | 0.0379786627034385 |
| chr7 | 7868149 | 7868149 | Th-Naive_37825 | 0.0384447501992415 |
| chr7 | 109270004 | 109270004 | Th-Naive_39392 | 0.038985429528194 |
| chr10 | 88502844 | 88502844 | Th-Naive_5021 | 0.0396884535125665 |
| chr2 | 39465251 | 39465252 | Th-Naive_20706 | 0.0402268251611375 |
| chr22 | 37385833 | 37385833 | Th-Naive_26166 | 0.0403314045515066 |
| chr3 | 4643400 | 4643400 | Th-Naive_26607 | 0.0408175722883677 |
| chr16 | 56541059 | 56541059 | Th-Naive_15171 | 0.0428050185922718 |
| chr1 | 239810870 | 239810870 | Th-Naive_3445 | 0.042943294892046 |
| chr4 | 57752277 | 57752278 | Th-Naive_30390 | 0.0430025310598178 |
| chr14 | 101720202 | 101720203 | Th-Naive_12948 | 0.043449180153138 |
| chr15 | 43517602 | 43517602 | Th-Naive_13394 | 0.0439255342888094 |
| chr4 | 110122054 | 110122055 | Th-Naive_31123 | 0.0445211752771811 |
| chr1 | 99183409 | 99183410 | Th-Naive_1738 | 0.0453275262216033 |
| chr4 | 159425150 | 159425150 | Th-Naive_31797 | 0.0458044689884335 |
| chr9 | 31999844 | 31999844 | Th-Naive_42969 | 0.0459701763949464 |
| chr15 | 98173220 | 98173220 | Th-Naive_14304 | 0.0462009503828905 |
| chr5 | 162216747 | 162216747 | Th-Naive_34562 | 0.0468318947998114 |
| chr5 | 150948656 | 150948656 | Th-Naive_34409 | 0.0468886438103111 |
| chr6 | 24585318 | 24585319 | Th-Naive_35503 | 0.0469843089598167 |
| chr18 | 5102194 | 5102194 | Th-Naive_17613 | 0.0476560867609674 |
| chr13 | 74085504 | 74085505 | Th-Naive_10953 | 0.0488147843829195 |
| chr22 | 17210276 | 17210276 | Th-Naive_25758 | 0.0490414089037364 |
| chr5 | 64657939 | 64657940 | Th-Naive_33181 | 0.0493302692930027 |
| chr3 | 171994920 | 171994921 | Th-Naive_29115 | 0.0494573201028182 |
| chr19 | 54384040 | 54384041 | Th-Naive_19986 | 0.0516309547923184 |
| chr1 | 189296409 | 189296409 | Th-Naive_2641 | 0.0520440116446096 |
| chr3 | 128638009 | 128638010 | Th-Naive_28483 | 0.0551287827525886 |
| chr10 | 109220989 | 109220989 | Th-Naive_5315 | 0.0566167471419183 |
| chr9 | 102878369 | 102878369 | Th-Naive_43689 | 0.0576169219191064 |
| chr13 | 104666554 | 104666555 | Th-Naive_11440 | 0.0577892321091514 |
| chr13 | 104673555 | 104673556 | Th-Naive_11441 | 0.0585179562960007 |
| chr9 | 134874526 | 134874527 | Th-Naive_44292 | 0.0601768949341227 |
| chr10 | 24780936 | 24780936 | Th-Naive_4030 | 0.0630542180637371 |
| chr9 | 76149081 | 76149082 | Th-Naive_43244 | 0.0630839239592364 |
| chr19 | 36194408 | 36194409 | Th-Naive_19634 | 0.0798401121530054 |
| chr2 | 190693018 | 190693019 | Th-Naive_22901 | 0.0843805697643154 |

Flu B-Mem

| | | | | |
|---|---|---|---|---|
| chr18 | 55363569 | 55363569 | B-Mem_4137 | −0.1850729731702152 |
| chr9 | 87070309 | 87070309 | B-Mem_9604 | −0.128256112655997 |
| chr1 | 30018531 | 30018531 | B-Mem_162 | −0.1241457212500404 |
| chr12 | 48990086 | 48990086 | B-Mem_1933 | −0.1202713333396537 |
| chr6 | 121103535 | 121103535 | B-Mem_8171 | −0.1073420071180215 |
| chr17 | 75626606 | 75626606 | B-Mem_3871 | −0.104687952806071 |
| chr6 | 15489725 | 15489725 | B-Mem_7858 | −0.1041708040904163 |
| chr5 | 46110614 | 46110614 | B-Mem_7376 | −0.1034421582031041 |
| chr1 | 233027040 | 233027040 | B-Mem_718 | −0.1016840681303621 |
| chr17 | 75804412 | 75804412 | B-Mem_3873 | −0.1013021321677533 |
| chr11 | 95358167 | 95358167 | B-Mem_1612 | −0.1009053890736276 |
| chr4 | 5549331 | 5549331 | B-Mem_6678 | −0.0999445946864351 |
| chr6 | 1064475 | 1064475 | B-Mem_7807 | −0.0976021607019421 |
| chr22 | 45869514 | 45869514 | B-Mem_6011 | −0.0967144443212144 |
| chrX | 25944209 | 25944209 | B-Mem_9976 | −0.0960019756700188 |
| chr3 | 137915436 | 137915436 | B-Mem_6448 | −0.0944468660658424 |
| chr12 | 16219839 | 16219839 | B-Mem_1844 | −0.088866014814993 |
| chr7 | 21811203 | 21811203 | B-Mem_8440 | −0.0833927824361392 |
| chr22 | 50053284 | 50053284 | B-Mem_6047 | −0.0813662899806713 |
| chr16 | 28920531 | 28920531 | B-Mem_3331 | −0.0811742288931985 |
| chrX | 112597019 | 112597019 | B-Mem_10285 | −0.0808214160466668 |
| chr19 | 48492159 | 48492159 | B-Mem_4517 | −0.0802605751865791 |
| chr10 | 130076347 | 130076347 | B-Mem_1241 | −0.0797564675501295 |
| chr9 | 102897151 | 102897151 | B-Mem_9667 | −0.0797228468049294 |
| chr4 | 115740630 | 115740630 | B-Mem_6999 | −0.0793081606218367 |
| chr8 | 65979183 | 65979183 | B-Mem_9124 | −0.0780360976669898 |
| chrX | 33081705 | 33081705 | B-Mem_10010 | −0.0778438055520965 |
| chr9 | 26788500 | 26788500 | B-Mem_9481 | −0.0773700443272594 |
| chr14 | 37584618 | 37584618 | B-Mem_2620 | −0.0773649438211306 |
| chr10 | 411252 | 411252 | B-Mem_773 | −0.0761967771313567 |
| chr1 | 198705417 | 198705417 | B-Mem_576 | −0.0756113878869224 |
| chr5 | 161151335 | 161151335 | B-Mem_7715 | −0.07406718498914 |
| chr11 | 51109941 | 51109941 | B-Mem_1458 | −0.0737955031568807 |
| chr1 | 74863464 | 74863464 | B-Mem_298 | −0.0719318525854109 |
| chr11 | 41289646 | 41289646 | B-Mem_1416 | −0.0713402560018809 |
| chr15 | 17052261 | 17052261 | B-Mem_2890 | −0.0703368631556614 |
| chr5 | 110114154 | 110114154 | B-Mem_7539 | −0.0696978230258808 |
| chr7 | 76626135 | 76626135 | B-Mem_8628 | −0.0696387399486364 |
| chr10 | 30039012 | 30039012 | B-Mem_906 | −0.0622326196131807 |
| chr4 | 41070860 | 41070860 | B-Mem_6813 | −0.0622287034175046 |
| chrX | 29839664 | 29839664 | B-Mem_9995 | −0.0600806100495808 |
| chrX | 87394803 | 87394803 | B-Mem_10218 | −0.05880427475664 |
| chr12 | 33236608 | 33236608 | B-Mem_1894 | −0.0569731747296771 |
| chr7 | 118717965 | 118717965 | B-Mem_8740 | −0.0555380947018656 |
| chr9 | 953210 | 953210 | B-Mem_9393 | −0.0546398191055955 |
| chrX | 151694849 | 151694849 | B-Mem_10457 | −0.0537950493213252 |
| chr17 | 11896819 | 11896819 | B-Mem_3636 | −0.0534223260025934 |
| chr21 | 9821722 | 9821722 | B-Mem_5732 | −0.0531416012766163 |
| chr18 | 22613900 | 22613900 | B-Mem_4016 | −0.0526880828957437 |
| chr18 | 32471954 | 32471954 | B-Mem_4047 | −0.0525596621983575 |
| chrX | 10579733 | 10579733 | B-Mem_9897 | −0.0523352427453889 |
| chr9 | 37652048 | 37652048 | B-Mem_9511 | −0.0520417345606835 |
| chr3 | 146798572 | 146798572 | B-Mem_6477 | 0.0520527719152896 |
| chr18 | 5868709 | 5868709 | B-Mem_3958 | 0.052420355909235 |
| chr2 | 208146665 | 208146665 | B-Mem_5228 | 0.05301288853278 |
| chr1 | 95479977 | 95479977 | B-Mem_352 | 0.0549486435372422 |
| chr7 | 153309832 | 153309832 | B-Mem_8844 | 0.0569181119088511 |
| chr6 | 116741295 | 116741295 | B-Mem_8164 | 0.0586107154941564 |
| chr5 | 118009666 | 118009666 | B-Mem_7568 | 0.0588736953176673 |
| chr10 | 28360589 | 28360589 | B-Mem_895 | 0.0588899835070693 |
| chr2 | 19606695 | 19606695 | B-Mem_4669 | 0.0594331464531996 |
| chr12 | 74968189 | 74968189 | B-Mem_2016 | 0.059820212580807 |
| chr19 | 53978510 | 53978510 | B-Mem_4553 | 0.0609612771585469 |
| chr12 | 7356923 | 7356923 | B-Mem_1813 | 0.0614628814167649 |
| chr8 | 3098266 | 3098266 | B-Mem_8904 | 0.065079475958794 |
| chr7 | 136919465 | 136919465 | B-Mem_8787 | 0.0650908296779128 |
| chr2 | 40299133 | 40299133 | B-Mem_4745 | 0.0658808164293339 |
| chrX | 27671700 | 27671700 | B-Mem_9982 | 0.0664949092962986 |
| chr15 | 91427387 | 91427387 | B-Mem_3128 | 0.0669686861135644 |
| chr2 | 240376538 | 240376538 | B-Mem_5381 | 0.0682114966066695 |
| chr11 | 54987257 | 54987257 | B-Mem_1461 | 0.0690103090657162 |
| chr22 | 43896851 | 43896851 | B-Mem_5995 | 0.069861621982745 |
| chr12 | 19275244 | 19275244 | B-Mem_1857 | 0.0700573707914187 |
| chr21 | 42832757 | 42832757 | B-Mem_5836 | 0.0707907293532936 |
| chr8 | 87893923 | 87893923 | B-Mem_9191 | 0.0738322154871175 |
| chr13 | 73766010 | 73766010 | B-Mem_2392 | 0.0742674221566563 |
| chr6 | 40434529 | 40434529 | B-Mem_7959 | 0.0751866551182456 |
| chr2 | 31815788 | 31815788 | B-Mem_4727 | 0.0761242962056084 |
| chrX | 143941527 | 143941527 | B-Mem_10420 | 0.0776102736120881 |
| chr3 | 168450716 | 168450716 | B-Mem_6542 | 0.0776375577188679 |
| chr12 | 111687281 | 111687281 | B-Mem_2122 | 0.0807536566323693 |
| chr9 | 106128670 | 106128670 | B-Mem_9677 | 0.0810172503428461 |
| chr19 | 53739914 | 53739914 | B-Mem_4548 | 0.0832366923345135 |
| chr13 | 26054320 | 26054320 | B-Mem_2259 | 0.0844735108360239 |
| chrX | 28690593 | 28690593 | B-Mem_9987 | 0.1014168501758847 |
| chr13 | 63473456 | 63473456 | B-Mem_2358 | 0.1017402438589971 |
| chr9 | 43181346 | 43181346 | B-Mem_9527 | 0.1047503614279131 |
| chr15 | 60855063 | 60855063 | B-Mem_3018 | 0.1050171424024504 |
| chr6 | 56066037 | 56066037 | B-Mem_8001 | 0.1065046595274764 |
| chr13 | 26592024 | 26592024 | B-Mem_2260 | 0.1065170901865401 |
| chr8 | 7138497 | 7138497 | B-Mem_8919 | 0.1097952684042206 |
| chr4 | 74528621 | 74528621 | B-Mem_6896 | 0.1123980989502226 |
| chr5 | 58685790 | 58685790 | B-Mem_7395 | 0.1145098322432748 |
| chr11 | 114658104 | 114658104 | B-Mem_1671 | 0.1190713519378678 |
| chr12 | 39447551 | 39447551 | B-Mem_1909 | 0.1223882802329933 |
| chr3 | 62565347 | 62565347 | B-Mem_6246 | 0.1328081913598128 |
| chr8 | 18431075 | 18431075 | B-Mem_8961 | 0.1341075613820948 |
| chr3 | 13882075 | 13882075 | B-Mem_6106 | 0.1400452471770933 |
| chr10 | 29005615 | 29005615 | B-Mem_897 | 0.1487630206351157 |
| chr1 | 210161191 | 210161191 | B-Mem_627 | 0.1637392645508109 |

Flu B-Naïve

| | | | | |
|---|---|---|---|---|
| chr4 | 68197897 | 68197897 | B-Naive_2638 | −0.317612755560379 |
| chr1 | 46512962 | 46512962 | B-Naive_121 | −0.3095673909965493 |
| chr17 | 18967211 | 18967211 | B-Naive_1434 | −0.2648493997745763 |
| chr13 | 77757537 | 77757537 | B-Naive_900 | −0.2519893802561223 |
| chr16 | 71033623 | 71033623 | B-Naive_1332 | −0.2406810077346156 |
| chr1 | 178939259 | 178939259 | B-Naive_229 | −0.2396689583271389 |
| chr7 | 60290562 | 60290562 | B-Naive_3176 | −0.2353746501521259 |
| chr11 | 54719720 | 54719720 | B-Naive_554 | −0.2317648835614884 |
| chr20 | 17202764 | 17202764 | B-Naive_2115 | −0.2002341094773726 |
| chr16 | 77031116 | 77031116 | B-Naive_1346 | −0.1904951059854862 |
| chr19 | 28789397 | 28789397 | B-Naive_1744 | −0.1712882255889408 |
| chr1 | 143494843 | 143494843 | B-Naive_187 | −0.1704673021411391 |
| chr3 | 85234268 | 85234268 | B-Naive_2471 | −0.1605096757274552 |
| chr15 | 82082116 | 82082116 | B-Naive_1163 | −0.1524533241528505 |
| chr19 | 14361785 | 14361785 | B-Naive_1724 | −0.144494136910049 |
| chr5 | 141144029 | 141144029 | B-Naive_2867 | −0.1408612543427861 |
| chr12 | 30183416 | 30183416 | B-Naive_693 | −0.1327728360293137 |
| chr2 | 51889293 | 51889293 | B-Naive_1910 | −0.1322138236448811 |
| chrX | 131546693 | 131546693 | B-Naive_3901 | −0.1297118529098077 |
| chrX | 137433790 | 137433790 | B-Naive_3920 | −0.1276778314437543 |
| chr11 | 65186583 | 65186583 | B-Naive_575 | −0.1205204719107679 |
| chr9 | 137546023 | 137546023 | B-Naive_3606 | −0.1196771128678959 |
| chr15 | 23911544 | 23911544 | B-Naive_1081 | −0.1178150749946978 |
| chr5 | 41870860 | 41870860 | B-Naive_2799 | −0.1169990598593119 |
| chrX | 120469146 | 120469146 | B-Naive_3878 | −0.1166060292794027 |
| chr7 | 62265837 | 62265837 | B-Naive_3180 | −0.1056341553444332 |
| chr3 | 1475443 | 1475443 | B-Naive_2383 | −0.103716623435225 |
| chrX | 153647760 | 153647761 | B-Naive_3960 | −0.0991330476457824 |
| chr11 | 28636131 | 28636131 | B-Naive_530 | −0.095399228390122 |
| chr4 | 110963803 | 110963803 | B-Naive_2668 | −0.0842785722027124 |
| chr11 | 77190883 | 77190883 | B-Naive_599 | −0.0835574078764346 |
| chr19 | 44760331 | 44760331 | B-Naive_1782 | −0.0834043441258274 |
| chr2 | 85474897 | 85474897 | B-Naive_1937 | −0.0823016943306935 |
| chrX | 151638765 | 151638765 | B-Naive_3954 | −0.0789624415915932 |
| chr16 | 67185371 | 67185371 | B-Naive_1325 | −0.0773983566236755 |
| chr15 | 99141310 | 99141310 | B-Naive_1195 | −0.0768794044554755 |
| chr21 | 20475005 | 20475005 | B-Naive_2260 | −0.0762646385594817 |
| chr16 | 87381323 | 87381323 | B-Naive_1373 | −0.0740824391352304 |
| chrX | 50801197 | 50801197 | B-Naive_3723 | −0.0739469248302957 |
| chr2 | 242046794 | 242046794 | B-Naive_2094 | −0.072723726804164 |
| chr7 | 57639163 | 57639163 | B-Naive_3171 | −0.0701084402246709 |
| chr9 | 29774610 | 29774610 | B-Naive_3469 | −0.0675762412691461 |
| chr21 | 36522531 | 36522531 | B-Naive_2279 | −0.0658959774827422 |
| chr16 | 68761044 | 68761044 | B-Naive_1330 | −0.0651479446822408 |
| chrX | 127679483 | 127679483 | B-Naive_3887 | −0.0649875544249999 |
| chrX | 86554831 | 86554831 | B-Naive_3811 | −0.0606126373973607 |
| chr3 | 162834934 | 162834934 | B-Naive_2522 | −0.0586715280117907 |
| chrX | 67677242 | 67677242 | B-Naive_3760 | −0.0558985600372618 |
| chr3 | 133472581 | 133472581 | B-Naive_2503 | −0.0508425356159355 |
| chr4 | 110705096 | 110705096 | B-Naive_2667 | −0.0506636285322745 |
| chr12 | 912764 | 912764 | B-Naive_673 | 0.049155024306447 |
| chr6 | 32587254 | 32587254 | B-Naive_2979 | 0.0504780865088502 |
| chr12 | 34349505 | 34349505 | B-Naive_698 | 0.0542497288616973 |
| chr14 | 70607381 | 70607381 | B-Naive_1007 | 0.0628604924937973 |
| chr17 | 18370386 | 18370386 | B-Naive_1432 | 0.0639173071876908 |
| chr3 | 13011206 | 13011206 | B-Naive_2400 | 0.064866105486062 |
| chr1 | 111904718 | 111904751 | B-Naive_178 | 0.069142350131715 |
| chr2 | 209579274 | 209579274 | B-Naive_2039 | 0.0706484645192067 |
| chrX | 142427897 | 142427897 | B-Naive_3931 | 0.0710732905753376 |
| chr2 | 69187154 | 69187154 | B-Naive_1923 | 0.0725073947477887 |
| chr14 | 22803342 | 22803342 | B-Naive_963 | 0.079167121553366 |
| chr10 | 41895445 | 41895445 | B-Naive_371 | 0.0794490746906152 |
| chr17 | 47734512 | 47734512 | B-Naive_1492 | 0.0798821659874212 |
| chr6 | 29847180 | 29847180 | B-Naive_2975 | 0.082550332458243 |
| chrX | 66867517 | 66867517 | B-Naive_3759 | 0.0837032969276457 |
| chr14 | 106000217 | 106000217 | B-Naive_1070 | 0.0843957106144783 |
| chr5 | 141429045 | 141429045 | B-Naive_2877 | 0.0843968924070235 |
| chr7 | 150427554 | 150427554 | B-Naive_3263 | 0.0844574111300984 |
| chr10 | 61943304 | 61943304 | B-Naive_389 | 0.0853937509700596 |
| chr16 | 53950311 | 53950311 | B-Naive_1308 | 0.088021240385689 |
| chr4 | 178673618 | 178673618 | B-Naive_2720 | 0.089182973118404 |
| chr19 | 54254182 | 54254182 | B-Naive_1826 | 0.0899360593487047 |
| chr19 | 56838387 | 56838387 | B-Naive_1833 | 0.0945047382868285 |
| chr4 | 102564671 | 102564671 | B-Naive_2662 | 0.094535891484076 |
| chr1 | 805477 | 805477 | B-Naive_0 | 0.0945748194497552 |
| chr22 | 26057064 | 26057064 | B-Naive_2322 | 0.1021128282355881 |
| chr15 | 97246420 | 97246420 | B-Naive_1192 | 0.1021387158821926 |
| chr16 | 46573522 | 46573522 | B-Naive_1290 | 0.1034615331108456 |
| chr2 | 105547004 | 105547004 | B-Naive_1959 | 0.1065082734570695 |
| chr10 | 132423849 | 132423849 | B-Naive_492 | 0.1073175194807227 |
| chr21 | 27540080 | 27540080 | B-Naive_2267 | 0.1084218310092162 |
| chr20 | 56177444 | 56177444 | B-Naive_2208 | 0.1174900202020786 |
| chr13 | 89380472 | 89380472 | B-Naive_910 | 0.1183491266265878 |
| chr7 | 56327201 | 56327201 | B-Naive_3162 | 0.1188103777705643 |
| chr8 | 24296500 | 24296500 | B-Naive_3330 | 0.1234196463412899 |
| chr2 | 122447012 | 122447012 | B-Naive_1979 | 0.12404431992535 |
| chr15 | 73804544 | 73804544 | B-Naive_1148 | 0.1314916390621923 |
| chr8 | 16776797 | 16776797 | B-Naive_3319 | 0.134212191224074 |
| chr16 | 84482441 | 84482441 | B-Naive_1359 | 0.1394378736156645 |
| chr9 | 23416089 | 23416089 | B-Naive_3462 | 0.1396885258671265 |
| chr3 | 140047940 | 140047940 | B-Naive_2511 | 0.1552148286448122 |
| chr20 | 60967621 | 60967621 | B-Naive_2225 | 0.1586449617670893 |
| chr17 | 3601379 | 3601379 | B-Naive_1399 | 0.2152324066590398 |
| chrX | 87381593 | 87381593 | B-Naive_3812 | 0.2245585588889414 |
| chrX | 12586865 | 12586865 | B-Naive_3636 | 0.2284298361896994 |
| chr2 | 146866444 | 146866444 | B-Naive_2002 | 0.2311728851980194 |
| chr9 | 44955708 | 44955708 | B-Naive_3482 | 0.267647129761567 |
| chr7 | 156160669 | 156160669 | B-Naive_3279 | 0.3196772572857082 |
| chrX | 37496834 | 37496834 | B-Naive_3701 | 0.3535555346764978 |
| chr4 | 81841857 | 81841857 | B-Naive_2651 | 0.3612087532918306 |

Flu_Monocyte

| | | | | |
|---|---|---|---|---|
| chr12 | 58110606 | 58110606 | Monocyte_1383 | −0.4518601830607582 |
| chr1 | 85993377 | 85993377 | Monocyte_254 | −0.3292571455950095 |
| chr22 | 18627679 | 18627679 | Monocyte_4170 | −0.2735839584548278 |
| chr3 | 196625575 | 196625575 | Monocyte_4618 | −0.2702668293212066 |
| chr15 | 74222448 | 74222448 | Monocyte_2121 | −0.2676228526814815 |
| chr1 | 120682909 | 120682909 | Monocyte_307 | −0.229425634550517 |
| chr17 | 15632302 | 15632302 | Monocyte_2573 | −0.2016207515171075 |
| chrX | 107493211 | 107493211 | Monocyte_7127 | −0.1939589939232623 |
| chr16 | 32812158 | 32812158 | Monocyte_2325 | −0.1869500097152186 |
| chr17 | 21239767 | 21239767 | Monocyte_2592 | −0.1788200520734944 |
| chr2 | 86549343 | 86549343 | Monocyte_3500 | −0.174235540526197 |
| chr8 | 43251709 | 43251709 | Monocyte_6286 | −0.1724213029777448 |
| chrX | 135686893 | 135686893 | Monocyte_7241 | −0.1715611960943816 |
| chr10 | 130875040 | 130875040 | Monocyte_840 | −0.1529334275401641 |
| chr2 | 235661190 | 235661190 | Monocyte_3784 | −0.1527686669276045 |
| chr12 | 26180001 | 26180001 | Monocyte_1305 | −0.1526265056967313 |
| chr4 | 170868371 | 170868371 | Monocyte_4958 | −0.140822277981723 |
| chr2 | 60937879 | 60937879 | Monocyte_3451 | −0.1384548006723075 |
| chr4 | 9388919 | 9388919 | Monocyte_4677 | −0.1375802773708139 |
| chr17 | 16844831 | 16844831 | Monocyte_2577 | −0.1314042192494833 |
| chr21 | 8841365 | 8841365 | Monocyte_4082 | −0.1267097758240418 |
| chr16 | 32741529 | 32741529 | Monocyte_2324 | −0.1191368410222232 |
| chrX | 72169406 | 72169406 | Monocyte_7022 | −0.1167552751983192 |
| chr7 | 61067828 | 61067828 | Monocyte_5901 | −0.1129837643334428 |
| chr20 | 28800228 | 28800228 | Monocyte_3908 | −0.1127590713105411 |
| chr6 | 50455585 | 50455585 | Monocyte_5529 | −0.1125510371395905 |
| chr9 | 16383906 | 16383906 | Monocyte_6524 | −0.1123204908141814 |
| chr22 | 15879367 | 15879367 | Monocyte_4162 | −0.1063633473057281 |
| chr16 | 78678720 | 78678720 | Monocyte_2459 | −0.1047948311440211 |
| chr4 | 12318749 | 12318749 | Monocyte_4682 | −0.1045659214236513 |
| chr11 | 2122361 | 2122361 | Monocyte_878 | −0.0943969334444185 |
| chr11 | 114093404 | 114093404 | Monocyte_1169 | −0.0936164807376486 |
| chr1 | 4681043 | 4681043 | Monocyte_31 | −0.0906883823316867 |
| chr19 | 53908659 | 53908659 | Monocyte_3276 | −0.0894200308485787 |
| chr19 | 35313654 | 35313654 | Monocyte_3185 | −0.0858004512056898 |
| chr6 | 31766624 | 31766624 | Monocyte_5494 | −0.0844564917738506 |
| chr7 | 119754396 | 119754396 | Monocyte_6053 | −0.081781421349675 |
| chr18 | 48939184 | 48939184 | Monocyte_2924 | −0.0809714512424184 |
| chr2 | 188885825 | 188885825 | Monocyte_3690 | −0.0788258616778954 |
| chr19 | 43763413 | 43763413 | Monocyte_3217 | −0.0757514375410021 |
| chr15 | 39208976 | 39208976 | Monocyte_2043 | −0.0743968160872581 |
| chr5 | 84042765 | 84042765 | Monocyte_5204 | −0.0654442611374253 |
| chr22 | 12282444 | 12282444 | Monocyte_4159 | −0.065225169030199 |
| chr6 | 69104371 | 69104371 | Monocyte_5562 | −0.0642204909987661 |
| chr10 | 46282313 | 46282313 | Monocyte_635 | −0.0600572455967634 |
| chr21 | 45111292 | 45111292 | Monocyte_4146 | −0.0600409311448825 |
| chr11 | 22453995 | 22453995 | Monocyte_933 | −0.0599390180884015 |
| chr5 | 113786796 | 113786796 | Monocyte_5256 | −0.0584362100764941 |
| chr4 | 3515783 | 3515783 | Monocyte_4642 | −0.0578889558263119 |
| chrX | 118156113 | 118156113 | Monocyte_7162 | 0.0580775336421926 |
| chr15 | 60843162 | 60843162 | Monocyte_2084 | 0.0595750646541692 |
| chr5 | 37910969 | 37910969 | Monocyte_5136 | 0.0597040568974571 |
| chr15 | 71634704 | 71634704 | Monocyte_2113 | 0.0630644099827761 |
| chr10 | 67439962 | 67439962 | Monocyte_676 | 0.0647757549231655 |
| chr11 | 49609354 | 49609354 | Monocyte_997 | 0.0667325129565271 |
| chr6 | 122276539 | 122276539 | Monocyte_5653 | 0.0698984870309311 |
| chr8 | 112311550 | 112311550 | Monocyte_6402 | 0.0721167487839951 |
| chr11 | 89724032 | 89724032 | Monocyte_1110 | 0.0725473123300441 |
| chr4 | 162384467 | 162384467 | Monocyte_4941 | 0.073244495390182 |
| chr2 | 219044510 | 219044510 | Monocyte_3741 | 0.0734093142429099 |
| chr4 | 59534540 | 59534540 | Monocyte_4770 | 0.0772090776692346 |
| chr18 | 46926602 | 46926602 | Monocyte_2910 | 0.0795596624368285 |
| chrX | 9340391 | 9340391 | Monocyte_6798 | 0.0831312408308571 |
| chr7 | 62178695 | 62178695 | Monocyte_5903 | 0.0836437661721297 |
| chr18 | 7152153 | 7152153 | Monocyte_2832 | 0.0838920284199554 |
| chr17 | 50238010 | 50238010 | Monocyte_2695 | 0.0892824123987708 |
| chrX | 103215552 | 103215561 | Monocyte_7109 | 0.0908799686109358 |
| chr1 | 161472200 | 161472200 | Monocyte_344 | 0.1033589742800078 |
| chr2 | 158376707 | 158376707 | Monocyte_3647 | 0.1052358239027369 |
| chr7 | 22055456 | 22055456 | Monocyte_5805 | 0.1065031972870335 |
| chrX | 69521549 | 69521549 | Monocyte_7011 | 0.1077041171299704 |
| chr1 | 96906806 | 96906806 | Monocyte_270 | 0.1111795068161342 |
| chrX | 67544040 | 67544040 | Monocyte_7001 | 0.1151837060532561 |
| chr5 | 126349458 | 126349458 | Monocyte_5284 | 0.1156008947806545 |
| chr12 | 129654880 | 129654880 | Monocyte_1538 | 0.1197632536752648 |
| chr9 | 109032084 | 109032084 | Monocyte_6665 | 0.1206623037188012 |
| chr3 | 118226461 | 118226461 | Monocyte_4477 | 0.1219455180802765 |
| chr12 | 16187413 | 16187413 | Monocyte_1282 | 0.122871839982612 |
| chr14 | 106077516 | 106077516 | Monocyte_1999 | 0.1271253410001605 |
| chr1 | 93850894 | 93850894 | Monocyte_265 | 0.1276603277899463 |
| chr4 | 136556330 | 136556330 | Monocyte_4899 | 0.1284092977760246 |
| chr8 | 109033164 | 109033164 | Monocyte_6398 | 0.1326334729461733 |
| chr2 | 202624543 | 202624543 | Monocyte_3709 | 0.133149142952899 |
| chr6 | 110476859 | 110476859 | Monocyte_5635 | 0.1338868016943366 |
| chr9 | 64797962 | 64797962 | Monocyte_6588 | 0.1398343775003467 |
| chr7 | 87134043 | 87134043 | Monocyte_5977 | 0.1552449012248098 |
| chr11 | 22063732 | 22063732 | Monocyte_931 | 0.1679937707685285 |
| chr10 | 114310963 | 114310963 | Monocyte_785 | 0.1722314149574683 |
| chrX | 57454015 | 57454015 | Monocyte_6978 | 0.1736602831133992 |
| chr1 | 118837476 | 118837476 | Monocyte_304 | 0.1773354008917899 |
| chrX | 71278897 | 71278897 | Monocyte_7016 | 0.1825333664715571 |
| chr16 | 64782012 | 64782012 | Monocyte_2417 | 0.1905058199356473 |
| chr20 | 13391738 | 13391738 | Monocyte_3857 | 0.1910818052155823 |
| chr9 | 129546002 | 129546002 | Monocyte_6719 | 0.1927508994421445 |
| chrX | 30914332 | 30914332 | Monocyte_6875 | 0.204314416406103 |
| chr4 | 478234 | 478234 | Monocyte_4622 | 0.2321647786095636 |
| chr12 | 92115087 | 92115087 | Monocyte_1426 | 0.262443859412887 |
| chr5 | 135755741 | 135755741 | Monocyte_5295 | 0.3494776130640482 |
| chr10 | 49326554 | 49326554 | Monocyte_645 | 0.3935026040111137 |
| chr2 | 90237563 | 90237563 | Monocyte_3508 | 0.4178655177302308 |

Flu_NK-cell2

| | | | | |
|---|---|---|---|---|
| chrY | 9655573 | 9655573 | NK-cell2_6166 | −0.2742890857673282 |
| chr15 | 37308939 | 37308939 | NK-cell2_1710 | −0.2002103408551296 |
| chr2 | 235311549 | 235311549 | NK-cell2_3096 | −0.1912234903604514 |
| chr8 | 87137282 | 87137282 | NK-cell2_5316 | −0.1861747595888231 |
| chr4 | 103429400 | 103429400 | NK-cell2_4030 | −0.1852399215789156 |
| chr16 | 67290369 | 67290369 | NK-cell2_1989 | −0.1756098186615975 |
| chr2 | 175164317 | 175164317 | NK-cell2_3010 | −0.1687511226752972 |
| chr10 | 40613078 | 40613078 | NK-cell2_563 | −0.1518661925042383 |
| chr14 | 105999664 | 105999664 | NK-cell2_1659 | −0.1497317348175066 |
| chr5 | 45515342 | 45515342 | NK-cell2_4277 | −0.1492637586518281 |
| chr1 | 237770077 | 237770077 | NK-cell2_460 | −0.1429089121816413 |
| chr20 | 43017606 | 43017606 | NK-cell2_3261 | −0.1403927736809689 |
| chr10 | 113019779 | 113019779 | NK-cell2_682 | −0.1328251545305659 |
| chr11 | 70132159 | 70132159 | NK-cell2_923 | −0.1191687747644632 |
| chrX | 38540615 | 38540615 | NK-cell2_5811 | −0.1173392820770955 |
| chr15 | 87425387 | 87425387 | NK-cell2_1789 | −0.1155364338846222 |
| chrY | 12294733 | 12294733 | NK-cell2_6170 | −0.1139266047061712 |
| chrX | 19888269 | 19888269 | NK-cell2_5735 | −0.1093593065183064 |
| chrX | 6946137 | 6946137 | NK-cell2_5692 | −0.1082658757867931 |
| chr14 | 54389827 | 54389827 | NK-cell2_1538 | −0.1002029802725852 |
| chr12 | 29707681 | 29707681 | NK-cell2_1098 | −0.0960707073324249 |
| chr6 | 167996781 | 167996781 | NK-cell2_4781 | −0.0958200837521404 |
| chr7 | 52293473 | 52293473 | NK-cell2_4885 | −0.0956458791676027 |
| chr6 | 78731573 | 78731573 | NK-cell2_4650 | −0.0931591885735846 |
| chr4 | 32201732 | 32201732 | NK-cell2_3939 | −0.0898567279340333 |
| chrX | 136747616 | 136747616 | NK-cell2_6092 | −0.0861485328784807 |
| chrX | 83508617 | 83508617 | NK-cell2_5939 | −0.0857529311423721 |
| chr6 | 119700394 | 119700394 | NK-cell2_4708 | −0.0847137591786124 |
| chr3 | 159158342 | 159158342 | NK-cell2_3810 | −0.0799977844894311 |
| chr3 | 78769778 | 78769778 | NK-cell2_3701 | −0.0797380025839241 |
| chrX | 142203192 | 142203192 | NK-cell2_6115 | −0.0792042839456554 |
| chr2 | 24723079 | 24723079 | NK-cell2_2747 | −0.0748670853953187 |
| chr5 | 179443423 | 179443423 | NK-cell2_4510 | −0.0745210785845121 |
| chr14 | 28967400 | 28967400 | NK-cell2_1505 | −0.07413597163141 |
| chr8 | 14086972 | 14086972 | NK-cell2_5156 | −0.0727750389037341 |
| chr10 | 43351440 | 43351440 | NK-cell2_568 | −0.0711104139178209 |
| chrX | 133675376 | 133675376 | NK-cell2_6085 | −0.0689668042478371 |
| chr17 | 73991443 | 73991443 | NK-cell2_2258 | −0.0679830343802398 |
| chrX | 133657154 | 133657154 | NK-cell2_6084 | −0.066303515695802 |
| chr12 | 97609115 | 97609115 | NK-cell2_1210 | −0.0638776379532023 |
| chrX | 22784564 | 22784564 | NK-cell2_5756 | −0.0620315053864313 |
| chr5 | 137064625 | 137064625 | NK-cell2_4394 | −0.0618945169949909 |
| chrX | 20372157 | 20372157 | NK-cell2_5739 | −0.0613291785761295 |
| chr14 | 35315306 | 35315306 | NK-cell2_1514 | −0.0610155868089912 |
| chrX | 151054622 | 151054622 | NK-cell2_6142 | −0.0608073411387222 |
| chr8 | 10524256 | 10524256 | NK-cell2_5136 | −0.0577202409189476 |
| chr2 | 94622552 | 94622552 | NK-cell2_2853 | 0.0566215236855747 |
| chr8 | 103605484 | 103605484 | NK-cell2_5350 | 0.0568335329981517 |
| chr9 | 136254409 | 136254409 | NK-cell2_5668 | 0.0568731459716901 |
| chr10 | 55847236 | 55847236 | NK-cell2_587 | 0.0576640622167844 |
| chr4 | 58485821 | 58485821 | NK-cell2_3976 | 0.0593272862018465 |
| chr5 | 1967399 | 1967399 | NK-cell2_4172 | 0.0594245261414524 |
| chr10 | 55019478 | 55019478 | NK-cell2_585 | 0.0598945077343069 |
| chr5 | 134039489 | 134039489 | NK-cell2_4386 | 0.060366578603064 |
| chrX | 154704782 | 154704782 | NK-cell2_6160 | 0.0611638371899969 |
| chrX | 86346394 | 86346394 | NK-cell2_5948 | 0.0613262853700475 |
| chr6 | 84300824 | 84300824 | NK-cell2_4659 | 0.0614133428351465 |
| chr19 | 58606240 | 58606240 | NK-cell2_2674 | 0.0648882500260437 |
| chr18 | 72531995 | 72531995 | NK-cell2_2406 | 0.0665289715319782 |
| chr10 | 81976704 | 81976704 | NK-cell2_624 | 0.0677662294910037 |
| chr3 | 36853089 | 36853089 | NK-cell2_3619 | 0.0682129857263097 |
| chr2 | 124594378 | 124594378 | NK-cell2_2928 | 0.0702896791404853 |
| chr12 | 121785489 | 121785489 | NK-cell2_1266 | 0.0721086413136361 |
| chr2 | 64607763 | 64607763 | NK-cell2_2807 | 0.075509288365919 |
| chr4 | 30237156 | 30237156 | NK-cell2_3936 | 0.0757162075893877 |
| chr1 | 68137904 | 68137904 | NK-cell2_216 | 0.0775834779044649 |
| chr4 | 24331093 | 24331093 | NK-cell2_3925 | 0.0780960478320569 |
| chr11 | 6147031 | 6147031 | NK-cell2_779 | 0.0817951706703846 |
| chr14 | 76922743 | 76922743 | NK-cell2_1572 | 0.083663949467977 |
| chr7 | 84401427 | 84401427 | NK-cell2_4945 | 0.0836832351560266 |
| chr20 | 63248303 | 63248303 | NK-cell2_3338 | 0.0865262107703451 |
| chrX | 138270880 | 138270880 | NK-cell2_6100 | 0.0879163833583131 |
| chr2 | 31957123 | 31957123 | NK-cell2_2761 | 0.0886093433285692 |
| chr2 | 20636906 | 20636906 | NK-cell2_2738 | 0.0907463289725737 |
| chr11 | 34425545 | 34425545 | NK-cell2_826 | 0.0921931918412999 |
| chr4 | 49200924 | 49200924 | NK-cell2_3965 | 0.0955184136202006 |
| chr21 | 14794679 | 14794679 | NK-cell2_3360 | 0.0966809859830283 |
| chr19 | 17288658 | 17288658 | NK-cell2_2533 | 0.0985860305121312 |
| chr1 | 47169515 | 47169515 | NK-cell2_177 | 0.1014337239737995 |
| chr17 | 66976605 | 66976605 | NK-cell2_2245 | 0.1037004538016864 |
| chr9 | 137866746 | 137866746 | NK-cell2_5679 | 0.1053942292381077 |
| chr1 | 125074980 | 125074980 | NK-cell2_295 | 0.1065159373270488 |
| chr20 | 30492969 | 30492969 | NK-cell2_3223 | 0.1073594983256663 |
| chr8 | 31912448 | 31912448 | NK-cell2_5197 | 0.1126751023941931 |
| chr7 | 118681133 | 118681133 | NK-cell2_5009 | 0.1151042229863229 |
| chrX | 46311269 | 46311269 | NK-cell2_5840 | 0.1191220225294089 |
| chr21 | 38941420 | 38941420 | NK-cell2_3403 | 0.1303196761102698 |
| chrX | 20930595 | 20930595 | NK-cell2_5743 | 0.1326526581932279 |
| chr1 | 28302295 | 28302295 | NK-cell2_126 | 0.1366300104534654 |
| chr7 | 62664298 | 62664298 | NK-cell2_4910 | 0.1367190103431417 |
| chrX | 21675798 | 21675798 | NK-cell2_5748 | 0.1433564352611733 |
| chr4 | 131698776 | 131698776 | NK-cell2_4062 | 0.1477667168144044 |
| chrX | 151048743 | 151048743 | NK-cell2_6141 | 0.1485436370574815 |
| chr7 | 4792224 | 4792224 | NK-ccll2_4815 | 0.1489249526963931 |
| chr2 | 187319190 | 187319190 | NK-cell2_3024 | 0.168332217550018 |
| chr17 | 22842913 | 22842913 | NK-cell2_2128 | 0.173547627405367 |
| chr8 | 28442917 | 28442917 | NK-cell2_5191 | 0.1761808006350915 |
| chr12 | 991978 | 991978 | NK-cell2_1059 | 0.1870117541885508 |
| chr21 | 5100354 | 5100354 | NK-cell2_3348 | 0.2429142107309043 |
| chr1 | 97919432 | 97919432 | NK-cell2_250 | 0.2574604332458793 |

Flu Tc-Mem

| | | | | |
|---|---|---|---|---|
| chr7 | 58948549 | 58948549 | Tc-Mem_16495 | 0.2367355243832106 |
| chr4 | 139114549 | 139114549 | Tc-Mem_13640 | −0.227487340009986 |
| chr19 | 46039367 | 46039367 | Tc-Mem_8733 | −0.1993480066481103 |
| chr9 | 42723367 | 42723367 | Tc-Mem_18468 | −0.1769249555497635 |
| chr1 | 34568245 | 34568245 | Tc-Mem_381 | −0.1738184057529891 |
| chr9 | 11555051 | 11555051 | Tc-Mem_18286 | −0.164617845966974 |
| chr4 | 40834775 | 40834775 | Tc-Mem_13154 | −0.1609995254863595 |
| chr17 | 7406639 | 7406639 | Tc-Mem_7150 | −0.1569303831696496 |
| chr7 | 42796351 | 42796351 | Tc-Mem_16368 | −0.142746138627062 |
| chr14 | 102774463 | 102774463 | Tc-Mem_5658 | −0.1427012351766199 |
| chr19 | 23468147 | 23468147 | Tc-Mem_8527 | −0.1400016731025556 |
| chr8 | 75900796 | 75900796 | Tc-Mem_17734 | −0.1131985584591402 |
| chrX | 89897357 | 89897357 | Tc-Mem_19663 | −0.1125746636446964 |
| chr11 | 64172943 | 64172943 | Tc-Mem_3078 | −0.1120208020496708 |
| chr6 | 49201542 | 49201542 | Tc-Mem_15422 | −0.1112188640875631 |
| chr16 | 88172184 | 88172184 | Tc-Mem_7028 | −0.1078619906393579 |
| chr9 | 41284256 | 41284256 | Tc-Mem_18461 | −0.1013137761475483 |
| chr10 | 41562520 | 41562520 | Tc-Mem_1860 | −0.0979084235110108 |
| chr10 | 82409452 | 82409452 | Tc-Mem_2148 | −0.0938481686190068 |
| chr10 | 42301341 | 42301341 | Tc-Mem_1864 | −0.0925677597660408 |
| chr2 | 66010818 | 66010818 | Tc-Mem_9263 | −0.0908642420239594 |
| chr1 | 121845528 | 121845528 | Tc-Mem_913 | −0.0899079909815911 |
| chr1 | 111645991 | 111645991 | Tc-Mem_853 | −0.0888019609283161 |
| chr22 | 37404257 | 37404257 | Tc-Mem_11382 | −0.0844093110617368 |
| chr9 | 105870968 | 105870968 | Tc-Mem_18748 | −0.0840698619022286 |
| chr13 | 66708284 | 66708284 | Tc-Mem_4800 | −0.0809501982432144 |
| chr5 | 29698049 | 29698049 | Tc-Mem_14203 | −0.0802903310838887 |
| chr22 | 50806781 | 50806781 | Tc-Mem_11589 | −0.0788733518249771 |
| chr14 | 55211048 | 55211048 | Tc-Mem_5313 | −0.0770205224577158 |
| chr5 | 177725088 | 177725088 | Tc-Mem_15058 | −0.0769668456595294 |
| chr9 | 110395945 | 110395945 | Tc-Mem_18800 | −0.0765101088671252 |
| chr6 | 82392988 | 82392988 | Tc-Mem_15569 | −0.0761188283894026 |
| chr17 | 78543511 | 78543511 | Tc-Mem_7685 | −0.0760335177485797 |
| chr5 | 176859033 | 176859033 | Tc-Mem_15052 | −0.0747574920987386 |
| chr6 | 103113129 | 103113129 | Tc-Mem_15660 | −0.0734506447400472 |
| chr13 | 58357560 | 58357560 | Tc-Mem_4751 | −0.071810219349306 |
| chr21 | 26710659 | 26710659 | Tc-Mem_11021 | −0.0684506541454723 |
| chr5 | 176223890 | 176223890 | Tc-Mem_15045 | −0.0673318550204606 |
| chr16 | 19796799 | 19796799 | Tc-Mem_6522 | −0.0672100234069186 |
| chr7 | 91657199 | 91657199 | Tc-Mem_16680 | −0.0661451898129607 |
| chr10 | 38864811 | 38864811 | Tc-Mem_1851 | −0.0660240583713286 |
| chr9 | 115706970 | 115706970 | Tc-Mem_18825 | −0.0645880777649322 |
| chr6 | 32936871 | 32936871 | Tc-Mem_15321 | −0.0640328995269993 |
| chrX | 127608591 | 127608591 | Tc-Mem_19890 | −0.0640023334063934 |
| chr3 | 80894483 | 80894483 | Tc-Mem_12134 | −0.061534501496075 |
| chrX | 39610365 | 39610365 | Tc-Mem_19367 | −0.0606707137712422 |
| chr16 | 6521515 | 6521515 | Tc-Mem_6428 | −0.0574755475696729 |
| chr2 | 221708747 | 221708747 | Tc-Mem_10155 | 0.0576068431814428 |
| chr5 | 85877440 | 85877440 | Tc-Mem_14497 | 0.0578851196146755 |
| chr1 | 215803691 | 215803691 | Tc-Mem_1308 | 0.0591029716272297 |
| chrY | 19031640 | 19031640 | Tc-Mem_20142 | 0.0619430803625766 |
| chrY | 14468129 | 14468129 | Tc-Mem_20131 | 0.0620241860404521 |
| chr17 | 45922465 | 45922465 | Tc-Mem_7443 | 0.0624548063219825 |
| chrY | 4858180 | 4858180 | Tc-Mem_20102 | 0.0673602115707704 |
| chr22 | 12804270 | 12804270 | Tc-Mem_11183 | 0.0691271610891783 |
| chr1 | 59535689 | 59535689 | Tc-Mem_586 | 0.0691844267733482 |
| chr17 | 21928476 | 21928476 | Tc-Mem_7271 | 0.0727942930124053 |
| chr2 | 14026336 | 14026336 | Tc-Mem_8983 | 0.0738218521242935 |
| chr13 | 36307198 | 36307198 | Tc-Mem_4632 | 0.0745098833511771 |
| chrX | 10893896 | 10893896 | Tc-Mem_19165 | 0.0754000589352927 |
| chrY | 19119240 | 19119240 | Tc-Mem_20145 | 0.0755759812871281 |
| chr4 | 45075835 | 45075835 | Tc-Mem_13187 | 0.0763836139540005 |
| chr3 | 1649453 | 1649453 | Tc-Mem_11598 | 0.0774853649117378 |
| chr17 | 48728934 | 48728934 | Tc-Mem_7456 | 0.0783796411162747 |
| chr4 | 59423405 | 59423405 | Tc-Mem_13251 | 0.0808811418573452 |
| chr1 | 73013540 | 73013540 | Tc-Mem_660 | 0.081873547852984 |
| chr14 | 22270507 | 22270507 | Tc-Mem_5146 | 0.0840995043813961 |
| chr16 | 86932756 | 86932756 | Tc-Mem_7009 | 0.0850027616773286 |
| chr1 | 16775366 | 16775366 | Tc-Mem_216 | 0.0862602486020564 |
| chr15 | 78570503 | 78570503 | Tc-Mem_6126 | 0.0874067985282387 |
| chr4 | 19718511 | 19718511 | Tc-Mem_13023 | 0.0892401360714972 |
| chrX | 142376003 | 142376003 | Tc-Mem_20006 | 0.0901417678240241 |
| chr11 | 104996194 | 104996194 | Tc-Mem_3376 | 0.0924621537539657 |
| chrY | 3437151 | 3437151 | Tc-Mem_20100 | 0.0925612060230788 |
| chrY | 20158065 | 20158065 | Tc-Mem_20148 | 0.0936213566407104 |
| chr5 | 92272065 | 92272065 | Tc-Mem_14524 | 0.0937841690307318 |
| chr13 | 18457276 | 18457276 | Tc-Mem_4509 | 0.0969923162021279 |
| chr14 | 101712043 | 101712043 | Tc-Mem_5651 | 0.0995186031709984 |
| chr2 | 93818941 | 93818941 | Tc-Mem_9427 | 0.0998878793192743 |
| chrY | 6957467 | 6957467 | Tc-Mem_20107 | 0.1043070572129466 |
| chrX | 153056900 | 153056900 | Tc-Mem_20064 | 0.1060244217382462 |
| chr12 | 127706243 | 127706243 | Tc-Mem_4429 | 0.1073500266814034 |
| chrY | 3756315 | 3756315 | Tc-Mem_20101 | 0.1091082310517983 |
| chr16 | 28747368 | 28747368 | Tc-Mem_6582 | 0.1133884555315926 |
| chr19 | 735666 | 735666 | Tc-Mem_8261 | 0.1183934721652478 |
| chr4 | 157179295 | 157179295 | Tc-Mem_13732 | 0.1195081003745335 |
| chrY | 6998849 | 6998849 | Tc-Mem_20109 | 0.1294777917199961 |
| chr6 | 164447154 | 164447154 | Tc-Mem_15995 | 0.1313583547651602 |
| chrY | 3298779 | 3298779 | Tc-Mem_20098 | 0.1344346765936206 |
| chr6 | 167384709 | 167384709 | Tc-Mem_16028 | 0.1366778850354721 |
| chr3 | 136064324 | 136064324 | Tc-Mem_12460 | 0.1422559634099384 |
| chr17 | 23465780 | 23465780 | Tc-Mem_7281 | 0.1452147601262264 |
| chr16 | 33951788 | 33951788 | Tc-Mem_6612 | 0.1916500463685277 |
| chrY | 6965470 | 6965470 | Tc-Mem_20108 | 0.1940405226539986 |
| chr15 | 23225907 | 23225907 | Tc-Mem_5732 | 0.2275624859590214 |
| chr2 | 97603068 | 97603068 | Tc-Mem_9453 | 0.2547107630470558 |
| chrY | 5019521 | 5019521 | Tc-Mem_20104 | 0.276018553425396 |
| chr9 | 63041520 | 63041520 | Tc-Mem_18480 | 0.2859686396236415 |
| chrY | 11658142 | 11658142 | Tc-Mem_20117 | 0.2874129040081921 |
| chrY | 18667307 | 18667307 | Tc-Mem_20141 | 0.3441794441407285 |

Flu Tc-Naïve

| | | | | |
|---|---|---|---|---|
| chr11 | 86289393 | 86289393 | Tc-Naive_773 | −0.4901347693024532 |
| chr7 | 155206087 | 155206087 | Tc-Naive_4262 | −0.4400561462357914 |
| chr18 | 71953145 | 71953145 | Tc-Naive_2008 | −0.3184577612519166 |
| chr8 | 72926191 | 72926191 | Tc-Naive_4405 | −0.2984369766064245 |
| chr14 | 78013428 | 78013428 | Tc-Naive_1301 | −0.2820859069711893 |
| chr18 | 79617424 | 79617441 | Tc-Naive_2031 | −0.2797814144222276 |
| chr12 | 53097324 | 53097324 | Tc-Naive_955 | −0.2452512099630299 |
| chr4 | 188201293 | 188201293 | Tc-Naive_3499 | −0.2357960307026955 |
| chrX | 137429373 | 137429373 | Tc-Naive_5103 | −0.2352869854824991 |
| chr13 | 114199562 | 114199562 | Tc-Naive_1239 | −0.2249155486354044 |
| chr12 | 8307183 | 8307183 | Tc-Naive_891 | −0.2118510821763351 |
| chr9 | 102080797 | 102080797 | Tc-Naive_4631 | −0.1993491644328035 |
| chr19 | 17979497 | 17979497 | Tc-Naive_2104 | −0.193375014123016 |
| chrX | 131997995 | 131997995 | Tc-Naive_5083 | −0.1924438386919313 |
| chr8 | 141560670 | 141560670 | Tc-Naive_4492 | −0.188364995444199 |
| chr8 | 143201569 | 143201569 | Tc-Naive_4506 | −0.1856636126171401 |
| chr22 | 17075996 | 17075996 | Tc-Naive_2869 | −0.1842794017791898 |
| chr4 | 189446708 | 189446708 | Tc-Naive_3501 | −0.1730306197015617 |
| chr1 | 179596389 | 179596389 | Tc-Naive_269 | −0.1647929975276832 |
| chr19 | 42913694 | 42913694 | Tc-Naive_2156 | −0.1619016271167866 |
| chr11 | 128454003 | 128454003 | Tc-Naive_845 | −0.1503498542486771 |
| chr10 | 116721608 | 116721608 | Tc-Naive_554 | −0.1488603114987918 |
| chr19 | 1796430 | 1796430 | Tc-Naive_2047 | −0.1454194181177803 |
| chr13 | 56892215 | 56892215 | Tc-Naive_1162 | −0.1414750835820553 |
| chr16 | 18562559 | 18562559 | Tc-Naive_1572 | −0.1382024985801391 |
| chr1 | 17441389 | 17441389 | Tc-Naive_64 | −0.1258459747798568 |
| chr8 | 43536274 | 43536274 | Tc-Naive_4366 | −0.124470724481796 |
| chr5 | 91797372 | 91797372 | Tc-Naive_3626 | −0.1210834311401031 |
| chr9 | 4300253 | 4300257 | Tc-Naive_4530 | −0.1180837741665341 |
| chr16 | 31439188 | 31439188 | Tc-Naive_1596 | −0.1171134042420151 |
| chr10 | 133088962 | 133088962 | Tc-Naive_608 | −0.1120267545682661 |
| chr17 | 32374191 | 32374191 | Tc-Naive_1786 | −0.1094187831410223 |
| chr1 | 229008638 | 229008638 | Tc-Naive_338 | −0.1055667350648446 |
| chr12 | 102311297 | 102311297 | Tc-Naive_1007 | −0.0980745704810139 |
| chr5 | 15840783 | 15840783 | Tc-Naive_3542 | −0.0855963494591513 |
| chr1 | 199857473 | 199857473 | Tc-Naive_299 | −0.0766511808413144 |
| chr4 | 117516053 | 117516053 | Tc-Naive_3422 | −0.0705767070663073 |
| chr20 | 30897167 | 30897167 | Tc-Naive_2657 | −0.070279299373607 |
| chr16 | 34673485 | 34673485 | Tc-Naive_1605 | −0.0699145369442781 |
| chr13 | 35886770 | 35886770 | Tc-Naive_1128 | −0.0582489792014185 |
| chr20 | 18281062 | 18281062 | Tc-Naive_2621 | −0.0574528506498051 |
| chr14 | 65076014 | 65076014 | Tc-Naive_1284 | −0.0547762783831172 |
| chr7 | 132224092 | 132224092 | Tc-Naive_4223 | −0.0522852965748088 |
| chr8 | 39439701 | 39439701 | Tc-Naive_4356 | −0.0483721205194791 |
| chr5 | 148375080 | 148375080 | Tc-Naive_3705 | −0.0443318235322225 |
| chr16 | 36170998 | 36170998 | Tc-Naive_1612 | −0.0430179193349098 |
| chr6 | 123330423 | 123330423 | Tc-Naive_3970 | −0.0429736335606247 |
| chr20 | 28512382 | 28512382 | Tc-Naive_2644 | −0.0416724053145204 |
| chr1 | 24266678 | 24266678 | Tc-Naive_84 | −0.0400990196674143 |
| chr6 | 118796949 | 118796949 | Tc-Naive_3964 | −0.0361396444178378 |
| chr20 | 48618709 | 48618709 | Tc-Naive_2707 | −0.0332342830317794 |
| chr9 | 68781235 | 68781235 | Tc-Naive_4587 | −0.0307753399128255 |
| chr12 | 150808 | 150808 | Tc-Naive_867 | −0.0305688068923503 |
| chr14 | 75156908 | 75156908 | Tc-Naive_1295 | −0.0273408185966329 |
| chrX | 37378667 | 37378667 | Tc-Naive_4838 | −0.0272862240597005 |
| chr4 | 123149457 | 123149457 | Tc-Naive_3426 | 0.0227257025652261 |
| chr20 | 29292895 | 29292895 | Tc-Naive_2648 | 0.0268711622921113 |
| chr11 | 49050012 | 49050012 | Tc-Naive_695 | 0.0272182032104147 |
| chr1 | 37393978 | 37393978 | Tc-Naive_129 | 0.0294158051336307 |
| chr13 | 38438545 | 38438545 | Tc-Naive_1133 | 0.0320921269372558 |
| chr4 | 16266640 | 16266640 | Tc-Naive_3308 | 0.0367481919324493 |
| chr12 | 94882146 | 94882146 | Tc-Naive_999 | 0.0415433587401535 |
| chrX | 105297580 | 105297580 | Tc-Naive_5002 | 0.0465009850995324 |
| chr12 | 109385399 | 109385399 | Tc-Naive_1020 | 0.0470670522812137 |
| chr9 | 137977896 | 137977896 | Tc-Naive_4728 | 0.0471514215461736 |
| chr9 | 41235362 | 41235362 | Tc-Naive_4578 | 0.0503529967049391 |
| chr19 | 55068814 | 55068814 | Tc-Naive_2212 | 0.051436003454727 |
| chr1 | 223965984 | 223965984 | Tc-Naive_333 | 0.051978627407881 |
| chrX | 149720778 | 149720778 | Tc-Naive_5150 | 0.0546762479267406 |
| chr21 | 30111875 | 30111875 | Tc-Naive_2808 | 0.0575872505521957 |
| chrX | 36617524 | 36617524 | Tc-Naive_4834 | 0.066347310153476 |
| chr5 | 180135009 | 180135009 | Tc-Naive_3775 | 0.0702576781568548 |
| chr6 | 131494208 | 131494208 | Tc-Naive_3975 | 0.076186862308138 |
| chr10 | 7413268 | 7413268 | Tc-Naive_390 | 0.0803484011703335 |
| chr22 | 49357798 | 49357798 | Tc-Naive_2969 | 0.0835113670387675 |
| chr1 | 14500329 | 14500329 | Tc-Naive_49 | 0.0857125579973193 |
| chr21 | 22219738 | 22219738 | Tc-Naive_2800 | 0.0882581809665912 |
| chr19 | 45645022 | 45645022 | Tc-Naive_2165 | 0.0887759948619453 |
| chrX | 74731657 | 74731657 | Tc-Naive_4928 | 0.1163244428951893 |
| chr4 | 120321064 | 120321064 | Tc-Naive_3423 | 0.117311421665498 |
| chr6 | 70123868 | 70123868 | Tc-Naive_3903 | 0.1237579329941515 |
| chr7 | 122241953 | 122241953 | Tc-Naive_4203 | 0.1473344986925175 |
| chr19 | 39893939 | 39893939 | Tc-Naive_2146 | 0.1605658789327902 |
| chr16 | 60472624 | 60472624 | Tc-Naive_1647 | 0.1676950162034567 |
| chr8 | 4563161 | 4563161 | Tc-Naive_4294 | 0.2023590291961452 |
| chr5 | 21956977 | 21956977 | Tc-Naive_3550 | 0.2137655174927978 |
| chr1 | 23218549 | 23218549 | Tc-Naive_81 | 0.2211410220432426 |
| chr7 | 38316553 | 38316553 | Tc-Naive_4091 | 0.226039723110371 |
| chr3 | 145808128 | 145808128 | Tc-Naive_3163 | 0.2294734305977162 |
| chr2 | 139104123 | 139104123 | Tc-Naive_2436 | 0.2318988331628866 |
| chr2 | 94890833 | 94890833 | Tc-Naive_2358 | 0.2396380023815452 |
| chr11 | 22319879 | 22319879 | Tc-Naive_659 | 0.2459338890136868 |
| chr8 | 12660009 | 12660009 | Tc-Naive_4315 | 0.2463069827765982 |
| chrY | 11644176 | 11644176 | Tc-Naive_5174 | 0.261827961621497 |
| chr11 | 76914381 | 76914381 | Tc-Naive_756 | 0.2884965835020807 |
| chr4 | 3944142 | 3944142 | Tc-Naive_3271 | 0.3320075046242268 |
| chr6 | 19837281 | 19837281 | Tc-Naive_3812 | 0.3638762312661748 |
| chr5 | 21108899 | 21108899 | Tc-Naive_3549 | 0.5059279926314891 |
| chr10 | 93971753 | 93971753 | Tc-Naive_514 | 0.5684610308571978 |
| chr7 | 155570893 | 155570893 | Tc-Naive_4265 | 0.714434464554967 |

Flu Th-Mem

| | | | | |
|---|---|---|---|---|
| chr12 | 111764833 | 111764833 | Th-Mem_2818 | −0.2513752845670841 |
| chr4 | 178019584 | 178019584 | Th-Mem_9135 | −0.2374412341230944 |
| chr8 | 72714923 | 72714923 | Th-Mem_11701 | −0.2229572219128723 |
| chr7 | 55669946 | 55669946 | Th-Mem_10910 | −0.1719040916825812 |
| chr16 | 60792504 | 60792504 | Th-Mem_4438 | −0.170414892513713 |
| chrX | 114545482 | 114545482 | Th-Mem_13040 | −0.1675965845113007 |
| chr2 | 234826195 | 234826195 | Th-Mem_6834 | −0.1533332460363269 |
| chr21 | 22927469 | 22927469 | Th-Mem_7351 | −0.1449013204946041 |
| chrX | 85052251 | 85052251 | Th-Mem_12926 | −0.1438538128307954 |
| chr3 | 158029447 | 158029447 | Th-Mem_8333 | −0.1373060594843178 |
| chr7 | 27293258 | 27293258 | Th-Mem_10789 | −0.1359692435760533 |
| chr22 | 11874562 | 11874562 | Th-Mem_7487 | −0.1132105393467895 |
| chr17 | 13368726 | 13368726 | Th-Mem_4710 | 0.1115721537717929 |
| chr10 | 16869569 | 16869569 | Th-Mem_1142 | −0.1093576357574436 |
| chr1 | 74351462 | 74351462 | Th-Mem_453 | −0.1082821638875697 |
| chr20 | 29901346 | 29901346 | Th-Mem_7041 | −0.1039319628740746 |
| chr5 | 98080988 | 98080988 | Th-Mem_9547 | −0.1032916676105155 |
| chr2 | 7750654 | 7750654 | Th-Mem_5867 | −0.1015661709682163 |
| chr6 | 59345575 | 59345575 | Th-Mem_10194 | −0.1010673203727521 |
| chr2 | 4714355 | 4714355 | Th-Mem_5850 | −0.0999487626119412 |
| chr2 | 57380777 | 57380777 | Th-Mem_6106 | −0.0979986272171405 |
| chr6 | 64618069 | 64618069 | Th-Mem_10207 | −0.0971084289658771 |
| chr13 | 82997196 | 82997196 | Th-Mem_3225 | −0.096614482172376 |
| chr6 | 81352185 | 81352185 | Th-Mem_10273 | −0.0909536478366558 |
| chr5 | 174429078 | 174429078 | Th-Mem_9877 | −0.0897782069339239 |
| chr5 | 75896213 | 75896213 | Th-Mem_9480 | −0.0881987364975977 |
| chr5 | 160652477 | 160652477 | Th-Mem_9799 | −0.0879666664821709 |
| chr2 | 74588552 | 74588552 | Th-Mem_6180 | −0.0865653766208417 |
| chr12 | 3467218 | 3467218 | Th-Mem_2389 | −0.0857077041344052 |
| chr5 | 161609267 | 161609267 | Th-Mem_9803 | −0.0845251789224728 |
| chr13 | 64958271 | 64958271 | Th-Mem_3156 | −0.0776498601022846 |
| chr1 | 146544778 | 146544778 | Th-Mem_628 | −0.0740456932980339 |
| chrX | 57897563 | 57897563 | Th-Mem_12807 | −0.0732736980873118 |
| chrX | 5799675 | 5799675 | Th-Mem_12535 | −0.0727936513806046 |
| chr17 | 714023 | 714023 | Th-Mem_4618 | −0.0713810445648925 |
| chr2 | 33962549 | 33962549 | Th-Mem_6014 | −0.0709337305194277 |
| chrY | 21131099 | 21131099 | Th-Mem_13340 | −0.0703954273299855 |
| chr3 | 82498613 | 82498613 | Th-Mem_8109 | −0.0702835286981396 |
| chr10 | 87478300 | 87478300 | Th-Mem_1428 | −0.0700082949556907 |
| chrX | 116620748 | 116620748 | Th-Mem_13046 | −0.0687348972311209 |
| chr6 | 11380727 | 11380727 | Th-Mem_9992 | −0.0679951942759787 |
| chr6 | 35560297 | 35560297 | Th-Mem_10103 | −0.0675266861986872 |
| chrX | 142416849 | 142416849 | Th-Mem_13220 | −0.0670792088391815 |
| chr18 | 16545031 | 16545031 | Th-Mem_5166 | −0.064339039908275 |
| chr4 | 125944411 | 125944411 | Th-Mem_8964 | −0.0613160130931954 |
| chr12 | 59814195 | 59814195 | Th-Mem_2615 | −0.0578373271845346 |
| chr10 | 54486018 | 54486018 | Th-Mem_1293 | −0.0560279850531596 |
| chr1 | 94323892 | 94323892 | Th-Mem_525 | −0.0544486534263984 |
| chr3 | 61440178 | 61440178 | Th-Mem_8000 | 0.0529860607347435 |
| chr18 | 1749162 | 1749162 | Th-Mem_5105 | 0.0536614959104644 |
| chrX | 131772807 | 131772807 | Th-Mem_13137 | 0.0545725266700924 |
| chr3 | 28489668 | 28489668 | Th-Mem_7848 | 0.0547925266639605 |
| chr13 | 106852529 | 106852529 | Th-Mem_3323 | 0.055243681339992 |
| chrX | 6883477 | 6883477 | Th-Mem_12542 | 0.0555200079017541 |
| chrX | 86365702 | 86365702 | Th-Mem_12935 | 0.0571175321501879 |
| chr1 | 210779509 | 210779509 | Th-Mem_874 | 0.0572633803729136 |
| chr13 | 58828067 | 58828067 | Th-Mem_3146 | 0.0589217565611716 |
| chr2 | 210695033 | 210695033 | Th-Mem_6706 | 0.0593514697910268 |
| chr13 | 20356305 | 20356305 | Th-Mem_2951 | 0.0611248922209427 |
| chr16 | 59477214 | 59477214 | Th-Mem_4425 | 0.0622718013695167 |
| chr7 | 156113366 | 156113366 | Th-Mem_11307 | 0.0633276405763572 |
| chr3 | 64307473 | 64307473 | Th-Mem_8014 | 0.0654915167197199 |
| chr10 | 69483277 | 69483277 | Th-Mem_1339 | 0.0668579917986636 |
| chr21 | 44139892 | 44139907 | Th-Mem_7461 | 0.0670815526869821 |
| chr12 | 132895610 | 132895610 | Th-Mem_2937 | 0.0685995909173359 |
| chr2 | 240914723 | 240914723 | Th-Mem_6895 | 0.0714613977968399 |
| chr2 | 149748546 | 149748546 | Th-Mem_6497 | 0.0744953933944575 |
| chr14 | 42705974 | 42705974 | Th-Mem_3475 | 0.0747822626619906 |
| chr4 | 32462770 | 32462770 | Th-Mem_8684 | 0.0763794549414915 |
| chr9 | 120220849 | 120220849 | Th-Mem_12372 | 0.0778356053028297 |
| chr1 | 101106331 | 101106331 | Th-Mem_546 | 0.0789306589259203 |
| chr13 | 25530481 | 25530481 | Th-Mem_2989 | 0.0817793947801472 |
| chr11 | 45562189 | 45562189 | Th-Mem_1903 | 0.0859334091774853 |
| chr10 | 29170857 | 29170857 | Th-Mem_1187 | 0.09009681090515 |
| chr4 | 159717779 | 159717779 | Th-Mem_9070 | 0.0956139729799465 |
| chr10 | 63714194 | 63714194 | Th-Mem_1316 | 0.0959018154845995 |
| chrX | 8034151 | 8034151 | Th-Mem_12547 | 0.0970378979657179 |
| chr15 | 47310860 | 47310860 | Th-Mem_3904 | 0.0998725427303526 |
| chr8 | 68383259 | 68383259 | Th-Mem_11668 | 0.1018410816974217 |
| chrX | 37427331 | 37427331 | Th-Mem_12709 | 0.1029227957652453 |
| chr2 | 163035138 | 163035138 | Th-Mem_6540 | 0.1045003962840999 |
| chr3 | 109374270 | 109374270 | Th-Mem_8175 | 0.104791068735333 |
| chr11 | 52068688 | 52068688 | Th-Mem_1922 | 0.1048758292960239 |
| chr10 | 53824322 | 53824322 | Th-Mem_1291 | 0.1085483637003592 |
| chrX | 75029960 | 75029960 | Th-Mem_12878 | 0.1112988160618751 |
| chr7 | 145165785 | 145165785 | Th-Mem_11239 | 0.1211300577441658 |
| chr17 | 22437526 | 22437526 | Th-Mem_4762 | 0.1224422725797848 |
| chr17 | 42874372 | 42874372 | Th-Mem_4842 | 0.1262725497995576 |
| chrY | 16015779 | 16015779 | Th-Mem_13335 | 0.1270864528877182 |
| chr2 | 13033318 | 13033318 | Th-Mem_5894 | 0.1280469831706297 |
| chrX | 99230460 | 99230460 | Th-Mem_12968 | 0.1353311917103063 |
| chr2 | 157224907 | 157224907 | Th-Mem_6524 | 0.1382087784053448 |
| chr1 | 231240458 | 231240458 | Th-Mem_959 | 0.1402123763602086 |
| chr19 | 13974225 | 13974225 | Th-Mem_5560 | 0.1416090334288025 |
| chr16 | 1808069 | 1808069 | Th-Mem_4166 | 0.1433300908117127 |
| chr1 | 99513712 | 99513712 | Th-Mem_541 | 0.1451672313258552 |
| chrX | 148919276 | 148919276 | Th-Mem_13252 | 0.1488060702113103 |
| chr22 | 26079551 | 26079551 | Th-Mem_7546 | 0.1552692541138552 |
| chr8 | 88162410 | 88162410 | Th-Mem_11761 | 0.1587224645676351 |
| chr4 | 182161341 | 182161341 | Th-Mem_9157 | 0.2299782186632201 |

Flu Th-Naïve

| | | | | |
|---|---|---|---|---|
| chr9 | 1325772 | 1325772 | Th-Naive_4657 | −0.5690417340678428 |
| chr20 | 23099838 | 23099838 | Th-Naive_2729 | −0.5062499010658835 |
| chr9 | 67473119 | 67473119 | Th-Naive_4723 | −0.5039125292581652 |
| chr10 | 28501544 | 28501544 | Th-Naive_460 | −0.4768443303385832 |
| chr15 | 20424705 | 20424705 | Th-Naive_1423 | 0.4211829046921399 |
| chr10 | 46754130 | 46754130 | Th-Naive_488 | −0.3814412043059846 |
| chr4 | 174215456 | 174215456 | Th-Naive_3549 | −0.3727243219305354 |
| chr22 | 16137187 | 16137187 | Th-Naive_2969 | −0.3595341342424157 |
| chr16 | 9848543 | 9848543 | Th-Naive_1615 | −0.3331443562923221 |
| chr20 | 29408216 | 29408216 | Th-Naive_2736 | −0.2606575943336548 |
| chr14 | 69822807 | 69822807 | Th-Naive_1325 | −0.2590046650118299 |
| chrX | 58059057 | 58059057 | Th-Naive_5051 | −0.2565829600929072 |
| chr17 | 47295776 | 47295776 | Th-Naive_1924 | −0.1985224792926847 |
| chr8 | 92018494 | 92018494 | Th-Naive_4565 | −0.1966846161691532 |
| chr10 | 12062772 | 12062772 | Th-Naive_435 | −0.1707434174845643 |
| chr5 | 114599365 | 114599365 | Th-Naive_3745 | −0.1662069960796086 |
| chr10 | 117721299 | 117721299 | Th-Naive_583 | −0.1645159242675255 |
| chr2 | 147818580 | 147818580 | Th-Naive_2560 | −0.1626020582436199 |
| chr5 | 84981879 | 84981879 | Th-Naive_3703 | −0.1542376611335865 |
| chr9 | 131478575 | 131478575 | Th-Naive_4835 | −0.1529301914917754 |
| chr18 | 13506327 | 13506327 | Th-Naive_2037 | −0.1375982094099559 |
| chrX | 11599402 | 11599402 | Th-Naive_4904 | −0.1312516499151596 |
| chr13 | 112980998 | 112980998 | Th-Naive_1247 | −0.1299778679552738 |
| chr3 | 163455408 | 163455408 | Th-Naive_3281 | −0.1267598027624345 |
| chr10 | 88670725 | 88670725 | Th-Naive_538 | −0.1266452353397369 |
| chr17 | 72416635 | 72416635 | Th-Naive_1972 | −0.1251630043201883 |
| chr17 | 70386162 | 70386162 | Th-Naive_1966 | −0.1244832024217176 |
| chr5 | 146612671 | 146612671 | Th-Naive_3806 | −0.106874532487329 |
| chr7 | 135761150 | 135761150 | Th-Naive_4356 | −0.0942705203139295 |
| chr21 | 44254383 | 44254383 | Th-Naive_2945 | −0.0863714216372074 |
| chr22 | 37912284 | 37912284 | Th-Naive_3032 | −0.0796766818478301 |
| chr7 | 27840497 | 27840497 | Th-Naive_4182 | −0.0790619417626649 |
| chr3 | 9863010 | 9863010 | Th-Naive_3094 | −0.0604932131394441 |
| chr5 | 137958137 | 137958137 | Th-Naive_3778 | −0.0565706532270981 |
| chrX | 127135567 | 127135567 | Th-Naive_5242 | −0.0548892343106876 |
| chr16 | 3035222 | 3035222 | Th-Naive_1586 | −0.0544884599650964 |
| chr19 | 41089445 | 41089445 | Th-Naive_2270 | −0.0519715566109088 |
| chr20 | 21921511 | 21921511 | Th-Naive_2724 | −0.042478108198139 |
| chr7 | 56174283 | 56174283 | Th-Naive_4229 | −0.0389598718578591 |
| chr8 | 42177945 | 42177949 | Th-Naive_4490 | −0.03165638404067 |
| chr9 | 104218801 | 104218801 | Th-Naive_4780 | −0.0311722807586471 |
| chr9 | 120018258 | 120018258 | Th-Naive_4801 | −0.0297078749815247 |
| chr6 | 161993594 | 161993594 | Th-Naive_4114 | 0.0291952394633599 |
| chr13 | 25420327 | 25420327 | Th-Naive_1138 | 0.0331757090387295 |
| chr11 | 73957717 | 73957717 | Th-Naive_776 | 0.0344543629941608 |
| chr1 | 125087819 | 125087819 | Th-Naive_236 | 0.0431388264085277 |
| chr7 | 154140873 | 154140873 | Th-Naive_4394 | 0.0434815591795242 |
| chr10 | 85528100 | 85528100 | Th-Naive_534 | 0.0451931857928399 |
| chr12 | 126783067 | 126783067 | Th-Naive_1098 | 0.0456371420437609 |
| chrX | 146595143 | 146595143 | Th-Naive_5296 | 0.0466237692200405 |
| chr9 | 102068743 | 102068743 | Th-Naive_4778 | 0.0486417800738727 |
| chr2 | 1551095 | 1551095 | Th-Naive_2352 | 0.0505886760975672 |
| chr10 | 80946015 | 80946015 | Th-Naive_522 | 0.0521270510879328 |
| chr17 | 58228839 | 58228839 | Th-Naive_1949 | 0.053772491985875 |
| chr7 | 45941524 | 45941524 | Th-Naive_4209 | 0.054680939074431 |
| chr11 | 55564790 | 55564790 | Th-Naive_725 | 0.0548872679976458 |
| chrX | 112562410 | 112562410 | Th-Naive_5203 | 0.0584833749192277 |
| chr5 | 124305445 | 124305445 | Th-Naive_3758 | 0.0585845059546181 |
| chr13 | 112035052 | 112035052 | Th-Naive_1243 | 0.0588619782239664 |
| chr13 | 83654312 | 83654312 | Th-Naive_1204 | 0.0607933036119311 |
| chrX | 107913908 | 107913908 | Th-Naive_5189 | 0.0609386187458802 |
| chr4 | 146671565 | 146671565 | Th-Naive_3519 | 0.0640508556132577 |
| chr14 | 103091146 | 103091146 | Th-Naive_1405 | 0.0650324517957081 |
| chrX | 121571721 | 121571721 | Th-Naive_5228 | 0.0656523189594139 |
| chr14 | 79078802 | 79078802 | Th-Naive_1348 | 0.0694585353337249 |
| chr17 | 76568244 | 76568244 | Th-Naive_1987 | 0.0707211459038271 |
| chrX | 80175485 | 80175485 | Th-Naive_5112 | 0.0738810203397392 |
| chr5 | 176709247 | 176709247 | Th-Naive_3873 | 0.0761023394576042 |
| chr14 | 94850823 | 94850823 | Th-Naive_1377 | 0.0785770145792999 |
| chr7 | 99142887 | 99142887 | Th-Naive_4302 | 0.0926432934956826 |
| chr1 | 32754919 | 32754919 | Th-Naive_112 | 0.0927685647124738 |
| chr18 | 75800082 | 75800082 | Th-Naive_2099 | 0.0989707783642076 |
| chr6 | 70377691 | 70377691 | Th-Naive_4006 | 0.0993438897020032 |
| chr9 | 67216659 | 67216659 | Th-Naive_4722 | 0.1003805597005113 |
| chr6 | 86131518 | 86131518 | Th-Naive_4024 | 0.1015807543584278 |
| chr12 | 66059099 | 66059099 | Th-Naive_986 | 0.102600455251931 |
| chr8 | 144467769 | 144467769 | Th-Naive_4652 | 0.1076052409393061 |
| chr17 | 78563721 | 78563721 | Th-Naive_1991 | 0.1091464251169795 |
| chr4 | 104700387 | 104700387 | Th-Naive_3474 | 0.119379686714795 |
| chr1 | 147879702 | 147879702 | Th-Naive_249 | 0.1200787342118236 |
| chr1 | 237025006 | 237025006 | Th-Naive_391 | 0.1256121377988857 |
| chr2 | 48272187 | 48272187 | Th-Naive_2421 | 0.1327933213576217 |
| chr1 | 46505657 | 46505657 | Th-Naive_149 | 0.132858071486766 |
| chr2 | 95379454 | 95379454 | Th-Naive_2494 | 0.1404844651301636 |
| chr2 | 204341 | 204341 | Th-Naive_2348 | 0.1406143573335889 |
| chr21 | 46291146 | 46291146 | Th-Naive_2961 | 0.1575689151792032 |
| chr3 | 18391457 | 18391457 | Th-Naive_3109 | 0.1670686687202596 |
| chr17 | 6894272 | 6894272 | Th-Naive_1812 | 0.1708831893280304 |
| chr2 | 209194978 | 209194978 | Th-Naive_2615 | 0.181712132044888 |
| chr4 | 188612490 | 188612490 | Th-Naive_3569 | 0.1858547555174432 |
| chr9 | 76017902 | 76017902 | Th-Naive_4735 | 0.1897027913244852 |
| chr10 | 94256659 | 94256659 | Th-Naive_544 | 0.2063256100688054 |
| chrX | 95241263 | 95241263 | Th-Naive_5147 | 0.2226545406218776 |
| chr3 | 180631817 | 180631817 | Th-Naive_3296 | 0.2396358989057966 |
| chr19 | 1295426 | 1295426 | Th-Naive_2131 | 0.2627879562350783 |
| chr6 | 121877775 | 121877775 | Th-Naive_4070 | 0.2784677811256539 |
| chrX | 109582826 | 109582826 | Th-Naive_5195 | 0.293943390616702 |
| chr10 | 54407700 | 54407700 | Th-Naive_496 | 0.2997421878875085 |
| chr21 | 26520677 | 26520677 | Th-Naive_2904 | 0.3759722437635469 |
| chrX | 57200411 | 57200411 | Th-Naive_5048 | 0.4819686669374758 |

For example, to determine if a cell has been infected with HIV, memory B cells can be analyzed to determine if the DMR at human chromosome 10 position 49259640 is methylated. If not methylated, this indicates that the cell has been exposed or infected with HIV. For example, to determine if a cell has been infected with an OP, naïve T cell can be analyzed to determine if DMR at human chromosome 1 positions 1381268-1381294 is methylated. If not methylated, this indicates that the cell has been exposed to an OP.

By determining the methylation status of at least 20, at least 25, at least 30, at least 40, at least 50, at least 100, at least 200, at least 300, at least 400, at least 500, at least 600, at least 700, at least 800, at least 900, at least 1000, at least 5000, or at least 5400 DMRs in Table 1, in combination with determining an amount of accessible chromatin, one can determine whether a subject has been infected by or exposed to B. anthracis (Table 1, exposure BA), infected by or exposed to SARS-CoV-2 (Table 1, exposure COVID), infected by or exposed to influenza A (Table 1, exposure Flu), infected by or exposed to HIV-1 (Table 1, exposure HIV), infected by or exposed to MSSA or MRSA (Table 1, exposure MRSA), or exposed to organophosphate (Table 1, exposure OP). In some examples, the methylation status of the DMRs for two or more exposures are analyzed simultaneously or contemporaneously, such as 2, 3, 4, 5 or all 6 exposures.

### Obtaining Immune Cells

The methods utilize PBMCs, specifically particular immune cells. In some aspects, the method includes obtaining a blood sample from a subject. In some aspects, the method includes isolating or separating particular immune cells to be analyzed from a blood sample, for example using FACS. FACS can be used to identify particular immune cells based on cell-surface markers. In some aspects, B cells, monocytes, NK cells, CD8 memory T cells, CD8 naïve T cells, CD4 memory T cells, and CD4 naïve T cells are isolated from a blood or PBMC sample obtained from a subject. For example, detectably labeled antibodies specific for the following markers can be used to identify and separate immune cells: naïve helper T cells (CD3+, CD4+, CCR7+, CD45RA+), Memory helper T cells (CD3+, CD4+, CD45RA−), Naïve cytotoxic T cells (CD3+, CD8+, CCR7+, CD45RA+), Memory cytotoxic T cells (CD3+, CD8+, CD45RA−), B cells (CD3−, CD19+), Monocytes (CD3−, CD19−, CD14+), NK cells (CD3−, CD19−, CD14−, CD16+,CD56+), and other cells (CD3−, CD19−, CD14−, CD16−, CD56−). In some aspects, the methods can include separating B cells, monocytes, NK cells, CD8 memory T cells, CD8 naïve T cells, CD4 memory T cells, and CD4 naïve T cells into separate wells of a multi-well plate. In some aspects, cells are stored for future analysis, such as frozen. In some aspects, DNA is obtained or isolated from the isolated immune cells.

In some aspects, at least 10, at least 25, at least 50, at least 75, at least 100, or at least 1000 cells of each immune cell type are analyzed. In some aspects, no more than 1000, no more than 500, no more than 200, no more than 100, no more than 50 or no more than 10 cells of each immune cell type are analyzed, (such as 10-1000, 10-500, 10-250, 10-100, 50-100 or 50-250 cells).

### Measuring Methylation Status

The methylation status of DNA methylation markers, such as at least 20, at least 25, at least 30, at least 40, at least 50, at least 100, at least 200, at least 300, at least 400, at least 500, at least 600, at least 700, at least 800, at least 900, at least 1000, at least 5000, or at least 5400 of those disclosed herein, are determined in the isolated immune cells. In some examples, such an analysis is performed at the single cell level. Genome-wide methylation profiles in the CG context of the isolated immune cells can be determined. In some examples, two sub-clusters within B cells and NK cells are analyzed separately: Memory B cells (B-Mem), naïve B cells (B-Naïve), active natural killer cells (NK cell 1), and naïve natural killer cells (NK cell 2). Differentially methylated regions (DMRs) within each of these nine cell types (Memory B cells (B-Mem), naive B cells (B-Naïve), active natural killer cells (NK cell 1), naive natural killer cells (NK cell 2), memory cytotoxic T cells (Tc-Mem), naive cytotoxic T cells (Tc-Naïve), memory T cells (Th-Mem), naïve T cell (Th-Naïve), Monocytes) can be identified. Exemplary DNA methylation markers that can be evaluated are provided in Table 1. That is, the CpG sites in Table 1 (noted by their human chromosomal start and stop positions) can be evaluated to determine if they are, or are not, methylated at that position.

DNA methylation status can be determined using any available assay.

In one aspect, single-nucleus methylation sequencing (snmC-seq2) is used to determine the methylation status of a particular DNA methylation markers (see Table 1) for each cell within a population of isolated immune cells. Namely, such methods can be used to determine whether a particular region in the genome is methylated or not. For example, the isolated immune cells can be subjected to bisulfite conversion (e.g., exposed to bisulfite), which converts cytosines in the genomic sequence to a uracil if the cytosine is not methylated (if the cytosine is methylated, no change to uracil will occur). Following bisulfite conversion, the genomes of the immune cells are amplified and a library for each immune cell type generated and sequenced. The resulting sequences can be compared to a reference genome (e.g., a human genome not exposed to a pathogen or chemical (B. anthracis, SARS-CoV-2, influenza A, HIV-1, MRSA, MSSA, or an organophosphate). Detection of a thymine in the sequenced test immune cell as compared to a cytosine in the reference genome indicates methylation at that position in the test sample.

In one aspect, an Illumina™ DNA methylation array is utilized. In one aspect, a PCR protocol using relevant primers is utilized.

In one aspect, a molecular break light assay for DNA adenine methyltransferase activity is used. This assay is based on the specificity of the restriction enzyme DpnI for fully methylated (adenine methylation) GATC sites in an oligonucleotide labeled with a fluorophore and quencher. The adenine methyltransferase methylates the oligonucleotide making it a substrate for DpnI. Cutting of the oligonucleotide by DpnI gives rise to a fluorescence increase, thus indicating that the position is methylated. In one example, methylation-specific polymerase chain reaction (PCR) is used. This method is based on a chemical reaction of sodium bisulfite with DNA that converts unmethylated cytosines of CpG dinucleotides to uracil or UpG, followed by traditional PCR. However, methylated cytosines are not converted in this process, and thus primers are designed to overlap the CpG site of interest, which allows one to determine methylation status as methylated or unmethylated. In one example, whole genome bisulfite sequencing, also known as BS-Seq, is used. This is a genome-wide analysis of DNA methylation based on the sodium bisulfite conversion of genomic DNA, which is then sequenced. The sequences obtained are then re-aligned to the reference genome to determine methylation states of CpG dinucleotides based on mismatches resulting from the conversion of unmethylated cytosines into uracil. In one example, the Hpall tiny fragment Enrichment by Ligation-mediated PCR (HELP) assay is used, which is based on restriction enzymes' differential ability to recognize and cleave methylated and unmethylated CpG DNA sites. In one example, methyl sensitive southern blotting is used, which uses Southern blotting techniques to probe gene-specific differences in methylation using restriction digests. This method can be used to evaluate local methylation near the binding site for the probe. In one example, ChIP-on-chip assay is used. This method uses commercially prepared antibodies to bind to DNA methylation-associated proteins like MeCP2. In one example, restriction landmark genomic scanning is used, which is based upon restriction enzymes' differential recognition of methylated and unmethylated CpG sites. In one example methylated DNA immunoprecipitation (MeDIP) is used. Immunoprecipitation is used to isolate methylated DNA fragments for input into DNA detection methods such as DNA microarrays (MeDIP-chip) or DNA sequencing (MeDIP-seq). In one example, pyrosequencing of bisulfite treated DNA is used. In this method, an amplicon is generated by a normal forward primer but a biatenylated reverse primer to PCR the target methylation marker. A pyrosequencer then analyzes the sample by denaturing the DNA and adding one nucleotide at a time to the mix according to a sequence given by the user. If there is a mismatch, it is recorded and the percentage of DNA for which the mismatch is present is noted. This provides a percentage methylation per CpG island.

In some aspects, the genomic DNA to be analyzed is used directly, e.g., hybridized to a complimentary sequence (e.g., a synthetic polynucleotide sequence) that is attached to a solid support (e.g., one disposed within a microarray). In some examples, the genomic DNA to be analyzed is amplified by a PCR process. For example, prior to or concurrent with hybridization to an array, the sample may be amplified by a variety of mechanisms, such as those that employ PCR. The sample may be amplified on the array.

Electrical or other signals that can identify the methylation state of the DMRs in Table 1 can also be used to determine the methylation states of these biomarkers.

### Measuring Accessible Chromatin

The relative or absolute amount of accessible chromatin in the isolated immune cells can be determined. In some examples, such an analysis is performed at the single cell level. Analysis of chromatin accessibility can identify transcriptional regulatory sequences. Analysis of chromatin accessibility can be determined using any available assay. In one example, single-nucleus ATAC-seq is used, which is a combinatorial barcoding-assisted single-cell assay for transposase-accessible chromatin. In some examples, DNase-seq is used.

In some aspects, the method includes fragmenting chromatin from the isolated immune cells, for example using a transposase, and then the fragments sequenced. In some aspects, nuclei from the isolated immune cells are isolated prior to fragmenting. In some aspects, bar codes are added to the fragments. The sequenced fragments can be compared to fragments generated from control immune cells (or reference value(s) for such), such as a human genome not exposed to a pathogen or chemical (B. anthracis, SARS-CoV-2, influenza A, HIV-1, MRSA, MSSA, or an organophosphate).

### III. Statistical Analysis

Any statistical approach can be used to relate the methylation status to a particular of exposure or infection (B. anthracis, SARS-CoV-2, HIV-1, influenza A, MSSA/MRSA, and/or organophosphate), and can be regressed on the CpG markers using a linear regression model as described herein. Using regression model/analysis tools and methodologies, a number of exposure/infection prediction models are contemplated for use with specific genomic DNA samples and/or specific analysis techniques and/or specific individual populations. In one example, an identity transformation method is used, wherein the exposure/infection is identified using regression of the CpG status. In other examples, the exposure/infection type is transformed. In some examples, a weighted average of the CpGs is determined.

For example, Table 1 shows the relationship between the methylation status of DMRs, for particular combinations of exposures (BA, COVID, Flu, HIV, MRSA/MSSA, OP) and cell types (B-Mem, B-Naïve, NK-Active=NK1, NK-Naïve=NK2, Tc-Mem, Tc-Naïve, Th-Mem Th-Naïve, Monocyte). In Table 1, a positive weight value indicates that the DMR is methylated at that position for that exposure/infection and cell type, a negative weight indicates that the group is not methylated at that position for that cell type. Thus, one can determine the methylation status for each of the exposure conditions and cells types listed in Table 1, and based on the status (methylated or not) for each marker, be used in combination with the amount of accessible chromatin, to determine whether the cell (or subject from whom the cell was obtained) was exposed to a particular condition.

In one example, a prediction model is trained with the methylation status of at least 20, at least 25, at least 30, at least 40, at least 50, at least 100, at least 200, at least 300, at least 400, at least 500, at least 600, at least 700, at least 800, at least 900, at least 1000, at least 5000, or at least 5400 of the DMRs listed in Table 1 and applied to new samples.

### IV. Treatment

Once the particular type of exposure or infection is identified (B. anthracis, SARS-CoV-2, HIV-1, influenza A, MSSA, MRSA, and/or organophosphate), the method can include administering a therapeutically effective amount of one or more therapeutic agents based on the exposure identified, as outlined in Table 2.

TABLE 2
Exemplary treatments for B. anthracis, SARS-CoV-
2, influenza A, HIV-1, MRSA, or organophosphate

| Condition | Exemplary Treatments |
|---|---|
| Bacillus | ciprofloxacin, doxycycline, quinolone, penicillin (e.g., orally or i.v.) |
| anthracis/anthrax | |
| SARS-CoV-2/COVID | One or more antivirals, such as paxlovid, molnupiravir, and/or remdesivir. |
| | Such antivirals can be administered orally (paxlovid, molnupiravir) or iv |
| | (remdesivir). Can also include one or more immune modulators, such as |
| | Olumiant (baricitinib) and Actemra (tocilizumab). |
| Influenza | oseltamivir (Tamiflu ®) [oral], zanamivir (Relenza ®) [inhalation], or |
| | peramivir [i.v.] (Rapivab ®) |
| HIV-1/AIDs | Highly active antiretroviral therapy (HAART), which combines reverse |
| | transcriptase inhibitors (e.g., NRITs such as zidovudine, abacavir, |
| | lamivudine, emtricitabine; NtRTIs such as tenofovir and adefovir); NNRIs |
| | such as nevirapine, efavirenz, etravirine and rilpivirine) and protease |
| | inhibitors (e.g., lopinavir, indinavir, nelfinavir, amprenavir, ritonavir, |
| | darunavir and atazanavir) or integrase inhibitors (e.g., raltegravir, |
| | elvitegravir, dolutegravir, bictegravir, and cabotegravir). |
| MRSA | Vancomycin, alone or in combination with other beta-lactam agents; |
| | ceftaroline; teicoplanin; clindamycin, and/or linezolid. (e.g., IV, oral, or |
| | both). |
| MSSA | a Beta-lactam (β-lactam) antibiotic, such as penicillin and its derivatives |
| | (such as methicillin and oxacillin) and cephems such as the cephalosporins; |
| | flucloxacillin; triple antibiotic ointment |
| Organophosphate | Carbamate; anti-cholinergic drugs (e.g., atropine, oximes (such as |
| poisoning | pralidoxime, and diazepam)); cholinesterases (ChEs), such as human |
| | serum BChE (HuBChE); class II anti-arrhythimic agents |

### Example 1

### Materials and Methods

This example provides the materials and methods used to generate the data described herein. Additional details can be found in Wang et al., Human Immune Cell Epigenomic Signatures in Response to Infectious Diseases and Chemical Exposures, biorxiv, Jun. 30, 2023 (https://doi.org/10.1101/2023.06.29.546792) herein incorporated by reference in its entirely, including the supplemental materials.

### Fluorescence-Activated Cell Sorting of Immune Cell Types

Cells were sorted into 384-well plates by FACS using an antibody cocktail that can label seven different immune cell types in blood. This sorting collects Naive helper T cells (CD3+, CD4+, CCR7+, CD45RA+), Memory helper T cells (CD3+, CD4+, CD45RA−), Naive cytotoxic T cells (CD3+, CD8+, CCR7+, CD45RA+), Memory cytotoxic T cells (CD3+, CD8+, CD45RA−), B cells (CD3−, CD19+), Monocytes (CD3−, CD19−, CD14+), NK cells (CD3−, CD19−, CD14−, CD16+,CD56+), and other cells (CD3−, CD19−, CD14−, CD16−, CD56− based on the specific antibodies. The SONY Muti-Application Cell Sorter LE-MA900 Series was used to isolate single cells in 384-well PCR plates containing protein kinase. After cell sorting, the plates were spun down to capture the cells at the bottom of the well and then subjected to thermocycling at 50° C. for 20 minutes. The plates containing the DNA from the cells were subsequently stored at −20° C. or moved directly to library preparation.

### Library Preparation and Illumina Sequencing

For library preparation, we followed the previously described methods for bisulfite conversion and library preparation in snmC-seq2 (Luo et al., 2017, 2018). The snmC-seq2 libraries generated from the isolated immune cells were sequenced using an Illumina Novaseq 6000 instrument with S4 flow cells in the 150-bp paired-end mode. Freedom EVOware v2.7 was utilized for library preparation, while Illumina MiSeq control software v3.1.0.13 and NovaSeq 6000 control software v1.6.0/Real-Time Analysis (RTA) v3.4.4 were employed for sequencing.

### Quantification and Statistical Analysis

Single-cell methylation data processing (alignment, QC) For alignment and quality control (QC) of the single-cell methylation data, we employed the same mapping strategy used in a previous single-cell methylation project (Liu et al., 2021). Specifically, we utilized our in-house mapping pipeline, YAP (https://hq-1.gitbook.io/mc/), for all the mapping-related analysis. The pipeline includes the following main steps: (1) demultiplexing FASTQ files into single cells, (2) reads-level QC, (3) mapping, (4) BAM file processing and QC, and (5) generation of the final molecular profile. Detailed descriptions of these steps for snmC-seq2 can be found in the work by Luo et al. (2018). All the reads were mapped to the human hg38 genome, and we calculated the methylcytosine counts and total cytosine counts for two sets of genomic regions in each cell after mapping.

Low-quality cells were filtered out based on three metrics generated during mapping: mapping rate >50%, final mC reads >500,000, and global mCG>0.5. Chromosomes X, Y, and M were excluded from the analysis, and the remaining genome was divided into 5 kb bins to create a cell-by-bin matrix. In this matrix, each bin was assigned a hypomethylation score (hypo-score) calculated from the p-values of a binomial test, which indicates the probability of hypomethylation of that bin. The matrix was further binarized for downstream analysis using a hypo-score cutoff of >=0.95. Bins covered by fewer than 5 cells and those with an absolute z-score greater than 2 were filtered out. Additionally, we excluded bins that overlapped with the ENCODE blacklist using “bedtools intersect” (Dale, Pedersen, and Quinlan 2011; Quinlan and Hall 2010).

### Unsupervised Clustering

To perform unsupervised clustering, ALLCools (Liu et al., 2021) was utilized, which first conducted principal component analysis (PCA) on the 5 kb bin matrix. For each exposure, the top 32 principal components (PCs) were selected for clustering using the modules in scanpy (Wolf, Angerer, and Theis 2018). In the HIV-1 and influenza cohorts, a donor effect in the clustering results was observed with these PCs. Therefore, harmony (Korsunsky et al., n.d.) was applied to correct the donor effect on these PCs. Clustering was performed separately for control samples (‘HIV_pre’, ‘Flu_pre’, and ‘Ctrl’) and samples from the ‘MRSA/MSSA’, ‘BA’, ‘COVID-19’, and ‘OP’ groups, allowing for better comparison between the exposures and control samples.

To annotate the cells, both the single-cell methylation clustering results and cell surface markers were used. In almost every cohort, two clusters of B cells and NK cells were observed, which were distinguished by their global mCG levels. Therefore, these clusters were assigned as naive and memory B cells, naive and active NK cells. We also merged clusters with cell surface markers indicating memory CD4 and CD8 T cells, even if they exhibited multiple clusters in the T-SNE embedding.

### Differentially Methylated Regions (DMR) Identification

To identify DMRs associated with each immune cell type, PBMCs from healthy donors were utilized. Based on single-cell methylation and fluorescence-activated cell sorting (FACS), nine cell types were identified through clustering. These cell types were grouped based on their global mCG levels, and DMRs were called separately within high-mCG and low-mCG cell types. Methylpy (https://github.com/yupenghe/methylpy) was used for DMR calling, and the resulting DMRs were further annotated with genes and promoters.

For the identification of DMRs associated with each exposure, the control samples and samples from each exposure group were merged. Methylpy (https://github.com/yupenghe/methylpy) was used to identify DMRs between the control and exposure groups, as well as between different exposure groups. Once the primary set of DMRs were obtained, the methylation levels of all samples at these DMRs was calculated using “methylpy add-methylation-level”.

Additional filtering on the DMRs was performed by comparing the methylation levels among different sample groups using Student's t-test. Only DMRs with a minimum p-value less than 0.05 between any two groups were retained. For DMRs associated with MRSA/MSSA, BA, OP, and SARS-CoV-2, where external controls were used for DMR calling, we compared the methylation levels of exposure samples and control samples, as well as different cohorts of controls (HIV, Flu, and commercial controls). DMRs that showed significant differences (p-value <0.05) between the exposure group and all three control cohorts, but not significant differences (p-value >0.05) between any two control cohorts, were retained.

To visualize the complex heatmaps, we employed PyComplexHeatmap (https://github.com/DingWB/PyComplexHeatmap). Hypomethylated DMRs in the corresponding sample groups and cell types were labeled for better visualization. The heatmap rows were split according to sample groups, and the columns were split based on DMR groups and cell types. Within each subgroup, rows and columns were clustered using ward linkage and the Jaccard metric.

### Differentially Methylated Loci (DML) Identification

DSS (Park and Wu 2016) was used to call the DMLs in each exposure. The samples were grouped based on exposure types, and made pairwise comparisons to identify DMLs. To save memory and time, DSS was run on each chromosome, and the results were merged to calculate FDR using the statsmodels package. DMRs from methylpy and DMLs from DSS were compared by checking the heatmaps of mCG level at these sites in all samples, then the DMRs from methylpy were used for downstream analysis.

### Validation of DMRs by Shuffling the Samples

To validate that the identified DMRs for each exposure were not confounded by batch effects or other factors, we shuffled the group labels of the samples within each exposure and identified DMRs among the randomly assigned groups. We quantified the methylation levels of all samples at the DMRs from the random groups and performed t-tests on the methylation levels between each pair of groups.

### Motif Enrichment

We obtained the hypo- and hyper-DMRs reported by methylpy from the columns ‘hypermethylated_samples’ and ‘hypomethylated_samples’. HOMER was used to identify enriched motifs within these different sets of DMRs for each exposure. The results from HOMER's ‘knownResults.txt’ output files were used for downstream analysis. Only motif enrichments with a p-value <0.01 were retained. The motif enrichment results were visualized using scatterplots in seaborn.

### Differentially Methylated Genes (DMG) Identification

Pairwise differential methylation analysis of genes (DMGs) for each exposure was performed using ALLCools, following the tutorial (lhqing.github.io/ALLCools/cell_level/dmg/04-PairwiseDMG.html). Significantly differentially methylated genes were selected based on an FDR<0.01 and a delta mCG>0.05. The functional enrichment analysis of the DMGs was conducted using metascape (Zhou et al., 2019) (metascape.org).

### Functional Enrichment of DMRs

GREAT (great.stanford.edu/public/html/index.php) was used for the functional enrichment analysis of DMLs.

Integration with Single-Cell ATAC

Single-cell methylation data was integrated with single-cell ATAC-seq data from HIV-1, COVID-19, BA, and OP cohorts, as well as single-cell multiome data from influenza. This integration was performed using Canonical Correlation Analysis (CCA), where methylation cell annotations to the cells were transferred from the other modality. To generate the peaks and bigwig files for each cell type, SnapATAC2 was utilized (Zhang et al., 2021; Fang et al., 2021).

### Correlation of Single-Cell Methylation and Single-Cell ATAC

To assess the correlation between single-cell methylation and single-cell ATAC, the correlation between the hypo-score of each 5 kb bin and Tn5 insertions in each bin were calculated. This correlation was performed between cell types and within matched cell types.

### Prediction of Exposures Using Sub-Clusters, Genes and DMRs

To predict different exposures, three pieces of information were utilized: sub-clusters, genes, and differentially methylated loci (DMLs). For sub-cluster prediction, clustering within each sorted cell type from all exposures were performed and sub-clusters based on Leiden clustering identified. The samples were divided into training and test sets at a ratio of 6:4. Then, 1000 pseudo-individuals were generated by permuting the training and testing samples (700 as training and 300 as the test set). From each pseudo-individual, 500 cells were randomly sampled and their distribution across the sub-clusters calculated. This distribution was used to predict different exposures.

For gene-based prediction, we utilized the methylation level of genes. For each cell type, we generated 400 pseudo-individuals, with 300 as training samples and 100 as the test set. The samples were divided into training and testing sets at a ratio of 7:3. We calculated the gene body methylation level for all genes and used this information to predict different exposures. Similarly, for DML-based prediction, we utilized DMLs to predict different exposures. The process was similar to the gene-based prediction, where we quantified the DMLs by methylation level and used this information to predict different exposures within each cell type.

### Example 2

### Overview of the Exposome Cohorts

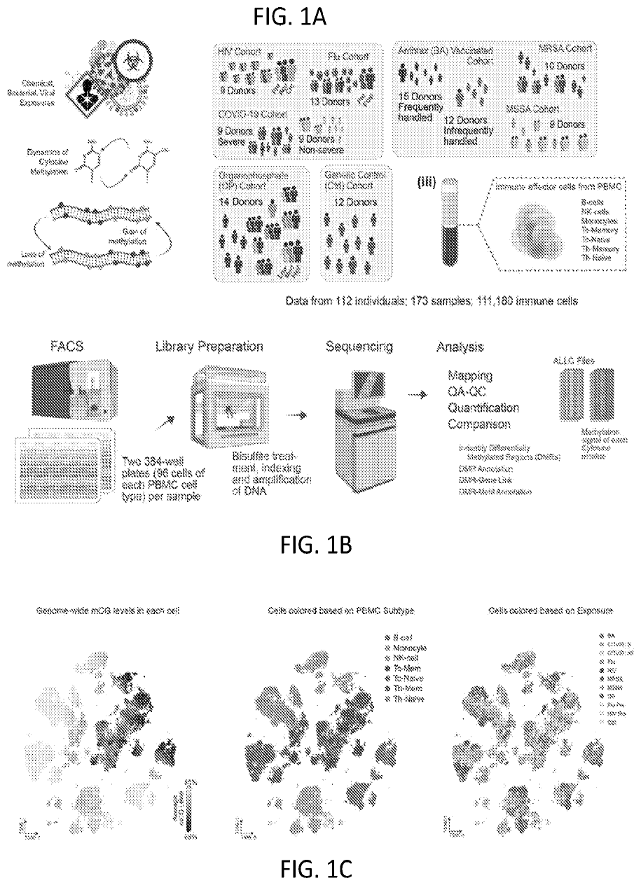

Three categories of exposures were investigated-viral, bacterial, and chemical (FIG. 1A). To examine viral exposure, the methylation patterns of individuals who were part of a prospective study on HIV-1 infection prevention were evaluated. Additionally, a cohort of volunteers who participated in a vaccine trial against the H3N2 flu virus and a group of patients who experienced SARS-CoV-2 infection with varying degrees of disease severity were analyzed. For bacterial exposures, the methylation profiles of patients infected with MRSA and MSSA, as well as vaccinated technicians who handled Bacillus anthracis, were examined. In terms of chemical exposure, samples were obtained from individuals with high levels of 3,5,6-trichloro-2-pyridinol (TCPY) resulting from exposure to chlorpyrifos—an organophosphate insecticide known to be neurotoxic and shares a mode of action with nerve agents.

Peripheral blood mononuclear cells (PBMCs) were collected from each of these cohorts, and seven major immune cell types were isolated and sorted (FIG. 1B). To account for potential batch effects, all seven immune cell types were sorted on the same 384-well plate. Subsequently, the methylomes of each single cell were profiled using snmC-seq² (Luo et al., 2017, 2018) and processed the data using ALLCools (Liu et al., 2021)—a custom bioinformatic pipeline for analyzing single-nucleus methylation data. Leiden clustering and t-distributed stochastic neighbor embedding (t-SNE) analysis were performed of the single nuclei based on their methylation profiles across all cells (FIG. 1C). While immune cell types were initially sorted using cell surface markers, the global mCG levels enabled unique sub-clusters within B cells and NK cells (FIG. 1C) to be identified. Cells from different exposures were not evenly distributed across these sub-clusters, indicating the presence of distinct epigenomic diversity associated with exposure types (FIG. 1C).

HIV Cohort: The iPrEx cohort (Iniciativa Profilaxis Pre-Exposici6n, or the PreP pre-exposure prophylaxis initiative) was a phase III clinical trial aimed at determining the efficacy of pre-exposure prophylaxis (PrEP), an antiretroviral treatment, for preventing HIV infection (Grant et al., 2010). The study enrolled high-risk individuals who were continuously monitored for the onset of HIV-1 infection. During the study, some participants became HIV-1 positive. We obtained samples from nine donors taken approximately −250 days (median of 258 days) before testing positive for HIV-1 (pre), on the day of the HIV-1 positive blood draw (acute), and approximately −200 days after treatment (chronic). We observed significant variation in the initial virus load and the load after 200 days during the chronic stage when standard HIV-1 treatment was provided. For this study, we PBMC samples from nine subjects at three time points were used.

### Influenza Cohort:

The BARDA-Vaccitech FLU010 Study with A/BELGIUM/4217/2015 (H3N2) challenge was designed to assess the protective capabilities of a vaccine candidate VTP-100 as a standalone influenza vaccine. Pre- and post-challenge samples were obtained from 18 donors who received the placebo vaccine with post-challenge sampling at 28 days after the challenge dose.

### COVID-19 Cohort:

Patients who were hospitalized following SARS-CoV-2 infection with similar COVID severities based on the WHO ordinal severity score between 4 and 7, were enrolled in this cohort. Some patients showed improvement with moderate disease (non-severe group), while others experienced clinical deterioration within a 14-day period (increased severity score) and required invasive mechanical ventilation or succumbed to the disease (severe group). Samples were obtained from nine patients in each group.

MRSA-MSSA Cohort: Samples from 19 patients who tested positive for either Methicillin-resistant Staphylococcus aureus (MRSA) or Methicillin Sensitive Staphylococcus aureus (MSSA) were obtained. Ten donors were infected with MRSA and 9 donors infected with MSSA. Among them, nine donors provided two or three longitudinal samples, resulting in a total of 27 samples, with 13 MRSA samples and 14 MSSA samples. The time intervals between the second and third time points ranged from 3 to 35 days since the first positive test.

Bacillus anthracis Cohort: PBMC samples from 27 persons who handled Bacillus anthracis in a controlled facility wearing personal protective equipment were obtained. These persons were trained technicians who worked within BSL3 facilities and were vaccinated against B. anthracis infection. The vaccine used was Anthrax Vaccine Adsorbed, distributed under the trade name BioThrax®. It is an inactivated, acellular vaccine primarily containing the non-pathogenic protective antigen (PA) protein. The vaccine is produced from cell-free filtrates of microaerophilic cultures of an avirulent, non-encapsulated strain of Bacillus anthracis. The donors also handled chemical weapon agents, precursor molecules to chemical agents, biological warfare agents, explosives, precursors to explosives, agricultural chemicals, or radioactive materials in a controlled environment.

Organophosphates Cohort: Organophosphates (OP) are a class of pesticides known to have a severe impact on the dopaminergic and serotonergic systems. A common form of this pesticide (Chlorpyrifos) has extensive use in the US. Exposure to chlorpyrifos, the most widely used OP in the US, was estimated based on levels of its urinary metabolite 3,5,6-trichloro-2-pyridinol (TCPY) and classified as high, moderate, or low (with undetectable levels of TCPy). DNA methylation patterns were analyzed in 18 high-exposure, six moderate-exposure, and three low-exposure samples.

Control Cohorts: PBMC samples were obtained from 12 healthy donors through a commercial vendor (Dx Biosamples LLC). These donors represented a range of ethnic diversity, age groups, and sexes. Together with the pre-HIV samples and the pre-influenza challenge samples mentioned above, 34 control PBMC samples were used.

De-identified human PBMC samples were obtained.

### Example 3

### Methylation Signatures in Immune Cells from Healthy Donors at Single-Cell Resolution

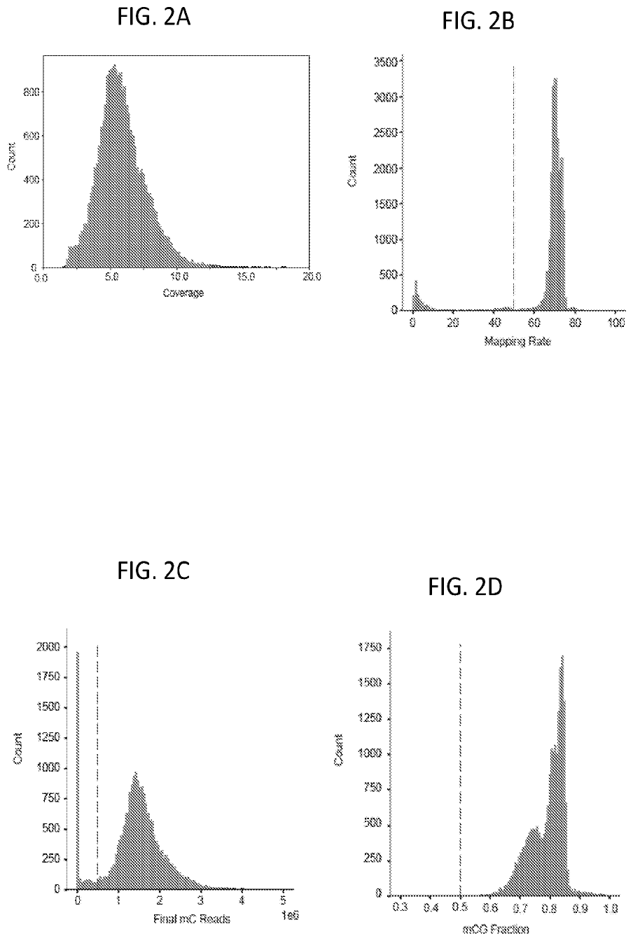

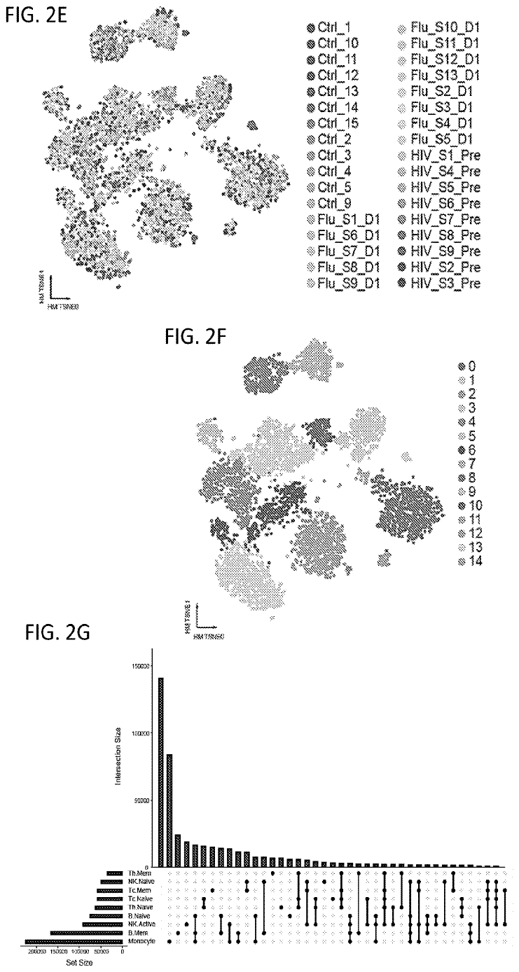

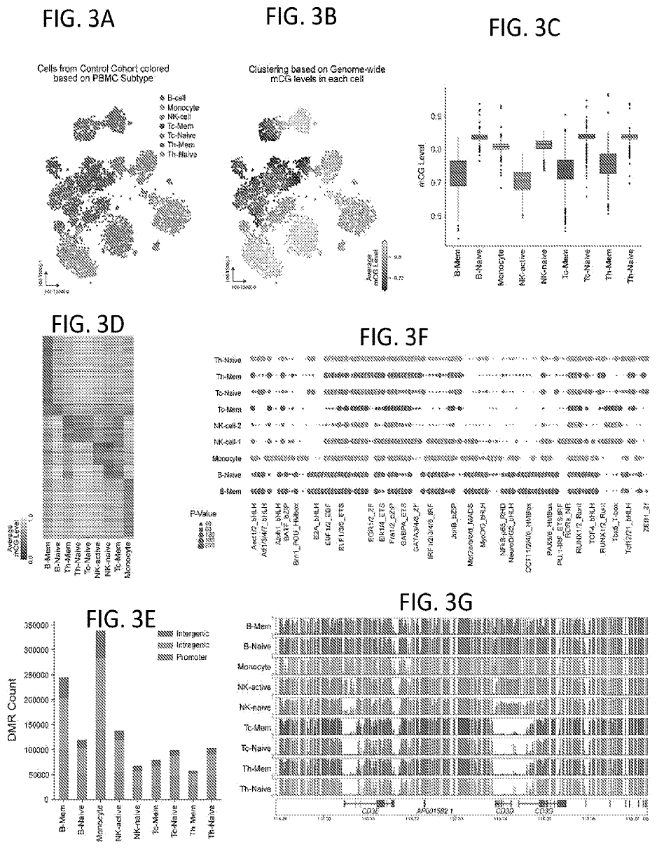

To elucidate the cell type-specific methylation signatures for major immune cell classes at single-cell resolution, peripheral blood mononuclear cells (PBMCs) were obtained from healthy donors across different cohorts. As outlined in in FIGS. 1A-1B, fluorescence-activated cell sorting (FACS) was performed to isolate seven major immune cell types from PBMCs, followed by single-cell methylation sequencing (Luo et al., 2017, 2018). Methylation reads from individual immune cells covered approximately 5%-10% of the genome (FIG. 2A) and underwent filtering based on read mapping rate, total read count, and global CpG methylation (mCG) level (FIG. 2B-2D). In total, 22,341 high-quality cells from healthy donors that passed the filtering step were obtained and used for subsequent analysis. To cluster these single cells, the genome was partitioned into 5 kb bins, generating a cell-by-bin matrix (Liu et al., 2021), upon which clustering and t-SNE were performed. After incorporating Harmony (Korsunsky et al., 2019) to account for donor effects, the results demonstrated well-mixed cells from different cohorts (FIG. 2E), affirming the reliability of our clustering outcomes. Cell type assignment was further refined based on cell surface markers and single-nucleus methylation clustering (FIGS. 3A, 2F). Additionally, by considering the global mCG levels of single nuclei, two subpopulations within B cells and NK cells were identified, denoted as memory B cells (B-Mem), naive B cells (B-Naive), activated NK cells (NK-Active), and naive NK cells (NK-Naive) (FIGS. 3A-3C), respectively. CD4 and CD8 naive T-cells exhibited higher global methylation levels and formed distinct clusters, while CD4 and CD8 memory T cells displayed more diverse methylation profiles and lower global mCG levels (FIGS. 3A-3C).

To identify regulatory elements in different immune cells, hypomethylated differentially methylated regions (DMRs) were detected for each cell type. Given the considerable variation in global mCG levels among these clusters (FIG. 3C), separate DMR calling was performed between cell types with low mCG and high mCG. In total, 495,479 DMRs were identified across the immune cell types, with most being unique to a specific cell type (FIG. 3D). Notably, sub-clusters of B cells and T cells shared only a small number of DMRs (FIG. 2G), while NK cells exhibited more DMRs shared with Tc-Mem cells than with other T cell subtypes (FIGS. 3D, 2G). To determine impact on gene regulation, each DMR was associated with underlying genomic features and observed significant enrichment in intragenic and promoter regions for each immune cell type (FIG. 3E). Motif analysis using DMRs revealed the enrichment of known lineage-specific transcription factor binding sites in corresponding cell types (FIG. 3F). For instance, TCF motifs were enriched in T cells, while the EBF1 motif showed enrichment in B cells (FIG. 3F). To further validate the accuracy of our data, the methylation status at T cell marker CD3 genes was plotted, which revealed hypomethylation in T cells and hypermethylation in other cell types (FIG. 3G).

### Example 4

### Impact of HIV-1 Infection on the Methylome

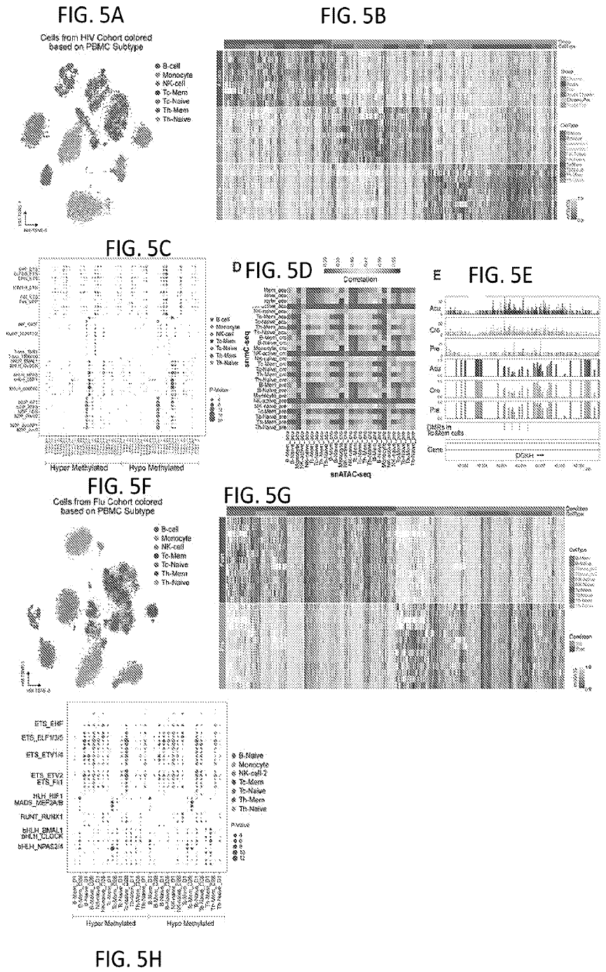

To identify the impact of HIV-1 infection on the methylome of immune cells, a comprehensive analysis was conducted using immune cells obtained from the same individuals at different stages: “pre” (before infection), “acute” (after diagnosis), and “chronic” (after treatment) (FIGS. 1A-1C). snmC-seq2 (Luo et al., 2017, 2018) was used to profile the methylome of these cells. Based on the methylation profiles, 13 clusters of immune cells were identified (FIG. 4A) and annotated based on both cell surface markers and methylation profiles. These cell clusters were evenly distributed across the three stages of the disease (FIG. 4B). Two sub-clusters of B cells and NK cells were identified based on methylation and cell surface markers, while other cell types were assigned based on cell surface markers (FIG. 5A). Similar to the analysis performed on healthy control samples, two clusters of B cells were classified as B-Mem and B-Naive, and two NK cell clusters were classified as NK-Active and NK-Naive, based on the global mCG level (FIG. 4C). The ratio between B-Mem and B-Naive remained consistent across the three stages of HIV-1 infection. However, the ratio between NK cell sub-clusters varied between the “acute,” “chronic,” and “pre” stages, with “pre” samples exhibiting fewer NK-Active cells and more NK-Naive cells (FIG. 4D) (P=0.0073, Chi-square test, comparing “acute” and “pre”; P=9.39e-06, comparing “chronic” and “pre”).

Significant changes in the genome-wide mCG levels of certain cell types were observed following HIV-1 infection. For instance, memory CD8 T cells exhibited an increase in global mCG levels from “pre” to “chronic” and “acute” stages (FIG. 4E). Other cell types such as Memory B cells, Naive B cells, and Naive NK cells also demonstrated significant global mCG changes between the “pre” and “acute” stages (FIG. 4E). Subsequently, differentially methylated regions (DMRs) were identified between the “pre,” “acute,” and “chronic” stages for these cell types. Interestingly, highly distinctive methylation patterns were observed between the three stages across all samples (FIG. 5B), indicating that these DMRs are highly consistent with disease progression. In total, 104,426 DMRs were identified between the three stages, with 103,460 (99.07%) of them being single CpG sites. Single CpG DMRs contained fewer CG contexts within 250 bp compared to other multi-CpG exposure DMRs (Wilcoxon Rank Sum Test, p-value=1.18e-130), while they contained more CGs than both multi-CpG (Wilcoxon Rank Sum Test, p-value=0.014) and single CpG cell type DMRs (Wilcoxon Rank Sum Test, p-value=0.0) (FIG. 4F). The largest number of hypomethylated DMRs (hypo-DMRs) was observed in memory CD8 T cells in the “acute” stage, while hypermethylated DMRs (hyper-DMRs) were predominant in the “pre” stage (FIG. 4G), consistent with the observed global mCG increase between the “pre” and “acute” and “chronic” stages of infection. Interestingly, the memory state of different cell types, including active NK cells, exhibited more DMRs compared to their naive forms (FIG. 4G).

To determine which transcription factors (TFs) may bind to these hypo-DMRs, enriched TF motifs were identified using HOMER (Heinz et al., 2010). ETS family motifs were found to be enriched in most cell types across the three stages of HIV-1 infection, whether in hypo-DMRs or hyper-DMRs (FIG. 5C), indicating a significant rearrangement of ETS family transcription factor binding following HIV-1 infection. For example, ETS family motifs were enriched in the hyper-DMRs of the “acute” and “chronic” stages in monocytes and naive CD8 T cells, while they were also enriched in the hypo-DMRs of the “pre” stage in these cell types (FIG. 5C). The enrichment of PU.1 and Fli1 motifs (FIG. 5C), which are transcription factors (Minderjahn et al., 2020) involved in regulating immune cell differentiation, indicates that immune cells may be undergoing differentiation into other lineages. RUNX motifs were enriched in hyper-DMRs of the “pre” stage in Naive NK cells, memory and naive CD8 T cells, while also being enriched in hypo-DMRs of the “acute” stage in these cell types. Together with other transcription factors, RUNX1/2 and ETS1 can alter chromatin state (Korinfskaya et al., 2021), potentially influencing T cell lineage determination. RUNX1 and RUNX3 have also been reported to drive the adaptive response of NK cells during MCMV infection (Rapp et al., 2017). Interestingly, motifs of transcription factors associated with circadian function were enriched in hypo-DMRs from the “pre” stage (FIG. 5C). These circadian transcription factors include CLOCK, BMAL1, bHLHE41, and NPAS2, indicating that circadian regulation may be altered in CD8 T cells after HIV-1 infection. Circadian transcription motifs were also enriched in hypo-DMRs of B cells in the “acute” stage. Additionally, CFCF motifs were observed enriched in naive B and naive CD4 T cells, indicating potential 3D genome reorganization in these cell types after HIV-1 infection (Rao et al., 2014; Nora et al., 2017).

Furthermore, differentially methylated genes (DMGs) were identified among the nine cell types between different HIV-1 groups. In pairwise comparisons of the “pre,” “acute,” and “chronic” stages, 288 DMGs were identified, with the majority found between the “pre” and “acute” stages in memory CD8 T cells (FIG. 4H). Among these DMGs, 112 genes exhibited hypomethylation in the “pre” stage, while 69 genes showed hypomethylation in the “acute” stage. Both groups of DMGs were enriched in genes with immune-related functions (FIG. 4I).

To validate the reliability of these methylation signatures and investigate potential regulatory mechanisms, DNA methylation data was integrated with single-cell ATAC-seq data. We mapped the cells from single-cell ATAC-seq to methylation clusters and transferred the cluster labels using Canonical Correlation Analysis (CCA) (Stuart et al., 2019). To validate the integration approach, we calculated the genome-wide correlation between these two modalities (FIG. 5D). Specifically, the genome was divided into 5 kb bins and the hypomethylation score calculated using single-cell methylation data and the number of Tn5 insertions using single-cell ATAC data (Liu et al., 2021). We then calculated the correlation between these two measurements for each bin. A strong correlation was observed between the two modalities (methylation and open chromatin) across all cell types, with the highest correlation observed in monocytes (FIG. 5D). Methylation in NK cells also showed a high correlation with chromatin accessibility in T cells (FIG. 5D). Interestingly, a loss of methylation and an increase in accessibility was detected after HIV-1 infection in memory CD8 T cells at the intron of DGKH (FIG. 5E), a gene previously reported to exhibit differential methylation between elite controllers (individuals able to maintain undetectable viral loads for at least 12 months without antiretroviral therapy) and individuals receiving antiretroviral therapy (Frias et al., 2021). Although this region experienced a loss of chromatin accessibility, the methylation level remained unchanged between the “acute” and “chronic” stages (FIG. 5E). When comparing all DMRs with differentially accessible regions (DARs) from the two modalities, a minimal overlap of only about 2% was observed, highlighting the improvement when using both technologies to profile the epigenomic remodeling associated with HIV-1 infection.

### Example 5

### Impact of Influenza Infection on the Methylome

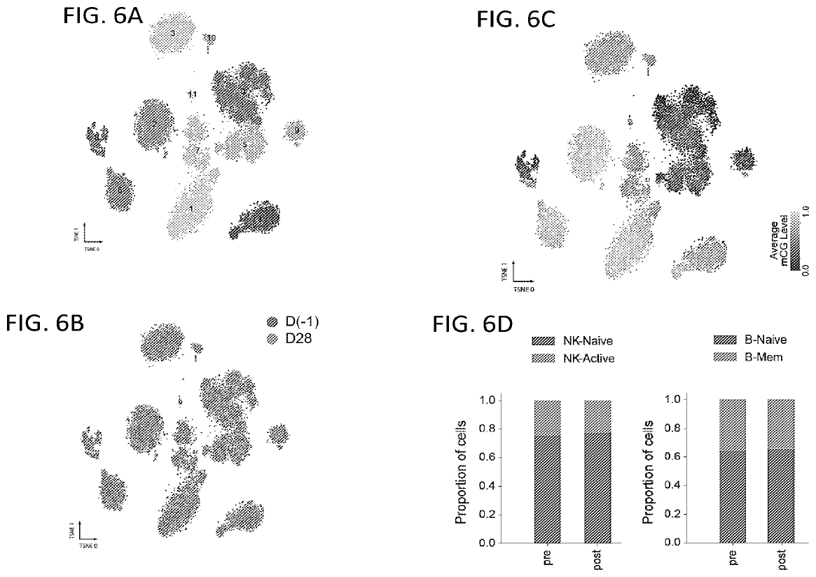

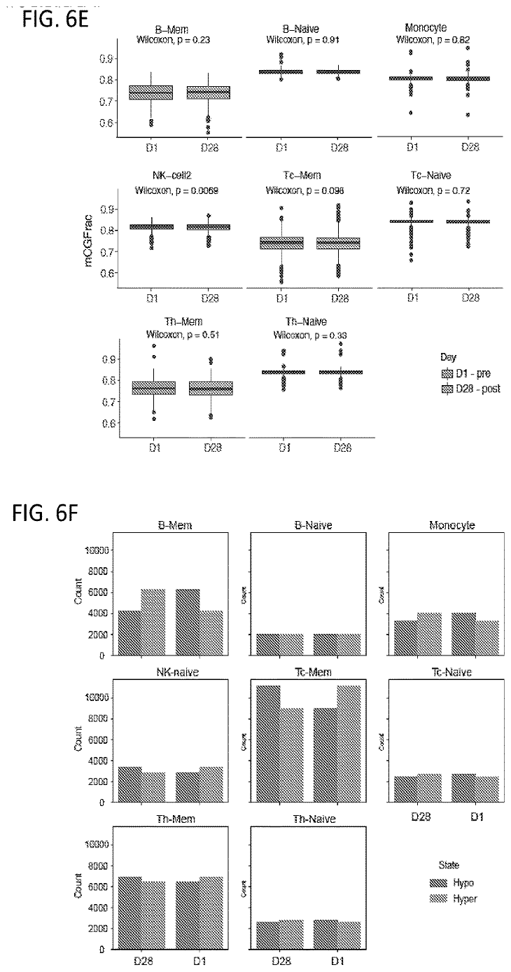

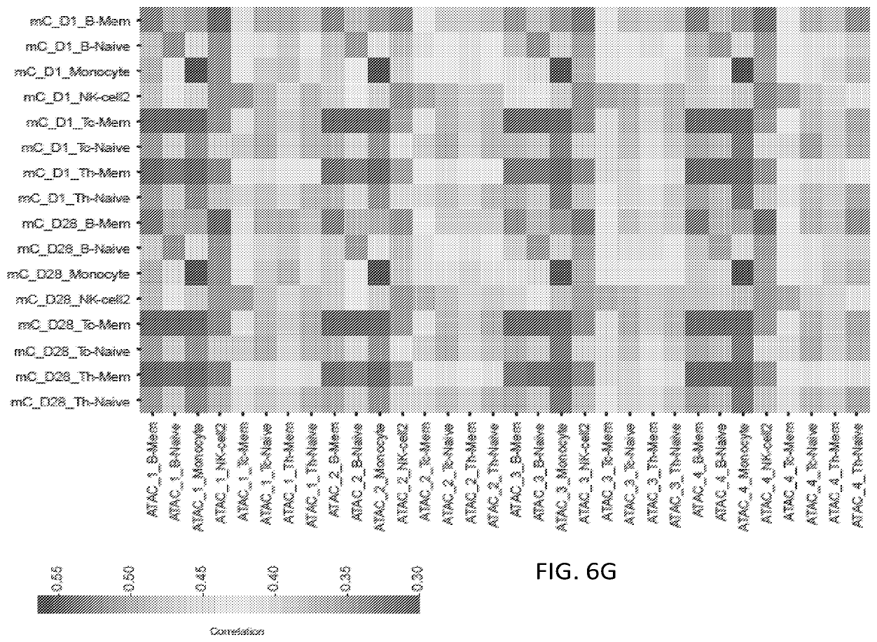

To investigate how influenza infection can affect the methylome of immune cell types, PBMCs collected from influenza virus-infected volunteers before and 28 days after infection, as part of an experimental influenza vaccine trial, were examined (FIGS. 1A-1C). Using single-cell methylation sequencing on these cells, 11 cell clusters were identified (FIG. 6A) and annotated into 9 cell types based on cell surface markers and methylation profiles (FIG. 5F). The distribution of cells from all patients was even among the clusters, indicating the absence of significant batch effects (FIG. 6B). Similar to the control and HIV cohorts, two sub-clusters of B cells and NK cells were observed, but the ratio between these sub-clusters varied significantly compared to the other cohorts. The ratio between B-Memory and B-Naive cells was 0.54:1, while the ratio between NK-Active and NK-Naive was 0.3:1 (FIG. 6D). Interestingly, NK-Active cells clustered together with memory CD8 T cells, which also exhibited lower global mCG levels (FIG. 5F), indicating similarity between NK and memory CD8 T cell methylomes.

The global mCG levels of the nine cell types did not show significant differences between the ‘pre’ and ‘post’ groups, except for some moderate changes in NK-Naive cells (FIG. 6E). Furthermore, no differentially methylated genes (DMGs) were identified between the “pre” and “post” groups, indicating that there were no drastic changes in gene body methylation 28 days after infection. To further characterize the genomic regions whose methylation is associated with influenza infection, differentially methylated regions (DMRs) were identified between the ‘pre’ and ‘post’ stages of influenza infection in all cell types. 71,995 DMRs were found to be associated with influenza infection in all cell types, with the vast majority (99.49%) being single CpG sites in CD4 memory T cells (FIG. 6F). These DMRs were equally distributed between hypo- and hyper-DMRs (FIG. 6F) and exhibited distinct patterns between the two groups (pre vs. post) (FIG. 5G).

To identify transcription factors that could bind to these DMRs, enriched motifs were identified (Heinz et al., 2010). ETS motifs were significantly enriched in both hypo- and hyper-DMRs from the ‘pre’ and ‘post’ stages in almost every cell type, except for memory B cells, active NK cells, and memory CD8 and CD4 T cells (FIG. 5H), suggesting rearrangement of ETS family transcription factor binding sites after influenza infection. Transcription factors such as PU.1, ETS1, and Fli were enriched in naive B and T cells, indicating differentiation of these naive immune cells after influenza infection. RUNX motifs were enriched in hypo-DMRs after influenza infection in naive NK cells, suggesting activation of these cells (Rapp et al., 2017). However, in naive CD8 and CD4 T cells, RUNX motifs were enriched in both hypo- and hyper-DMRs in the ‘pre’ and ‘post’ stages (FIG. 5H). Circadian transcription factor motifs were also enriched in hypo-DMRs from the ‘pre’ stage in memory and naive CD8 T cells, while they were enriched in hypo-DMRs from memory CD4 T cells after influenza infection. Enrichment of MEF2A, MEF2B, and MEF2C motifs were observed in hypo- and hyper-DMRs before influenza infection in memory B and CD8 T cells (FIG. 5H). Interestingly, MEF2 is necessary for the transcriptional activation of certain cytokines during peripheral T-cell activation (Pan et al., 2004). Similar to HIV-1 infections, the CTCF motif was enriched in naive B cells, monocytes, and NK-Naive cells, indicating potential 3D genome reorganization of these cell types after influenza infection (Rao et al., 2014; Nora et al., 2017).

To verify the quality of the single-cell methylation data and further validate the identified DMRs, the methylation data were integrated with the single-cell ATAC-seq data and transferred the cell type labels to the single-cell ATAC-seq cells. We calculated the genome-wide correlation between the hypomethylation score and the number of Tn5 insertions using 5 kb bins with the single-cell methylation and ATAC-seq data. The two modalities showed high correlation in B cells, monocytes, and NK cells, while the correlation was lower in T cells (FIG. 6G).

### Example 6

### Impact of SARS-CoV2 Infection

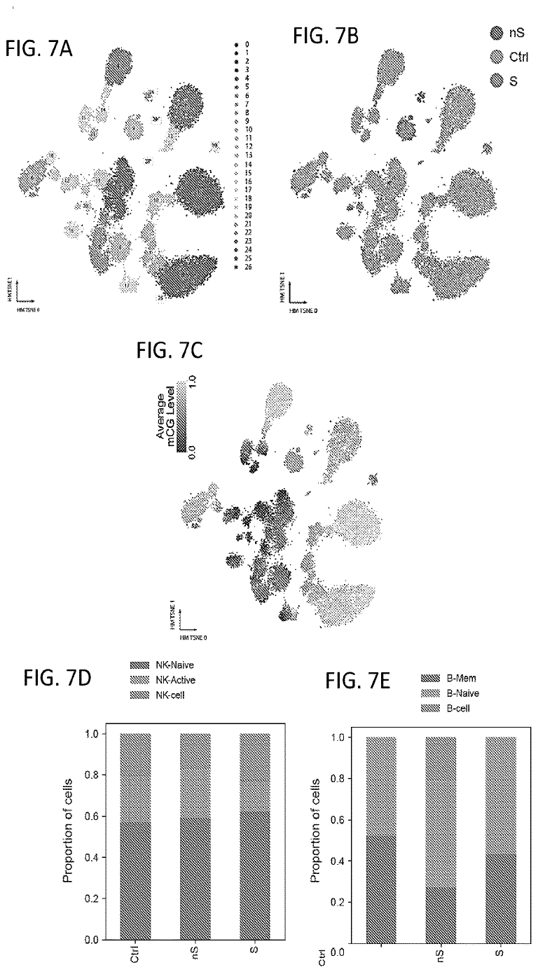

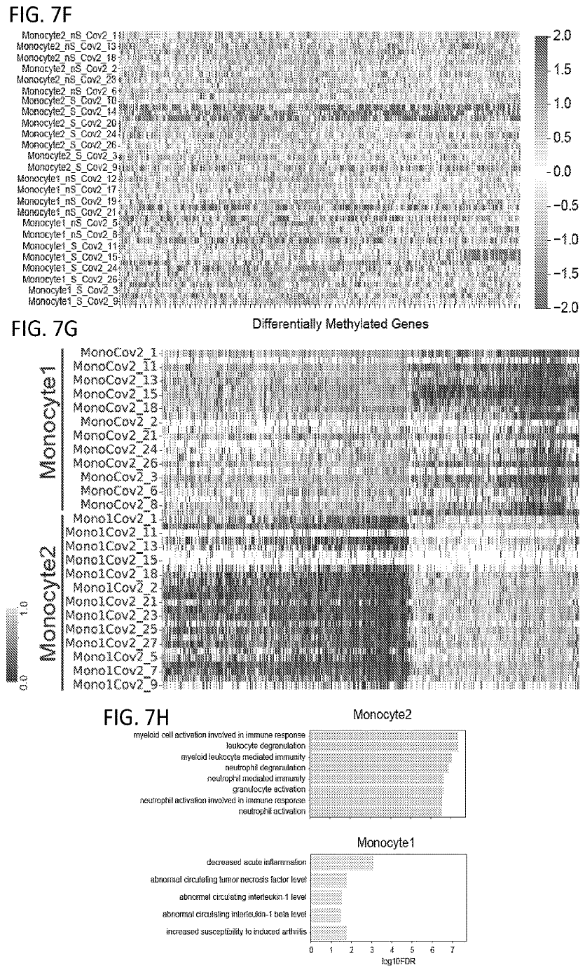

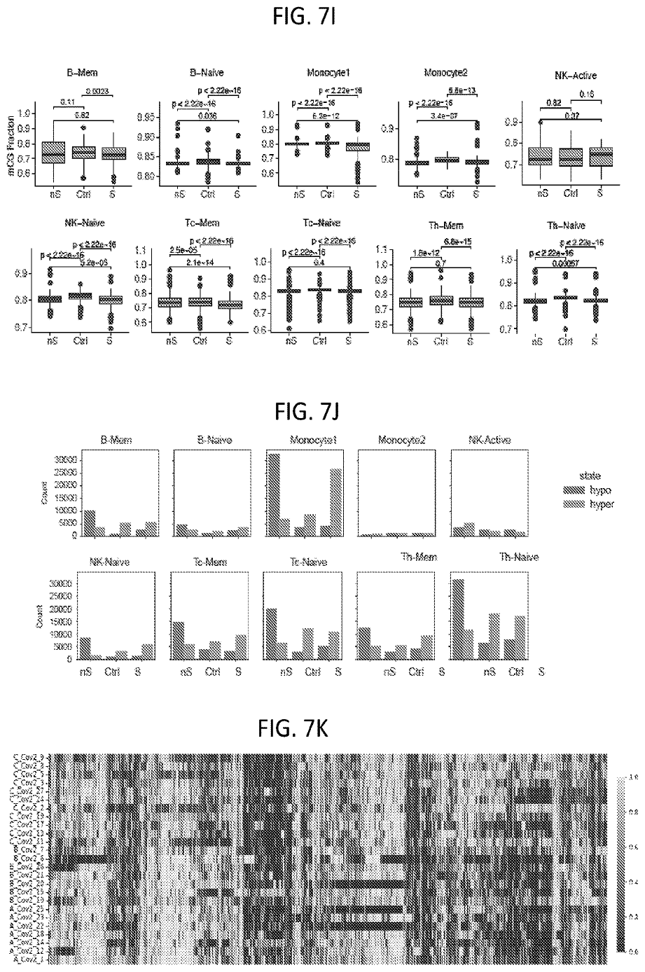

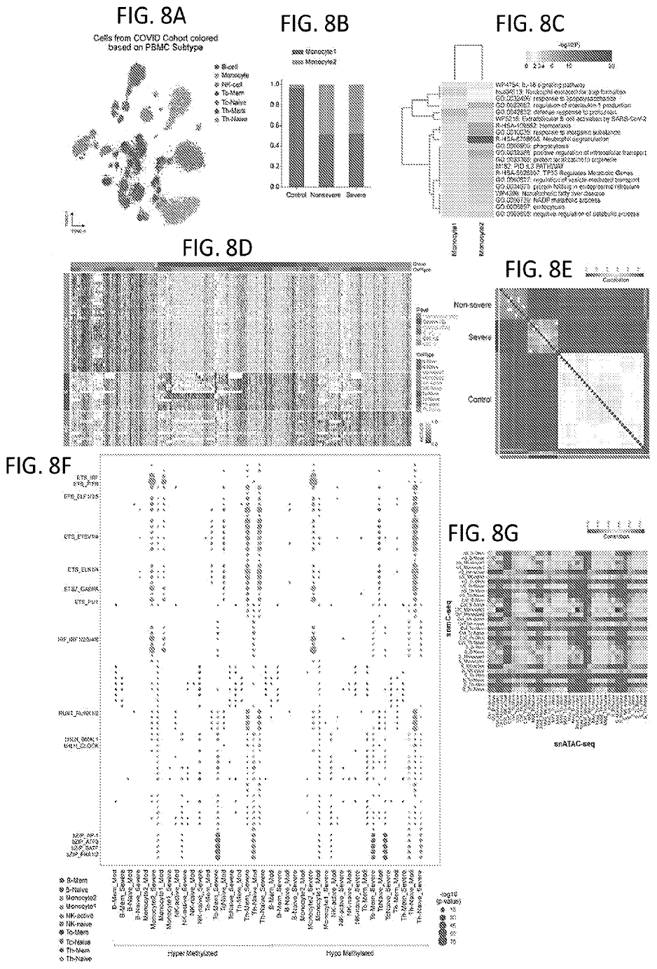

The SARS-CoV-2 virus, the pathogen causing COVID-19, continues to be a major global public health concern. However, little is known about the impact of SARS-CoV-2 infection on the dynamics of DNA methylation in immune cells. To examine this, the methylome of immune cells was profiled. Samples from patients with severe and non-severe disease were collected and 21 cell clusters identified (FIG. 7A), which were annotated to 10 cell types based on both cell surface markers and methylation profiles. In addition to two sub-clusters of B cells (B-Mem, B-Naive) and NK cells (NK-Active, NK-Naive), we also identified two populations of monocytes (FIG. 8A), present in both severe and non-severe disease patients FIG. 7B). Compared to the subclusters of B cells and NK cells, the genome-wide global mCG levels of these two clusters of monocytes are more similar (FIG. 7C). The ratios of cells in these two clusters of monocytes are comparable between severe and non-severe samples, while control monocytes are significantly enriched in one of the clusters (FIG. 8B) (P=2.05e-237, Chi-square test). We refer to the cluster of monocytes that are rare in control samples as ‘Monocyte2’, and the monocyte cluster that is abundant in control samples as ‘Monocytel’, a cluster also identified in other exposures. In addition to monocytes, the cell ratios between the two clusters of NK cells and B cells are also significantly different between non-severe, severe, and control samples (FIGS. 7D-7E) (P=2.76e-20, Chi-square test, comparison between B-Mem and B-Naive; P=3.51e-05, Chi-square test, comparison between NK-Active and NK-Naive).

To compare the differences between the two clusters of monocytes, we identified the differentially methylated genes (DMGs) and differentially methylated regions (DMRs) between them. 321 DMGs were identified between ‘Monocyte1’ and ‘Monocyte2’, of which 262 and 59 genes are hypomethylated in ‘Monocyte2’ and ‘Monocytel’, respectively (FIG. 7D). Methylation levels at these genes show distinctive patterns between the two clusters of monocytes (FIG. 7D). We also identified DMRs between the two clusters of monocytes, resulting in 118,186 DMRs, about 64,195 of which were single CpG sites. Methylation levels at these DMRs in ‘Monocytel’ and ‘Monocyte2’ across control, non-severe, and severe COVID-19 patients showed high reproducibility (FIG. 7G). We further examined gene functional enrichment of these DMRs for each cluster and found that hypo-DMRs in ‘Monocyte2’ are enriched in ‘myeloid cell activation’ and ‘leukocyte degranulation’, while hypo-DMRs in ‘Monocytel’ are enriched in functions related to ‘abnormal circulating IL-1 level’ (FIG. 7H). IL-1 has a pivotal role in the induction of cytokine storms due to uncontrolled immune responses in SARS-CoV-2 infection (Mardi et al., 2021). IL-1 inhibition improved clinical outcomes of severe COVID-19 patients, and this treatment is one of the CDC COVID-19 treatment guidelines.

To compare the methylomes of these immune cell types, global mCG was determined in control, non-severe, and severe COVID-19 samples. Global mCG was significantly different in the two clusters of monocytes, naive NK cells, naive CD4, and CD8 T cells (FIG. 7I). To further determine the methylation signatures associated with non-severe or severe COVID-19, the DMRs in each of the ten immune cell type clusters were identified. In total, 203,809 DMRs were identified, of which 84.06% (171,319) are single CpG sites. Consistent with the significant change of global mCG level in ‘Monocytel’, this sub-cluster had the largest number of DMRs compared to all other cell types, being hypomethylated in severe COVID-19 patients compared with control samples (FIG. 7J), indicating dynamic reshaping of the methylome in severe COVID-19 patients. Comparison of the methylation levels at these DMRs in control non-severe and severe COVID-19 samples revealed distinctive patterns between patient groups (FIG. 8D). Further correlation analysis of DMRs between these samples showed a higher correlation between non-severe and control samples than between non-severe and severe COVID-19 patients (FIG. 8E). To further verify that the DMRs are associated with COVID-19, the labels of the samples were shuffled and DMR analysis performed between the randomly grouped samples, which showed that the DMRs are less distinctive compared to the DMRs between COVID-19 samples and controls (FIG. 8E).

To further identify the transcription factor activities associated with non-severe or severe COVID-19, the enrichment of DNA binding motifs at DMRs were examined in the ten immune cell clusters. Control hypo-DMRs in Monocytel, which are also mostly hypermethylated in severe and non-severe COVID-19 samples, were significantly enriched in IRF and ETS motifs (FIG. 8F). The ETS family motifs were also enriched in the control hypo-DMRs and COVID-19 hyper-DMRs in naive CD4 and CD8 T cells (FIG. 8F). AP-1 family transcription factors are specifically enriched in the hypo-DMRs in COVID-19 patients and hyper-DMRs in controls in naive CD4 and CD8 T cells FIG. 8F), suggesting the activation of these cells after SARS-CoV-2 infection (Yukawa et al., 2020). These AP-1 motifs are also enriched in hypo-DMRs in monocytes in severe COVID-19 patients, and loss of methylation at AP-1 binding sites was also reported from monocyte-to-macrophage differentiation (Dekkers et al., 2019), indicating the potential differentiation of these monocytes. bHLH transcription factors, which include the circadian genes, are also enriched in most of the DMRs in different cell types, with their motifs being enriched in T cells in HIV-1 and influenza cohorts.

As described above, integration between the methylation data and single-cell ATAC-seq data was performed. To validate the integration approach and data quality, the genome-wide correlation was calculated between these two modalities (FIG. 8G). The two modalities (methylation and open chromatin) were strongly correlated across all cell types, with the strongest correlation observed in monocytes and the lowest correlation in different types of T cells (FIG. 8G). Surprisingly, only −5% of the DMRs in each cell type overlapped with a peak in the corresponding cell type in ATAC-seq data, indicating that most of the methylation changes associated with COVID-19 are in inaccessible chromatin.

### Example 7

### Impact of MRSA and MSSA Infection on the Methylome

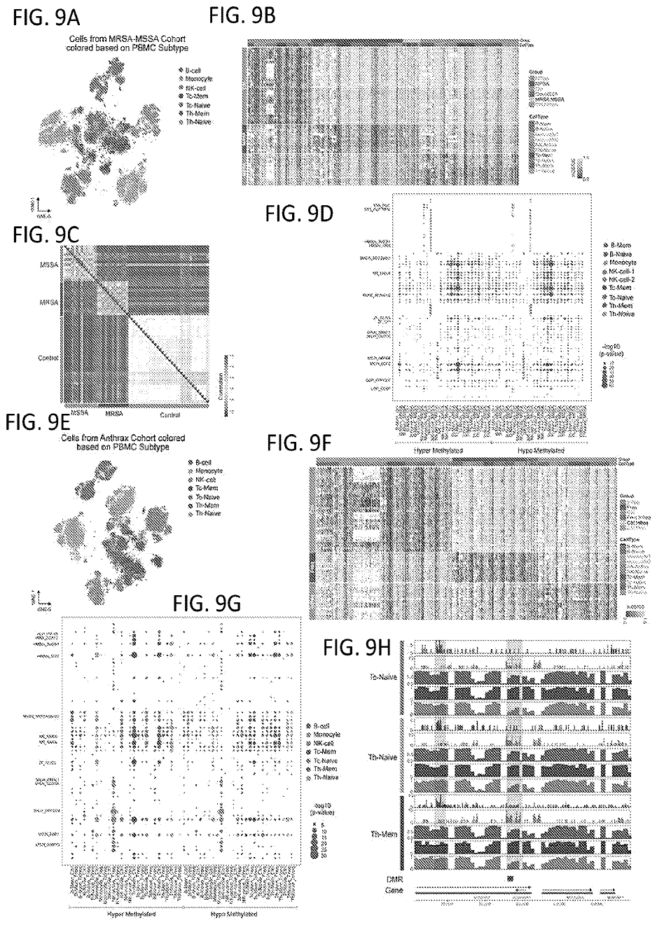

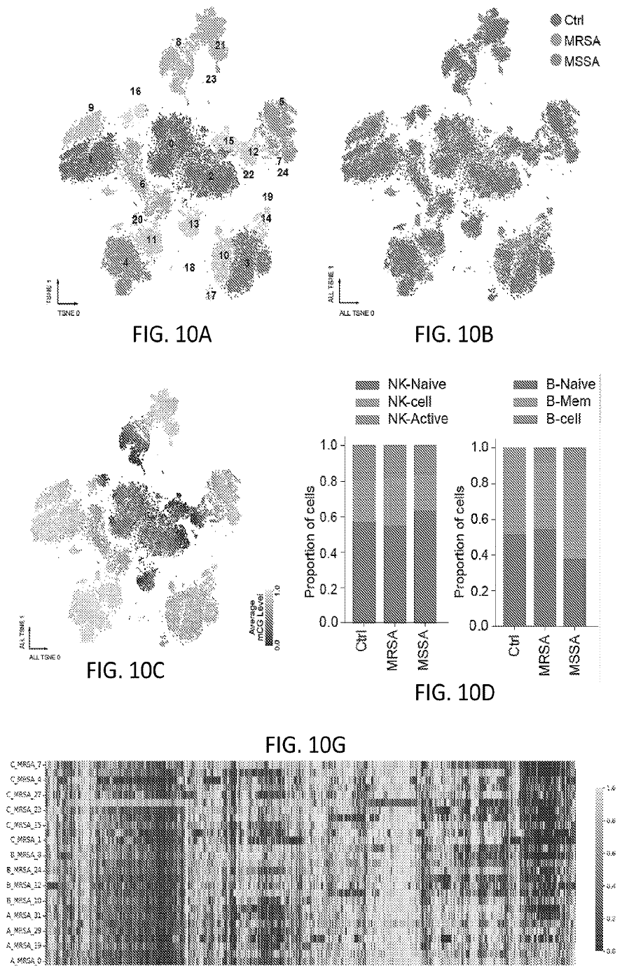

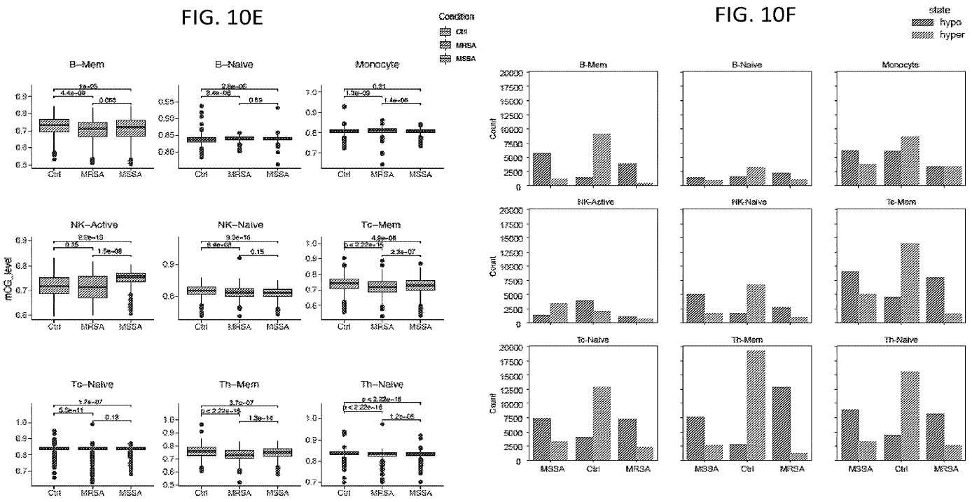

Methicillin-resistant Staphylococcus aureus (MRSA) and methicillin-susceptible Staphylococcus aureus (MSSA) are both caused by the same strain of bacteria which differ in their susceptibility to antibiotics. To better understand the impact of this infection on the immune cell methylome, snmC-seq2 was performed on the MRSA-MSSA cohort described above. Based on the global methylation profile of the cells, 25 clusters were identified, which were annotated to 9 cell types based on both cell surface markers and methylation profiles (FIGS. 9A, 10A). These included two sub-clusters of B cells and NK cells, which were labeled as, B-Mem, B-Naive, NK-Active, and NK-Naive, respectively (FIG. 10C). These clusters were distributed evenly among MRSA, MSSA patients, and control samples (FIG. 10B). The number of cells in the two sub-clusters of NK cells was comparable in the three cohorts (FIG. 10D), while we captured more memory B cells in the MSSA cohort compared to the other two (FIG. 10D) (P=9.36e-10, Chi-square test).

Large changes in global mCG levels were observed between MRSA, MSSA, and controls in all nine cell types (FIG. 10E). To further characterize the methylation signatures correlated with MRSA or MSSA infection, DMRs between MRSA, MSSA, and control samples were identified. 134,868 DMRs were identified between the three groups, the majority of which were from memory CD8 T cells (FIG. 10F), which also showed a significant change in global mCG (FIG. 10E). Similar to other exposures, the majority (93.46%) of DMRs were single CpG sites. Visualization of the methylation levels at these DMRs demonstrated high reproducibility, showing a clear distinction between patient and control samples (FIG. 9B). The correlation of the methylation level of samples from the three cohorts at these DMRs confirmed that the samples from the three groups are very distinctive from each other at the DMRs (FIG. 9C). To further validate that the MRSA or MSSA-associated DMRs identified are not caused by heterogeneity between individuals, the labels of the samples were shuffled as described above and identified the DMRs between these random groups. The DMRs between random groups are much less distinctive than MRSA or MSSA-associated DMRs (FIG. 10G).

To identify transcription factors that can potentially bind at these DMRs, the enrichment of DNA binding motifs was investigated. Unlike the virus infections, both RUNX and ETS motifs are only enriched in the hypo-DMRs of MSSA and hyper-DMRs in controls in active NK cells, with some of these motifs enriched in hypo-DMRs of controls and hyper-DMRs of MRSA in monocytes (FIG. 9D). Homeobox and MADS family motifs are generally in all cell types and in both hypo- and hyper-DMRs, indicating reshuffling of the binding sites after MRSA or MSSA infections.

### Example 8

### Impact of Frequency of Exposures in High-Risk Facilities on the Methylome

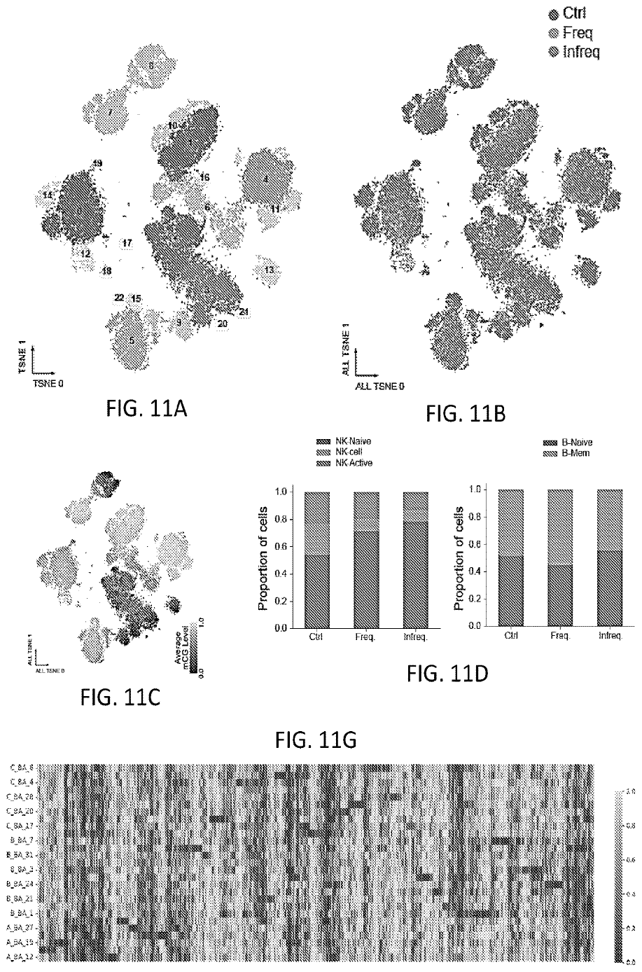

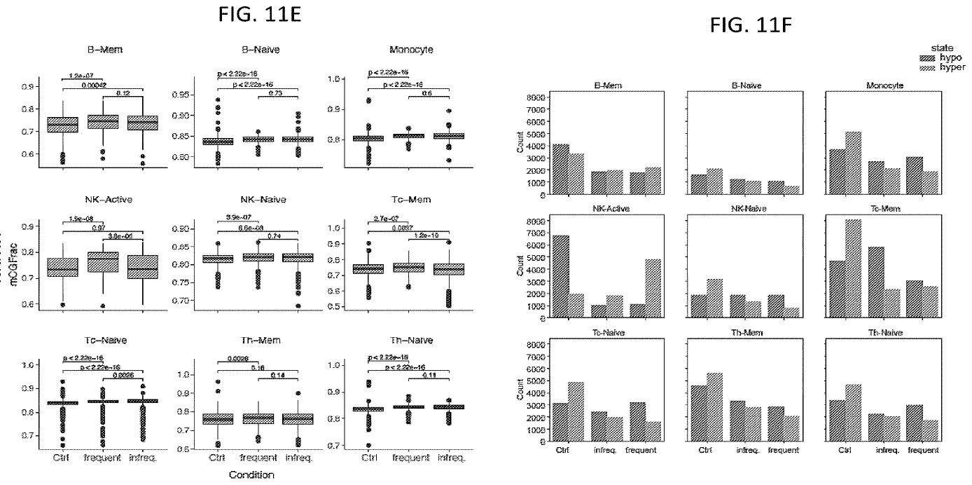

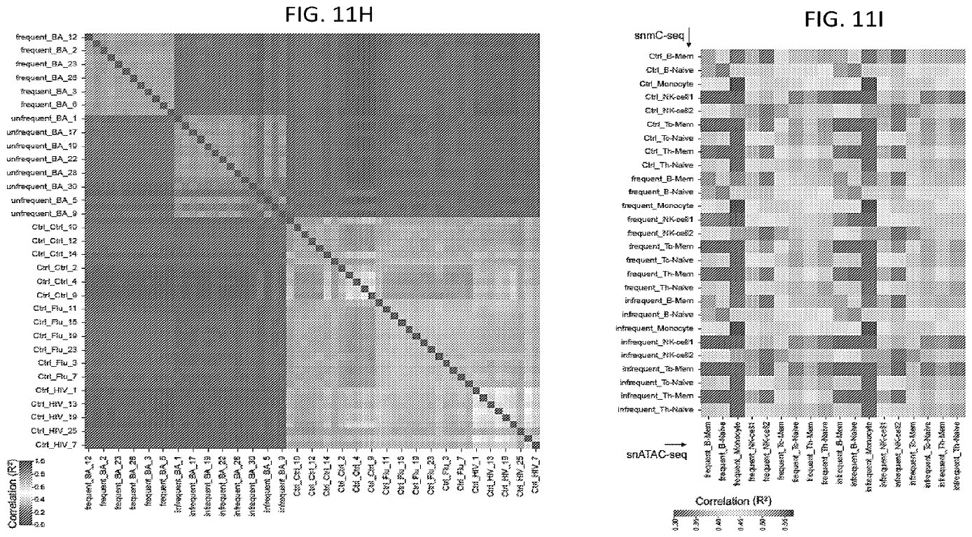

Bacillus anthracis (BA) is the causative agent of anthrax. In the BA cohort, PMBCs were collected from individuals who received BA vaccines and are frequently or infrequently working in BSL3 facilities, dealing with infectious pathogens or chemicals. Methylation profiles of immune cells identified 23 clusters, further categorized into 9 cell types based on cell surface markers and methylation profiles (FIGS. 9E, 11A). Immune cells from technicians who handled anthrax, both frequently and infrequently, clustered together across all cell types (FIG. 11B). Based on global mCG levels (FIG. 11C), two clusters of B cells (B-Mem and B-Naive) and two clusters of NK cells (NK-Active and NK-Naive) were identified. The ratio of NK-Active cells in the ‘frequent’ or ‘infrequent’ groups was significantly lower than in the control samples (P=1.74e-64, Chi-square test), while the ratio of memory and naive B cells was comparable across the three cohorts (FIG. 11D).

Significant differences in global mCG levels were observed among these immune cell clusters across the three groups (FIG. 11E). To explore the epigenetic characteristics of immune cells in individuals handling anthrax frequently or infrequently in BSL3 facilities, differentially methylated regions (DMRs) were identified between these groups compared to a control population. 76,757 DMRs were found among the three groups, with 72,837 (95.12%) consisting of single CpG sites. Control samples displayed the highest number of hypo- or hyper-DMRs in all cell types (FIG. 11F), suggesting that the observed differences were mainly between the control and BA cohorts. Visualization of the methylation levels at these DMRs clearly distinguished the two groups and the control cohort (FIG. 9B). A permutation test similar to the one described above was performed, and it was found that DMRs between random groups were less distinctive ((FIG. 11G). The correlation of methylation levels at these DMRs in donors from the three groups also exhibited higher correlations within groups (FIG. 11H).

To identify potential transcription factors that could bind to these DMRs, an enrichment analysis of DNA binding motifs for each group was performed. Unlike viral infections and similar to the MRSA and MSSA cohort, both RUNX and ETS motifs were not enriched in the DMRs across all cell types (FIG. 9G). Circadian transcription factors showed significant enrichment only in hypo-DMRs from control samples in active NK cells (FIG. 9G). Homeobox family transcription factor motifs were enriched in hypo-DMRs in active NK cells from control samples (FIG. 9G). These homeobox genes act as transcriptional regulators of embryonic development and cell differentiation and have also been reported to regulate NK cell function (Sunwoo et al., 2008; Wu, Zhang, and Zhang, 2011), suggesting potential immune cell differentiation.

The methylation data was integrated with single-cell ATAC-seq data. To validate the integration approach and data quality, the genome-wide correlation was calculated between these two modalities. Similar correlations to other exposures were observed between the two modalities across all cell types (FIG. 11I). Simultaneous loss of methylation and gain of accessibility, as well as gain of methylation and loss of accessibility, was observed in the ‘frequent’ group at a locus containing a long non-coding RNA (lncRNA) and a pseudogene (FIG. 9H). Only approximately 5.5% of the DMRs in each cell type overlapped with a peak in the corresponding cell type in the ATAC-seq data, indicating that most of the methylation changes associated with working frequency in high-risk facilities occur in closed chromatin regions.

### Example 9

### Impact of Chemical Exposure on the Methylome

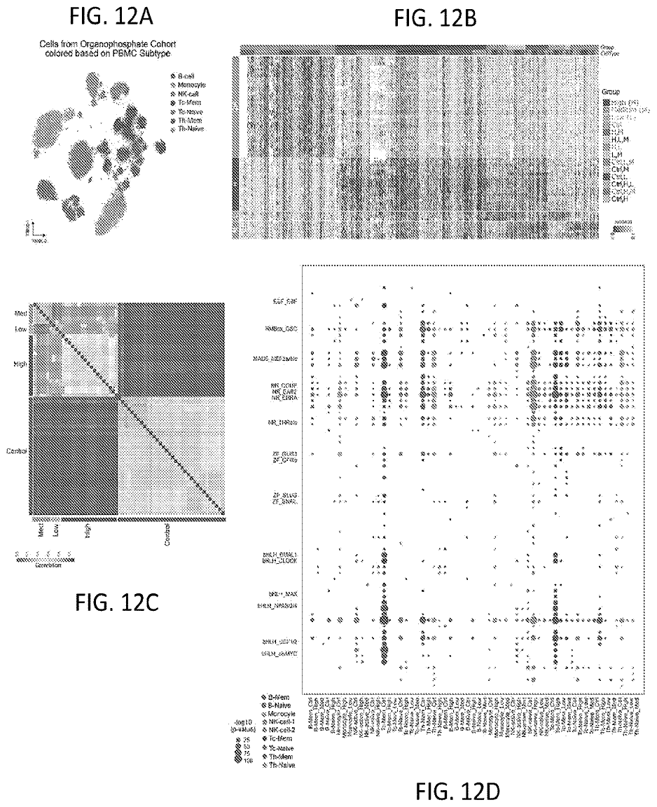

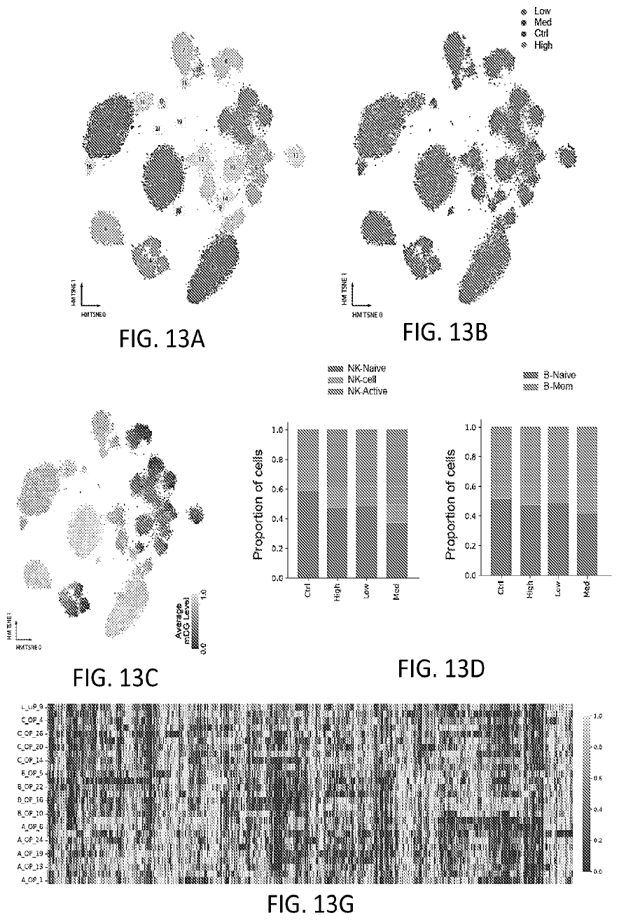

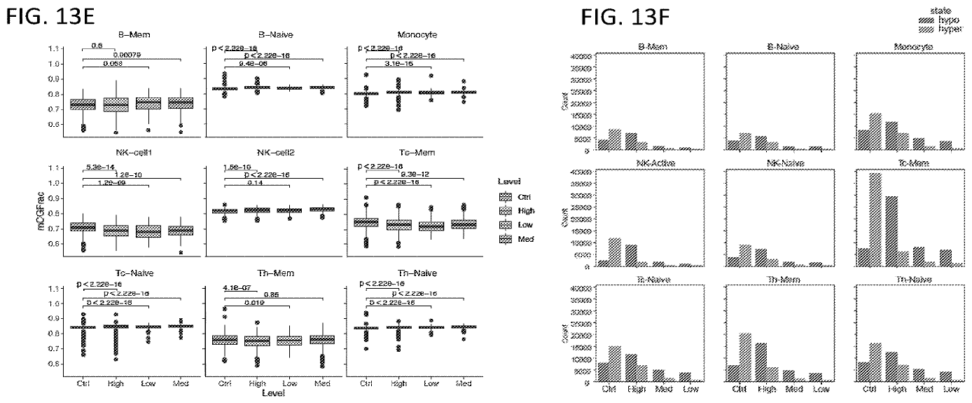

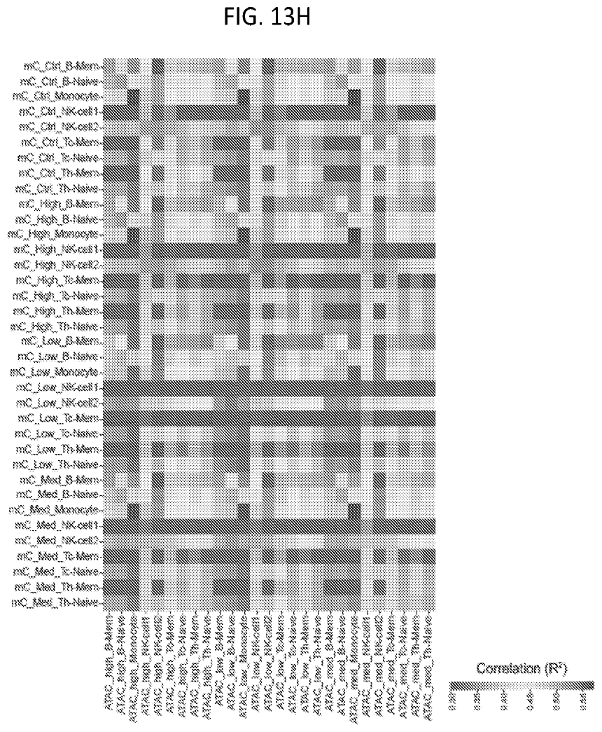

Organophosphates (OP) include insecticides that can harm humans and animals if exposed for too long or at high levels. However, little is known about how this chemical can affect the methylome of immune cells when exposed at different levels. We performed snmC-seq2 on the PBMCs of people exposed to OP at different levels (Low, Med and High). These immune cells clustered into 22 clusters and were further annotated to 9 cell types based on both cell surface markers and methylation profiles (FIGS. 12A, 13A). Cells from patients with different levels of exposure to OP are evenly distributed across the cell types (FIG. 13B). As with other exposures, B-Mem, B-Naive, NK-Active, and NK-Naive were identified based on their global methylation levels (FIG. 13C). The proportions of the two clusters of B cells are comparable across different levels of exposure, while exposure groups have significantly more NK-Active cells than in control samples (FIG. 13D) (P=1.44e-56, Chi-square test).

We explored the association of methylation changes with different levels of OP exposure and observed significant changes in the global mCG levels in most of the cell types (FIG. 13E). DMRs were identified between the different exposure groups and control samples in all nine cell types, resulting in 198,807 DMRs among the four groups (Low, Med, High, and Control). Memory CD8 T cells had the highest number of DMRs compared to other cell types (FIG. 13F). Visualization of the methylation levels at these DMRs showed distinct methylation patterns between these groups (FIG. 12B), particularly between OP exposure and control. A similar permutation test was performed as described above and the DMRs between random groups were much less distinctive (FIG. 13G). The correlation analysis of these samples at the DMRs revealed weak distinctions between the ‘Low’, ‘Med’, and ‘High’ group samples, while the distinction between control samples was evident (FIG. 12C).

To further investigate which transcription factors might be affected in these nine cell types, the enriched motifs at these DMRs across all cell types were determined. Hyper-DMRs in control samples and hypo-DMRs in OP exposure groups were enriched in MADS, RN, and Homeobox family transcription factor motifs in all cell types (FIG. 12D), indicating that the impact of this chemical exposure on the methylome does not exhibit cell type specificity.

Integration between the methylation data and single-cell ATAC-seq data was performed. To validate the integration approach and data quality, we calculated the genome-wide correlation between these two modalities. Similar correlations to other exposures between the two modalities in all cell types was observed (FIG. 13H). Consistent with other exposures, the majority of DMRs associated with OP were located in inaccessible chromatin regions.

### Example 10

### Comparison of Methylation Signatures from all Exposures

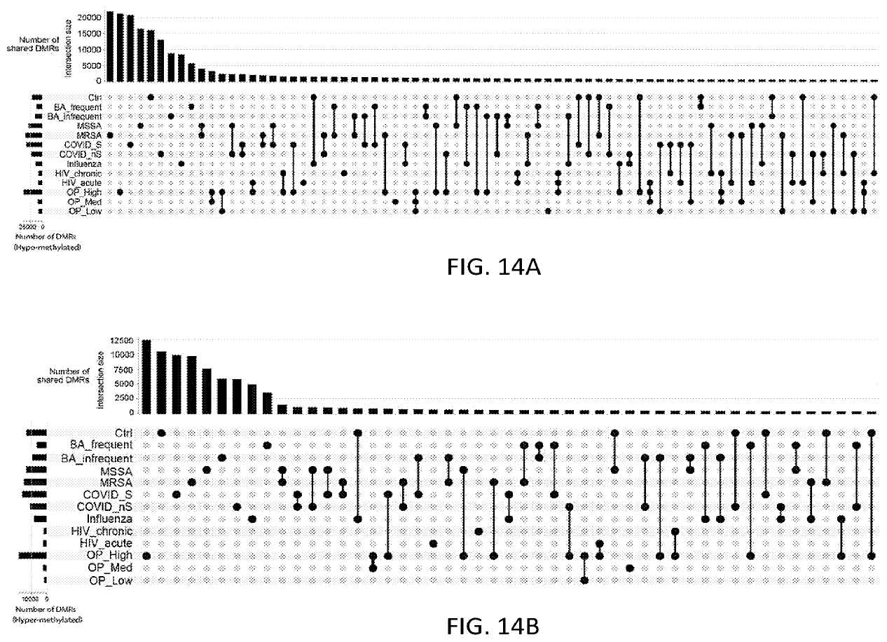

To compare the methylation signatures across different exposures, the methylation files from the same exposure were merged and the differentially methylated regions (DMRs) among all the groups identified using methylpy. This strategy does not rely on control samples and allows for a general comparison of the methylation levels across all the samples. For instance, in Naive B cells, 188,563 hypo-DMRs were identified across all exposures, most of which were unique to a specific exposure (FIG. 14A). Interestingly, MRSA and MSSA shared more DMRs than any other two exposures, which aligns with the similarities observed between these two exposures (FIG. 14A). Different levels of OP exposures shared more hypo-DMRs compared to other comparisons, indicating a similar effect of varying levels of OP exposures. However, bacterial exposures and virus exposures did not show a higher number of shared DMRs within the group compared to between groups, as MRSA/MSSA and COVID-19 samples shared more DMRs than any two exposures within bacteria or virus (FIG. 14A).

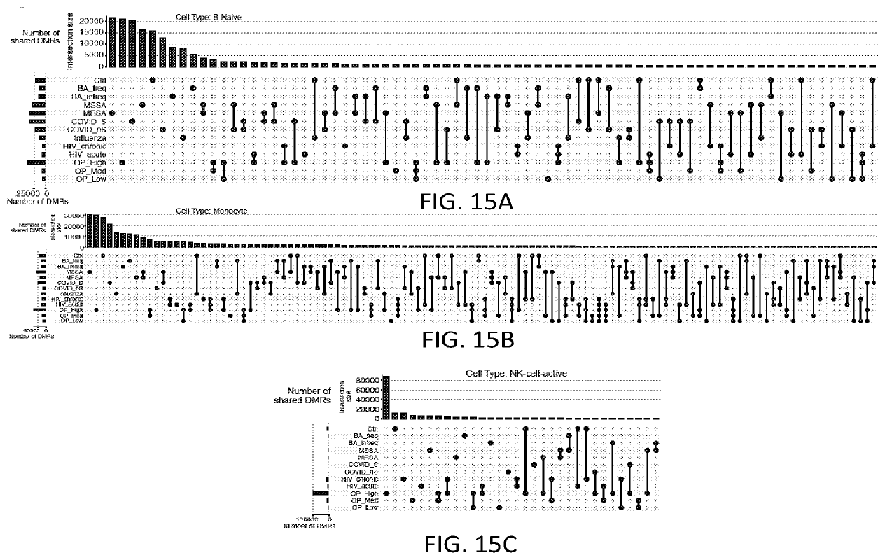

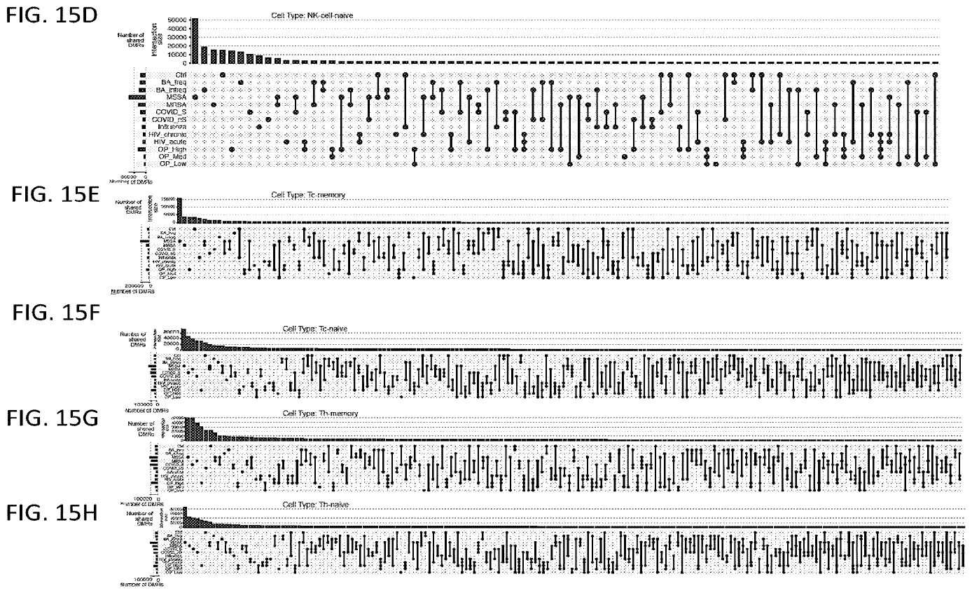

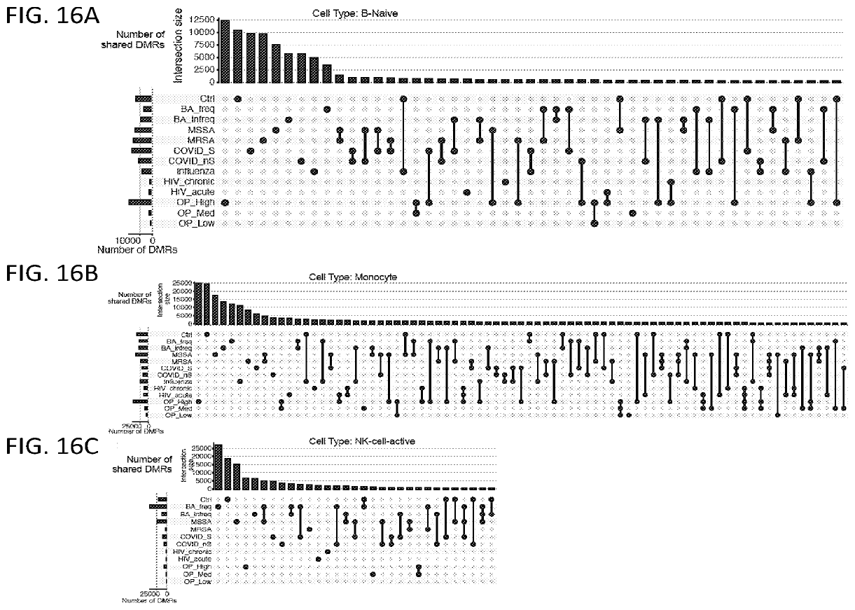

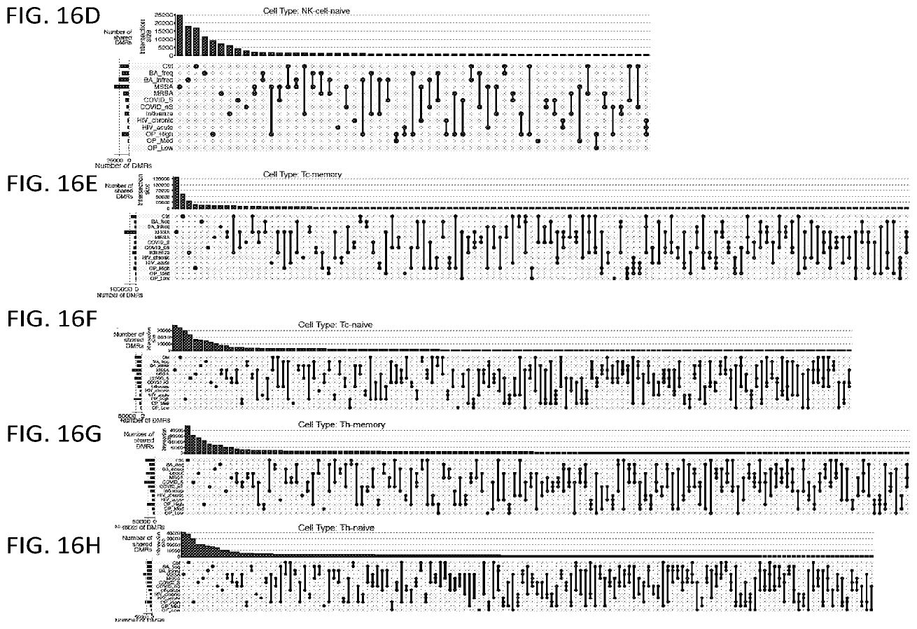

A similar analysis was performed for hyper-DMRs among all exposures, which yielded comparable results to the hypo-DMRs (FIG. 14B). The same analysis was applied to all other cell types, which demonstrated similar patterns of similarities in DMRs among all exposures for both hypo-DMRs (FIGS. 15A-15H) and hyper-DMRs (FIGS. 16A-16H).

### Example 11

### Predicting Exposure Types with Single-Cell Methylomes

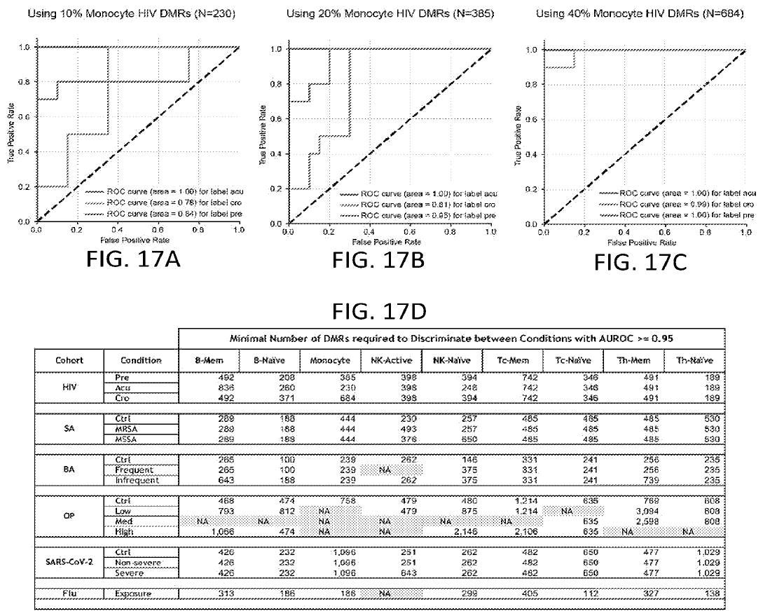

Considering that 790,662 differentially methylated regions (DMRs) were associated with various exposures in the nine immune cell types, it was hypothesized that some of these DMRs could accurately predict different exposures. Multiple rounds of training were performed to identify the optimal number of DMRs for accurate prediction. As an example, DMRs associated with HIV-1 infection were used to predict its three stages (pre, acute, chronic). We divided the individual donors into training and test cohorts and created 150 and 50 pseudo-individuals by combining single cells through permutation. Using logistic regression, a model with the 150 pseudo-individuals were trained in the training cohort for each cell type and tested the model with pseudo-individuals in the test cohort. Remarkably, the model was able to perfectly predict the different stages of HIV-1 infection. Next, the number of DMRs required for accurate prediction was minimized by selecting the top weighted DMRs in the primary prediction at the 10% quantiles. Surprisingly, perfect prediction of different HIV-1 infection stages was achieved using only 30% of the DMRs, and the pre-stage could be predicted with just 10% of the DMRs (FIGS. 17A-17C).

The prediction analysis was expanded to other exposures, and the results demonstrated that most exposures were predicted with an area under the curve (AUC) greater than 0.95 in receiver operating characteristic (ROC) curves using only a few thousand DMRs (FIG. 17D). Exemplary minimum discriminative DMRs are provided in Table 1, which can serve as biomarkers for diagnosing different exposures using readily accessible cells.

### REFERENCES

1. Bloom et al., 2019. “Infectious Disease Threats in the Twenty-First Century: Strengthening the Global Response.” Frontiers in Immunology. https://doi.org/10.3389/fimmu.2019.00549.
2. Bower et al., 2022. “What Is Anthrax?” Pathogens 11 (6). https://doi.org/10.3390/pathogens11060690.
3. Calle-Fabregat et al., 2020. “Understanding the Relevance of DNA Methylation Changes in Immune Differentiation and Disease.” Genes 11 (1). https://doi.org/10.3390/genesl1010110.
4. Chang et al., 2021. “Human DNA methylation signatures differentiate persistent from resolving MRSA bacteremia.” Proceedings of the National Academy of Sciences 118 (10): e2000663118.
5. Dale et al., 2011. “Pybedtools: A Flexible Python Library for Manipulating Genomic Datasets and Annotations.” Bioinformatics 27 (24): 3423-24.
6. Dekkers et al., 2019. “Human Monocyte-to-Macrophage Differentiation Involves Highly Localized Gain and Loss of DNA Methylation at Transcription Factor Binding Sites.” Epigenetics & Chromatin 12 (1): 34.
7. Fang, et al., 2021. “Comprehensive Analysis of Single Cell ATAC-Seq Data with SnapATAC.” Nature Communications 12 (1): 1337.
8. Frias et al., 2021. “HIV-Specific CD8 T Cells from Elite Controllers Have an Epigenetic Imprint That Preserves Effector Functions.” bioRxiv. https://doi.org/10.1101/2021.09.28.459512.
9. Gao et al., 2015. “GATA Family Transcriptional Factors: Emerging Suspects in Hematologic Disorders.” Experimental Hematology & Oncology 4 (October): 28.
10. Grant et al., 2010. “Preexposure Chemoprophylaxis for HIV Prevention in Men Who Have Sex with Men.” The New England Journal of Medicine 363 (27): 2587-99.
11. Heinz et al., 2010. “Simple Combinations of Lineage-Determining Transcription Factors Prime Cis-Regulatory Elements Required for Macrophage and B Cell Identities.” Molecular Cell 38 (4): 576-89.
12. Hertz-Picciotto et al., 2018. “Organophosphate Exposures during Pregnancy and Child Neurodevelopment: Recommendations for Essential Policy Reforms.” PLoS Medicine 15 (10): e1002671.
13. Hollenhorst et al., 2011. “Genomic and Biochemical Insights into the Specificity of ETS Transcription Factors.” Annual Review of Biochemistry 80: 437-71.
14. Hosokawa et al., 2020. “How transcription factors drive choice of the T cell fate.” Nature Reviews. Immunology 21 (3): 1-15.
15. Ji et al., 2010. “Comprehensive Methylome Map of Lineage Commitment from Haematopoietic Progenitors.” Nature 467 (7313): 338-42.
16. Konigsberg et al., 2021. “Host Methylation Predicts SARS-CoV-2 Infection and Clinical Outcome.” Communication & Medicine 1 (1): 42.
17. Korinfskaya et al., 2021. “Runx Transcription Factors in T Cells-What Is Beyond Thymic Development?” Frontiers in Immunology 12 (August): 701924.
18. Korsunsky et al., “Fast, Sensitive, and Accurate Integration of Single Cell Data with Harmony.” https://doi.org/10.1101/461954.
19. Korsunsky et al., 2019. “Fast, Sensitive and Accurate Integration of Single-Cell Data with Harmony.” Nature Methods 16 (12): 1289-96.
20. Liao et al., 2013. “The Subread Aligner: Fast, Accurate and Scalable Read Mapping by Seed-and-Vote.” Nucleic Acids Research 41 (10): e108.
21. Li et al., 2009. “The Sequence Alignment/Map Format and SAMtools.” Bioinformatics 25 (16): 2078-79.
22. Liu et al., 2021. “DNA Methylation Atlas of the Mouse Brain at Single-Cell Resolution.” Nature 598 (7879): 120-28.
23. Liu et al., 2021. “Single-cell analysis of COVID-19, sepsis, and HIV infection reveals hyperinflammatory and immunosuppressive signatures in monocytes.” Cell Reports 37 (1): 109793.
24. Luo et al., 2017. “Single-cell methylomes identify neuronal subtypes and regulatory elements in mammalian cortex.” Science 357 (6351): 600-604.
25. Luo et al., 2018. “Robust Single-Cell DNA Methylome Profiling with snmC-seq2.” Nature Communications 9 (1): 3824.
26. Mardi et al., 2021. “Interleukin-1 in COVID-19 Infection: Immunopathogenesis and Possible Therapeutic Perspective.” Viral Immunology 34 (10): 679-88.
27. Martin et al., 2018. “Environmental Influences on the Epigenome: Exposure-Associated DNA Methylation in Human Populations.” Annual Review of Public Health 39 (April): 309-33.
28. McLaren, Paul J., and Jacques Fellay. 2021. “HIV-1 and Human Genetic Variation.” Nature Reviews. Genetics 22 (10): 645-57.
29. McLean, Cory Y., Dave Bristor, Michael Hiller, Shoa L. Clarke, Bruce T. Schaar, Craig B. Lowe, Aaron M. Wenger, and Gill Bejerano. 2010. “GREAT Improves Functional Interpretation of Cis-Regulatory Regions.” Nature Biotechnology 28 (5): 495-501.
30. Meier, Ute-Christiane, Rachel E. Owen, Elizabeth Taylor, Andrew Worth, Nikolai Naoumov, Christian Willberg, Kwok Tang, et al. 2005. “Shared Alterations in NK Cell Frequency, Phenotype, and Function in Chronic Human Immunodeficiency Virus and Hepatitis C Virus Infections.” Journal of Virology 79 (19): 12365-74.
31. Minderjahn, Julia, Andreas Schmidt, Andreas Fuchs, Rudolf Schill, Johanna Raithel, Magda Babina, Christian Schmidl, et al. 2020. “Mechanisms governing the pioneering and redistribution capabilities of the non-classical pioneer PU.1.” Nature Communications 11 (1): 402.
32. Morbach, H., E. M. Eichhorn, J. G. Liese, and H. J. Girschick. 2010. “Reference Values for B Cell Subpopulations from Infancy to Adulthood.” Clinical and Experimental Immunology 162 (2): 271-79.
33. Nandhini et al., 2022. “Recent Developments in Methicillin-Resistant Staphylococcus Aureus (MRSA) Treatment: A Review.” Antibiotics (Basel, Switzerland) 11 (5). https://doi.org/10.3390/antibiotics11050606.
34. Pacis et al., 2015. “Bacterial Infection Remodels the DNA Methylation Landscape of Human Dendritic Cells.” https://doi.org/10.1101/016022.
35. Paglia et al., 2016. “Runxl Regulates Myeloid Precursor Differentiation Into Osteoclasts Without Affecting Differentiation Into Antigen Presenting or Phagocytic Cells in Both Males and Females.” Endocrinology 157 (8): 3058-69.
36. Pan et al., 2004. “Myocyte Enhancer Factor 2 Mediates Calcium-Dependent Transcription of the Interleukin-2 Gene in T Lymphocytes: A Calcium Signaling Module That Is Distinct from but Collaborates with the Nuclear Factor of Activated T Cells (NFAT).” The Journal of Biological Chemistry 279 (15): 14477-80.
37. Park, Yongseok, and Hao Wu. 2016. “Differential Methylation Analysis for BS-Seq Data under General Experimental Design.” Bioinformatics 32 (10): 1446-53.
38. Powell et al., 2016. “Opportunistic Infections in HIV-Infected Patients Differ Strongly in Frequencies and Spectra between Patients with Low CD4+ Cell Counts Examined Postmortem and Compensated Patients Examined Antemortem Irrespective of the HAART Era.” PloS One 11 (9): e0162704.
39. Qin et al., 2021. “The Role of Host Cell DNA Methylation in the Immune Response to Bacterial Infection.” Frontiers in Immunology 12 (July): 696280.
40. Quinlan, Aaron R., and Ira M. Hall. 2010. “BEDTools: A Flexible Suite of Utilities for Comparing Genomic Features.” Bioinformatics 26 (6): 841-42.
41. Rapp et al., 2017. “Core-Binding Factor β and Runx Transcription Factors Promote Adaptive Natural Killer Cell Responses.” Science Immunology 2 (18). https://doi.org/10.1126/sciimmunol.aan3796.
42. Roy et al., 2021. “DNA Methylation Signatures Reveal That Distinct Combinations of Transcription Factors Specify Human Immune Cell Epigenetic Identity.” Immunity 54 (11): 2465-80.e5.
43. Sachs et al., 2022. “The Lancet Commission on Lessons for the Future from the COVID-19 Pandemic.” The Lancet 400 (10359): 1224-80.
44. Schultz et al., 2016. “Corrigendum: Human Body Epigenome Maps Reveal Noncanonical DNA Methylation Variation.” Nature 530 (7589): 242.
45. Sharrocks, A. D. 2001. “The ETS-Domain Transcription Factor Family.” Nature Reviews. Molecular Cell Biology 2 (11): 827-37.
46. Sosa-Hernández, Victor A., Jiram Torres-Ruíz, Rodrigo Cervantes-Díaz, Sandra Romero-Ramírez, José C. Páez-Franco, David E. Meza-Sánchez, Guillermo Juárez-Vega, et al. 2020. “B Cell Subsets as Severity-Associated Signatures in COVID-19 Patients.” Frontiers in Immunology 11. https://doi.org/10.3389/fimmu.2020.611004.
47. Stuart et al., 2019. “Comprehensive Integration of Single-Cell Data.” Cell 177 (7): 1888-1902.e21.
48. Sunwoo et al., 2008. “Distal-Less Homeobox Transcription Factors Regulate Development and Maturation of Natural Killer Cells.” Proceedings of the National Academy of Sciences of the United States of America 105 (31): 10877-82.
49. Swartz, M. N. 2001. “Recognition and Management of Anthrax—an Update.” The New England Journal of Medicine 345 (22): 1621-26.
50. Tanigawa et al., 2022. “WhichTF Is Functionally Important in Your Open Chromatin Data?” PLoS Computational Biology 18 (8): e1010378.
51. Tokars et al., 2018. “Seasonal Incidence of Symptomatic Influenza in the United States.” Clinical Infectious Diseases: An Official Publication of the Infectious Diseases Society of America 66 (10): 1511-18.
52. Unaids, and UNAIDS. 2022. “In Danger: UNAIDS Global AIDS Update 2022.” https://doi.org/10.18356/9789210019798.
53. Vanderbeke et al., 2021. “Monocyte-Driven Atypical Cytokine Storm and Aberrant Neutrophil Activation as Key Mediators of COVID-19 Disease Severity.” Nature Communications 12 (1): 4117.
54. Wang, Chen, Lydia Kay Lutes, Coline Barnoud, and Christoph Scheiermann. 2022. “The Circadian Immune System.” Science Immunology 7 (72): eabm2465.
55. Wolf et al., Thesis. 2018. “SCANPY: Large-Scale Single-Cell Gene Expression Data Analysis.” Genome Biology 19 (1): 15.
56. Wu et al., 2011. “HMBOXI Negatively Regulates NK Cell Functions by Suppressing the NKG2D/DAP10 Signaling Pathway.” Cellular & Molecular Immunology 8 (5): 433-40.
57. Yukawa et al., 2020. “AP-1 Activity Induced by Co-Stimulation Is Required for Chromatin Opening during T Cell Activation.” The Journal of Experimental Medicine 217 (1). https://doi.org/10.1084/jem.20182009.
58. Zalinger et al., 2017. “Role of the Inflammasome-Related Cytokines Il-1 and 11-18 during Infection with Murine Coronavirus.” Journal of Neurovirology 23 (6): 845-54.
59. Zhang et al., 2021. “A Single-Cell Atlas of Chromatin Accessibility in the Human Genome.” Cell 184 (24): 5985-6001.e19.
60. Zhou et al., 2019. “Metascape Provides a Biologist-Oriented Resource for the Analysis of Systems-Level Datasets.” Nature Communications 10 (1): 1523.

In view of the many possible embodiments to which the principles of the disclosure may be applied, it should be recognized that the illustrated embodiments are only examples of the invention and should not be taken as limiting the scope of the invention. Rather, the scope of the invention is defined by the following claims. We therefore claim as our invention all that comes within the scope and spirit of these claims.

1. A method of determining whether a subject has been exposed to or infected with Bacillus anthracis, SARS-CoV-2, human immunodeficiency virus type 1 (HIV-1), influenza A, methicillin-resistant Staphylococcus aureus (MSSA), or methicillin-resistant Staphylococcus aureus (MRSA), or has been exposed to an organophosphate (OP), comprising:
determining methylation status of at least 20, at least 25, at least 30, at least 40, at least 50, at least 100, at least 500, at least 1000, at least 5000, at least 5400, or all differentially methylated regions (DMRs) in genomic DNA of isolated immune cells obtained from the subject, wherein the immune cells and DMRs are provided in Table 1;
optionally determining an amount of accessible chromatin in the isolated immune cells; and
determining whether the subject has been exposed to or infected with Bacillus anthracis, SARS-CoV-2, HIV-1, influenza A, MSSA, or MRSA, or has been exposed to an organophosphate (OP), based on the measured methylation status of the DMRs and the amount of accessible chromatin.

2. The method of claim 1, further comprising treating genomic DNA of the isolated immune cells with bisulfite to convert unmethylated cytosines of CpG dinucleotides to uracil, treating genomic DNA of the isolated immune cells with a transposase to generate fragments of the genomic DNA, or both.

3. The method of claim 1, wherein determining whether a subject has been exposed to or infected with Bacillus anthracis, SARS-CoV-2, HIV-1, influenza A, MSSA, or MRSA, or has been exposed to an OP, comprises applying a statistical prediction algorithm to the determined methylation status, the amount of accessible chromatin, or both.

4. The method of claim 3, wherein applying the statistical prediction algorithm comprises (a) obtaining a linear combination of the methylation marker status of the at least 20, at least 25, at least 30, at least 40, at least 50, at least 100, at least 500, at least 1000, at least 5000, at least 5400, or all DMRs, and (b) applying a transformation to the linear combination to determining whether a subject has been exposed to or infected with Bacillus anthracis, SARS-CoV-2, HIV-1, influenza A, MSSA, or MRSA, or has been exposed to an OP.

5. The method of claim 1, wherein the isolated immune cells comprise memory B cells, naive B cells, active natural killer cells, naive natural killer cells, memory cytotoxic T cells, naive cytotoxic T cells, memory T cells, naïve T cell, and monocytes.

6. The method of claim 1, wherein the method determines whether the subject has been exposed to or is infected with Bacillus anthracis, and determining methylation status comprises:
determining methylation status of at least 20, at least 25, at least 30, at least 40, at least 50, or at least 100 of the DMRs in B memory cells in Table 1 listed as BA B-Mem;
determining methylation status of at least 20, at least 25, at least 30, at least 40, at least 50, or at least 100 of the DMRs in naïve B cells in Table 1 listed as BA B-Naive;
determining methylation status of at least 20, at least 25, at least 30, at least 40, at least 50, or at least 100 of the DMRs in monocytes in Table 1 listed as BA monocyte;
determining methylation status of at least at least 20, at least 25, at least 30, at least 40, at least 50, or at least 100 of the DMRs in active natural killer cells in Table1 listed as BA NK-cell1;
determining methylation status of at least at least 20, at least 25, at least 30, at least 40, at least 50, or at least 100 of the DMRs in naive natural killer cells in Table 1 listed as BA NK-cell2;
determining methylation status of at least at least 20, at least 25, at least 30, at least 40, at least 50, or at least 100 of the DMRs in memory cytotoxic T cells in Table 1 listed as BA Tc-mem;
determining methylation status of at least at least 20, at least 25, at least 30, at least 40, at least 50, or at least 100 of the DMRs in naive cytotoxic T cells in Table 1 listed as BA Tc-naive;
determining methylation status of at least at least 20, at least 25, at least 30, at least 40, at least 50, or at least 100 of the DMRs in memory T cells in Table 1 listed as BA Th-mem; and/or
determining methylation status of at least at least 20, at least 25, at least 30, at least 40, at least 50, or at least 100 of the DMRs in naïve T cells in Table 1 listed as BA Th-naive,
wherein detection of the DMRs in Table 1 (for BA exposure) indicates that the subject has been exposed to or is infected with Bacillus anthracis.

7. The method of claim 5, further comprising administering to the subject a therapeutically effective amount of one or more of ciprofloxacin, doxycycline, quinolone, and penicillin, when it is indicated that the subject has been exposed to or is infected with Bacillus anthracis.

8. The method of claim 1, wherein the method determines whether the subject has been exposed to or is infected with SARS-CoV-2, and determining methylation status comprises:
determining methylation status of at least 20, at least 25, at least 30, at least 40, at least 50, or at least 100 of the DMRs in B memory cells in Table 1 listed as COVID B-Mem;
determining methylation status of at least 20, at least 25, at least 30, at least 40, at least 50, or at least 100 of the DMRs in naïve B cells in Table 1 listed as COVID B-Naive;
determining methylation status of at least 20, at least 25, at least 30, at least 40, at least 50, or at least 100 of the DMRs in monocytes in Table 1 listed as COVID monocyte;
determining methylation status of at least at least 20, at least 25, at least 30, at least 40, at least 50, or at least 100 of the DMRs in active natural killer cells in Table1 listed as COVID NK-cell1;
determining methylation status of at least at least 20, at least 25, at least 30, at least 40, at least 50, or at least 100 of the DMRs in naive natural killer cells in Table 1 listed as COVID NK-cell2;
determining methylation status of at least at least 20, at least 25, at least 30, at least 40, at least 50, or at least 100 of the DMRs in memory cytotoxic T cells in Table 1 listed as COVID Tc-mem;
determining methylation status of at least at least 20, at least 25, at least 30, at least 40, at least 50, or at least 100 of the DMRs in naive cytotoxic T cells in Table 1 listed as COVID Tc-naive;
determining methylation status of at least at least 20, at least 25, at least 30, at least 40, at least 50, or at least 100 of the DMRs in memory T cells in Table 1 listed as COVID Th-mem; and/or
determining methylation status of at least at least 20, at least 25, at least 30, at least 40, at least 50, or at least 100 of the DMRs in naïve T cells in Table 1 listed as COVID Th-naive,
wherein detection of the DMRs in Table 1 (for COVID exposure) indicates that the subject has been exposed to or is infected with SARS-CoV-2.

9. The method of claim 8, further comprising administering to the subject a therapeutically effective amount of one or more of Paxlovid, molnupiravir, remdesivir, baricitinib and tocilizumab, when it is indicated that the subject has been exposed to or is infected with SARS-CoV-2.

10. The method of claim 1, wherein the method determines whether the subject has been exposed to or is infected with influenza A, and determining methylation status comprises:
determining methylation status of at least 20, at least 25, at least 30, at least 40, at least 50, or at least 100 of the DMRs in B memory cells in Table 1 listed as Flu B-Mem;
determining methylation status of at least 20, at least 25, at least 30, at least 40, at least 50, or at least 100 of the DMRs in naïve B cells in Table 1 listed as Flu B-Naive;
determining methylation status of at least 20, at least 25, at least 30, at least 40, at least 50, or at least 100 of the DMRs in monocytes in Table 1 listed as Flu monocyte;
determining methylation status of at least at least 20, at least 25, at least 30, at least 40, at least 50, or at least 100 of the DMRs in active natural killer cells in Table1 listed as Flu NK-cell1;
determining methylation status of at least at least 20, at least 25, at least 30, at least 40, at least 50, or at least 100 of the DMRs in naive natural killer cells in Table 1 listed as Flu NK-cell2;
determining methylation status of at least at least 20, at least 25, at least 30, at least 40, at least 50, or at least 100 of the DMRs in memory cytotoxic T cells in Table 1 listed as Flu Tc-mem;
determining methylation status of at least at least 20, at least 25, at least 30, at least 40, at least 50, or at least 100 of the DMRs in naive cytotoxic T cells in Table 1 listed as Flu Tc-naive;
determining methylation status of at least at least 20, at least 25, at least 30, at least 40, at least 50, or at least 100 of the DMRs in memory T cells in Table 1 listed as Flu Th-mem; and/or
determining methylation status of at least at least 20, at least 25, at least 30, at least 40, at least 50, or at least 100 of the DMRs in naïve T cells in Table 1 listed as Flu Th-naive,
wherein detection of the DMRs in Table 1 (for Flu exposure) indicates that the subject has been exposed to or is infected with influenza A.

11. The method of claim 10, further comprising administering to the subject a therapeutically effective amount of one or more of oseltamivir, zanamivir, and peramivir, when it is indicated that the subject has been exposed to or is infected with influenza A.

12. The method of claim 1, wherein the method determines whether the subject has been exposed to or is infected with HIV-1, and determining methylation status comprises:
determining methylation status of at least 20, at least 25, at least 30, at least 40, at least 50, or at least 100 of the DMRs in B memory cells in Table 1 listed as HIV B-Mem;
determining methylation status of at least 20, at least 25, at least 30, at least 40, at least 50, or at least 100 of the DMRs in naïve B cells in Table 1 listed as HIV B-Naive;
determining methylation status of at least 20, at least 25, at least 30, at least 40, at least 50, or at least 100 of the DMRs in monocytes in Table 1 listed as HIV monocyte;
determining methylation status of at least at least 20, at least 25, at least 30, at least 40, at least 50, or at least 100 of the DMRs in active natural killer cells in Table1 listed as HIV NK-cell1;
determining methylation status of at least at least 20, at least 25, at least 30, at least 40, at least 50, or at least 100 of the DMRs in naive natural killer cells in Table 1 listed as HIV NK-cell2;
determining methylation status of at least at least 20, at least 25, at least 30, at least 40, at least 50, or at least 100 of the DMRs in memory cytotoxic T cells in Table 1 listed as HIV Tc-mem;
determining methylation status of at least at least 20, at least 25, at least 30, at least 40, at least 50, or at least 100 of the DMRs in naive cytotoxic T cells in Table 1 listed as HIV Tc-naive;
determining methylation status of at least at least 20, at least 25, at least 30, at least 40, at least 50, or at least 100 of the DMRs in memory T cells in Table 1 listed as HIV Th-mem; and/or
determining methylation status of at least at least 20, at least 25, at least 30, at least 40, at least 50, or at least 100 of the DMRs in naïve T cells in Table 1 listed as HIV Th-naive,
wherein detection of the DMRs in Table 1 (for HIV exposure) indicates that the subject has been exposed to or is infected with HIV-1.

13. The method of claim 12, further comprising administering to the subject a therapeutically effective amount of a highly active antiretroviral therapy (HAART), when it is indicated that the subject has been exposed to or is infected with HIV-1.

14. The method of claim 1, wherein the method determines whether the subject has been exposed to or is infected with MSSA or MRSA, and determining methylation status comprises:
determining methylation status of at least 20, at least 25, at least 30, at least 40, at least 50, or at least 100 of the DMRs in B memory cells in Table 1 listed as MRSA B-Mem;
determining methylation status of at least 20, at least 25, at least 30, at least 40, at least 50, or at least 100 of the DMRs in naive B cells in Table 1 listed as MRSA B-Naive;
determining methylation status of at least 20, at least 25, at least 30, at least 40, at least 50, or at least 100 of the DMRs in monocytes in Table 1 listed as MRSA monocyte;
determining methylation status of at least at least 20, at least 25, at least 30, at least 40, at least 50, or at least 100 of the DMRs in active natural killer cells in Table1 listed as MRSA NK-cell1;
determining methylation status of at least at least 20, at least 25, at least 30, at least 40, at least 50, or at least 100 of the DMRs in naive natural killer cells in Table 1 listed as MRSA NK-cell2;
determining methylation status of at least at least 20, at least 25, at least 30, at least 40, at least 50, or at least 100 of the DMRs in memory cytotoxic T cells in Table 1 listed as MRSA Tc-mem;
determining methylation status of at least at least 20, at least 25, at least 30, at least 40, at least 50, or at least 100 of the DMRs in naive cytotoxic T cells in Table 1 listed as MRSA Tc-naive;
determining methylation status of at least at least 20, at least 25, at least 30, at least 40, at least 50, or at least 100 of the DMRs in memory T cells in Table 1 listed as MRSA Th-mem; and/or
determining methylation status of at least at least 20, at least 25, at least 30, at least 40, at least 50, or at least 100 of the DMRs in naive T cells in Table 1 listed as MRSA Th-naive,
wherein detection of the DMRs in Table 1 (for MRSA exposure) indicates that the subject has been exposed to or is infected with MSSA or MRSA.

15. The method of claim 13, further comprising administering to the subject a therapeutically effective amount of one or more of vancomycin, ceftaroline, teicoplanin, clindamycin, and linezolid, when it is indicated that the subject has been exposed to or is infected with MRSA, or further comprising administering to the subject a therapeutically effective amount of one or more of a beta-lactam antibiotic (such as penicillin and its derivatives (such as methicillin and oxacillin) and cephems such as the cephalosporins), flucloxacillin, and a triple antibiotic ointment, when it is indicated that the subject has been exposed to or is infected with MSSA.

16. The method of claim 1, wherein the method determines whether the subject has been exposed to one or more OPs, and determining methylation status comprises:
determining methylation status of at least 20, at least 25, at least 30, at least 40, at least 50, or at least 100 of the DMRs in B memory cells in Table 1 listed as OP B-Mem;
determining methylation status of at least 20, at least 25, at least 30, at least 40, at least 50, or at least 100 of the DMRs in naive B cells in Table 1 listed as OP B-Naive;
determining methylation status of at least 20, at least 25, at least 30, at least 40, at least 50, or at least 100 of the DMRs in monocytes in Table 1 listed as OP monocyte;
determining methylation status of at least at least 20, at least 25, at least 30, at least 40, at least 50, or at least 100 of the DMRs in active natural killer cells in Table1 listed as OP NK-cell1;
determining methylation status of at least at least 20, at least 25, at least 30, at least 40, at least 50, or at least 100 of the DMRs in naive natural killer cells in Table 1 listed as OP NK-cell2;
determining methylation status of at least at least 20, at least 25, at least 30, at least 40, at least 50, or at least 100 of the DMRs in memory cytotoxic T cells in Table 1 listed as OP Tc-mem;
determining methylation status of at least at least 20, at least 25, at least 30, at least 40, at least 50, or at least 100 of the DMRs in naive cytotoxic T cells in Table 1 listed as OP Tc-naive;
determining methylation status of at least at least 20, at least 25, at least 30, at least 40, at least 50, or at least 100 of the DMRs in memory T cells in Table 1 listed as OP Th-mem; and/or
determining methylation status of at least at least 20, at least 25, at least 30, at least 40, at least 50, or at least 100 of the DMRs in naive T cells in Table 1 listed as OP Th-naive,
wherein detection of the DMRs in Table 1 (for OP exposure) indicates that the subject has been exposed to one or more OPs.

17. The method of claim 16, further comprising administering to the subject a therapeutically effective amount of one or more of carbamate, anti-cholinergic drugs (e.g., atropine, oximes (such as pralidoxime, and diazepam)); cholinesterases (ChEs), such as human serum BChE (HuBChE); and class II anti-arrhythimic agents, when it is indicated that the subject has been exposed to one or more organophosphates (OPs).

18. The method of claim 1, wherein isolated immune cells are obtained from a blood sample.

19. The method of claim 1, wherein the subject is a human.

20. The method of claim 1, wherein determining the methylation status or determining an amount of accessible chromatin comprises hybridizing the genomic DNA to polynucleotides attached to a solid support.

21. The method of claim 20, wherein the polynucleotides attached to a solid support comprise at least 20, at least 25, at least 30, at least 40, at least 50, at least 100, at least 500, at least 1000, at least 5000, or at least 5400 of the differentially methylated regions in Table 1.

22. The method of claim 1, further comprising obtaining the isolated immune cells from the subject.

23. The method of claim 1, further comprising extracting genomic DNA from the isolated immune cells.

24. The method of claim 1, further comprising selecting the subject, wherein the subject has been or is suspected of having been exposed to or infected with Bacillus anthracis, SARS-CoV-2, influenza A, HIV-1, or MSSA, MRSA, or exposed to an OP.

25. The method of claim 1, further comprising reporting whether the subject has been exposed to or infected with Bacillus anthracis, SARS-CoV-2, influenza A, HIV-1, or MSSA, MRSA, or exposed to an OP.

26. The method of claim 25, wherein the reporting comprises preparing a written or electronic report.

27. A kit, comprising:
nucleic acid probes and/or primers for detecting at least 20, at least 25, at least 30, at least 40, at least 50, at least 100, at least 500, at least 1000, at least 5000, or at least 5400 of the differentially methylated regions in Table 1,
optionally nucleic acid probes and/or primers for determining an amount of accessible chromatin in human immune cells, and
optionally sodium bisulfite, transposase, or both.
